(19) United States
(12) Patent Application Publication
(10) Pub. No.: US 2025/0127046 A1
(43) Pub. Date: Apr. 17, 2025

(54) COMPOSITION, ORGANIC ELECTROLUMINESCENT ELEMENT AND METHOD FOR PRODUCING SAME, DISPLAY DEVICE, AND LIGHTING DEVICE

(71) Applicant: Mitsubishi Chemical Corporation, Tokyo (JP)

(72) Inventors: Hideki GOROHMARU, Tokyo (JP); Hideji KOMATSU, Tokyo (JP); Yanjun LI, Tokyo (JP); Daisuke HIRO, Tokyo (JP); Koichiro IIDA, Tokyo (JP); Yoshimasa BANDO, Tokyo (JP); Yuki OHSHIMA, Tokyo (JP); Kazuki OKABE, Tokyo (JP); Tsukasa HASEGAWA, Tokyo (JP); Mami YAMASHITA, Tokyo (JP)

(73) Assignee: Mitsubishi Chemical Corporation, Tokyo (JP)

(21) Appl. No.: 18/652,887

(22) Filed: May 2, 2024

Related U.S. Application Data
(63) Continuation of application No. PCT/JP2022/040871, filed on Nov. 1, 2022.

(30) Foreign Application Priority Data
Nov. 12, 2021 (JP) 2021-184743
Nov. 12, 2021 (JP) 2021-184745

(51) Int. Cl. H10K 85/60; H10K 50/10; H10K 71/12; H10K 85/10

(52) U.S. Cl. CPC H10K 85/6572; H10K 85/111; H10K 85/115; H10K 85/151; H10K 85/626; H10K 85/658; H10K 50/10; H10K 71/12

(57) ABSTRACT

A composition comprising: a carbazole compound having a crosslinking group and represented by formula (71) or (72) below; and an electron accepting compound having a crosslinking group and represented by formula (81) below.

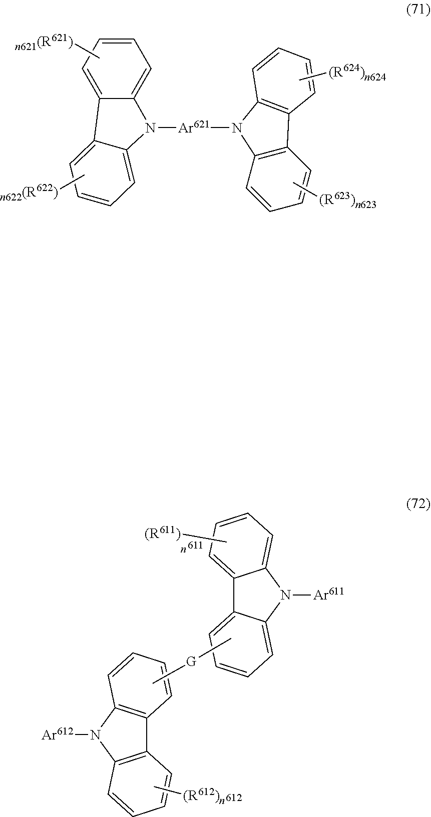

### TECHNICAL FIELD

The present invention relates to a composition, an organic electroluminescent element, a method for producing the same, a display device, and a lighting device.

### BACKGROUND ART

In recent years, thin-film organic electroluminescent elements using organic thin films have been developed instead of electroluminescent elements using inorganic materials. Generally, an organic electroluminescent element (OLED) includes, between an anode and a cathode, a charge injection layer, a charge transport layer, an organic light-emitting layer, an electron transport layer, etc. Materials suitable for these layers are being developed, and emission colors including red, green, and blue colors are being developed.

Examples of the method for forming an organic layer in an organic electroluminescent element include a vacuum vapor deposition method and a wet deposition method (coating method). One advantage of the vacuum vapor deposition method is that, since multilayer deposition can be easily performed, injection of charges from the anode and/or the cathode can be improved and excitons can be easily confined in the light-emitting layer. Meanwhile, the wet deposition method does not require a vacuum process and has the advantages that the method can be easily applied to large-area elements and that, by using a coating solution containing a mixture of a plurality of materials having different functions, a layer containing the plurality of materials having different functions can be easily formed. Therefore, in recent years, research and development of organic electroluminescent elements using the coating method has been actively performed.

PTL 1 describes an organic electroluminescent element including a polymer having a crosslinking group as a charge injection material and an electron accepting compound having a crosslinking group. PTL 2 describes an organic electroluminescent element including a composition containing a fluorene-based aryldiamine compound having a crosslinking group and an electron accepting compound. PTL 3 describes an organic electroluminescent element including a composition containing a carbazole-based arylamine compound having a crosslinking group and an electron accepting compound. PTL 4 discloses an organic electroluminescent element containing a compound having in its molecule at least one polymerizable substituent and at least two carbazole groups.

### CITATION LIST

### Patent Literature

PTL 1: WO2019/059331
PTL 2: WO2019/231257
PTL 3: US2021/0151685A1
PTL 4: JP2008-244471A

### SUMMARY OF INVENTION

### Technical Problem

Generally, when an arylamine organic electron donor and an organic electron acceptor are mixed at an appropriate ratio to allow N atoms included in the arylamine to partially form an ion complex with the organic electron acceptor, the formation of the ion complex allows a hole injection barrier from the anode to be lowered. Therefore, attention is being given to a material forming a stable ion complex.

With the materials and techniques disclosed in PTL 1 to PTL 3, an ion complex is formed from an organic electron acceptor and an organic electron donor such as an arylamine polymer, a low-molecular weight arylamine compound, or a carbazole-based arylamine compound. However, the reduction in the driving voltage of the organic electroluminescent element is insufficient.

PTL 4 discloses a biscarbazole compound including an oxetane crosslinking group, and an organic electron acceptor including no crosslinking group is used as a photopolymerization initiator. In this case, prevention of diffusion of the organic electron acceptor to a light-emitting layer is insufficient, and the luminous efficiency and the driving lifetime cannot be improved.

It is an object of the invention to provide an organic electroluminescent element having a low driving voltage, high luminous efficiency, and a long driving lifetime.

### Solution to Problem

The present inventors have found that the above object can be achieved by using a hole injection layer and/or a hole transport layer containing a crosslinking reaction product of a carbazole compound having a crosslinking group and an electron accepting compound having a crosslinking group, and thus the present invention has been completed.

The present invention has the following gist.

[1] A composition comprising: a carbazole compound having a crosslinking group and represented by formula (71) or (72) below; and an electron accepting compound having a crosslinking group and represented by formula (81) below:

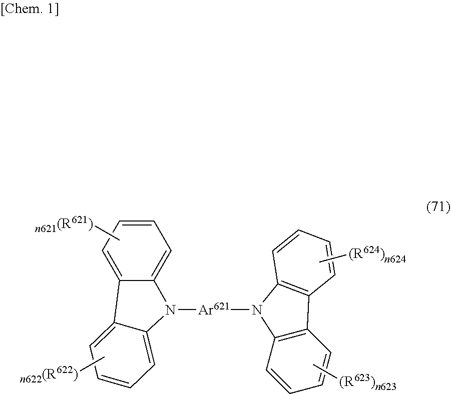

(wherein, in formula (71),
Ar⁶²¹ represents a divalent aromatic hydrocarbon group having 6 to 50 carbon atoms and optionally having a substituent;
R⁶²¹, R⁶²², R⁶²³, and R⁶²⁴ are each independently a deuterium atom, a halogen atom, a monovalent aromatic hydrocarbon group having 6 to 50 carbon atoms and optionally having and/or a crosslinking group, or a crosslinking group;
n621, n622, n623, and n624 are each independently an integer of 0 to 4,
provided that the sum of n621, n622, n633, and n624 is 1 or more; and
the compound represented by formula (71) has at least two crosslinking groups),

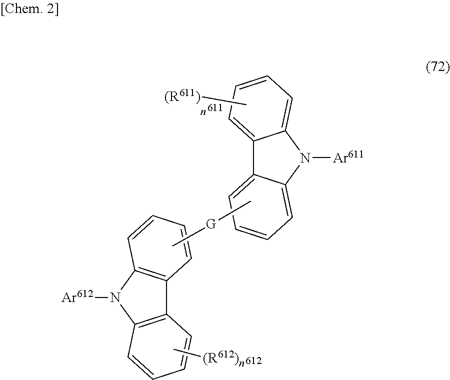

(wherein, in formula (72),
Ar⁶¹¹ and Ar⁶¹² each independently represent a divalent aromatic hydrocarbon group having 6 to 50 carbon atoms and optionally having a substituent and/or a crosslinking group;
R⁶¹¹ and R⁶¹² are each independently a deuterium atom, a halogen atom, a monovalent aromatic hydrocarbon group having 6 to 50 carbon atoms and optionally having a substituent and/or a crosslinking group, or a crosslinking group;
G represents a single bond or a divalent aromatic hydrocarbon group having 6 to 50 carbon atoms and optionally having a substituent and/or a crosslinking group;
n611 and n612 are each independently an integer of 0 to 4; and
the compound represented by formula (72) has at least two crosslinking groups),

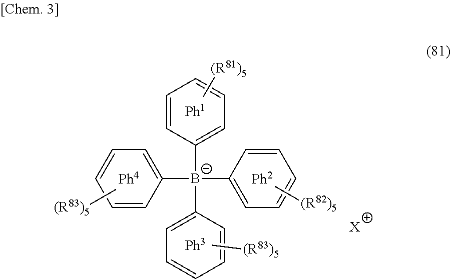

(wherein, in formula (81), five R⁸¹'s, five R⁸²'s, five R⁸³'s, and five R⁸⁴'s are each independent; R⁸¹'s to R⁸⁴'s each independently represent a hydrogen atom, a deuterium atom, a halogen atom, an aromatic hydrocarbon group having 6 to 50 carbon atoms and optionally having a substituent and/or a crosslinking group, an aromatic heterocyclic group having 3 to 50 carbon atoms and optionally having a substituent and/or a crosslinking group, a fluorine-substituted alkyl group having 1 to 12 carbon atoms, or a crosslinking group;
Ph¹, Ph², Ph³, are Ph⁴ are symbols representing four benzene rings;
the compound represented by formula (81) has a crosslinking group; and
X⁺ represents a counter cation).
   [2] The composition according to [1], wherein Ar⁶²¹ in formula (71) above is a divalent group formed by bonding a plurality of structures selected from 1 to 4 benzene rings each optionally having a substituent and 1 or 2 fluorene rings each optionally having a substituent in any order in a linear or branched manner.
   [3] The composition according to [1] or [2], wherein Ar⁶²¹ in formula (71) above has at least one partial structure selected from the following formulas (71-1) to (71-11) and (71-21) to (71-24):

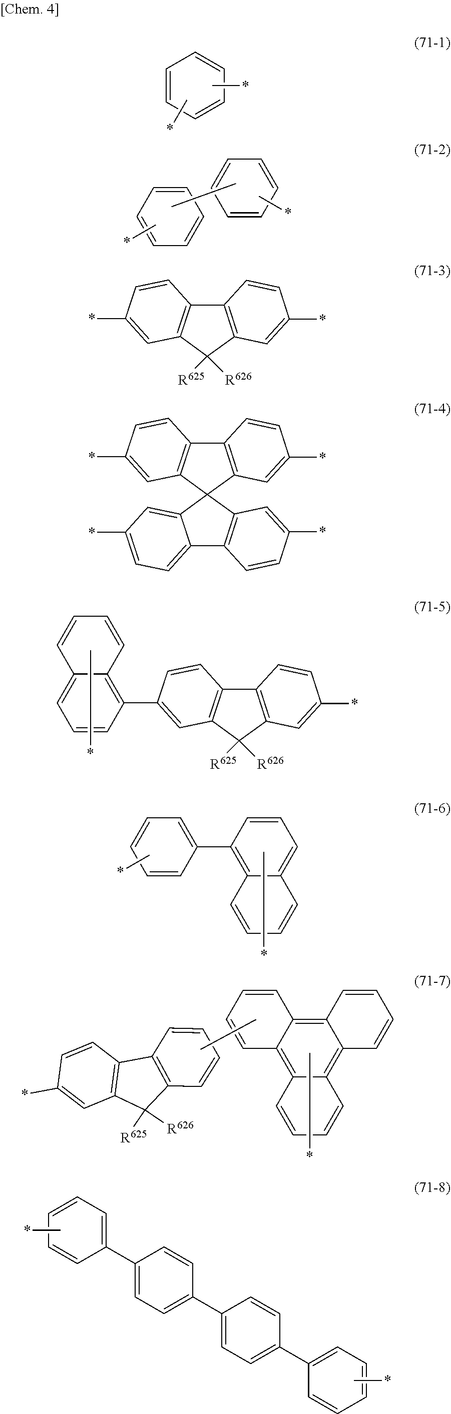

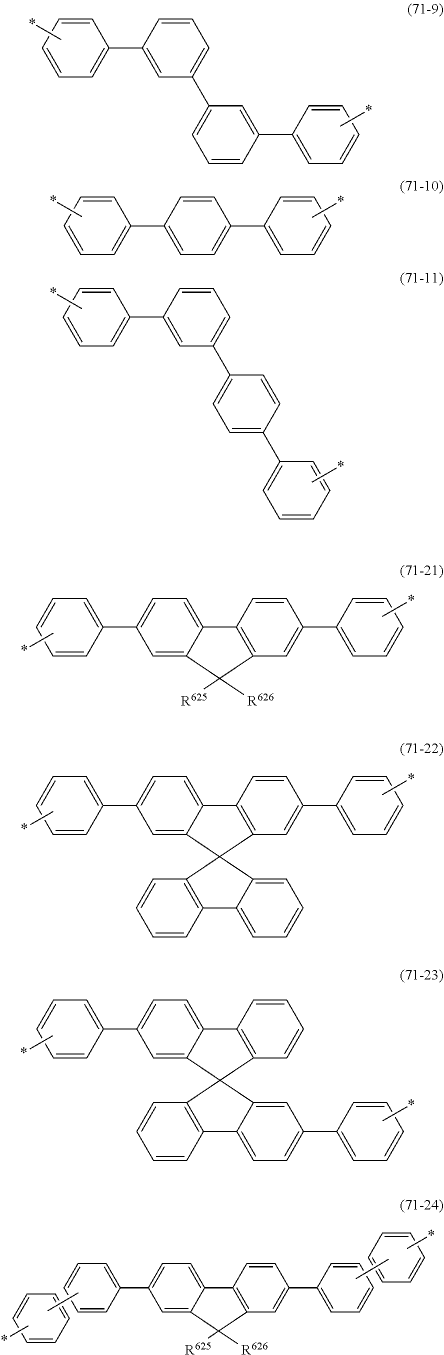

(wherein, in each of formulas (71-1) to (71-11) and (71-21) to (71-24) above,
each * represents a bond to an adjacent structure or a hydrogen atom; when two *'s are present, at least one of the two *'s represents a position of bonding to an adjacent structure; when four *'s are present, at least one of any two of the four *'s represents a position of bonding to an adjacent structure;
R⁶²⁵ and R⁶²⁶ each independently represent an alkyl group having 6 to 12 carbon atoms, an alkenyl group, an alkynyl group, an alkoxy group, an aryloxy group, an alkoxycarbonyl group, an acyl group, a halogen atom, a haloalkyl group, an alkylthio group, an arylthio group, a silyl group, a siloxy group, a cyano group, an aralkyl group, or a monovalent aromatic hydrocarbon group having 6 to 30 carbon atoms; and R⁶²⁵ and R⁶²⁶ may be bonded together to form a ring).
   [4] The composition according to any one of [1] to [3], wherein R⁶²¹, R⁶²², R⁶²³, and R⁶²⁴ in formula (71) above are each independently an aromatic hydrocarbon group having 6 to 50 carbon atoms and optionally having a crosslinking group or a crosslinking group.
   [5] The composition according to any one of [1] to [4], wherein, in formula (71) above, n621 and n623 are each 1; n622 and n624 are each 0; and R⁶²¹ and R⁶²³ are each independently an aromatic hydrocarbon group having 6 to 50 carbon atoms and substituted with a crosslinking group or a crosslinking group.
   [6] The composition according to any one of [1] to [5], wherein Ar⁶¹¹ and Ar⁶¹² in formula (72) above are each independently a phenyl group having a crosslinking group or a monovalent group that includes a plurality of benzene rings bonded together in a linear or branched manner and that has a crosslinking group.
   [7] The composition according to any one of [1] to [6], wherein at least one of Ar⁶¹¹ and Ar⁶¹² in formula (72) above has at least one partial structure selected from the following formulas (72-1) to (72-6):

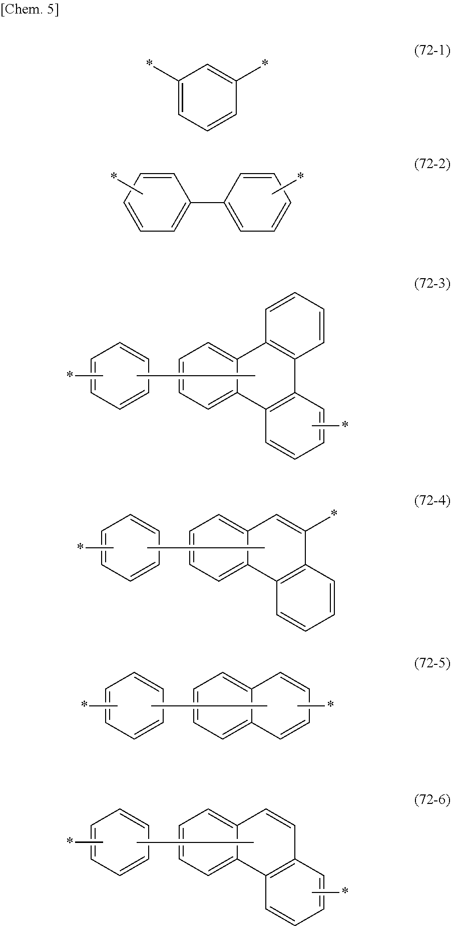

(wherein, in each of formulas (72-1) to (72-6) above, each * represents a bond to an adjacent structure or a hydrogen atom; and at least one of two *'s represents a position of bonding to an adjacent structure).
[8] The composition according to any one of [1] to [7], wherein, in formula (72) above, n611 and n612 are each 0.
[9] The composition according to any one of [1] to [8], wherein, in formula (72) above, G is a single bond.
[10] The composition according to any one of [1] to [9], wherein, in formula (81) above, at least one of -Ph¹-(R⁸¹)₅, -Ph²-(R⁸²)₅, -Ph³-(R⁸³)₅, and -Ph⁴-(R³⁴)₅ is a group represented by the following formula (84) and having four fluorine atoms:

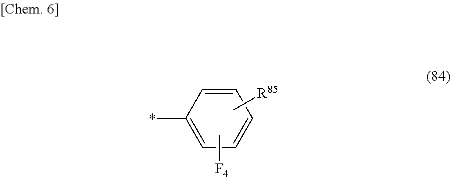

(wherein, in formula (84), * represents a bond to boron B in formula (81);
F₄ represents substitution with four fluorine atoms; and
R⁸⁵ represents an aromatic hydrocarbon group optionally having a substituent and/or a crosslinking group or a crosslinking group).
   [11] The composition according to any one of [1] to [10], wherein the electron accepting compound represented by formula (81) above has at least two crosslinking groups.
   [12] The composition according to any one of [1] to [11], further comprising a polymer having an arylamine structure as a repeating unit, the repeating unit being represented by formula (50) below:

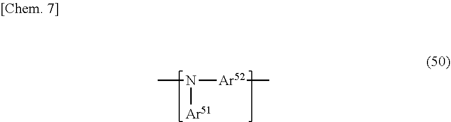

(wherein, in formula (50),
Ar⁵¹ represents an aromatic hydrocarbon group, an aromatic heterocyclic group, or a group in which a plurality of groups selected from aromatic hydrocarbon groups and aromatic heterocyclic groups are linked together;
Ar⁵² represents a divalent aromatic hydrocarbon group, a divalent aromatic heterocyclic group, or a divalent group in which a plurality of groups selected from the group consisting of divalent aromatic hydrocarbon groups and divalent aromatic heterocyclic groups are linked together directly or via a linking group;
Ar⁵¹ and Ar⁵² may form a ring via a single bond or a linking group; and
Ar⁵¹ and Ar⁵² may each optionally have a substituent and/or a crosslinking group).
   [13] The composition according to [12], wherein the polymer has a crosslinking group.
   [14] The composition according to any one of [1] to [13], wherein each crosslinking group included in the compound represented by formula (71) or (72) above, each crosslinking group included in the compound represented by formula (81) above, and each crosslinking group included in the polymer having the repeating unit represented by formula (50) above are each independently selected from formulas (X1) to (X18) in the following group T of crosslinking groups:

### <Group T of Crosslinking Groups>

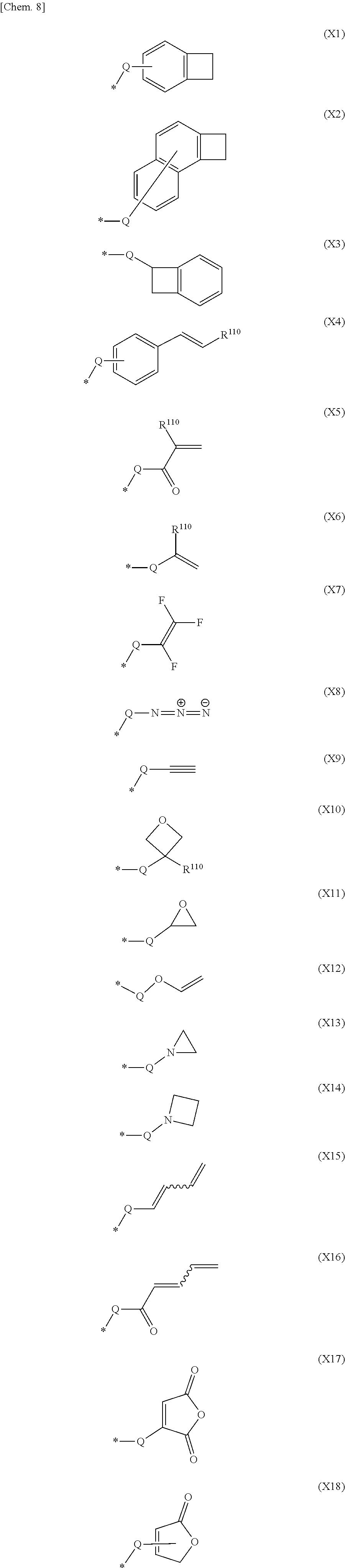

(wherein, in formulas (X1) to (X18), Q represents a direct bond or a linking group;
* represents a bonding position;
R¹¹⁰ in each of formulas (X4), (X5), (X6), and (X10) represents a hydrogen atom or an alkyl group optionally having a substituent;
each of the benzene rings and the naphthalene ring in formula (X1) to (X4) may optionally have a substituent, and any of the substituents may be bonded together to form a ring; and
each cyclobutene ring in formula (X1) to (X3) may optionally have a substituent).
   [15] The composition according to [14], wherein each crosslinking group included in the compound represented by formula (71) or (72) above, each crosslinking group included in the compound represented by formula (81) above, and each crosslinking group included in the polymer having an arylamine structure as a repeating unit are each independently selected from formulas (X1) to (X4) in the group T of crosslinking groups.
   [16] The composition according to any one of [1] to [15], wherein the repeating unit represented by formula (50) is a repeating unit represented by formula (54), (55), (56), or (57) below:

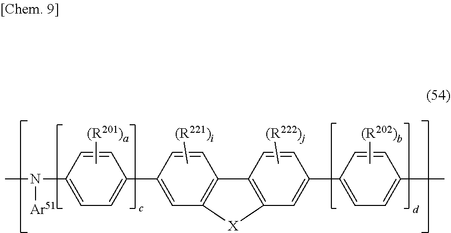

(wherein, in formula (54),
Ar⁵¹ is the same as Ar⁵¹ in formula (50) above;
X is —C(R²⁰⁷)(R²⁰⁸)—, —N(R²⁰⁹)—, or —C(R²¹¹)(R²¹²)—C(R²¹³)(R²¹⁴)—;
R²⁰¹, R²⁰², R²²¹, and R²²² are each independently an alkyl group optionally having a substituent and/or a crosslinking group;
R²⁰⁷ to R²⁰⁹ and R²¹¹ to R²¹⁴ are each independently a hydrogen atom, an alkyl group optionally having a substituent and/or a crosslinking group, an aralkyl group optionally having a substituent and/or a crosslinking group, or an aromatic hydrocarbon group optionally having a substituent and/or a crosslinking group;
a and b are each independently an integer of 0 to 4;
c is an integer of 0 to 3;
d is an integer of 0 to 4; and
i and j are each independently an integer of 0 to 3),

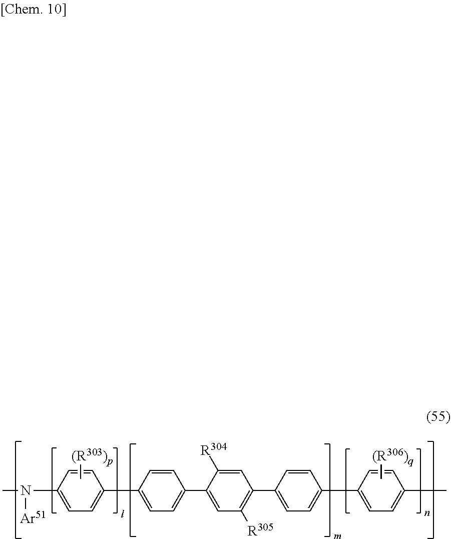

(wherein, in formula (55),
Ar⁵¹ is the same as Ar⁵¹ in formula (54) above;
R³⁰³ and R³⁰⁶ each independently represent an alkyl group optionally having a substituent and/or a crosslinking group;
R³⁰⁴ and R³⁰⁵ each independently represent an alkyl group optionally having a substituent and/or a crosslinking group, an alkoxy group optionally having a substituent and/or a crosslinking group, or an aralkyl group optionally having a substituent and/or a crosslinking group;
l is 0 or 1;
m is 1 or 2;
n is 0 or 1;
p is 0 or 1; and
q is 0 or 1),

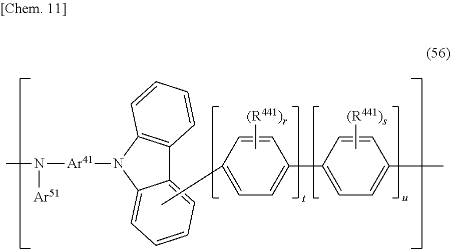

(wherein, in formula (56),
Ar⁵¹ is the same as Ar⁵¹ in formula (54) above;
Ar⁴¹ represents a divalent aromatic hydrocarbon group optionally having a substituent, a divalent aromatic heterocyclic group optionally having a substituent, or a divalent group in which a plurality of groups selected from the group consisting of divalent aromatic hydrocarbon groups and divalent aromatic heterocyclic groups are linked together directly or via a linking group;
R⁴⁴¹ and R⁴⁴² each independently represent an alkyl group optionally having a substituent;
t is 1 or 2;
u is 0 or 1;
r and s are each independently an integer of 0 to 4; and
when a twisted structure is essential, r×t+s×u is 1),

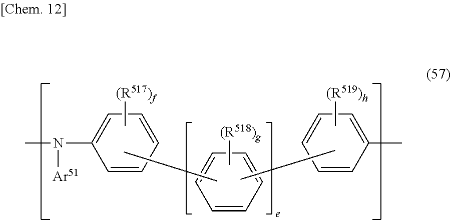

(wherein, in formula (57),
Ar⁵¹ is the same as Ar⁵¹ in formula (50) above;
R⁵¹⁷ to R⁵¹⁹ each independently represent an alkyl group optionally having a substituent and/or a crosslinking group, an alkoxy group optionally having a substituent and/or a crosslinking group, an aralkyl group optionally having a substituent and/or a crosslinking group, an aromatic hydrocarbon group optionally having a substituent and/or a crosslinking group, or an aromatic heterocyclic group optionally having a substituent and/or a crosslinking group;
f, g, and h each independently represent an integer of 0 to 4;
e represents an integer of 0 to 3, provided that, when g is 1 or more, e is 1 or more; and
f+e×g+h is 1 or more).
   [17] The composition according to [16], wherein the polymer having an arylamine structure as a repeating unit further includes a repeating unit represented by formula (60) below as the repeating unit represented by formula (50) above in addition to the repeating unit represented by formula (54), (55), (56), or (57) above:

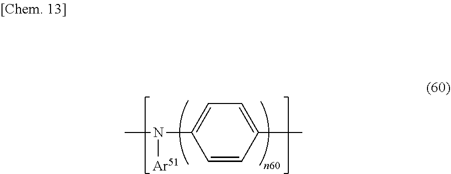

(wherein, in formula (60),
Ar⁵¹ is the same as Ar⁵¹ in formula (50) above; and
n60 represents an integer of 1 to 5).
   [18] The composition according to any one of [1] to [17], wherein each substituent included in the compound represented by formula (71) or (72) above, each substituent included in the compound represented by formula (81) above, each substituent including in the polymer having the repeating unit represented by formula (50) above, and each substituent included in the group T of crosslinking groups are each independently selected from the following substituent group Z:

### <Substituent Group Z>

alkyl groups having 1 to 24 carbon atoms,
alkenyl group having 2 to 24 carbon atoms,
alkynyl groups having 2 to 24 carbon atoms,
alkoxy groups having 1 to 24 carbon atoms,
aryloxy groups and heteroaryloxy groups having 4 to 36 carbon atoms,
alkoxycarbonyl groups having 2 to 24 carbon atoms,
dialkylamino groups having 2 to 24 carbon atoms,
diarylamino groups having 10 to 36 carbon atoms,
arylalkylamino groups having 7 to 36 carbon atoms,
acyl groups having 2 to 24 carbon atoms, halogen atoms,
haloalkyl groups having 1 to 12 carbon atoms,
alkylthio groups having 1 to 24 carbon atoms,
arylthio groups having 4 to 36 carbon atoms,
silyl groups having 2 to 36 carbon atoms,
siloxy groups having 2 to 36 carbon atoms,
a cyano group,
aromatic hydrocarbon groups having 6 to 36 carbon atoms, and
aromatic heterocyclic groups having 4 to 36 carbon atoms,
wherein each of the above substituents may have a linear, branched, or cyclic structure, and wherein, when any of the substituents are adjacent to each other, the substituents adjacent to each other may be bonded together to form a ring.
   [19] The composition according to any one of [1] to [18], further comprising a solvent.
   [20]A composition comprising: a carbazole compound having a crosslinking group and represented by formula (71) or (72) below; and a polymer having an arylamine structure as a repeating unit, wherein the polymer having an arylamine structure as a repeating unit has a structure represented by formula (50) below as a repeating unit and also has a crosslinking group, and wherein the structure represented by formula (50) has a partial structure represented by formula (63) below:

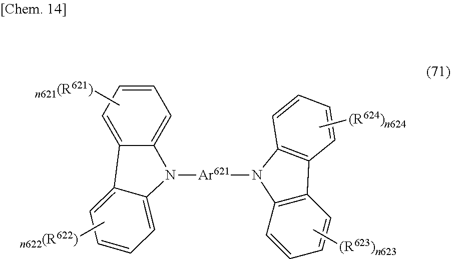

(wherein, in formula (71),
Ar⁶²¹ represents a divalent aromatic hydrocarbon group having 6 to 50 carbon atoms and optionally having a substituent;
R⁶²¹, R⁶²², R⁶²³, and R⁶²⁴ each independently represent a deuterium atom, a halogen atom, a monovalent aromatic hydrocarbon group having 6 to 50 carbon atoms and optionally having and/or a crosslinking group, or a crosslinking group;
n621, n622, n623, and n624 are each independently an integer of 0 to 4,
provided that the sum of n621, n622, n633, and n624 is 1 or more; and
the compound represented by formula (71) has at least two crosslinking groups),

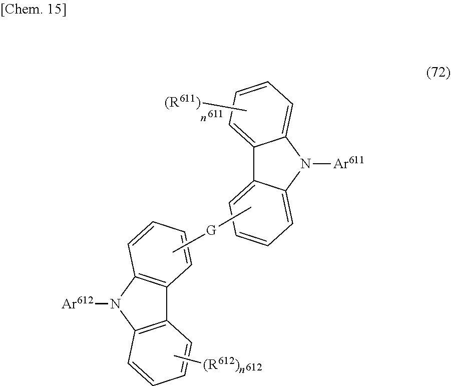

(wherein, in formula (72),
Ar⁶¹¹ and Ar⁶¹² each independently represent a divalent aromatic hydrocarbon group having 6 to 50 carbon atoms and optionally having a substituent and/or a crosslinking group;
R⁶¹¹ and R⁶¹² are each independently a deuterium atom, a halogen atom, a monovalent aromatic hydrocarbon group having 6 to 50 carbon atoms and optionally having a substituent and/or a crosslinking group, or a crosslinking group;
G represents a single bond or a divalent aromatic hydrocarbon group having 6 to 50 carbon atoms and optionally having a substituent and/or a crosslinking group;
n⁶¹¹ and n⁶¹² are each independently an integer of 0 to 4; and
the compound represented by formula (72) has at least two crosslinking groups),

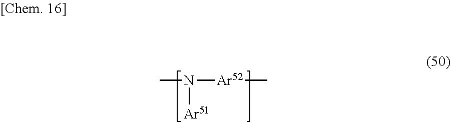

(wherein, in formula (50),
Ar⁵¹ represents an aromatic hydrocarbon group, an aromatic heterocyclic group, or a group in which a plurality of groups selected from aromatic hydrocarbon groups and aromatic heterocyclic groups are linked together;
Ar⁵² represents a divalent aromatic hydrocarbon group, a divalent aromatic heterocyclic group, or a divalent group in which a plurality of groups selected from the group consisting of divalent aromatic hydrocarbon groups and divalent aromatic heterocyclic groups are linked together directly or via a linking group;
Ar⁵¹ and Ar⁵² may form a ring via a single bond or a linking group;
Ar⁵¹ and Ar⁵² may each optionally have a substituent and/or a crosslinking group; and
Ar⁵² has the partial structure represented by formula (63) below,

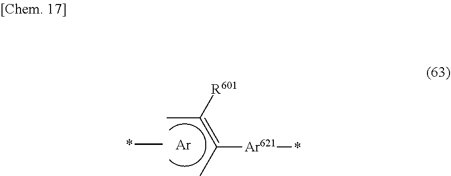

(wherein, in formula (63),
R⁶⁰¹ represents an alkyl group optionally having a substituent or a crosslinking group;
Ar⁶²¹ represents a divalent aromatic hydrocarbon group optionally having a substituent and/or a crosslinking group or a divalent aromatic heterocyclic group optionally having a substituent and/or a crosslinking group;
ring Ar represents an aromatic hydrocarbon structure optionally having a substituent and/or a crosslinking group or a divalent aromatic heterocyclic structure optionally having a substituent and/or a crosslinking group; and
* represents a position bonded to an adjacent atom).
   [21] The composition according to [20], wherein the repeating unit represented by formula (50) above is a repeating unit represented by formula (54), (55), (56), or (57) below:

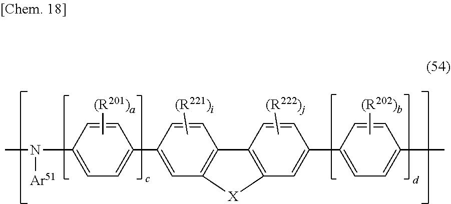

(wherein, in formula (54),
Ar⁵¹ is the same as Ar⁵¹ in formula (50) above;
X is —C(R²⁰⁷)(R²⁰⁸)—, —N(R²⁰⁹)—, or —C(R²¹¹)(R²¹²)—C(R²¹³)(R²¹⁴)—;
R²⁰¹, R²⁰², R²²¹, and R²²² are each independently an alkyl group optionally having a substituent and/or a crosslinking group;
R²⁰⁷ to R²⁰⁹ and R²¹¹ to R²¹⁴ are each independently a hydrogen atom, an alkyl group optionally having a substituent and/or a crosslinking group, an aralkyl group optionally having a substituent and/or a crosslinking group, or an aromatic hydrocarbon group optionally having a substituent and/or a crosslinking group;
a and b are each independently an integer of 0 to 4;
c is an integer of 0 to 3;
d is an integer of 0 to 4;
i and j are each independently an integer of 0 to 3; and
a×c+b×d+i+j is 1 or more),

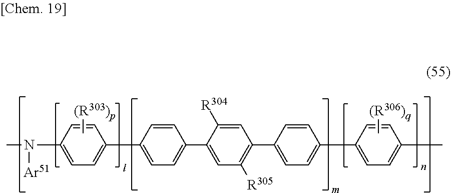

(wherein, in formula (55),
Ar⁵¹ is the same as Ar⁵¹ in formula (54) above;
R³⁰³ and R³⁰⁶ each independently represent an alkyl group optionally having a substituent and/or a crosslinking group;
R³⁰⁴ and R³⁰⁵ each independently represent an alkyl group optionally having a substituent and/or a crosslinking group, an alkoxy group optionally having a substituent and/or a crosslinking group, or an aralkyl group optionally having a substituent and/or a crosslinking group;
l is 0 or 1;
m is 1 or 2;
n is 0 or 1;
p is 0 or 1; and
q is 0 or 1),

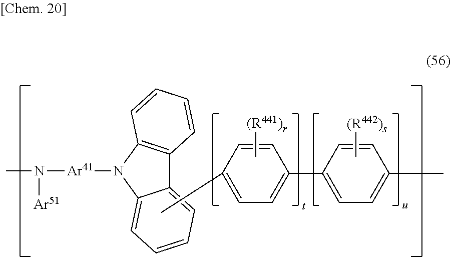

(wherein, in formula (56),
Ar⁵¹ is the same as Ar⁵¹ in formula (54) above;
Ar⁴¹ represents a divalent aromatic hydrocarbon group optionally having a substituent, a divalent aromatic heterocyclic group optionally having a substituent, or a divalent group in which a plurality of groups selected from the group consisting of divalent aromatic hydrocarbon groups and divalent aromatic heterocyclic groups are linked together directly or via a linking group;
R⁴⁴¹ and R⁴⁴² each independently represent an alkyl group optionally having a substituent;
t is 1 or 2;
u is 0 or 1;
r and s are each independently an integer of 0 to 4; and
when a twisted structure is essential, r×t+s×u is 1),

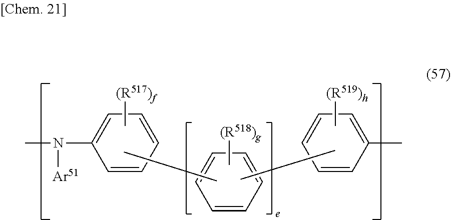

(wherein, in formula (57),
Ar⁵¹ is the same as Ar⁵¹ in formula (50) above;
R⁵¹⁷ to R⁵¹⁹ each independently represent an alkyl group optionally having a substituent and/or a crosslinking group, an alkoxy group optionally having a substituent and/or a crosslinking group, an aralkyl group optionally having a substituent and/or a crosslinking group, an aromatic hydrocarbon group optionally having a substituent and/or a crosslinking group, or an aromatic heterocyclic group optionally having a substituent and/or a crosslinking group;
f, g, and h each independently represent an integer of 0 to 4;
e represents an integer of 0 to 3,
provided that, when g is 1 or more, e is 1 or more; and
f+e×g+h is 1 or more).
   [22] The composition according to [21], wherein the repeating unit represented by formula (54) above is a repeating unit represented by formula (62) below:

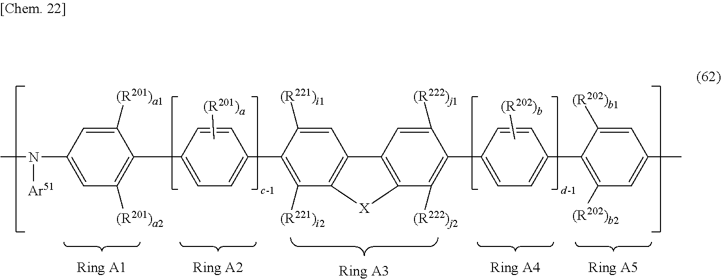

(wherein, in formula (62),
Ar⁵¹, X, R²⁰¹, R²⁰², R²²¹, R²²², a, b, c, d, i and j are the same as Ar⁵¹, X, R²⁰¹, R²⁰², R²²¹, R²²², a, b, i and j in formula (54) above;
c is an integer of 1 to 3;
d is an integer of 1 to 4;
a¹, a², b¹, b², i¹, i², j¹, and j² are each independently 0 or 1; and
any of the following conditions (1) and (2) is satisfied:
(1) at least one of a¹, a², a, b¹, b², and b is 1 or more, provided that, when c is 1, a is not defined and that, when d is 1, b is not defined;
(2) at least one of i¹, i², j¹, and j² is 1, and
wherein Ring A1 is a divalent benzene ring optionally having R²⁰¹ at a specific position;
Ring A2 is a divalent group including c−1 benzene rings linked together and each optionally having R²⁰¹, provided that, when c=2, Ring A2 is a single divalent benzene ring;
Ring A3 is a divalent condensed ring having a biphenyl structure further bonded through X;
Ring A4 is a divalent group including d−1 benzene rings linked together and each optionally having R²⁰¹, provided that, when d=2, Ring A4 is a single divalent benzene ring; and
Ring A5 is a divalent benzene ring optionally having R²⁰² at a specific position).
   [23] The composition according to any one of [20] to [22], wherein the polymer having an arylamine structure as a repeating unit further includes a repeating unit represented by formula (60) below as the repeating unit represented by formula (50) above in addition to the repeating unit represented by formula (54), (55), (56), or (57) above:

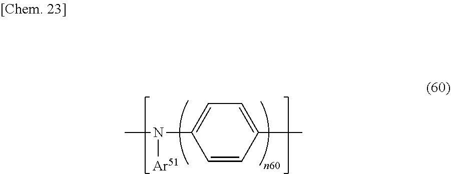

(wherein, in formula (60),
Ar⁵¹ is the same as Ar⁵¹ in formula (50) above; and
n60 represents an integer of 1 to 5).
   [24] The composition according to any one of [20] to [23], wherein Ar⁶²¹ in formula (71) above is a divalent group formed by bonding a plurality of structures selected from 1 to 4 benzene rings each optionally having a substituent and 1 or 2 fluorene rings each optionally having a substituent in any order in a linear or branched manner.
   [25] The composition according to any one of [20] to [24], wherein Ar⁶²¹ in formula (71) above has at least one partial structure selected from the following formulas (71-1) to (71-11) and (71-21) to (71-24):

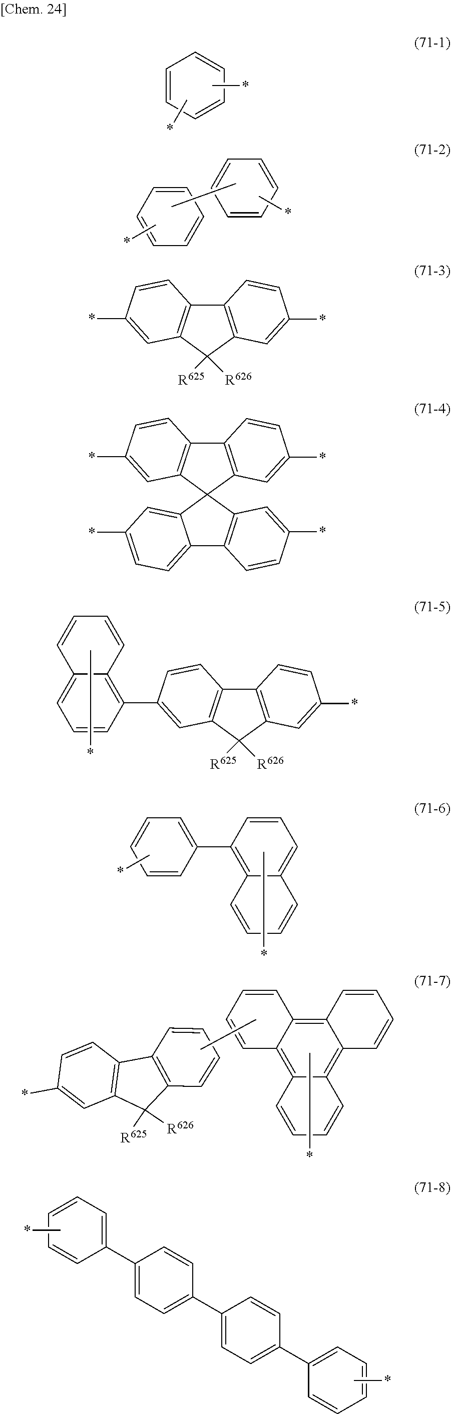

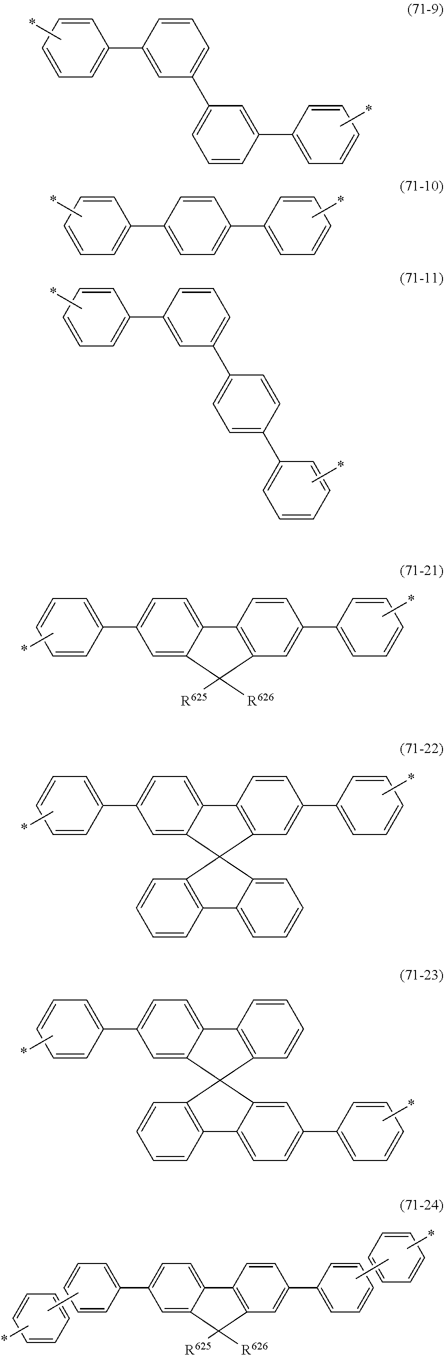

(wherein, in each of formulas (71-1) to (71-11) and (71-21) to (71-24) above,
each * represents a bond to an adjacent structure or a hydrogen atom; when two *'s are present, at least one of the two *'s represents a position of bonding to an adjacent structure; when four *'s are present, at least one of any two of the four *'s represents a position of bonding to an adjacent structure;
R⁶²⁵ and R⁶²⁶ each independently represent an alkyl group having 6 to 12 carbon atoms, an alkenyl group, an alkynyl group, an alkoxy group, an aryloxy group, an alkoxycarbonyl group, an acyl group, a halogen atom, a haloalkyl group, an alkylthio group, an arylthio group, a silyl group, a siloxy group, a cyano group, an aralkyl group, or a monovalent aromatic hydrocarbon group having 6 to 30 carbon atoms; and R⁶²⁵ and R⁶²⁶ may be bonded together to form a ring).
   [26] The composition according to any one of [20] to [25], wherein R⁶²¹, R⁶²², R⁶²³, and R⁶²⁴ in formula (71) above are each independently an aromatic hydrocarbon group having 6 to 50 carbon atoms and optionally having a crosslinking group or a crosslinking group.
   [27] The composition according to any one of [20] to [26], wherein, in formula (71) above, n621 and n623 are each 1; n622 and n624 are each 0; and R⁶²¹ and R⁶²³ are each independently an aromatic hydrocarbon group having 6 to 50 carbon atoms and substituted with a crosslinking group or a crosslinking group.
   [28] The composition according to any one of [20] to [27], wherein Ar⁶¹¹ and Ar⁶¹² in formula (72) above are each independently a phenyl group having a crosslinking group or a monovalent group that includes a plurality of benzene rings bonded together in a linear or branched manner and that has a crosslinking group.
   [29] The composition according to any one of [20] to [28], wherein at least one of Ar⁶¹¹ and Ar⁶¹² in formula (72) above has at least one partial structure selected from the following formulas (72-1) to (72-6):

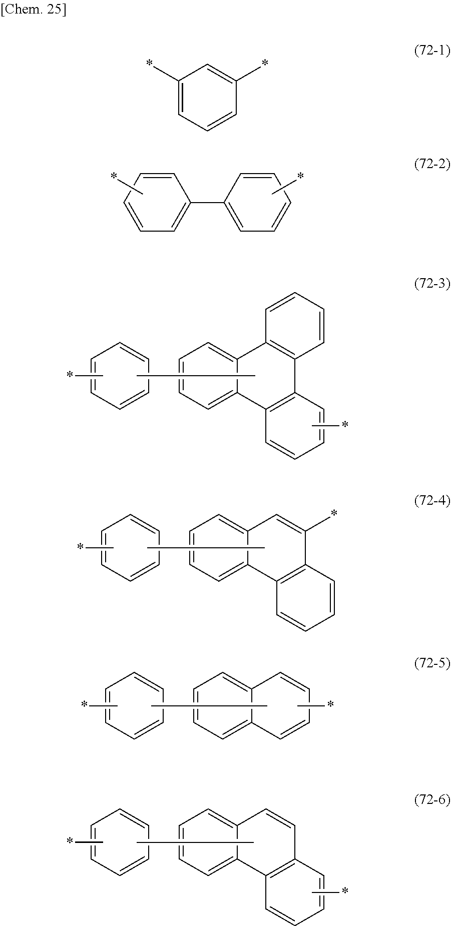

(wherein, in each of formulas (72-1) to (72-6) above, each * represents a bond to an adjacent structure or a hydrogen atom; and at least one of two *'s represents a position of bonding to an adjacent structure).
[30] The composition according to any one of [20] to [29], wherein, in formula (72) above, n⁶¹¹ and n⁶¹² are each 0.
[31] The composition according to any one of [20] to [30], wherein, in formula (72) above, G is a single bond.
[32] The composition according to any one of [20] to[31], further comprising an electron accepting compound having a crosslinking group and represented by formula (81) below:

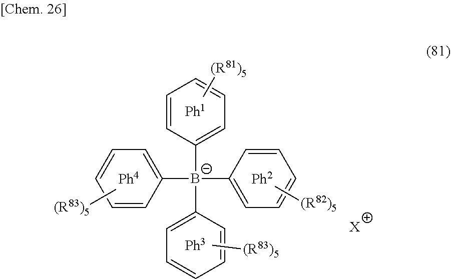

(wherein, in formula (81), five R⁸¹'s, five R⁸²'s, five R⁸³'s, and five R⁸⁴'s are each independent; R⁸¹'s to R⁸⁴'s each independently represent a hydrogen atom, a deuterium atom, a halogen atom, an aromatic hydrocarbon group having 6 to 50 carbon atoms and optionally having a substituent and/or a crosslinking group, an aromatic heterocyclic group having 3 to 50 carbon atoms and optionally having a substituent and/or a crosslinking group, a fluorine-substituted alkyl group having 1 to 12 carbon atoms, or a crosslinking group;
Ph¹, Ph², Ph³, are Ph⁴ are symbols representing four benzene rings;
the compound represented by formula (81) has a crosslinking group; and
X⁺ represents a counter cation).
   [33] The composition according to [32], wherein, in formula (81) above, at least one of -Ph¹-(R⁸¹)₅, -Ph²-(R⁸²)₅, -Ph³-(R⁸³)₅, and -Ph⁴-(R⁸⁴)₅ is a group represented by the following formula (84) and having four fluorine atoms:

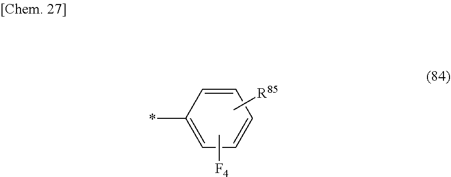

(wherein, in formula (84), * represents a bond to boron B in formula (81);
F₄ represents substitution with four fluorine atoms; and
R⁸⁵ represents an aromatic hydrocarbon group optionally having a substituent and/or a crosslinking group or a crosslinking group).
   [34] The composition according to any one of [32] to [33], wherein the electron accepting compound represented by formula (81) above has at least two crosslinking groups.
   [35] The composition according to any one of [20] to [34], wherein each crosslinking group included in the compound represented by formula (71) or (72) above, each crosslinking group included in the compound represented by formula (81) above, and each crosslinking group included in the polymer having an arylamine structure as a repeating unit are each independently selected from formulas (X1) to (X18) in the following group T of crosslinking groups:

### <Group T of Crosslinking Groups>

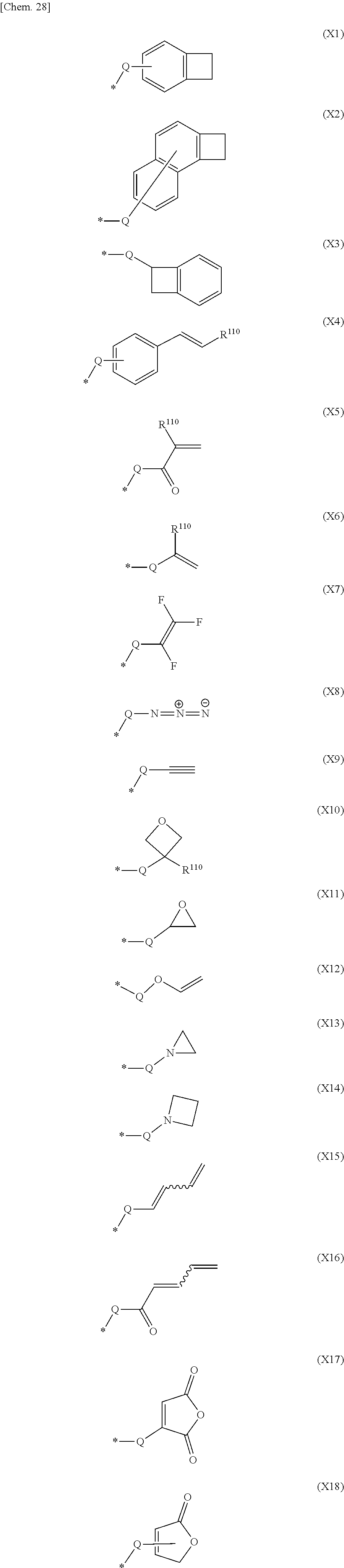

(wherein, in formulas (X1) to (X18), Q represents a direct bond or a linking group;
* represents a bonding position;
R¹¹⁰ in each of formulas (X4), (X5), (X6), and (X10) represents a hydrogen atom or an alkyl group optionally having a substituent;
each of the benzene rings and the naphthalene ring in formula (X1) to (X4) may optionally have a substituent, and any of the substituents may be bonded together to form a ring; and
each cyclobutene ring in formula (X1) to (X3) may optionally have a substituent).
   [36] The composition according to [35], wherein each crosslinking group included in the compound represented by formula (71) or (72) above, each crosslinking group included in the compound represented by formula (81) above, and each crosslinking group included in the polymer having an arylamine structure as a repeating unit are each independently selected from formulas (X1) to (X4) in the group T of crosslinking groups.
   [37] The composition according to any one of [20] to [36], wherein each substituent included in the compound represented by formula (71) or (72) above, each substituent included in the compound represented by formula (81) above, each substituent included in the polymer having an arylamine structure as a repeating unit, and each substituent included in the group T of crosslinking groups are each independently selected from the following substituent group Z:
   <Substituent Group z>
alkyl groups having 1 to 24 carbon atoms,
alkenyl group having 2 to 24 carbon atoms,
alkynyl groups having 2 to 24 carbon atoms,
alkoxy groups having 1 to 24 carbon atoms,
aryloxy groups and heteroaryloxy groups having 4 to 36 carbon atoms,
alkoxycarbonyl groups having 2 to 24 carbon atoms,
dialkylamino groups having 2 to 24 carbon atoms,
diarylamino groups having 10 to 36 carbon atoms,
arylalkylamino groups having 7 to 36 carbon atoms,
acyl groups having 2 to 24 carbon atoms,
halogen atoms,
haloalkyl groups having 1 to 12 carbon atoms,
alkylthio groups having 1 to 24 carbon atoms,
arylthio groups having 4 to 36 carbon atoms,
silyl groups having 2 to 36 carbon atoms,
siloxy groups having 2 to 36 carbon atoms,
a cyano group,
aromatic hydrocarbon groups having 6 to 36 carbon atoms, and
aromatic heterocyclic groups having 4 to 36 carbon atoms,
wherein each of the above substituents may have a linear, branched, or cyclic structure, and wherein, when any of the substituents are adjacent to each other, the substituents adjacent to each other may be bonded together to form a ring.
   [38] The composition according to any one of [20] to [37], further comprising a solvent.
   [39]A method for producing an organic electroluminescent element including an anode and a cathode that are disposed on a substrate and further including an organic layer between the anode and the cathode, the method comprising the step of forming the organic layer by a wet deposition method using the composition according to [19] or [38].
   [40] The method for producing an organic electroluminescent element according to [39], wherein the organic layer is disposed between the anode and a light-emitting layer.
   [41] An organic electroluminescent element produced by the organic electroluminescent element production method according to [39] or [40].
   [42] An organic electroluminescent element comprising an anode and a cathode that are disposed on a substrate and further comprising an organic layer between the anode and the cathode, wherein the organic layer contains a crosslinking reaction product of a carbazole compound having a crosslinking group and represented by formula (71) or (72) below and an electron accepting compound having a crosslinking group and represented by formula (81) below:

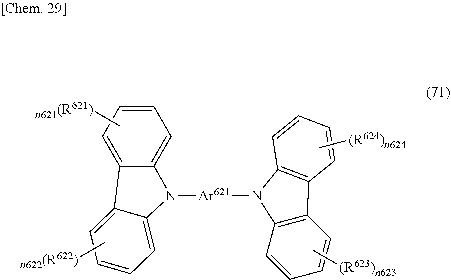

(wherein, in formula (71),
Ar⁶²¹ represents a divalent aromatic hydrocarbon group having 6 to 50 carbon atoms and optionally having a substituent;
R⁶²¹, R⁶²², R⁶²³, and R⁶²⁴ each independently represent a deuterium atom, a halogen atom, a monovalent aromatic hydrocarbon group having 6 to 50 carbon atoms and optionally having and/or a crosslinking group, or a crosslinking group;
n621, n622, n623, and n624 are each independently an integer of 0 to 4,
provided that the sum of n621, n622, n633, and n624 is 1 or more; and
the compound represented by formula (71) has at least two crosslinking groups),

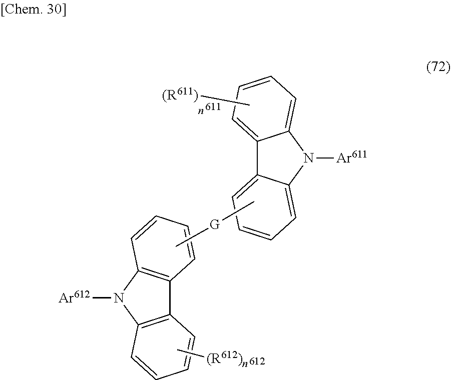

(wherein, in formula (72),
Ar⁶¹¹ and Ar⁶¹² each independently represent a divalent aromatic hydrocarbon group having 6 to 50 carbon atoms and optionally having a substituent and/or a crosslinking group;
R⁶¹¹ and R⁶¹² are each independently a deuterium atom, a halogen atom, a monovalent aromatic hydrocarbon group having 6 to 50 carbon atoms and optionally having a substituent and/or a crosslinking group, or a crosslinking group;
G represents a single bond or a divalent aromatic hydrocarbon group having 6 to 50 carbon atoms and optionally having a substituent and/or a crosslinking group;
n⁶¹¹ and n⁶¹² are each independently an integer of 0 to 4; and
the compound represented by formula (72) has at least two crosslinking groups),

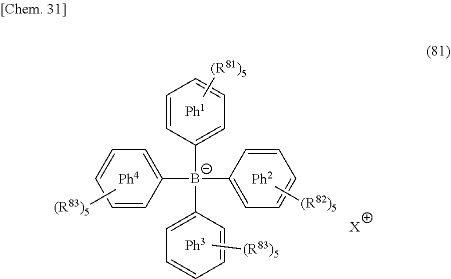

(wherein, in formula (81), five R⁸¹'s, five R⁸²'s, five R⁸³'s, and five R⁸⁴'s are each independent; R⁸¹'s to R⁸⁴'s each independently represent a hydrogen atom, a deuterium atom, a halogen atom, an aromatic hydrocarbon group having 6 to 50 carbon atoms and optionally having a substituent and/or a crosslinking group, an aromatic heterocyclic group having 3 to 50 carbon atoms and optionally having a substituent and/or a crosslinking group, a fluorine-substituted alkyl group having 1 to 12 carbon atoms, or a crosslinking group;
Ph¹, Ph², Ph³, are Ph⁴ are symbols representing four benzene rings;
the compound represented by formula (81) has a crosslinking group; and
X⁺ represents a counter cation).
   [43] An organic electroluminescent element comprising an anode and a cathode that are disposed on a substrate and further comprising an organic layer between the anode and the cathode, wherein the organic layer contains a crosslinking reaction product of a carbazole compound having a crosslinking group and represented by formula (71) or (72) below and a polymer having an arylamine structure as a repeating unit and having a crosslinking group, the repeating unit being a repeating structure represented by formula (50) below, wherein the structure represented by formula (50) has a partial structure represented by formula (63) below:

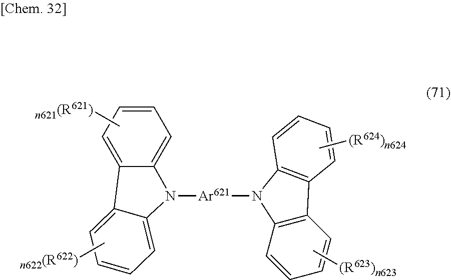

(wherein, in formula (71),
Ar⁶²¹ represents a divalent aromatic hydrocarbon group having 6 to 50 carbon atoms and optionally having a substituent;
R⁶²¹, R⁶²², R⁶²³, and R⁶²⁴ each independently represent a deuterium atom, a halogen atom, a monovalent aromatic hydrocarbon group having 6 to 50 carbon atoms and optionally having and/or a crosslinking group, or a crosslinking group;
n621, n622, n623, and n624 are each independently an integer of 0 to 4,
provided that the sum of n621, n622, n633, and n624 is 1 or more; and
the compound represented by formula (71) has at least two crosslinking groups),

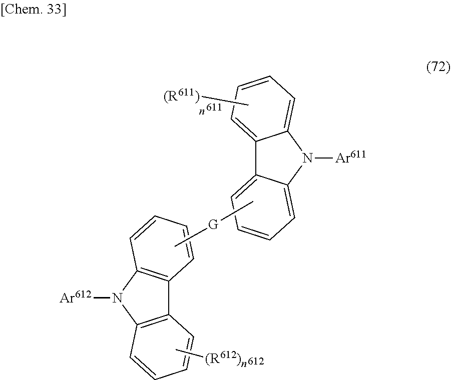

(wherein, in formula (72),
Ar⁶¹¹ and Ar⁶¹² each independently represent a divalent aromatic hydrocarbon group having 6 to 50 carbon atoms and optionally having a substituent and/or a crosslinking group;
R⁶¹¹ and R⁶¹² are each independently a deuterium atom, a halogen atom, a monovalent aromatic hydrocarbon group having 6 to 50 carbon atoms and optionally having a substituent and/or a crosslinking group, or a crosslinking group;
G represents a single bond or a divalent aromatic hydrocarbon group having 6 to 50 carbon atoms and optionally having a substituent and/or a crosslinking group;
n⁶¹¹ and n⁶¹² are each independently an integer of 0 to 4; and
the compound represented by formula (72) has at least two crosslinking groups),

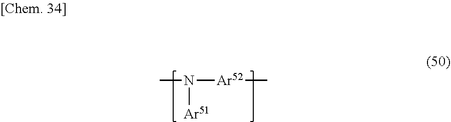

(wherein, in formula (50),
Ar⁵¹ represents an aromatic hydrocarbon group, an aromatic heterocyclic group, or a group in which a plurality of groups selected from aromatic hydrocarbon groups and aromatic heterocyclic groups are linked together;
Ar⁵² represents a divalent aromatic hydrocarbon group, a divalent aromatic heterocyclic group, or a divalent group in which a plurality of groups selected from the group consisting of divalent aromatic hydrocarbon groups and divalent aromatic heterocyclic groups are linked together directly or via a linking group;
Ar⁵¹ and Ar⁵² may form a ring via a single bond or a linking group;
Ar⁵¹ and Ar⁵² may each optionally have a substituent and/or a crosslinking group; and
Ar⁵² has the partial structure represented by formula

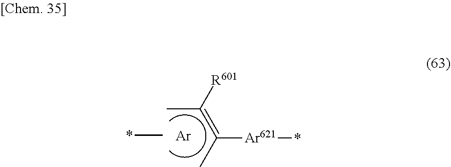

(wherein, in formula (63),
R⁶⁰¹ represents an alkyl group optionally having a substituent or a crosslinking group;
Ar⁶²¹ represents a divalent aromatic hydrocarbon group optionally having a substituent and/or a crosslinking group or a divalent aromatic heterocyclic group optionally having a substituent and/or a crosslinking group;
ring Ar represents an aromatic hydrocarbon structure optionally having a substituent and/or a crosslinking group or a divalent aromatic heterocyclic structure optionally having a substituent and/or a crosslinking group; and
* represents a position bonded to an adjacent atom).
   [44]A display device comprising the organic electroluminescent element according to any one of [41] to [43].
   [45]A lighting device comprising the organic electroluminescent element according to any one of [41] to [43].

### Advantageous Effects of Invention

The present invention can provide an organic electroluminescent element having a low driving voltage, high luminous efficiency, and a long driving lifetime.

### BRIEF DESCRIPTION OF DRAWING

FIG. 1 is a schematic cross-sectional view showing an example of the structure of the organic electroluminescent element of the invention.

### DESCRIPTION OF EMBODIMENTS

Aspects of embodiments of the composition, the organic electroluminescent element, the method for producing the same, the display device, and the lighting device of the present invention will be described in detail. The following description provides examples (representative examples) of the aspects of the invention, i.e., primary embodiments. However, the invention is not limited to the details of these embodiments so long as they do not depart from the scope thereof.

In the present invention, the phrase “optionally having a substituent” means that one or more substituents may be included.

### Definitions

In the detailed descriptions of the structures of the carbazole compound having a crosslinking group, the electron accepting compound having a crosslinking group, a high-molecular weight charge transport compound, and a charge transport polymer, partial structures common to these compounds are the following structures, unless otherwise specified.

### <Aromatic Hydrocarbon Group>

The aromatic hydrocarbon group is any of monovalent, divalent, trivalent, and higher valent aromatic hydrocarbon ring structures and is selected according to the bonding state in the structure of a compound to be described later.

Generally, no particular limitation is imposed on the number of carbon atoms in the structure of the aromatic hydrocarbon ring. However, the number of carbon atoms is preferably 6 or more and 60 or less. The upper limit of the number of carbon atoms is more preferably 48 or less and still more preferably 30 or less. Specific examples of the aromatic hydrocarbon group include: 6-membered monocyclic groups and condensed ring groups including 2 to 5 rings such as a benzene ring, a naphthalene ring, an anthracene ring, a phenanthrene ring, a perylene ring, a tetracene ring, a pyrene ring, a benzpyrene ring, a chrysene ring, a triphenylene ring, an acenaphthene ring, a fluoranthene ring, and a fluorene ring; and structures in which a plurality of groups selected from the above groups are linked together.

A structure including a plurality of aromatic hydrocarbon rings linked together is generally a structure including 2 to 10 aromatic hydrocarbon rings linked together and preferably a structure including 2 to 5 aromatic hydrocarbon rings linked together. In the structure including a plurality of aromatic hydrocarbon rings linked together, groups having the same structure may be linked together, or groups having different structures may be linked together.

The aromatic hydrocarbon ring structure is preferably a benzene ring, a biphenyl ring, i.e., a structure including two benzene rings linked together, a terphenyl ring, i.e., a structure including three benzene rings linked together, a quaterphenyl ring, i.e., a structure including four benzene rings linked together, a naphthalene ring, or a fluorene ring.

### <Aromatic Heterocyclic Group>

The aromatic heterocyclic group is any of monovalent, divalent, trivalent, and higher valent aromatic heterocyclic structures and is selected according to the bonding state in the structure of a compound to be described later.

Generally, no particular limitation is imposed on the number of carbon atoms in the structure of the aromatic heterocycle. However, the number of carbon atoms is preferably 3 or more and 60 or less. The upper limit of the number of carbon atoms is more preferably 48 or less and still more preferably 30 or less. Specific examples of the aromatic heterocyclic group include: divalent 5- and 6-membered monocyclic groups and divalent condensed ring groups including 2 to 4 rings such as a furan ring, a benzofuran ring, a thiophene ring, a benzothiophene ring, a pyrrole ring, a pyrazole ring, an imidazole ring, an oxadiazole ring, an indole ring, a carbazole ring, a pyrroloimidazole ring, a pyrrolopyrazole ring, a pyrrolopyrrole ring, a thienopyrrole ring, a thienothiophene ring, a furopyrrole ring, a furofuran ring, a thienofuran ring, a benzisoxazole ring, a benzisothiazole ring, a benzimidazole ring, a pyridine ring, a pyrazine ring, a pyridazine ring, a pyrimidine ring, a triazine ring, a quinoline ring, an isoquinoline ring, a cinnoline ring, a quinoxaline ring, a phenanthridine ring, a benzimidazole ring, a perimidine ring, a quinazoline ring, a quinazolinone ring, and an azulene ring; and groups in which a plurality of groups selected from the above groups are linked together.

When a plurality of aromatic heterocycles are linked together, heterocycles having the same structure may be linked together, or heterocycles having different structures may be linked together. The structure including a plurality of aromatic heterocycles linked together is generally a structure including 2 to 10 aromatic heterocycles linked together and preferably a structure including 2 to 5 aromatic heterocycles linked together.

The aromatic heterocyclic structure is preferably a thiophene ring, a benzothiophene ring, a pyrimidine ring, a triazine ring, a carbazole ring, a dibenzofuran ring, or a dibenzothiophene ring.

### <Crosslinking Group>

The crosslinking group is a group that reacts with another crosslinking group located near the crosslinking group when heated and/or irradiated with active energy rays to thereby form a new chemical bond. In this case, the crosslinking groups reacted with each other may be the same or may be different.

No particular limitation is imposed on the crosslinking group. Examples of the crosslinking group include groups including an alkenyl group, groups including a conjugated diene structure, groups including an alkynyl group, groups including an oxirane structure, groups including an oxetane structure, groups including an aziridine structure, an azido group, groups including a maleic anhydride structure, groups including an alkenyl group bonded to an aromatic ring, and a cyclobutene ring fused to an aromatic ring. Preferred specific examples of the crosslinking group include groups represented by formulas (X1) to (X18) in the following group T of crosslinking groups.

### <Group T of Crosslinking Groups>

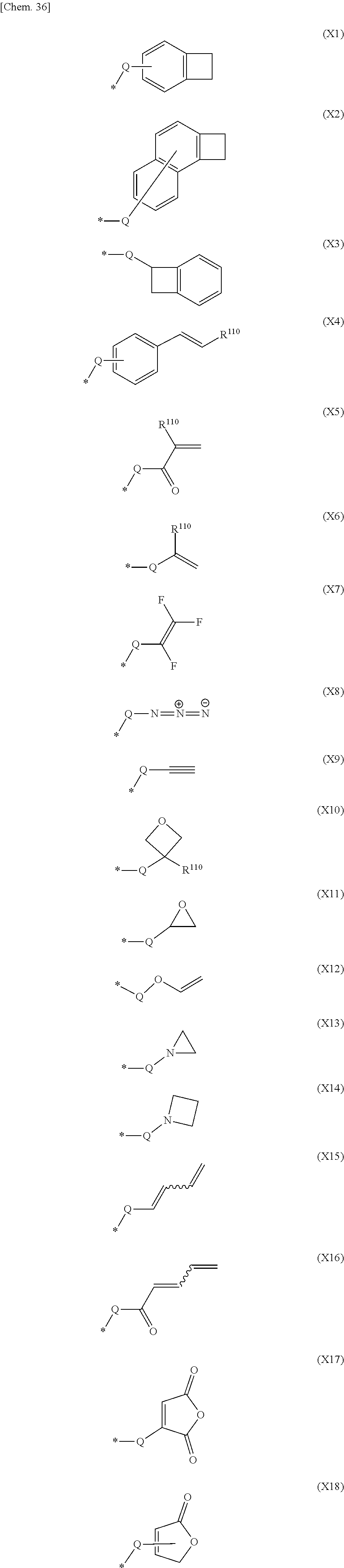

(In formulas (X1) to (X18), Q represents a direct bond or a linking group, and

* represents a bonding position.

In formulas (X4), (X5), (X6), and (X10), R¹¹⁰ represents a hydrogen atom or an alkyl group optionally having a substituent.

In formulas (X1) to (X4), the benzene rings and the naphthalene ring may each optionally have a substituent. Any of the substituents may be bonded together to form a ring.

In formulas (X1) to (X3), each cyclobutene ring may optionally have a substituent.)

When Q is a linking group, no particular limitation is imposed on the linking group. However, the linking group is preferably an alkylene group, a divalent oxygen atom, or a divalent aromatic hydrocarbon group optionally having a substituent.

The alkylene group is generally an alkylene group having 1 to 12 carbon atoms, preferably an alkylene group having 1 to 8 carbon atoms, and more preferably an alkylene group having 1 to 6 carbon atoms.

The number of carbon atoms in the divalent aromatic hydrocarbon group is generally 6 or more and is generally 36 or less, preferably 30 or less, and more preferably 24 or less. The structure of the aromatic hydrocarbon ring is preferably a benzene ring, and the optional substituent can be selected from a substituent group Z described later.

Q is preferably a divalent aromatic hydrocarbon group optionally having a substituent because the reactivity of the crosslinking group can be increased while the performance of the element is maintained.

The alkyl group represented by R¹¹⁰ has a linear, branched, or cyclic structure, and the number of carbon atoms in the alkyl group is 1 or more and is preferably 24 or less, more preferably 12 or less, and still more preferably 8 or less.

The optional substituents on the benzene and naphthalene rings in formulas (X1) to (X4) and R¹¹⁰ in formulas (X4), (X6), and (X10) are each preferably an alkyl group, an aromatic hydrocarbon group, an alkyloxy group, or an aralkyl group.

The alkyl group serving as a substituent has a linear, branched, or cyclic structure, and the number of carbon atoms in the alkyl group is preferably 24 or less, more preferably 12 or less, and still more preferably 8 or less and is preferably 1 or more.

The number of carbon atoms in the aromatic hydrocarbon group serving as a substituent is preferably 24 or less, more preferably 18 or less, and still more preferably 12 or less and is preferably 6 or more. The aromatic hydrocarbon group may further optionally have the above-described alkyl group as a substituent.

The number of carbon atoms in the alkyloxy group serving as a substituent is preferably 24 or less, more preferably 12 or less, and still more preferably 8 or less and is preferably 1 or more.

The number of carbon atoms in the aralkyl group serving as a substituent is preferably 30 or less, more preferably 24 or less, and still more preferably 14 or less and is preferably 7 or more. Preferably, the alkylene group included in the aralkyl group has a linear or branched structure. The aryl group included in the aralkyl group may further optionally have the above-described alkyl group as a substituent.

The optional substituents on the cyclobutene rings in formulas (X1), (X2), and (X3) are each preferably an alkyl group. The alkyl group serving as a substituent has a linear, branched, or cyclic structure, and the number of carbon atoms in the alkyl group is preferably 24 or less, more preferably 12 or less, and still more preferably 8 or less and is preferably 1 or more.

The crosslinking group is preferably any of the crosslinking groups represented by formulas (X1) to (X3) because their polarity is small, their influence on charge transportability is small, and the crosslinking reaction can be initiated only by heat.

In the crosslinking group represented by formula (X1), the cyclobutene ring undergoes ring-opening by heat, and the ring-opened groups are bonded together to form a crosslinked structure as shown by a formula below. In the following description, the linking groups Q in formulas (X1) to (X4) etc. are omitted.

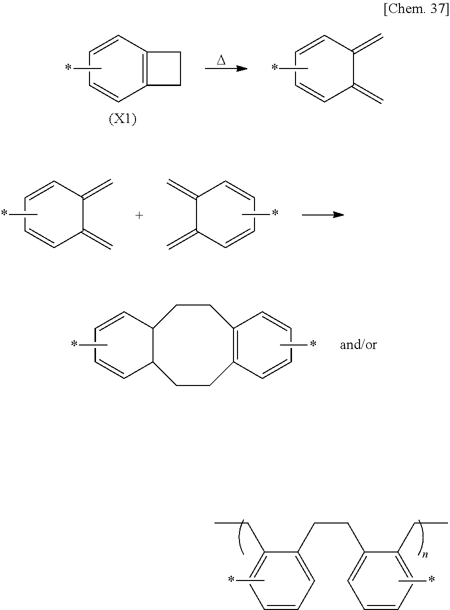

In the crosslinking group represented by formula (X2), the cyclobutene ring undergoes ring-opening by heat, and the ring-opened groups are bonded together to form a crosslinked structure as shown by the following formula.

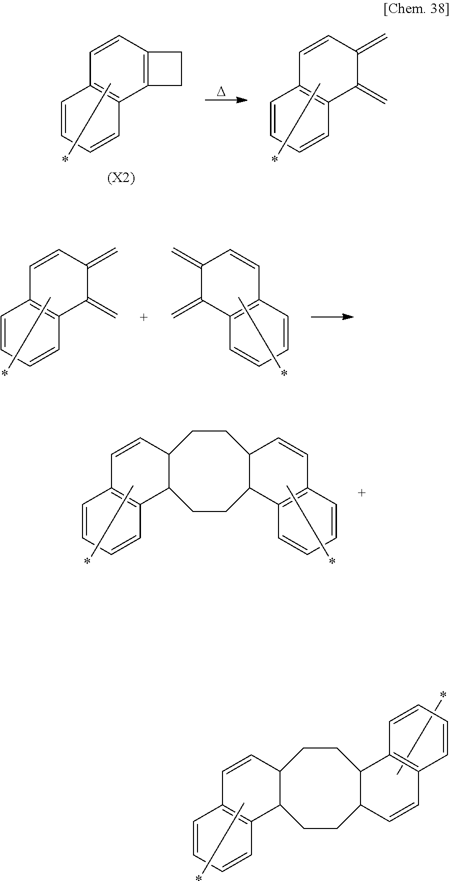

In the crosslinking group represented by formula (X3), the cyclobutene ring undergoes ring-opening by heat, and the ring-opened groups are bonded together to form a crosslinked structure as shown by the following formula.

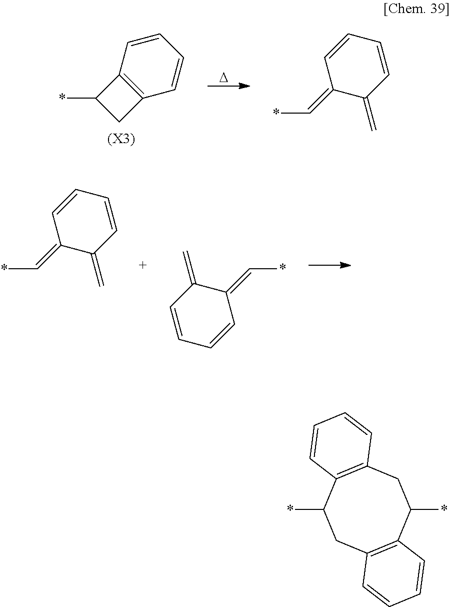

In the crosslinking group represented by any of formulas (X1) to (X3), the cyclobutene ring undergoes ring-opening by heat. When a double bond is present near the ring-opened group, the ring-opened group reacts with the double bond to form a crosslinked structure.

An example in which the ring-opened group obtained as a result of ring-opening of the crosslinking group represented by formula (X1) and the crosslinking group represented by formula (X4) and having a double bond form a crosslinked structure is shown below.

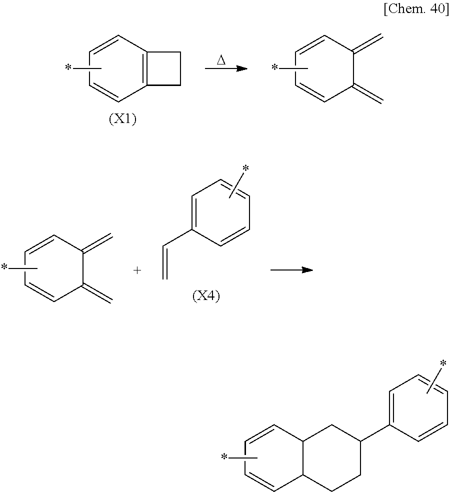

Examples of the group having a double bond reactable with the crosslinking group represented by any of formulas (X1) to (X3) include, in addition to the crosslinking group represented by formula (X4), crosslinking groups represented by formulas (X5), (X6), (X12), (X15), (X16), (X17), and (X18). When any of these groups each having a double bond is used as the crosslinking group in the electron accepting compound, it is preferable that the crosslinking group represented by any of formulas (X1) to (X3) is included in a component such as a hole transport compound that is included in a hole injection layer and/or a hole transport layer, because the possibility that a crosslinked structure is formed increases.

The crosslinking group is preferably a radical polymerizable crosslinking group represented by any of formulas (X4), (X5), and (X6) because their polarity is small and they are unlikely to impede charge transport.

In view of increasing the electron acceptability, the crosslinking group is preferably a crosslinking group represented by formula (X7). With the crosslinking group represented by formula (X7), the following crosslinking reaction proceeds.

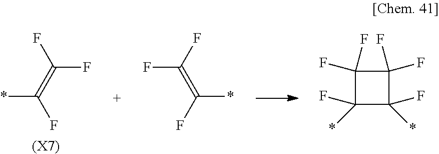

The crosslinking group represented by any of formulas (X8) and (X9) is preferred because of their high reactivity. When the crosslinking group represented by formula (X8) and the crosslinking group represented by formula (X9) are used, the following crosslinking reaction proceeds.

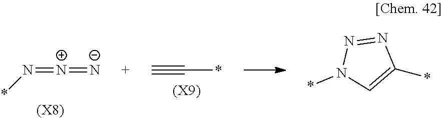

The crosslinking group is preferably a cationic polymerizable crosslinking group represented by any of formulas (X10), (X11), and (X12) because of their high reactivity.

From the viewpoint of improving the stability after crosslinking and the performance of the element, it is preferable that at least one of the high-molecular weight charge transport compound, the low-molecular weight charge transport compound, and the electron accepting compound contained in the composition of the invention and described later has the crosslinking group represented by any of formulas (X1) to (X4), and it is more preferable that at least one of them has the crosslinking group represented by formula (X2) or (X4). In the crosslinking group represented by formula (X4), R¹¹⁰ is preferably a substituent, and preferred examples of the substituent are as described above.

### <Substituents>

Each of the substituents can be any group, unless otherwise specified. However, it is preferable that each substituent is selected from a substituent group Z described below. When it is stated that the optional substituent is selected from the substituent group Z or that it is preferable to select the optional substituent from the substituent group Z, preferred examples of the substituent are those described in the substituent group Z.

The substituent group Z is the group consisting of alkyl groups, alkenyl groups, alkynyl groups, alkoxy groups, aryloxy groups, heteroaryloxy groups, alkoxycarbonyl groups, dialkylamino groups, diarylamino groups, arylalkylamino groups, acyl groups, halogen atoms, haloalkyl groups, alkylthio groups, arylthio groups, silyl groups, siloxy groups, a cyano group, aromatic hydrocarbon groups, and aromatic heterocyclic groups. These substituents may each include a linear, branched, or cyclic structure.

### <Substituent Group Z>

Among the substituent group Z, structures described in the following are preferred.
Alkyl groups having 1 to 24 carbon atoms,
alkenyl groups having 2 to 24 carbon atoms,
alkynyl groups having 2 to 24 carbon atoms,
alkoxy groups having 1 to 24 carbon atoms,
aryloxy groups and heteroaryloxy groups having 4 to 36 carbon atoms,
alkoxycarbonyl groups having 2 to 24 carbon atoms,
dialkylamino groups having 2 to 24 carbon atoms,
diarylamino groups having 10 to 36 carbon atoms,
arylalkylamino groups having 7 to 36 carbon atoms,
acyl groups having 2 to 24 carbon atoms,
halogen atoms,
haloalkyl groups having 1 to 12 carbon atoms,
alkylthio groups having 1 to 24 carbon atoms,
arylthio groups having 4 to 36 carbon atoms,
silyl groups having 2 to 36 carbon atoms,
siloxy groups having 2 to 36 carbon atoms,
a cyano group,
aromatic hydrocarbon groups having 6 to 36 carbon atoms, and
aromatic heterocyclic groups having 4 to 36 carbon atoms.

These substituent may each include a linear, branched, or cyclic structure. When any of the substituents are adjacent to each other, the substituents adjacent to each other may be bonded together to form a ring.

More specific examples of the substituent group Z include the following structures.

Linear, branched, and cyclic alkyl groups having 1 or more carbon atoms and preferably 4 or more carbon atoms and having 24 or less carbon atoms, preferably 12 or less carbon atoms, still more preferably 8 or less carbon atoms, and yet more preferably 6 or less carbon atoms. Specific examples include a methyl group, an ethyl group, a n-propyl group, an i-propyl group, a n-butyl group, an i-butyl group, a sec-butyl group, a tert-butyl group, a n-hexyl group, a cyclohexyl group, and a dodecyl group.

Linear, branched, and cyclic alkyl groups having generally 2 or more carbon atoms and having generally 24 or less carbon atoms and preferably 12 or less carbon atoms. Specific examples include a vinyl group.

Linear and branched alkynyl groups having generally 2 or more carbon atoms and generally 24 or less carbon atoms and preferably 12 or less carbon atoms. Specific examples include an ethynyl group.

Alkoxy groups having 1 or more carbon atoms and having 24 or less carbon atoms and preferably 12 or less carbon atoms. Specific examples include a methoxy group and an ethoxy group.

Aryloxy groups and heteroaryloxy groups having 4 or more carbon atoms and preferably 5 carbon atoms and having 36 or less carbon atoms and preferably 24 or less carbon atoms. Specific examples include a phenoxy group, a naphthoxy group, and a pyridyloxy group.

Alkoxycarbonyl groups having 2 or more carbon atoms and having 24 or less carbon atoms and preferably 12 or less carbon atoms. Specific examples include a methoxycarbonyl group and an ethoxycarbonyl group.

Dialkylamino groups having 2 or more carbon atoms and having 24 or less carbon atoms and preferably 12 or less carbon atoms. Specific examples include a dimethylamino group and a diethylamino group.

Diarylamino groups having 10 or more carbon atoms and preferably 12 or more carbon atoms and having 36 or less carbon atoms and preferably 24 or less carbon atoms. Specific examples include a diphenylamino group, a ditolylamino group, and an N-carbazolyl group.

Arylalkylamino groups having 7 or more carbon atoms and having 36 or less carbon atoms and preferably 24 or less carbon atoms. Specific examples include a phenylmethylamino group.

Acyl groups having 2 or more carbon atoms and having 24 or less carbon atoms and preferably 12 or less carbon atoms. Specific examples include an acetyl group and a benzoyl group.

Halogen atoms such as a fluorine atom and a chlorine atom. A fluorine atom is preferred.

Haloalkyl groups having 1 or more carbon atoms and having 12 or less carbon atoms and preferably 6 or less carbon atoms. Specific examples include a trifluoromethyl group.

Alkylthio groups having 1 or more carbon atoms and having generally 24 carbon atoms and preferably 12 or less carbon atoms. Specific examples include a methylthio group and an ethylthio group.

Arylthio groups having 4 or more carbon atoms and preferably 5 or more carbon atoms and having 36 or less carbon atoms and preferably 24 or less carbon atoms. Specific examples include a phenylthio group, a naphthylthio group, and a pyridylthio group.

Silyl groups having generally 2 or more carbon atoms and preferably 3 or more carbon atoms and having generally 36 or less carbon atoms and preferably 24 or less carbon atoms. Specific examples include a trimethylsilyl group and a triphenylsilyl group.

Siloxy groups having 2 or more carbon atoms and preferably 3 or more carbon atoms and having generally 36 or less carbon atoms and preferably 24 or less carbon atoms. Specific examples include a trimethylsiloxy group and a triphenylsiloxy group.

A cyano group.

Aromatic hydrocarbon groups having 6 or more carbon atoms and having 36 or less carbon atoms and preferably 24 or less carbon atoms. Specific examples include a phenyl group, a naphthyl group, groups including a plurality of phenyl groups linked together.

Aromatic heterocyclic groups having 3 or more carbon atoms and preferably 4 or more carbon atoms and having 36 or less carbon atoms and preferably 24 or less carbon atoms. Specific examples include a thienyl group and a pyridyl group.

Each of the above substituents may include a linear, branched, or cyclic structure.

When any of these substituents are adjacent to each other, the substituents adjacent to each other may be bonded together to form a ring. A preferred size of the ring is a 4-membered ring, a 5-membered ring, or a 6-membered ring, and specific examples include a cyclobutane ring, a cyclopentane ring, and a cyclohexane ring.

Among the substituent group Z, alkyl groups, alkoxy groups, aromatic hydrocarbon groups, and aromatic heterocyclic groups are preferred.

The substituents in the substituent group Z may each further optionally have an additional substituent. Examples of the optional additional substituent include those in the substituent group Z and crosslinking groups. Preferably, these substituents do not have an additional substituent. When these substituents each have an additional substituent, the additional substituent is preferably an alkyl group having 8 or less carbon atoms, an alkoxy group having 8 or less carbon atoms, or a phenyl group and more preferably an alkyl group having 6 or less carbon atoms, an alkoxy group having 6 or less carbon atoms, or a phenyl group. From the viewpoint of charge transportability, it is more preferable that these substituents have no additional substituent.

When the additional substituent optionally included in each of the substituents in the substituent group Z is a crosslinking group, the crosslinking group is preferably a crosslinking group selected from the group T of crosslinking groups. Each substituent preferably having an additional crosslinking group is an alkyl group or an aromatic hydrocarbon group.

### <Charge Transport Material>

The charge transport material in the invention is a material capable of transporting holes and/or electrons. The carbazole compound having a crosslinking group and the high-molecular weight charge transport compound in the invention described later are each a charge transport material. The charge transport material in the invention is preferably a hole transport material and is also preferably a material oxidized by the electron accepting compound to form a cation radical. In the invention, the high-molecular weight charge transport compound is preferably a high-molecular weight hole transport compound and is also preferably a polymer including an arylamine structure as a repeating unit. In this case, the charges are generally holes, and the charge transport means hole transport. A charge transport film is a hole transport film, and a charge injection layer is a hole injection layer.

### [Composition of Invention]

A composition of aspect 1 of the invention is a composition containing a carbazole compound having a crosslinking group and represented by formula (71) or (72) below (this compound may be hereinafter referred to as the “carbazole compound in the invention”) and an electron accepting compound having a crosslinking group and represented by formula (81) below (this compound may be hereinafter referred to as the “electron accepting compound in the invention”).

A composition of aspect 2 of the invention is a composition containing the carbazole compound having a crosslinking group and represented by formula (71) or (72) below and a polymer having an arylamine structure as a repeating unit. The polymer having an arylamine structure as a repeating unit has a structure represented by formula (50) below as the repeating unit and also has a crosslinking group. The structure represented by formula (50) has a partial structure represented by formula (63).

Aspect 3 of the invention is an organic electroluminescent element including an anode and a cathode that are disposed on a substrate and further including an organic layer between the anode and the cathode. In the organic electroluminescent element, the organic layer contains a crosslinking reaction product of the carbazole compound having a crosslinking group and represented by formula (71) or (72) below and the electron accepting compound having a crosslinking group and represented by formula (81) below.

Aspect 4 of the invention is an organic electroluminescent element including an anode and a cathode that are disposed on a substrate and further including an organic layer between the anode and the cathode. In the organic electroluminescent element, the organic layer contains a crosslinking reaction product of the carbazole compound having a crosslinking group and represented by formula (71) or (72) below and a polymer having an arylamine structure as a repeating unit and having a crosslinking group, the repeating unit being represented by formula (50) below.

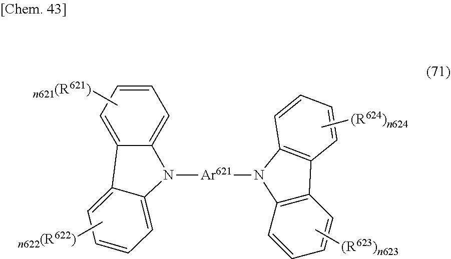

(In formula (71),
Ar⁶²¹ represents a divalent aromatic hydrocarbon group having 6 to 50 carbon atoms and optionally having a substituent;
R⁶²¹, R⁶²², R⁶²³, and R⁶²⁴ are each independently a deuterium atom, a halogen atom, a monovalent aromatic hydrocarbon group having 6 to 50 carbon atoms and optionally having and/or a crosslinking group, or a crosslinking group;
n621, n622, n623, and n624 are each independently an integer of 0 to 4,
provided that the sum of n621, n622, n633, and n624 is 1 or more; and
the compound represented by formula (71) has at least two crosslinking groups.)

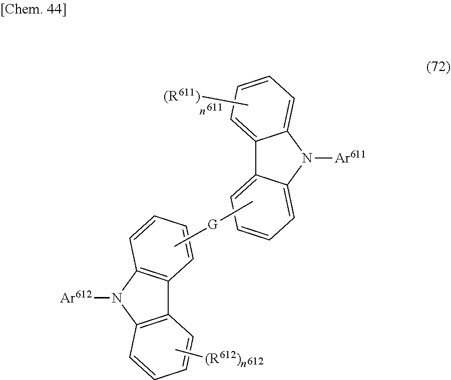

(In formula (72),
Ar⁶¹¹ and Ar⁶¹² each independently represent a divalent aromatic hydrocarbon group having 6 to 50 carbon atoms and optionally having a substituent and/or a crosslinking group;
R⁶¹¹ and R⁶¹² are each independently a deuterium atom, a halogen atom, a monovalent aromatic hydrocarbon group having 6 to 50 carbon atoms and optionally having a substituent and/or a crosslinking group, or a crosslinking group;
G represents a single bond or a divalent aromatic hydrocarbon group having 6 to 50 carbon atoms and optionally having a substituent and/or a crosslinking group;
n⁶¹¹ and n⁶¹² are each independently an integer of 0 to 4; and
the compound represented by formula (72) has at least two crosslinking groups.)

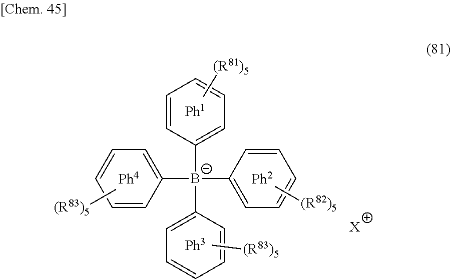

(In formula (81), five R⁸¹'s, five R⁸²'s, five R⁸³'s, and five R³⁴'s are each independent; R⁸¹'s to R³⁴'s each independently represent a hydrogen atom, a deuterium atom, a halogen atom, an aromatic hydrocarbon group having 6 to 50 carbon atoms and optionally having a substituent and/or a crosslinking group, an aromatic heterocyclic group having 3 to 50 carbon atoms and optionally having a substituent and/or a crosslinking group, a fluorine-substituted alkyl group having 1 to 12 carbon atoms, or a crosslinking group;
Ph¹, Ph², Ph³, are Ph⁴ are symbols representing four benzene rings;
the compound represented by formula (81) has a crosslinking group; and
X⁺ represents a counter cation.)

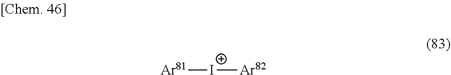

(In formula (83), Ar⁸¹ and Ar¹² are each independently an aromatic hydrocarbon group having 6 to 30 carbon atoms and optionally having a substituent.)

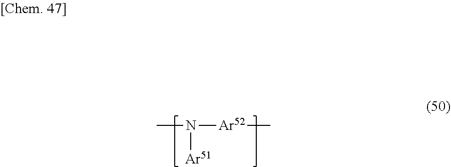

(In formula (50),
Ar⁵¹ represents an aromatic hydrocarbon group, an aromatic heterocyclic group, or a group in which a plurality of groups selected from aromatic hydrocarbon groups and aromatic heterocyclic groups are linked together;
Ar⁵² represents a divalent aromatic hydrocarbon group, a divalent aromatic heterocyclic group, or a divalent group in which a plurality of groups selected from the group consisting of divalent aromatic hydrocarbon groups and divalent aromatic heterocyclic groups are linked together directly or via a linking group;
Ar⁵¹ and Ar⁵² may form a ring via a single bond or a linking group;
Ar⁵¹ and Ar⁵² may each optionally have a substituent and/or a crosslinking group; and
Ar⁵² has the partial structure represented by formula (63) below.)

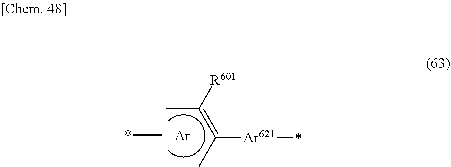

(In formula (63),
R⁶⁰¹ represents an alkyl group optionally having a substituent or a crosslinking group;
Ar⁶²¹ represents a divalent aromatic hydrocarbon group optionally having a substituent and/or a crosslinking group or a divalent aromatic heterocyclic group optionally having a substituent and/or a crosslinking group;
ring Ar represents an aromatic hydrocarbon structure optionally having a substituent and/or a crosslinking group or a divalent aromatic heterocyclic structure optionally having a substituent and/or a crosslinking group; and
* represents a position bonded to an adjacent atom.)

### <Carbazole Compound Having Crosslinking Group>

The carbazole compound in the invention is a compound represented by formula (71) or (72) below and is contained in the composition of the invention as a charge transport material. The carbazole compound in the invention has at least two crosslinking groups.

In the present description, the carbazole compound in the invention may be referred to as the carbazole compound having a crosslinking group.

### <Carbazole Compound Represented by Formula (71)>

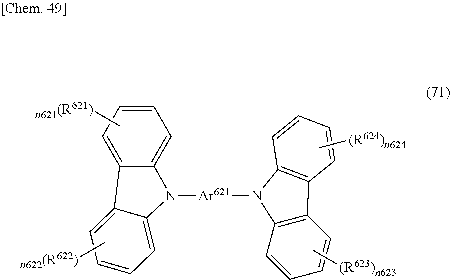

(In formula (71),
Ar⁶²¹ represents a divalent aromatic hydrocarbon group having 6 to 50 carbon atoms and optionally having a substituent;
R⁶²¹, R⁶²², R⁶²³, and R⁶²⁴ are each independently a deuterium atom, a halogen atom, a monovalent aromatic hydrocarbon group having 6 to 50 carbon atoms and optionally having and/or a crosslinking group, or a crosslinking group;
n621, n622, n623, and n624 are each independently an integer of 0 to 4,
provided that the sum of n621, n622, n633, and n624 is 1 or more; and
the compound represented by formula (71) has at least two crosslinking groups.)

### (Ar⁶²¹)

Ar⁶²¹ represents a divalent aromatic hydrocarbon group optionally having a substituent, and the number of carbon atoms in Ar⁶²¹ is 6 to 50.

The number of carbon atoms in the aromatic hydrocarbon group is preferably 6 to 50, more preferably 6 to 30, and still more preferably 6 to 18. Specific examples of the aromatic hydrocarbon group include: divalent groups each including an aromatic hydrocarbon ring structure having generally 6 or more carbon atoms and having generally 30 or less carbon atoms, preferably 18 or less carbon atoms, and still more preferably 14 or less carbon atoms such as a benzene ring, a naphthalene ring, a fluorene ring, an anthracene ring, a tetraphenylene ring, a phenanthrene ring, a chrysene ring, a pyrene ring, a benzanthracene ring, and a perylene ring; and divalent groups each having a structure obtained by bonding a plurality of structures selected from the above structures in a linear or branched manner. The structure including a plurality of aromatic hydrocarbon rings linked together is generally a structure including 2 to 8 rings linked together and preferably a structure including 2 to 5 rings linked together. When a plurality of aromatic hydrocarbon rings are linked together, the aromatic hydrocarbon rings linked together may have the same structure or may have different structures.

The aromatic hydrocarbon group is preferably a divalent group formed by bonding, in any order in a linear or branched manner, a plurality of structures selected from 1 to 4 benzene rings, 1 or 2 naphthalene rings, 1 or 2 fluorene rings, 1 or 2 phenanthrene rings, and one tetraphenylene ring, a 1,4-phenylene group, a 1,3-phenylene group, a 2,7-fluorenylene group, or a divalent spirofluorene group, more preferably a divalent group formed by bonding, in any order in a linear or branched manner, a plurality of structures selected from 1 to 4 benzene rings and 1 or 2 fluorene rings, and particularly preferably a divalent group formed by bonding, in the following order in a linear manner, 1 or 2 phenylene groups, a 2,7-fluorenylene group, and 1 or 2 phenylene groups, a phenylene group, a biphenylene group, a p-terphenylene group, or a 2,7-fluorenylene group. The fluorene structure may optionally have substituents at the 9- and 9′ positions, and the optional substituents are each preferably a group selected from the substituent group Z.

Each of the above aromatic hydrocarbon structures may optionally have a substituent. The optional substituent is as described above. Specifically, the substituent can be selected from the substituent group Z. Preferred substituents are the preferred substituents in the substituent group Z.

### (Partial Structure of Ar⁶²¹)

Ar⁶²¹ has preferably at least one partial structure selected from the following formulas (71-1) to (71-11) and (71-21) to (71-24) from the viewpoint that the stability of the compound against electric charges tends to be improved and has more preferably at least one partial structure selected from the following formula (71-1) to (71-7) from the viewpoint of the solubility and durability of the compound.

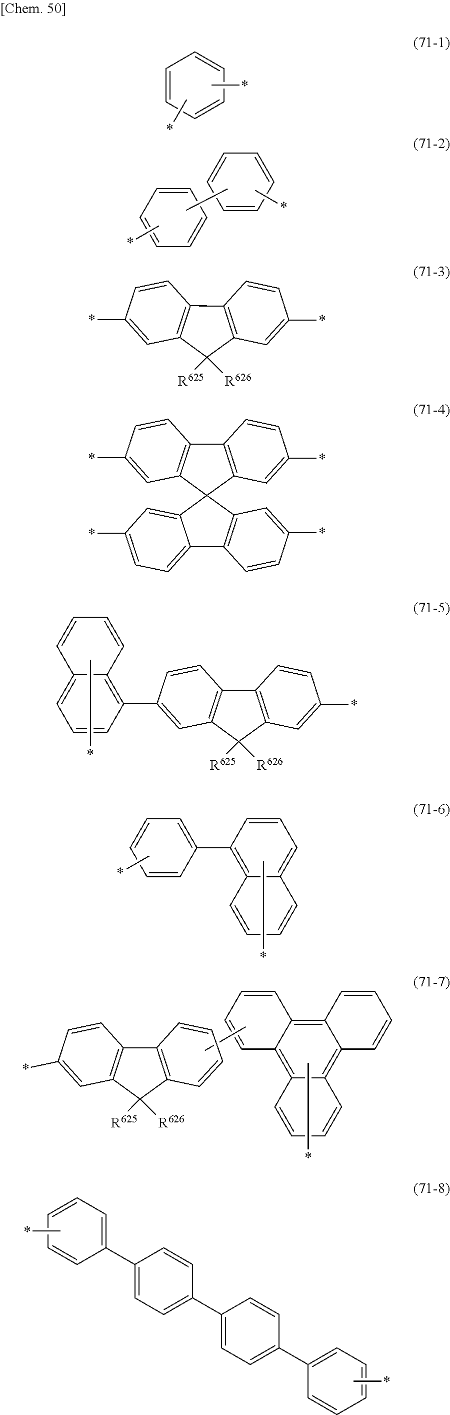

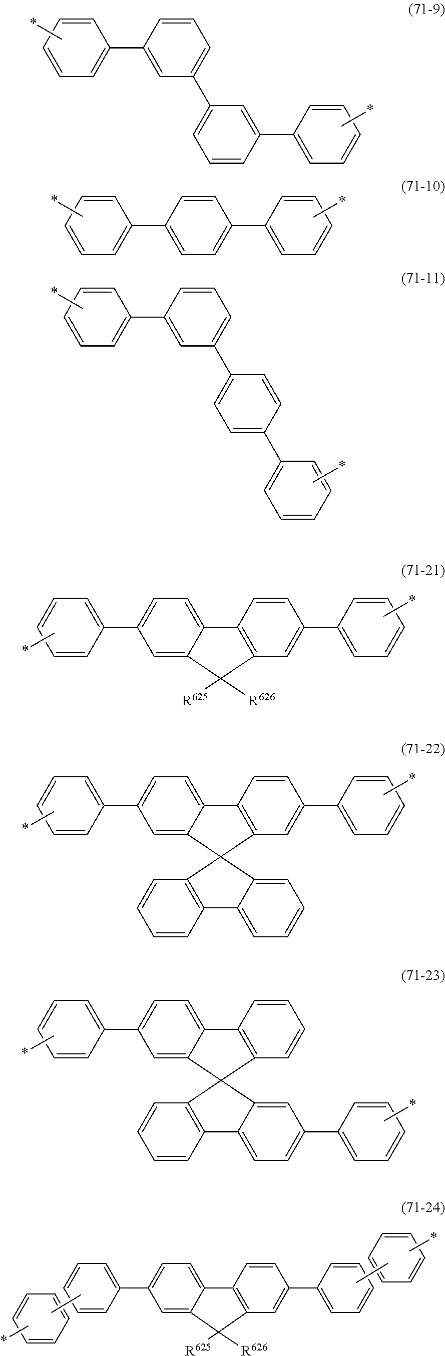

(In each of formulas (71-1) to (71-11) and (71-21) to (71-24) above,
each * represents a bond to an adjacent structure or a hydrogen atom; when two *'s are present, at least one of them represents a position of bonding to an adjacent structure; when four *'s are present, at least one of any two of the four *'s represents a position of bonding to an adjacent structure.
R⁶²⁵ and R⁶²⁶ each independently represent an alkyl group having 6 to 12 carbon atoms, an alkenyl group, an alkynyl group, an alkoxy group, an aryloxy group, an alkoxycarbonyl group, an acyl group, a halogen atom, a haloalkyl group, an alkylthio group, an arylthio group, a silyl group, a siloxy group, a cyano group, an aralkyl group, or a monovalent aromatic hydrocarbon group having 6 to 30 carbon atoms. R⁶²⁵ and R⁶²⁶ may be bonded together to form a ring.)

The aromatic hydrocarbon ring structures represented by R⁶²⁵ and R⁶²⁶ are each more preferably a phenyl group or a group including a plurality of phenyl groups linked together.

Each of these groups may optionally have a substituent. The optional substituent is as described above. Specifically, the substituent can be selected from the substituent group Z. Preferred substituents are the preferred substituents in the substituent group Z.

The partial structure is more preferably a structure selected from formulas (71-1) to (71-7), still more preferably a structure selected from formulas (71-1) to (71-5), and particularly preferably a structure selected from formulas (71-1) to (71-4). The structure represented by formula (71-3) is most preferred because good charge transportability is obtained.

Formula (71-1) is preferably a 1,3-phenylene group or a 1,4-phenylene group.

Formula (71-2) is preferably the following formula (71-2-2).

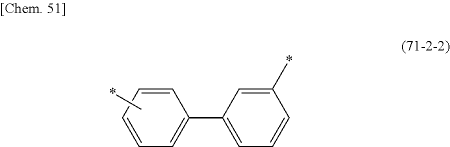

Formula (71-2) is more preferably the following formula (71-2-3).

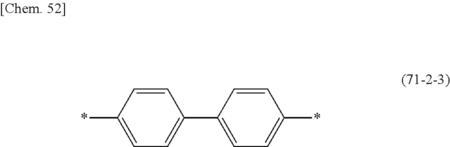 [SMILES: *c1ccc(-c2ccc(*)cc2)cc1]

From the viewpoint of the solubility and durability of the compound, it is preferable that Ar⁶²¹ has, as a partial structure, a partial structure represented by formula (71-1) and a partial structure represented by formula (71-2).

The partial structure including a partial structure represented by formula (71-1) and a partial structure represented by formula (71-2) is more preferably a partial structure represented by at least one selected from formulas (71-8) to (71-11) above, each of which represents a structure including a plurality of structures selected from partial structures represented by formula (71-1) and partial structures represented by formula (71-2).

A partial structure including a partial structure represented by formula (71-1) and a partial structure represented by formula (71-3) or (71-4) is more preferably a partial structure represented by at least one selected from formulas (71-21) to (71-24) above, each of which represents a structure including a plurality of structures selected from partial structures represented by formula (71-1) and partial structures represented by formulas (71-3) and (71-4).

In the present invention, a compound including, between the carbazole rings, a fluorene ring having a substituent having good charge transportability is preferred, and it is preferable to include a fluorene ring as Ar⁶²¹.

(R⁶²¹, R⁶²², R⁶²³, and R⁶²⁴)

R⁶²¹, R⁶²², R⁶²³, and R⁶²⁴ each independently represent a deuterium atom, a halogen atom, a monovalent aromatic hydrocarbon group having 6 to 50 carbon atoms and optionally having a substituent and/or a crosslinking group, or a crosslinking group.

The halogen atom is particularly preferably a fluorine atom.

When R⁶²¹, R⁶²², R⁶²³, and R⁶²⁴ are each independently a monovalent aromatic hydrocarbon group having 6 to 50 carbon atoms and optionally having a substituent and/or a crosslinking group, R⁶²¹, R⁶²², R⁶²³, and R⁶²⁴ may each be independently an aromatic hydrocarbon group having 6 to 50 carbon atoms and having both a substituent and a crosslinking group.

Preferably, R⁶²¹, R⁶²², R⁶²³, and R⁶²⁴ are each independently an aromatic hydrocarbon group having 6 to 50 carbon atoms and optionally having a crosslinking group or a crosslinking group.

The number of carbon atoms in the aromatic hydrocarbon group is preferably 6 to 50, more preferably 6 to 30, and still more preferably 6 to 18. Specific examples of the aromatic hydrocarbon group include: monovalent groups each including an aromatic hydrocarbon ring structure having generally 6 or more carbon atoms and having generally 30 or less carbon atoms, preferably 18 or less carbon atoms, and still more preferably 14 or less carbon atoms such as a benzene ring, a naphthalene ring, an anthracene ring, a tetraphenylene ring, a phenanthrene ring, a chrysene ring, a pyrene ring, a benzanthracene ring, and a perylene ring; and monovalent groups each having a structure obtained by bonding a plurality of structures selected from the above structures in a linear or branched manner. The structure including a plurality of aromatic hydrocarbon rings linked together is generally a structure including 2 to 8 aromatic hydrocarbon rings linked together and is preferably a structure including 2 to 5 aromatic hydrocarbon rings linked together. When a plurality of aromatic hydrocarbon rings are linked together, the aromatic hydrocarbon rings linked together may have the same structure or may have different structures.

Each of these aromatic hydrocarbon groups may optionally have a substituent and/or a crosslinking group. The optional substituent on each aromatic hydrocarbon group is as described above. Specifically, the optional substituent can be selected from the substituent group Z. Preferred substituents are the preferred substituents in the substituent group Z. The optional crosslinking group and crosslinking group on each aromatic hydrocarbon group is as described above. Specifically, the optional crosslinking group can be selected from the group T of crosslinking groups. Preferred crosslinking groups are the preferred crosslinking groups in the group T of crosslinking groups.

From the viewpoint of the solubility and durability of the compound, R⁶²¹, R⁶²², R⁶²³, and R⁶²⁴ each have preferably at least one partial structure selected from formulas (71-1) to (71-3) above, more preferably at least one partial structure selected from a 1,3-phenylene group, a 1,4-phenylene group, and formulas (71-1) and (71-2), and particularly preferably a 1,3-phenylene group, a 1,4-phenylene group, or a partial structure represented by formula (71-2-2).

### (Crosslinking Groups)

The compound represented by formula (71) has at least two crosslinking groups. The crosslinking groups are as described above. Specifically, each crosslinking group can be selected from the group T of crosslinking groups. Preferred crosslinking groups are the preferred crosslinking groups in the group T of crosslinking groups.

As for the positions of the crosslinking groups on the compound represented by formula (71), it is preferable that at least one R⁶²¹ and at least one R⁶²³ are each substituted with a crosslinking group or are each a crosslinking group, and it is more preferable that only two, i.e., one R⁶²¹ and one R⁶²³, are each substituted with a crosslinking group or are each a crosslinking group.

A structure in which R⁶²¹ and R⁶²³ each have a crosslinking group is the same as a structure in which R⁶²² and R⁶²⁴ each have a crosslinking group because of the symmetry of the compound represented by formula (71).

(n621, n622, n623, and n624)

n621, n622, n623, and n624 are each independently an integer of 0 to 4, provided that n621+n622+n623+n624 is 1 or more.

n621, n622, n623, and n624 are each independently an integer of 0 to 2 and are each more preferably 0 or 1.

Since the compound represented by formula (71) has crosslinking groups, n621 and n623 are each preferably 1 or more and are each preferably 2 or less and still more preferably 1. It is particularly preferable that n621 and n623 are each 1 and n622 and n624 are each 0.

In the compound represented by formula (71), it is particularly preferable that n621 and n623 are each 1, that n622 and n624 are each 0, and that R⁶²¹ and R⁶²³ are each independently an aromatic hydrocarbon group having 6 to 50 carbon atoms and substituted with a crosslinking group or a crosslinking group.

### <Carbazole Compound Represented by Formula (72)>

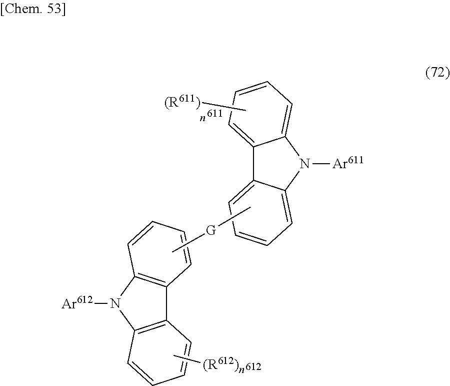

(In formula (72),
Ar⁶¹¹ and Ar⁶¹² each independently represent a divalent aromatic hydrocarbon group having 6 to 50 carbon atoms and optionally having a substituent and/or a crosslinking group;
R⁶¹¹ and R⁶¹² are each independently a deuterium atom, a halogen atom, a monovalent aromatic hydrocarbon group having 6 to 50 carbon atoms and optionally having a substituent and/or a crosslinking group, or a crosslinking group;
G represents a single bond or a divalent aromatic hydrocarbon group having 6 to 50 carbon atoms and optionally having a substituent and/or a crosslinking group;
n⁶¹¹ and n⁶¹² are each independently an integer of 0 to 4; and
the compound represented by formula (72) has at least two crosslinking groups.)
   (Ar⁶¹¹ and Ar⁶¹²)

Ar⁶¹¹ and Ar⁶¹² each independently represent a divalent aromatic hydrocarbon group having 6 to 50 carbon atoms and optionally having a substituent and/or a crosslinking group.

The number of carbon atoms in the aromatic hydrocarbon group is preferably 6 to 50, more preferably 6 to 30, and still more preferably 6 to 18. Specific examples of the aromatic hydrocarbon group include: monovalent groups each including an aromatic hydrocarbon structure having generally 6 or more carbon atoms and having generally 30 or less carbon atoms, preferably 18 or less carbon atoms, and still more preferably 14 or less carbon atoms such as a benzene ring, a naphthalene ring, an anthracene ring, a tetraphenylene ring, a phenanthrene ring, a chrysene ring, a pyrene ring, a benzanthracene ring, and a perylene ring; and monovalent groups in which a plurality of structures selected from the above structures are bonded together in a linear or branched manner. The structure including a plurality of aromatic hydrocarbon rings linked together is generally a structure including 2 to 8 aromatic hydrocarbon rings linked together and preferably a structure including 2 to 5 aromatic hydrocarbon rings linked together. When a plurality of aromatic hydrocarbon rings are linked together, the aromatic hydrocarbon rings linked together may have the same structure or different structures.

Ar⁶¹¹ and Ar⁶¹² are each independently preferably
a phenyl group,
a monovalent group in which one or a plurality of benzene rings and at least one naphthalene ring are bonded together in a linear or branched manner,
a monovalent group in which one or a plurality of benzene rings and at least one phenanthrene ring are bonded together in a linear or branched manner, or
a monovalent group in which one or a plurality of benzene rings and at least one tetraphenylene ring are bonded together in a linear or branched manner, and
more preferably a monovalent group in which one or a plurality of benzene rings are bonded together in a linear or branched manner. In all the cases, the rings may be bonded in any order.

As described above, the number of benzene, naphthalene, phenanthrene, and tetraphenylene rings bonded together is generally 2 to 8 and preferably 2 to 5. Particularly preferred are a monovalent structure in which 1 to 4 benzene rings are linked together, a monovalent structure in which 1 to 4 benzene rings and a naphthalene ring are linked together, a monovalent structure in which 1 to 4 benzene rings and a phenanthrene ring are linked together, and a monovalent structure in which 1 to 4 benzene rings and a tetraphenylene ring are linked together.

Each of these aromatic hydrocarbon groups may optionally have a substituent and/or a crosslinking group. The optional substituent on each aromatic hydrocarbon group is as described above. Specifically, each optional substituent can be selected from the substituent group Z. Preferred substituents are the preferred substituents in the substituent group Z. The optional crosslinking groups and crosslinking groups on the aromatic hydrocarbon groups are as described above. Specifically, each crosslinking group can be selected from the group T of crosslinking groups. Preferred crosslinking groups are the preferred crosslinking groups in the group T of crosslinking groups.

Ar⁶¹¹ and Ar⁶¹² are each independently preferably a phenyl group having a crosslinking group or a monovalent group that includes a plurality of benzene rings bonded together in a linear or branched manner and that has a crosslinking group because the stability of the film quality is high.

From the viewpoint of the solubility and durability of the compound, it is preferable that at least one of Ar⁶¹¹ and Ar⁶¹² has at least one partial structure selected from the following formulas (72-1) to (72-6).

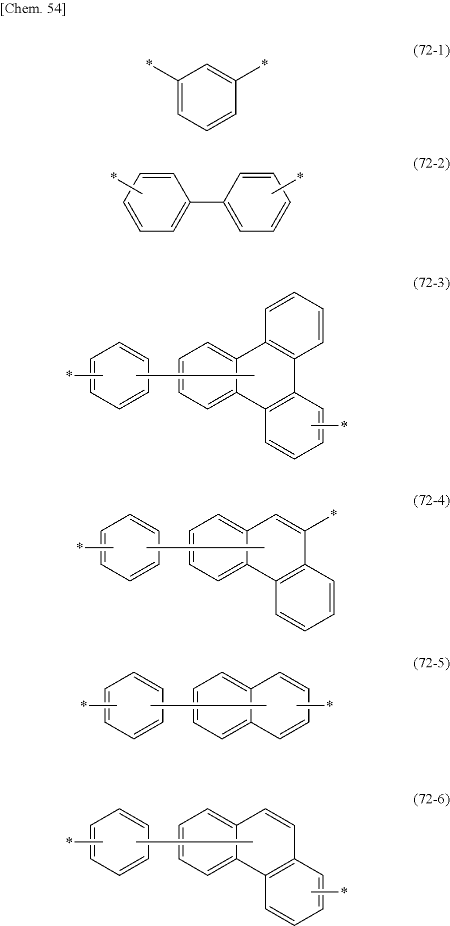

(In each of formulas (72-1) to (72-6) above, two *'s each represent a bond to an adjacent structure or a hydrogen atom, and at least one of the two *'s represents a position of bonding to an adjacent structure.)

In the following description, the definition of the * is the same unless otherwise specified.

More preferably, at least one of Ar⁶¹¹ and Ar⁶¹² has at least one partial structure selected from formulas (72-1) to (72-4).

Still more preferably, each of Ar⁶¹¹ and Ar⁶¹² has at least one partial structure selected from formulas (72-1) to (72-3).

Particularly preferably, each of Ar⁶¹¹ and Ar⁶¹² has at least one partial structure selected from formulas (72-1) to (72-2).

Formula (72-2) is preferably the following formula (72-2-2).

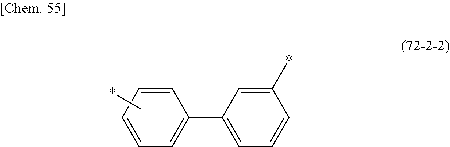

Formula (72-2) is still more preferably the following formula (72-2-3).

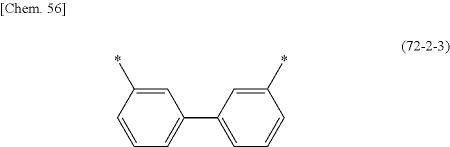 [SMILES: *c1cccc(-c2cccc(*)c2)c1]

From the viewpoint of the solubility and durability of the compound, a preferred partial structure included in at least one of Ar⁶¹¹ and Ar⁶¹² is a partial structure including a partial structure represented by formula (72-1) and a partial structure represented by formula (72-2).

(R⁶¹¹ and R⁶¹²)

R⁶¹¹ and R⁶¹² are each independently a deuterium atom, a halogen atom such as a fluorine atom, or a monovalent aromatic hydrocarbon having 6 to 30 carbon atoms and optionally having a substituent and/or a crosslinking group.

The aromatic hydrocarbon group is a monovalent group having an aromatic hydrocarbon structure having preferably 6 to 30 carbon atoms, more preferably 6 to 18 carbon atoms, and still more preferably 6 to 10 carbon atoms.

The aromatic hydrocarbon group may optionally have a substituent and/or a crosslinking group. The optional substituent on the aromatic hydrocarbon group is as described above. Specifically, the optional substituent can be selected from the substituent group Z. Preferred substituents are the preferred substituents in the substituent group Z. The optional crosslinking group and crosslinking groups on the aromatic hydrocarbon group is as described above. Specifically, the crosslinking group can be selected from the group T of crosslinking groups. Preferred crosslinking groups are the preferred crosslinking groups in the group T of crosslinking groups.

(n⁶¹¹ and n⁶¹²)

n⁶¹¹ and n⁶¹² are each independently an integer of 0 to 4. n⁶¹¹ and n⁶¹² are each independently preferably 0 to 2 and more preferably 0 or 1.

### (Substituents and Crosslinking Groups)

When Ar⁶¹¹, Ar⁶¹², R⁶¹¹, and R⁶¹² are each a monovalent or divalent aromatic hydrocarbon group, the optional substituent is preferably a substituent selected from the substituent group Z. The optional crosslinking group is preferably a crosslinking group selected from the group T of crosslinking groups. As for the positions of the crosslinking groups, it is preferable that Ar⁶¹¹ and a structure selected from Ar⁶¹¹ and R⁶¹¹ when n⁶¹¹ is 1 or more each have at least one crosslinking group and that Ar⁶¹² and a structure selected from Ar⁶¹² and R⁶¹² when n⁶¹² is 1 or more each have at least one crosslinking group, and it is more preferable that Ar⁶¹¹ and Ar⁶¹² each have at least one crosslinking group. The number of crosslinking groups included in the compound represented by formula (72) is preferably 2 or more and 4 or less, more preferably 2 or more and 3 or less, and most preferably 2.

### (G)

G represents a single bond or a divalent aromatic hydrocarbon group having 6 to 50 carbon atoms and optionally having a substituent and/or a crosslinking group.

The number of carbon atoms in the aromatic hydrocarbon group is preferably 6 to 50, more preferably 6 to 30, and still more preferably 6 to 18. Specific examples of the aromatic hydrocarbon group include: divalent groups each having an aromatic hydrocarbon structure having generally 6 or more carbon atoms and having generally 30 or less carbon atoms, preferably 18 or less carbon atoms, and still more preferably 14 or less carbon atoms such as a benzene ring, a naphthalene ring, an anthracene ring, a tetraphenylene ring, a phenanthrene ring, a chrysene ring, a pyrene ring, a benzanthracene ring, and a perylene ring; and divalent groups in which a plurality of structures selected from the above structures are bonded together in a linear or branched manner. The structure including a plurality of aromatic hydrocarbon rings linked together is generally a structure including 2 to 8 aromatic hydrocarbon rings linked together and preferably a structure including 2 to 5 aromatic hydrocarbon rings linked together. When a plurality of aromatic hydrocarbon rings are linked together, the plurality of aromatic hydrocarbon rings may have the same structure or different structures.

G is preferably
a single bond,
a phenylene group,
a divalent group in which a plurality of benzene rings are bonded together in a linear or branched manner,
a divalent group in which one or a plurality of benzene rings and at least one naphthalene ring are bonded together in a linear or branched manner,
a divalent group in which one or a plurality of benzene rings and at least one phenanthrene ring are bonded together in a linear or branched manner, or
a divalent group in which one or a plurality of benzene rings and at least one tetraphenylene ring are bonded together in a linear or branched manner, and
more preferably a divalent group in which a plurality of benzene rings are bonded together in a linear or branched manner. In all the cases, the rings may be bonded in any order.

As described above, the number of benzene, naphthalene, phenanthrene, and tetraphenylene rings bonded together is generally 2 to 8 and preferably 2 to 5. Particularly preferred are a divalent structure in which 1 to 4 benzene rings are linked together, a divalent structure in which 1 to 4 benzene rings and a naphthalene ring are linked together, a divalent structure in which 1 to 4 benzene rings and a phenanthrene ring are linked together, and a divalent structure in which 1 to 4 benzene rings and a tetraphenylene ring are linked together.

Each of these aromatic hydrocarbon groups may optionally have a substituent and/or a crosslinking group. The optional substituent on each aromatic hydrocarbon group is as described above. Specifically, the optional substituent can be selected from the substituent group Z. Preferred substituents are the preferred substituents in the substituent group Z. The optional crosslinking group and crosslinking groups on each aromatic hydrocarbon group is as described above. Specifically, the crosslinking group can be selected from the group T of crosslinking groups. Preferred crosslinking groups are the preferred crosslinking groups in the group T of crosslinking groups.

G is preferably a single bond because the stability and transportability during charge transport are good and the performance of the element is improved.

### (Molecular Weight)

From the viewpoint of thermal stability, the molecular weight of the carbazole compound in the invention is preferably 600 or more, more preferably 800 or more, still more preferably 1000 or more, and particularly preferably 1200 or more and is preferably 5000 or less, more preferably 4000 or less, still more preferably 3000 or less, and particularly preferably 2500 or less.

### (Specific Examples)

Specific examples of the carbazole compound in the invention are shown below. However, the invention is not limited thereto.

### :Specific Examples of the Carbazole Compound Represented by Formula (71):

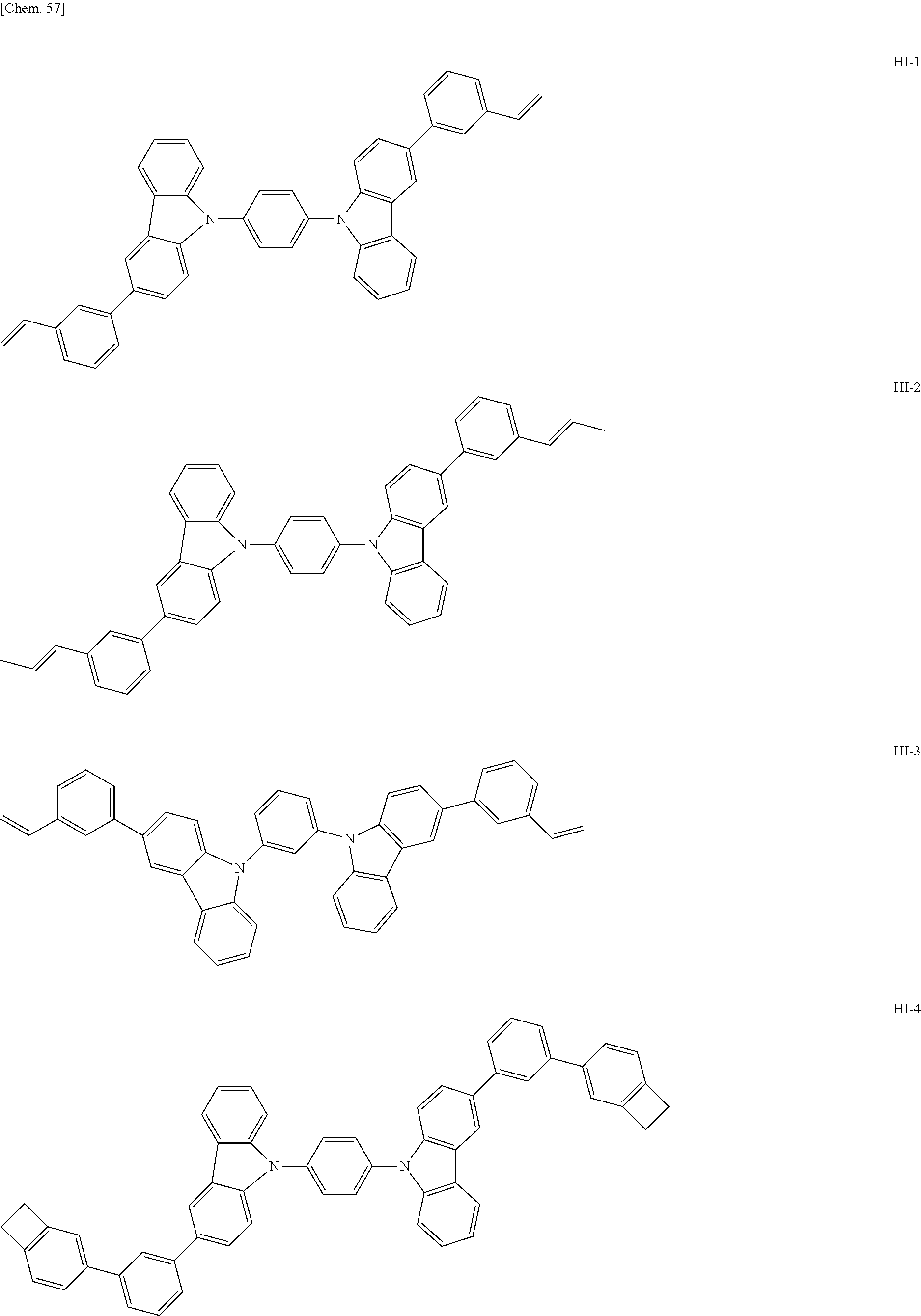

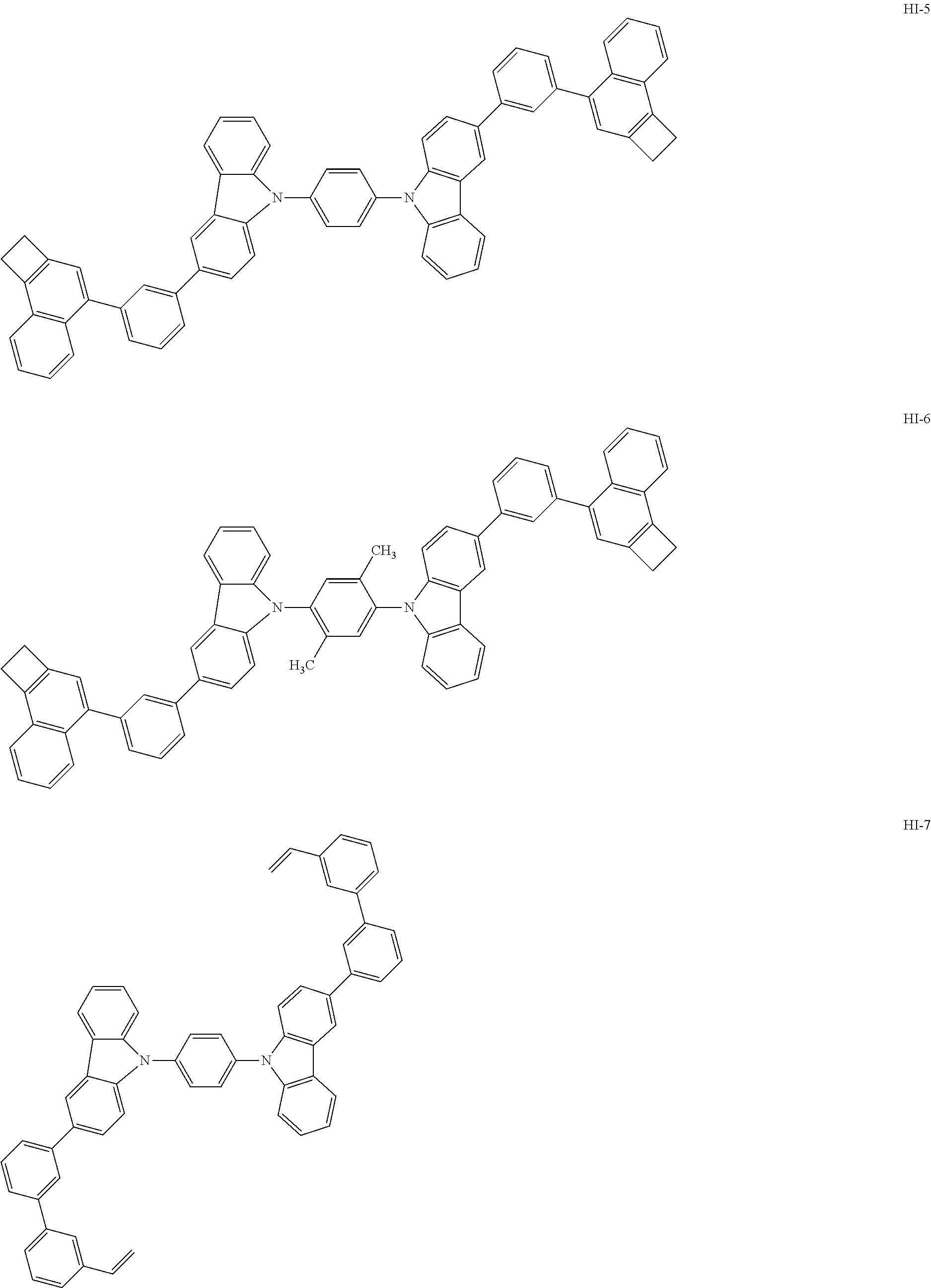 [SMILES: c1cc(-c2ccc3c(c2)c2ccccc2n3-c2ccc(-n3c4ccccc4c4cc(-c5cccc(-c6cc7c(c8ccccc68)CC7)c5)ccc43)cc2)cc(-c2cc3c(c4ccccc24)CC3)c1 ; Cc1cc(-n2c3ccccc3c3cc(-c4cccc(-c5cc6c(c7ccccc57)CC6)c4)ccc32)c(C)cc1-n1c2ccccc2c2cc(-c3cccc(-c4cc5c(c6ccccc46)CC5)c3)ccc21 ; C=Cc1cccc(-c2cccc(-c3ccc4c(c3)c3ccccc3n4-c3ccc(-n4c5ccccc5c5cc(-c6cccc(-c7cccc(C=C)c7)c6)ccc54)cc3)c2)c1]

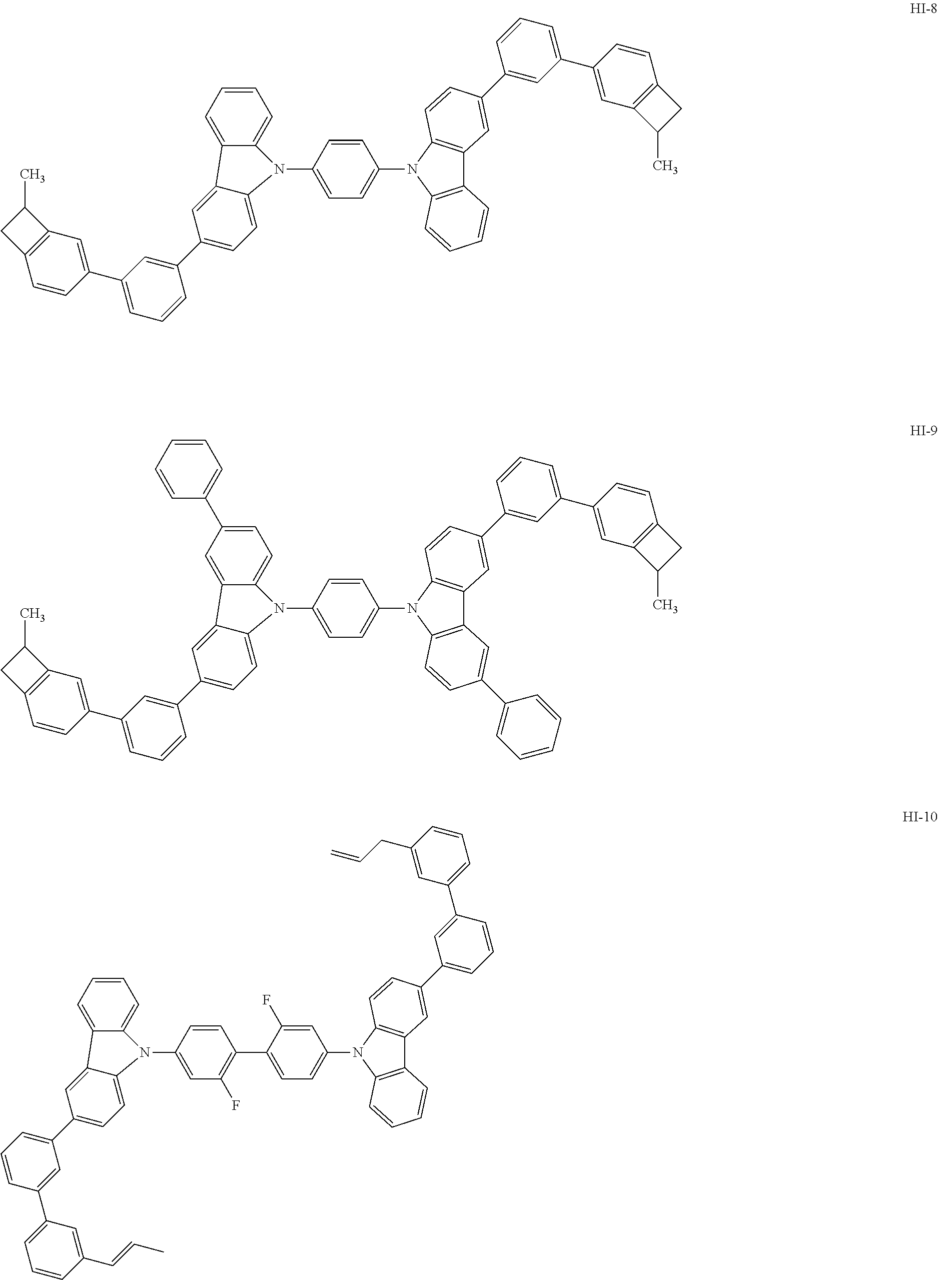 [SMILES: CC1Cc2ccc(-c3cccc(-c4ccc5c(c4)c4ccccc4n5-c4ccc(-n5c6ccccc6c6cc(-c7cccc(-c8ccc9c(c8)C(C)C9)c7)ccc65)cc4)c3)cc21 ; CC1Cc2ccc(-c3cccc(-c4ccc5c(c4)c4cc(-c6ccccc6)ccc4n5-c4ccc(-n5c6ccc(-c7ccccc7)cc6c6cc(-c7cccc(-c8ccc9c(c8)C(C)C9)c7)ccc65)cc4)c3)cc21 ; C=CCc1cccc(-c2cccc(-c3ccc4c(c3)c3ccccc3n4-c3ccc(-c4ccc(-n5c6ccccc6c6cc(-c7cccc(-c8cccc(/C=C/C)c8)c7)ccc65)cc4F)c(F)c3)c2)c1]

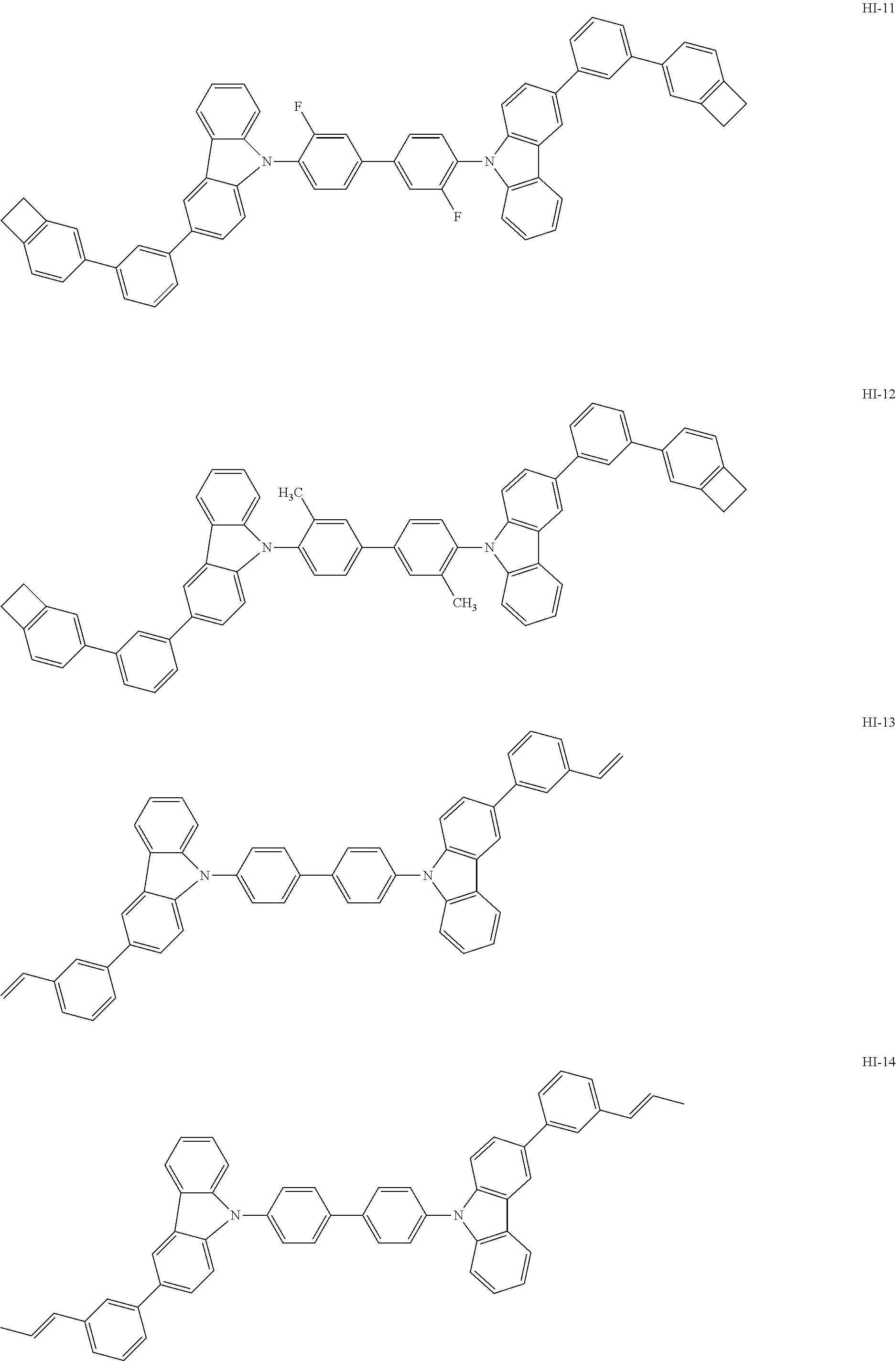

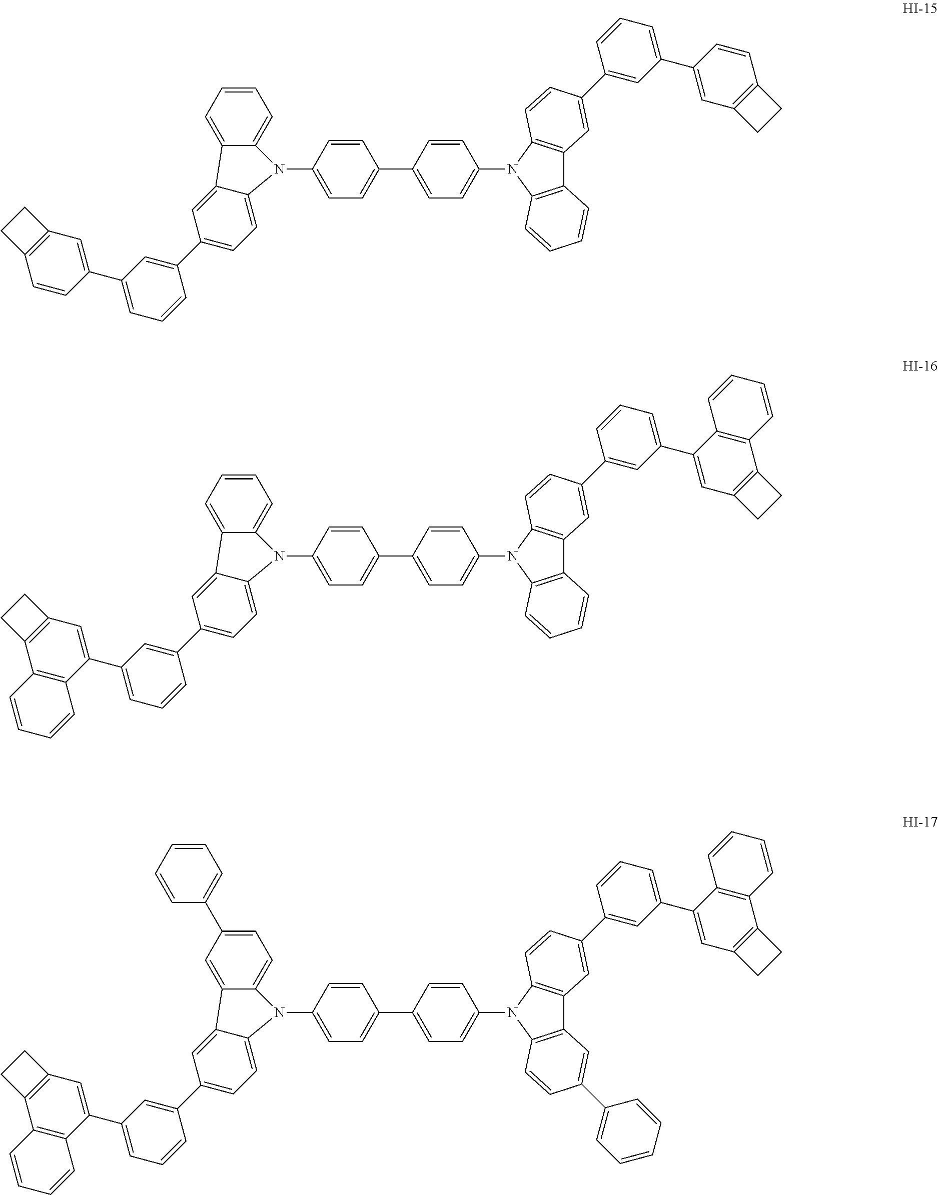 [SMILES: c1cc(-c2ccc3c(c2)CC3)cc(-c2ccc3c(c2)c2ccccc2n3-c2ccc(-c3ccc(-n4c5ccccc5c5cc(-c6cccc(-c7ccc8c(c7)CC8)c6)ccc54)cc3)cc2)c1 ; c1cc(-c2ccc3c(c2)c2ccccc2n3-c2ccc(-c3ccc(-n4c5ccccc5c5cc(-c6cccc(-c7cc8c(c9ccccc79)CC8)c6)ccc54)cc3)cc2)cc(-c2cc3c(c4ccccc24)CC3)c1 ; c1ccc(-c2ccc3c(c2)c2cc(-c4cccc(-c5cc6c(c7ccccc57)CC6)c4)ccc2n3-c2ccc(-c3ccc(-n4c5ccc(-c6ccccc6)cc5c5cc(-c6cccc(-c7cc8c(c9ccccc79)CC8)c6)ccc54)cc3)cc2)cc1]

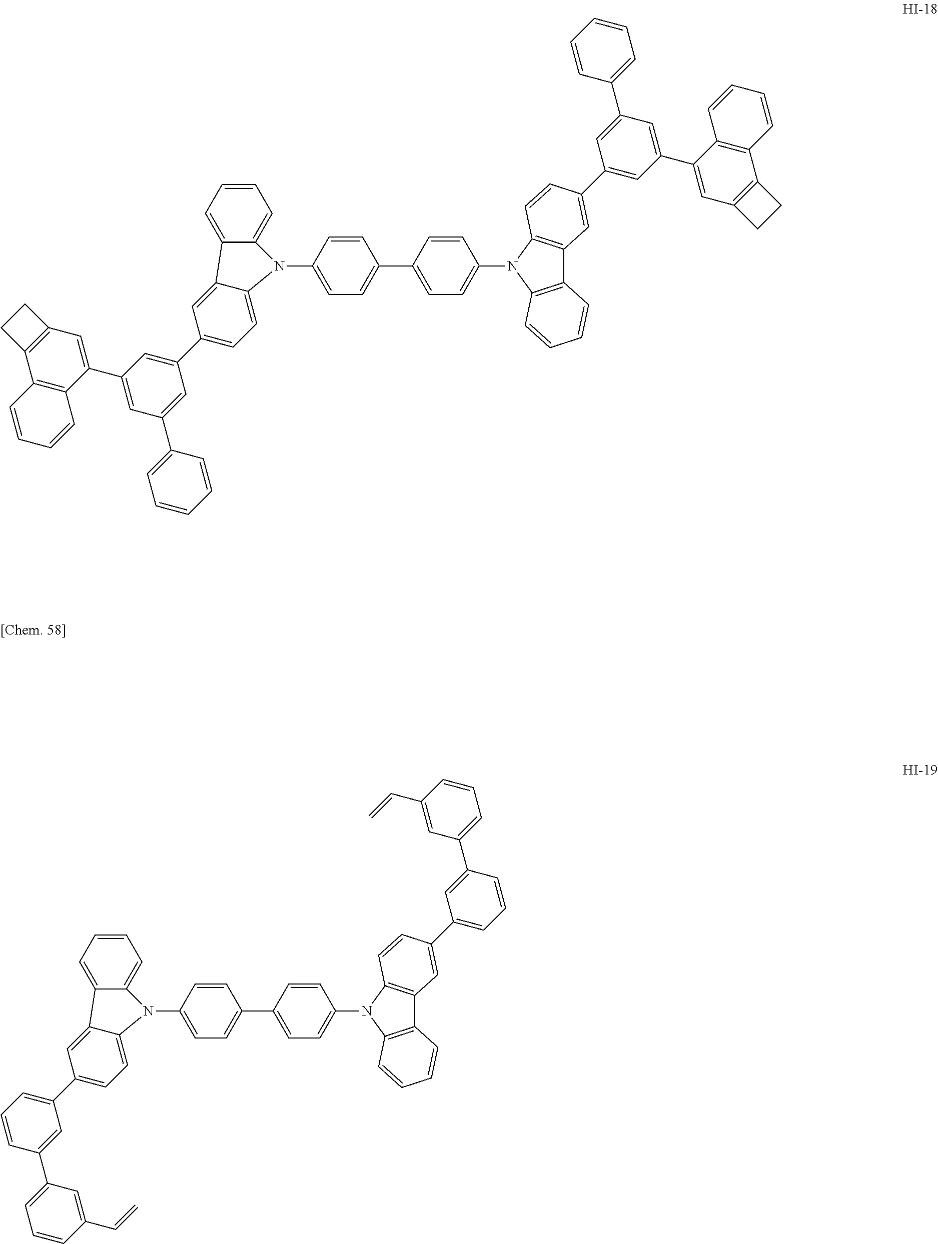 [SMILES: c1ccc(-c2cc(-c3ccc4c(c3)c3ccccc3n4-c3ccc(-c4ccc(-n5c6ccccc6c6cc(-c7cc(-c8ccccc8)cc(-c8cc9c(c%10ccccc8%10)CC9)c7)ccc65)cc4)cc3)cc(-c3cc4c(c5ccccc35)CC4)c2)cc1 ; C=Cc1cccc(-c2cccc(-c3ccc4c(c3)c3ccccc3n4-c3ccc(-c4ccc(-n5c6ccccc6c6cc(-c7cccc(-c8cccc(C=C)c8)c7)ccc65)cc4)cc3)c2)c1]

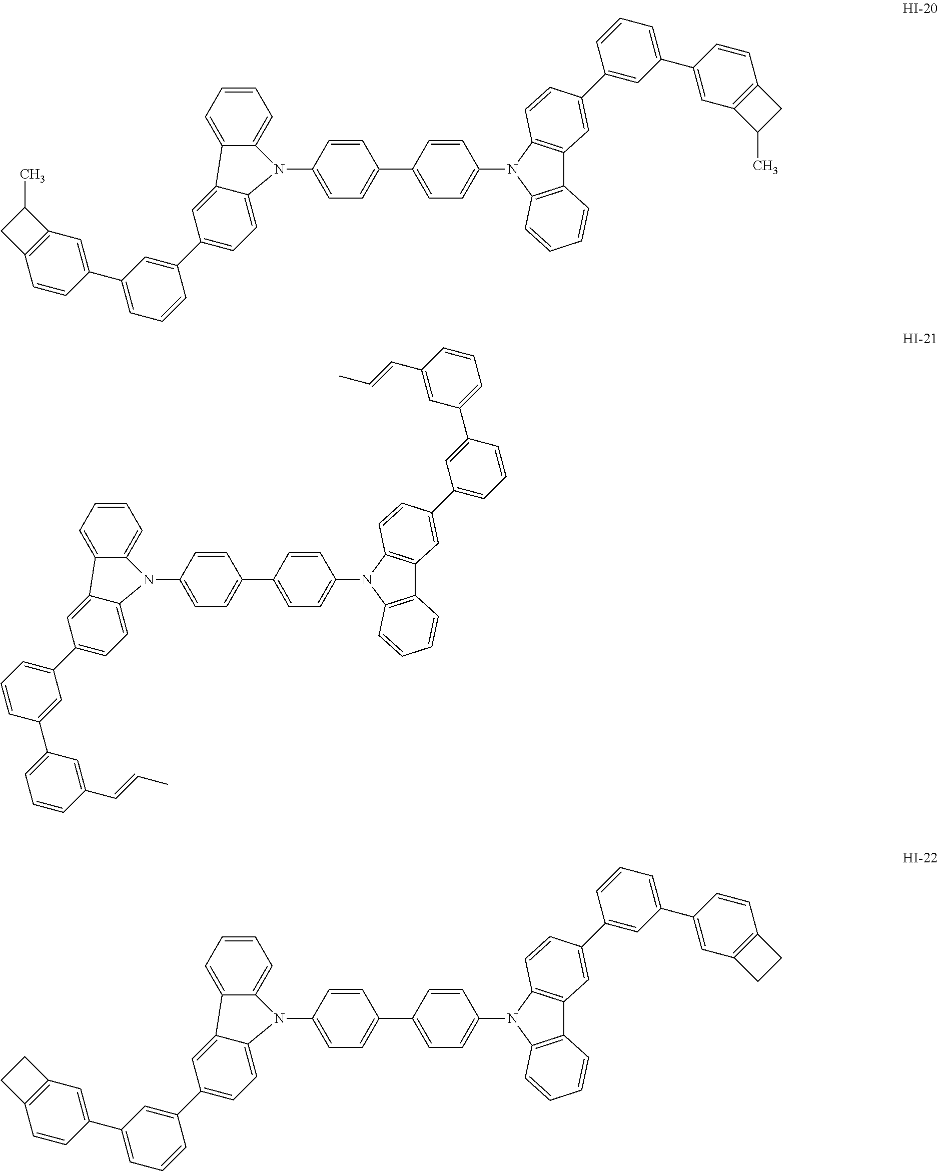 [SMILES: CC1Cc2ccc(-c3cccc(-c4ccc5c(c4)c4ccccc4n5-c4ccc(-c5ccc(-n6c7ccccc7c7cc(-c8cccc(-c9ccc%10c(c9)C(C)C%10)c8)ccc76)cc5)cc4)c3)cc21 ; C/C=C/c1cccc(-c2cccc(-c3ccc4c(c3)c3ccccc3n4-c3ccc(-c4ccc(-n5c6ccccc6c6cc(-c7cccc(-c8cccc(/C=C/C)c8)c7)ccc65)cc4)cc3)c2)c1 ; c1cc(-c2ccc3c(c2)CC3)cc(-c2ccc3c(c2)c2ccccc2n3-c2ccc(-c3ccc(-n4c5ccccc5c5cc(-c6cccc(-c7ccc8c(c7)CC8)c6)ccc54)cc3)cc2)c1]

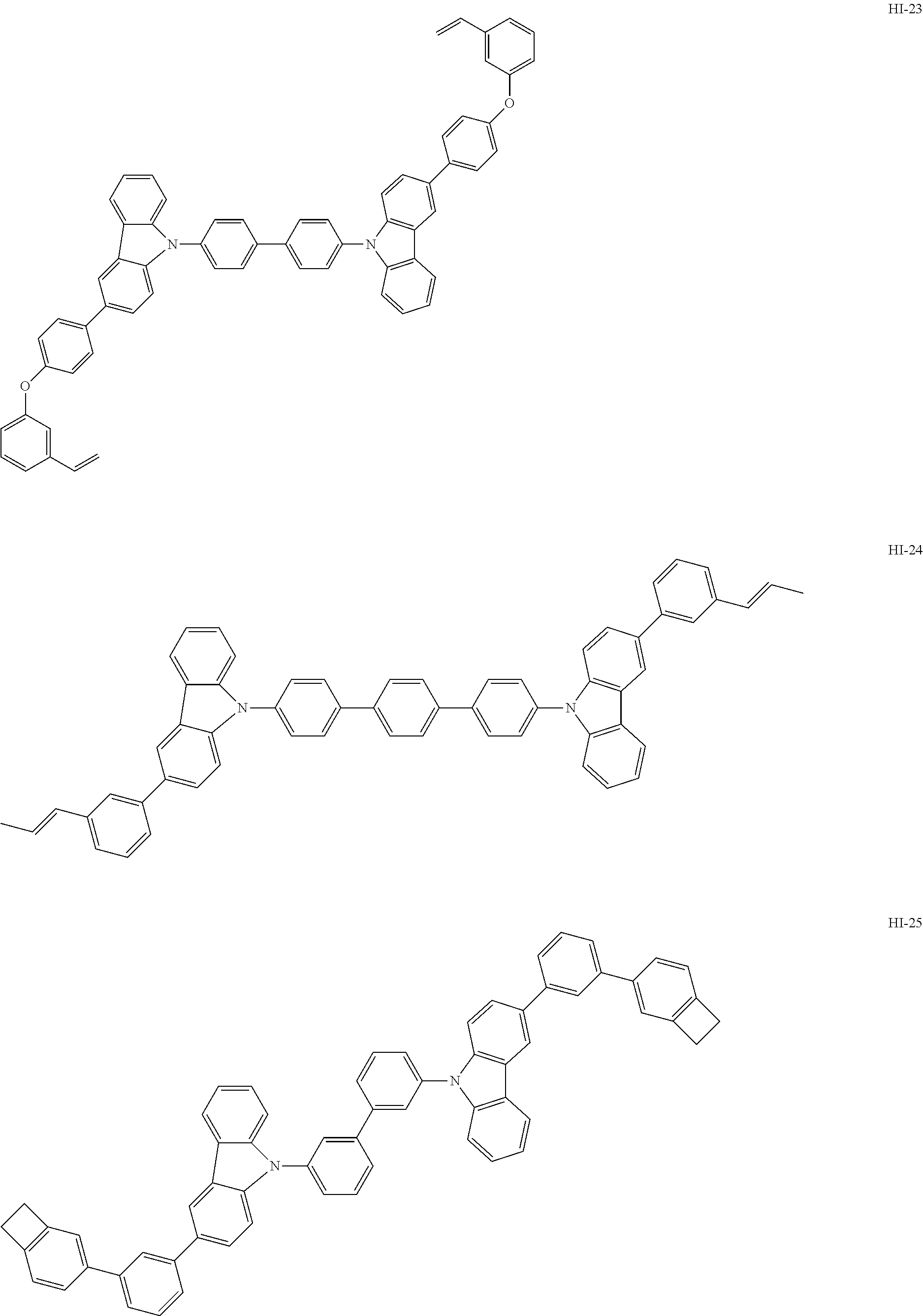 [SMILES: C=Cc1cccc(Oc2ccc(-c3ccc4c(c3)c3ccccc3n4-c3ccc(-c4ccc(-n5c6ccccc6c6cc(-c7ccc(Oc8cccc(C=C)c8)cc7)ccc65)cc4)cc3)cc2)c1 ; C/C=C/c1cccc(-c2ccc3c(c2)c2ccccc2n3-c2ccc(-c3ccc(-c4ccc(-n5c6ccccc6c6cc(-c7cccc(/C=C/C)c7)ccc65)cc4)cc3)cc2)c1 ; c1cc(-c2ccc3c(c2)CC3)cc(-c2ccc3c(c2)c2ccccc2n3-c2cccc(-c3cccc(-n4c5ccccc5c5cc(-c6cccc(-c7ccc8c(c7)CC8)c6)ccc54)c3)c2)c1]

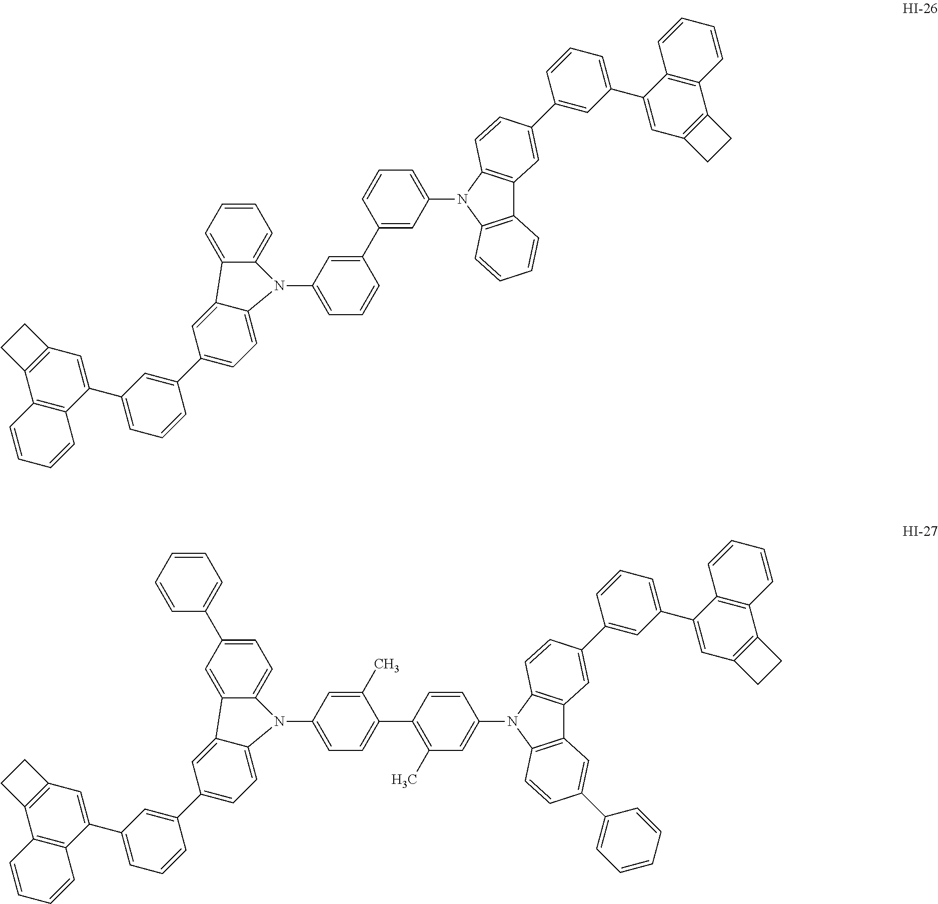 [SMILES: c1cc(-c2ccc3c(c2)c2ccccc2n3-c2cccc(-c3cccc(-n4c5ccccc5c5cc(-c6cccc(-c7cc8c(c9ccccc79)CC8)c6)ccc54)c3)c2)cc(-c2cc3c(c4ccccc24)CC3)c1 ; Cc1cc(-n2c3ccc(-c4ccccc4)cc3c3cc(-c4cccc(-c5cc6c(c7ccccc57)CC6)c4)ccc32)ccc1-c1ccc(-n2c3ccc(-c4ccccc4)cc3c3cc(-c4cccc(-c5cc6c(c7ccccc57)CC6)c4)ccc32)cc1C]

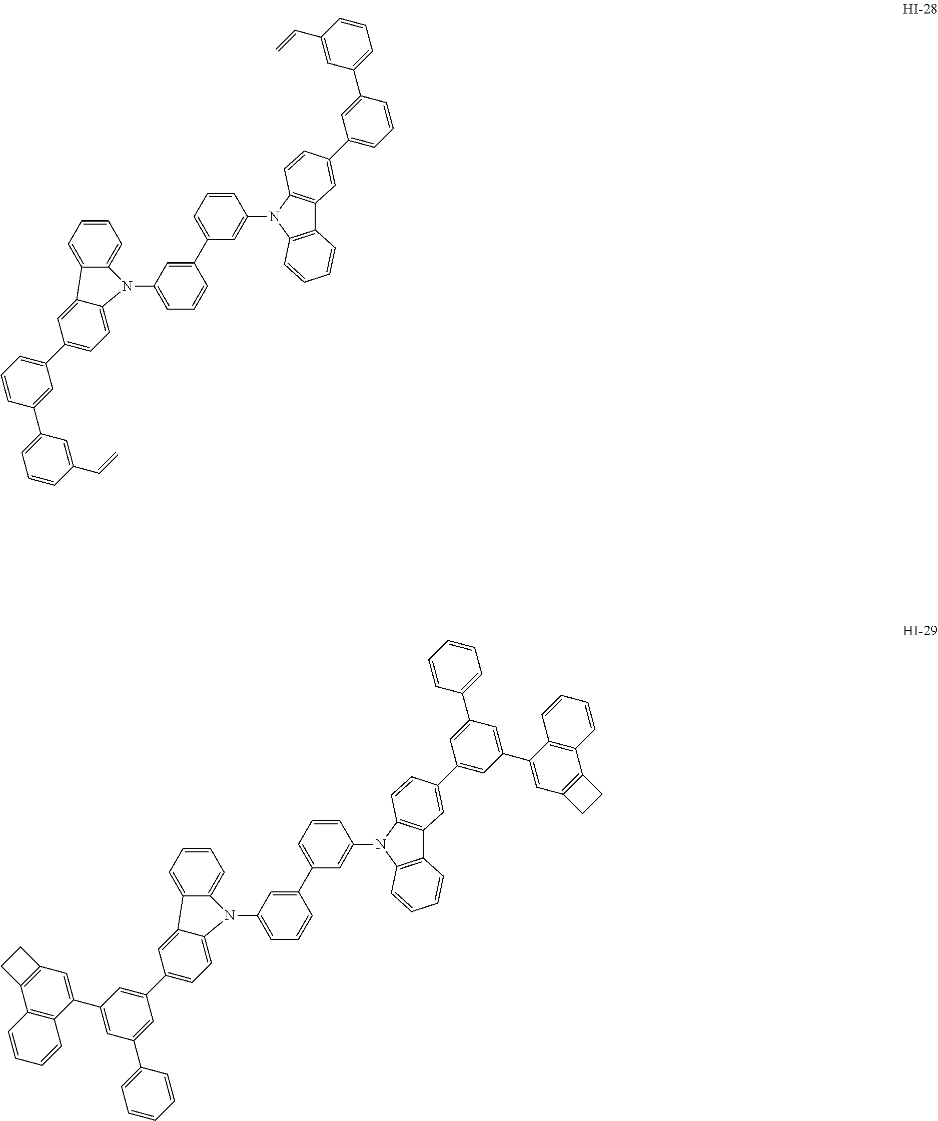 [SMILES: C=Cc1cccc(-c2cccc(-c3ccc4c(c3)c3ccccc3n4-c3cccc(-c4cccc(-n5c6ccccc6c6cc(-c7cccc(-c8cccc(C=C)c8)c7)ccc65)c4)c3)c2)c1 ; c1ccc(-c2cc(-c3ccc4c(c3)c3ccccc3n4-c3cccc(-c4cccc(-n5c6ccccc6c6cc(-c7cc(-c8ccccc8)cc(-c8cc9c(c%10ccccc8%10)CC9)c7)ccc65)c4)c3)cc(-c3cc4c(c5ccccc35)CC4)c2)cc1]

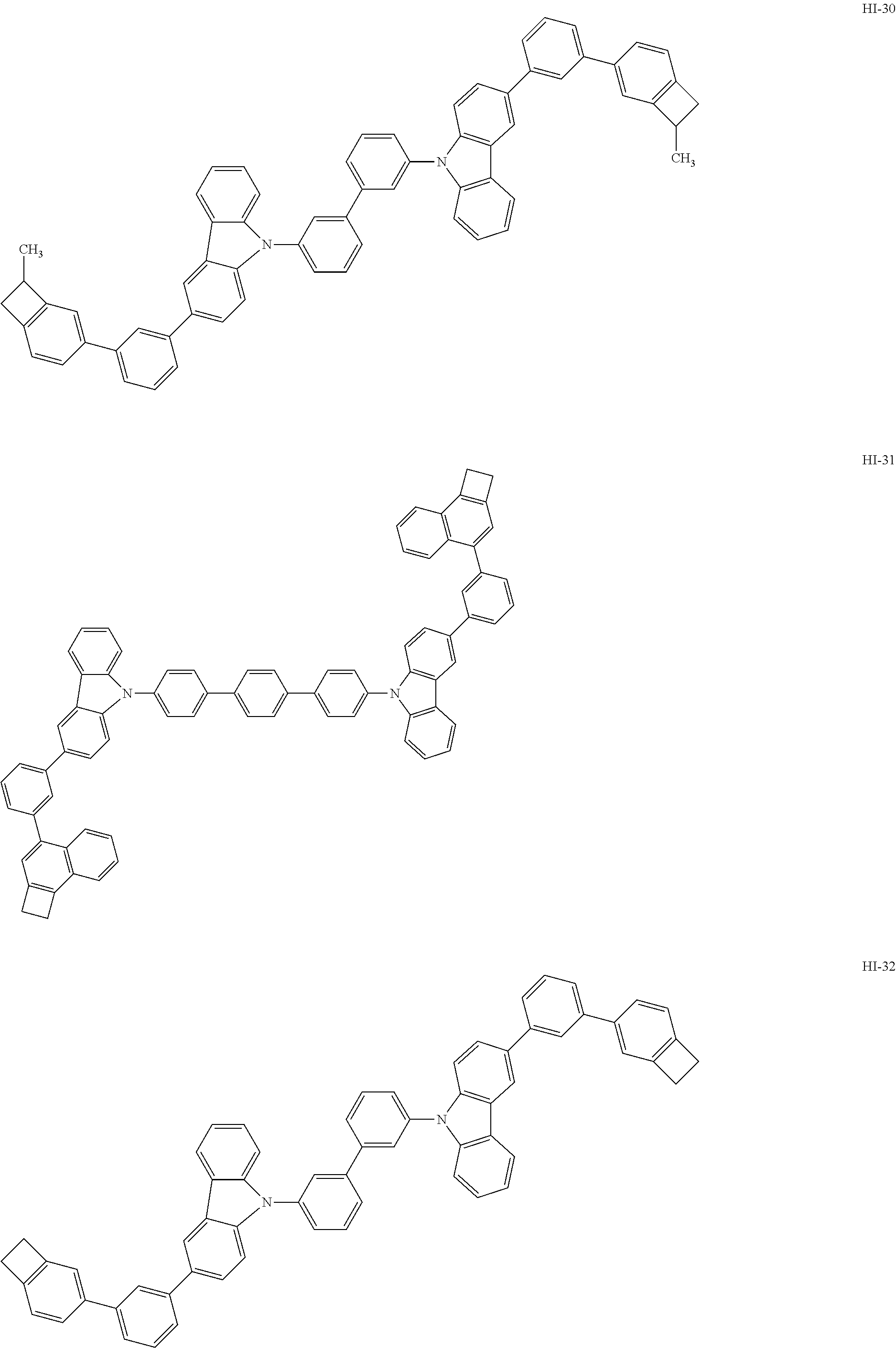 [SMILES: CC1Cc2ccc(-c3cccc(-c4ccc5c(c4)c4ccccc4n5-c4cccc(-c5cccc(-n6c7ccccc7c7cc(-c8cccc(-c9ccc%10c(c9)C(C)C%10)c8)ccc76)c5)c4)c3)cc21 ; c1cc(-c2ccc3c(c2)c2ccccc2n3-c2ccc(-c3ccc(-c4ccc(-n5c6ccccc6c6cc(-c7cccc(-c8cc9c(c%10ccccc8%10)CC9)c7)ccc65)cc4)cc3)cc2)cc(-c2cc3c(c4ccccc24)CC3)c1 ; c1cc(-c2ccc3c(c2)CC3)cc(-c2ccc3c(c2)c2ccccc2n3-c2cccc(-c3cccc(-n4c5ccccc5c5cc(-c6cccc(-c7ccc8c(c7)CC8)c6)ccc54)c3)c2)c1]

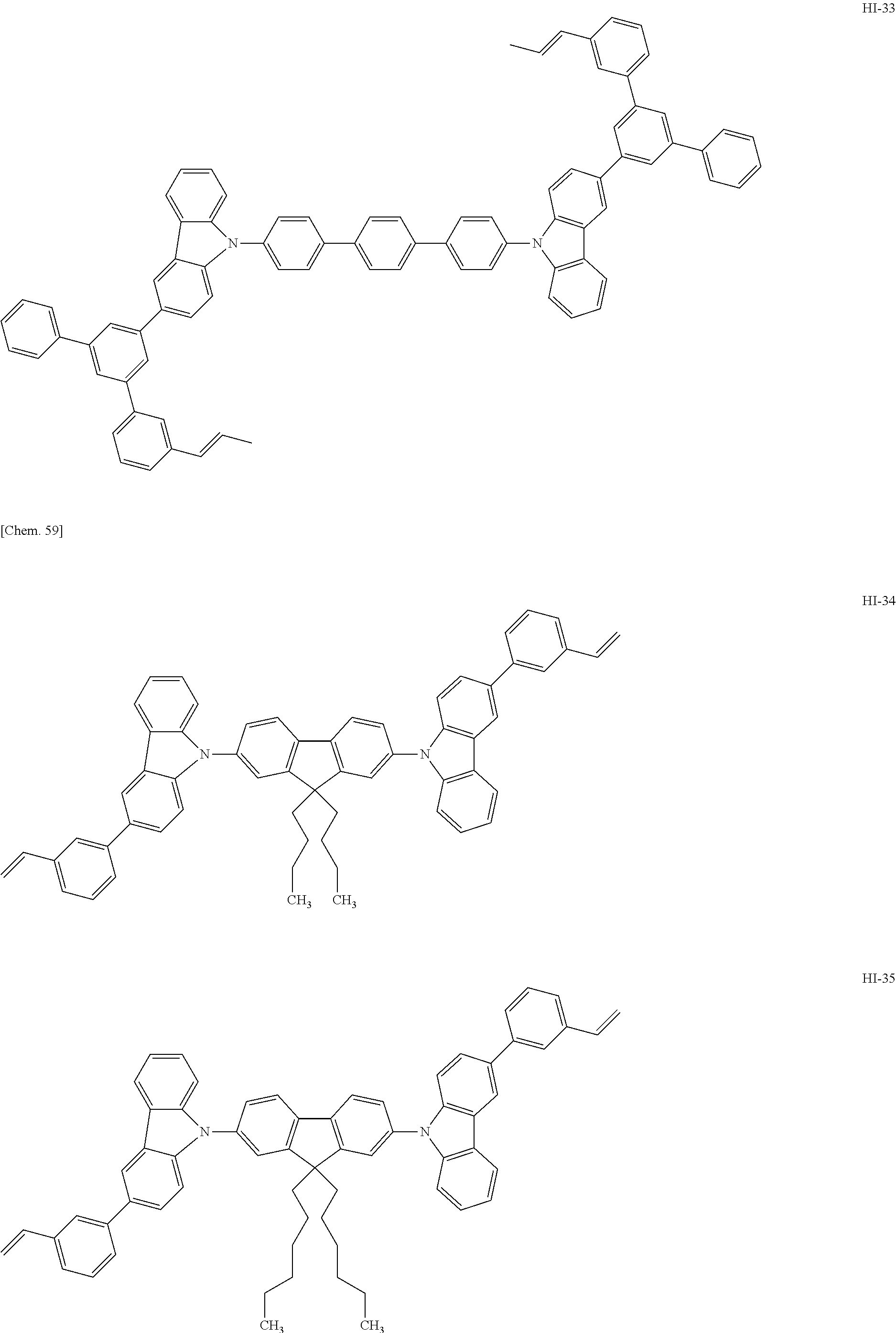

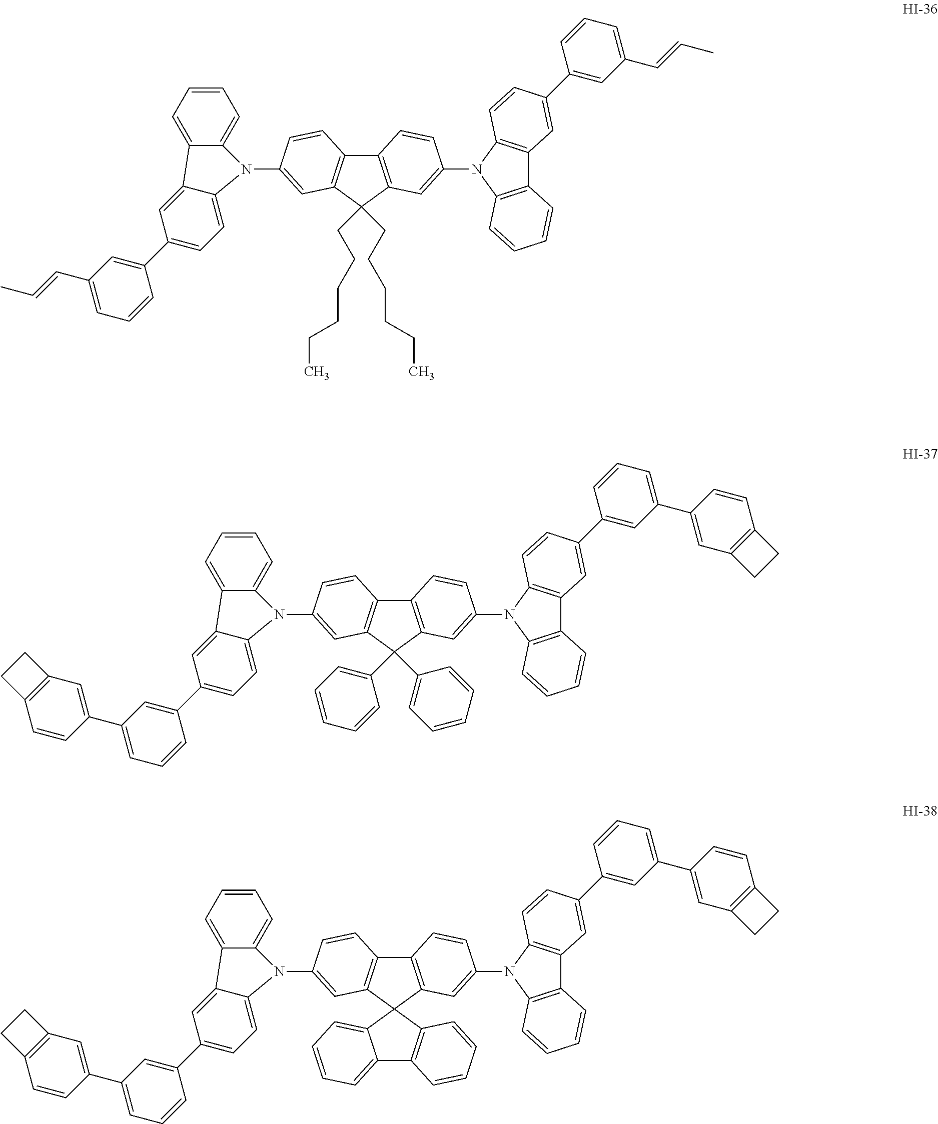 [SMILES: C/C=C/c1cccc(-c2ccc3c(c2)c2ccccc2n3-c2ccc3c(c2)C(CCCCCC)(CCCCCC)c2cc(-n4c5ccccc5c5cc(-c6cccc(/C=C/C)c6)ccc54)ccc2-3)c1 ; c1ccc(C2(c3ccccc3)c3cc(-n4c5ccccc5c5cc(-c6cccc(-c7ccc8c(c7)CC8)c6)ccc54)ccc3-c3ccc(-n4c5ccccc5c5cc(-c6cccc(-c7ccc8c(c7)CC8)c6)ccc54)cc32)cc1 ; c1cc(-c2ccc3c(c2)CC3)cc(-c2ccc3c(c2)c2ccccc2n3-c2ccc3c(c2)C2(c4ccccc4-c4ccccc42)c2cc(-n4c5ccccc5c5cc(-c6cccc(-c7ccc8c(c7)CC8)c6)ccc54)ccc2-3)c1]

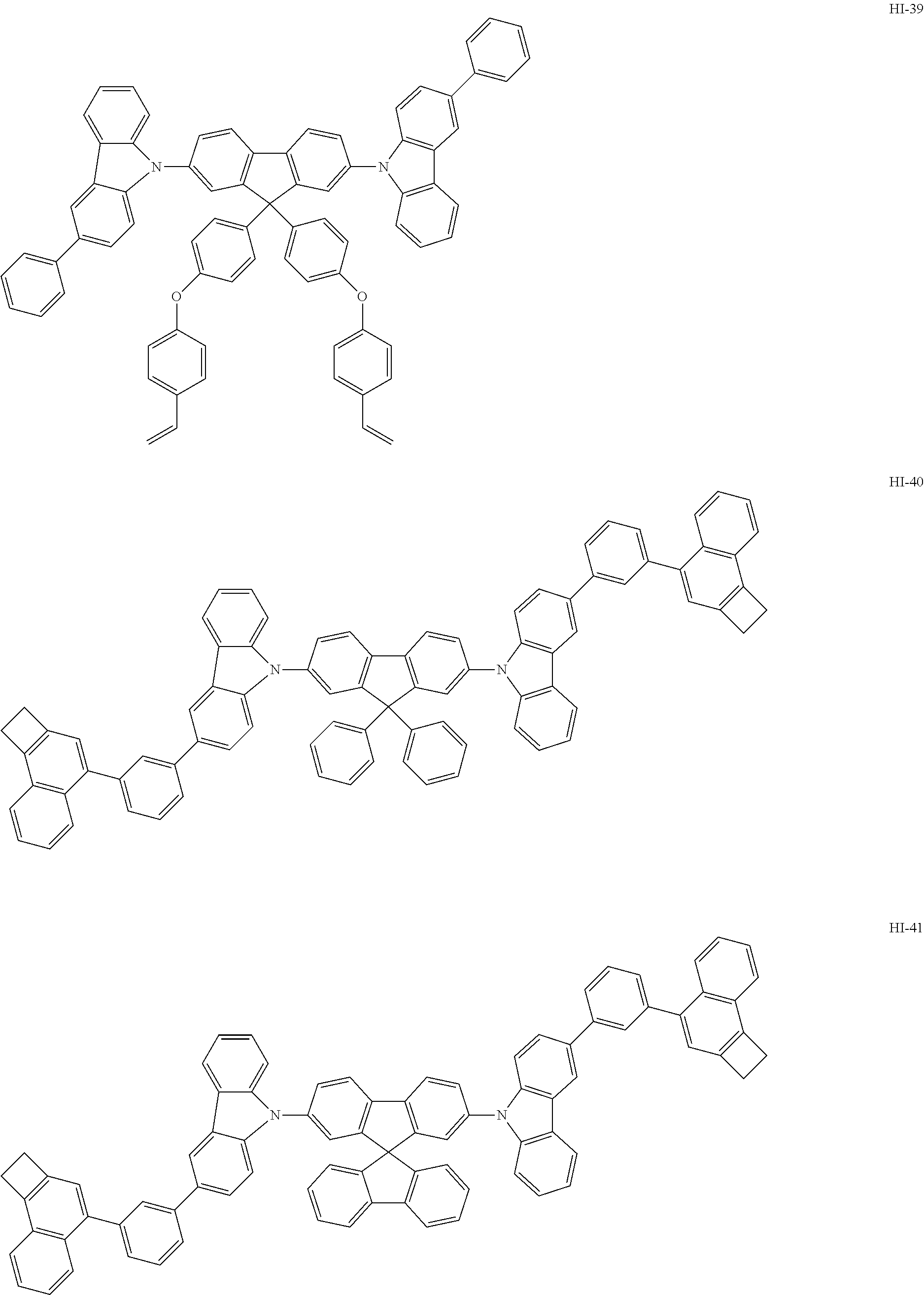 [SMILES: C=Cc1ccc(Oc2ccc(C3(c4ccc(Oc5ccc(C=C)cc5)cc4)c4cc(-n5c6ccccc6c6cc(-c7ccccc7)ccc65)ccc4-c4ccc(-n5c6ccccc6c6cc(-c7ccccc7)ccc65)cc43)cc2)cc1 ; c1ccc(C2(c3ccccc3)c3cc(-n4c5ccccc5c5cc(-c6cccc(-c7cc8c(c9ccccc79)CC8)c6)ccc54)ccc3-c3ccc(-n4c5ccccc5c5cc(-c6cccc(-c7cc8c(c9ccccc79)CC8)c6)ccc54)cc32)cc1 ; c1cc(-c2ccc3c(c2)c2ccccc2n3-c2ccc3c(c2)C2(c4ccccc4-c4ccccc42)c2cc(-n4c5ccccc5c5cc(-c6cccc(-c7cc8c(c9ccccc79)CC8)c6)ccc54)ccc2-3)cc(-c2cc3c(c4ccccc24)CC3)c1]

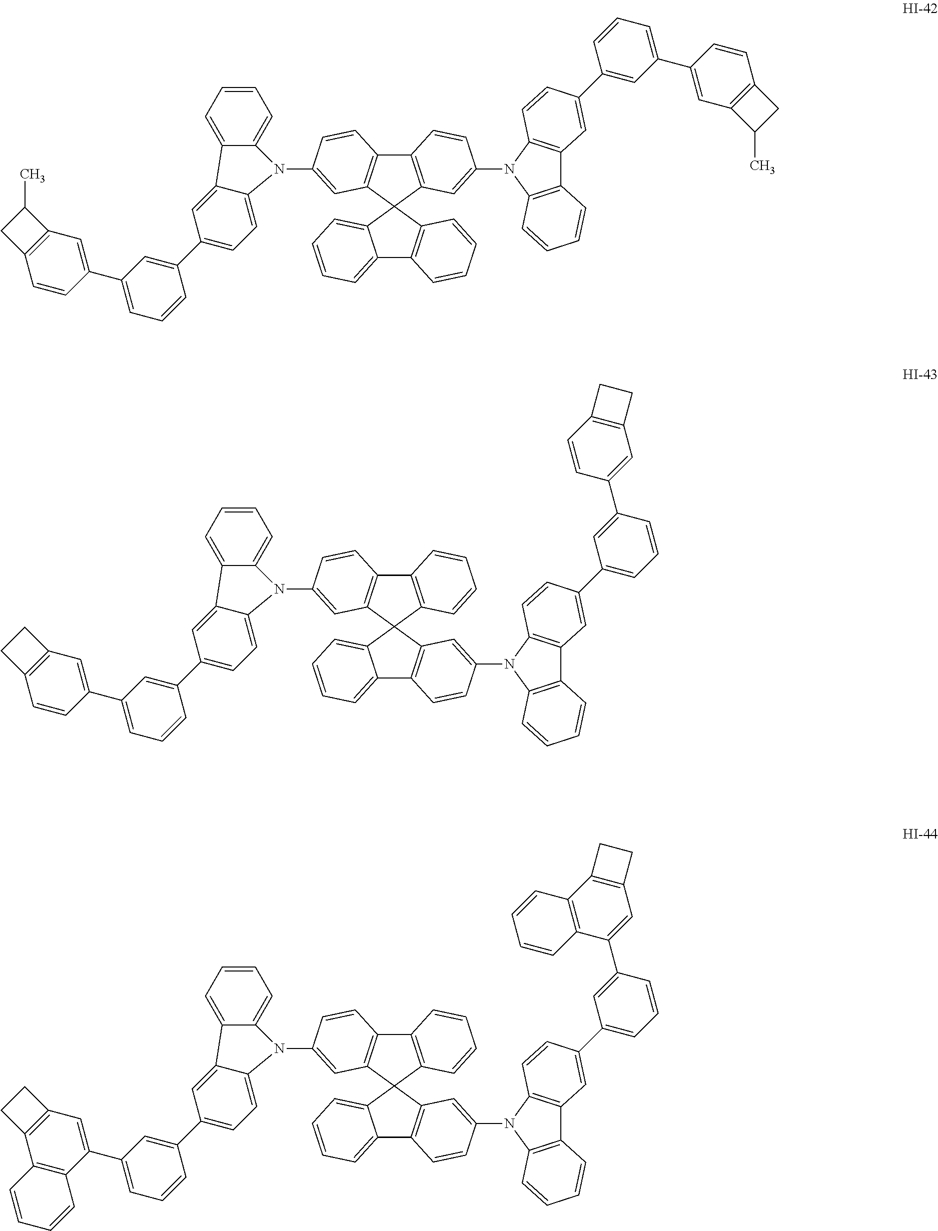 [SMILES: CC1Cc2ccc(-c3cccc(-c4ccc5c(c4)c4ccccc4n5-c4ccc5c(c4)C4(c6ccccc6-c6ccccc64)c4cc(-n6c7ccccc7c7cc(-c8cccc(-c9ccc%10c(c9)C(C)C%10)c8)ccc76)ccc4-5)c3)cc21 ; c1cc(-c2ccc3c(c2)CC3)cc(-c2ccc3c(c2)c2ccccc2n3-c2ccc3c(c2)C2(c4ccccc4-3)c3ccccc3-c3ccc(-n4c5ccccc5c5cc(-c6cccc(-c7ccc8c(c7)CC8)c6)ccc54)cc32)c1 ; c1cc(-c2ccc3c(c2)c2ccccc2n3-c2ccc3c(c2)C2(c4ccccc4-3)c3ccccc3-c3ccc(-n4c5ccccc5c5cc(-c6cccc(-c7cc8c(c9ccccc79)CC8)c6)ccc54)cc32)cc(-c2cc3c(c4ccccc24)CC3)c1]

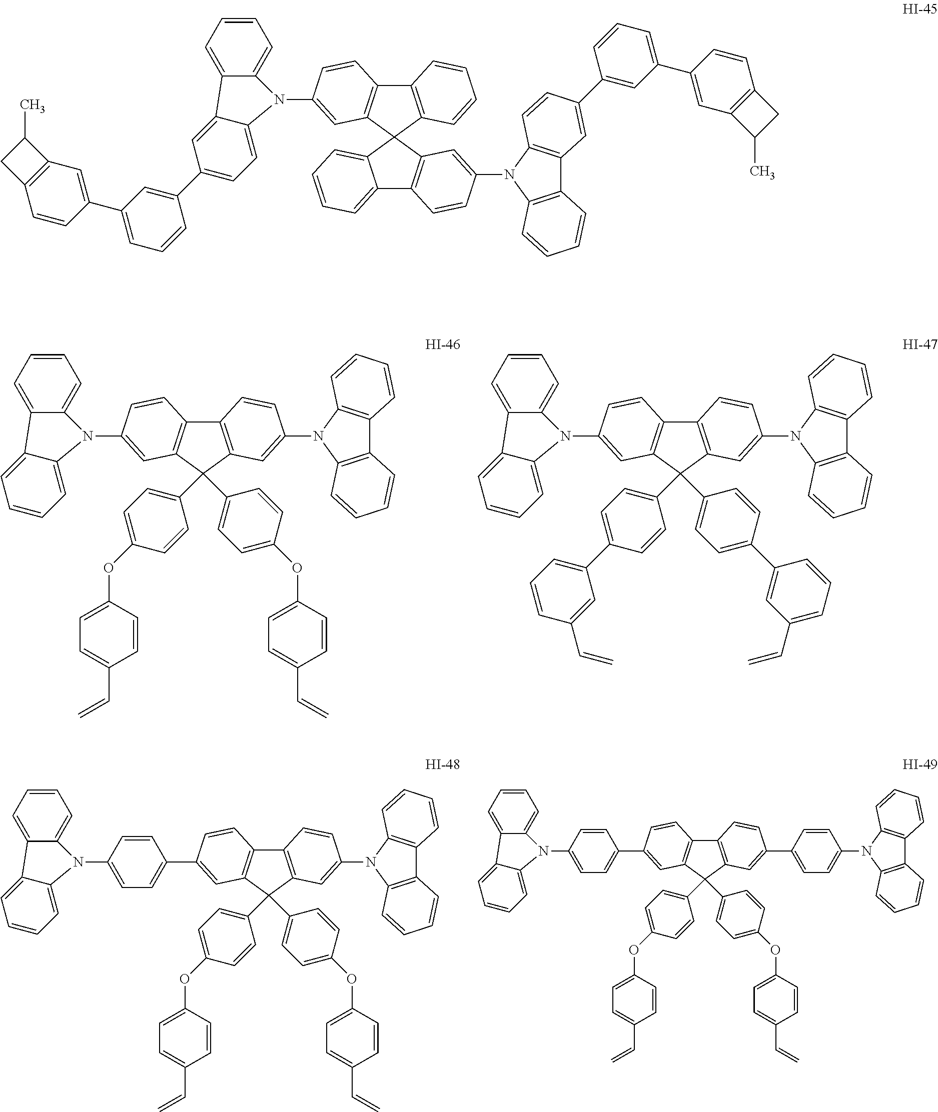

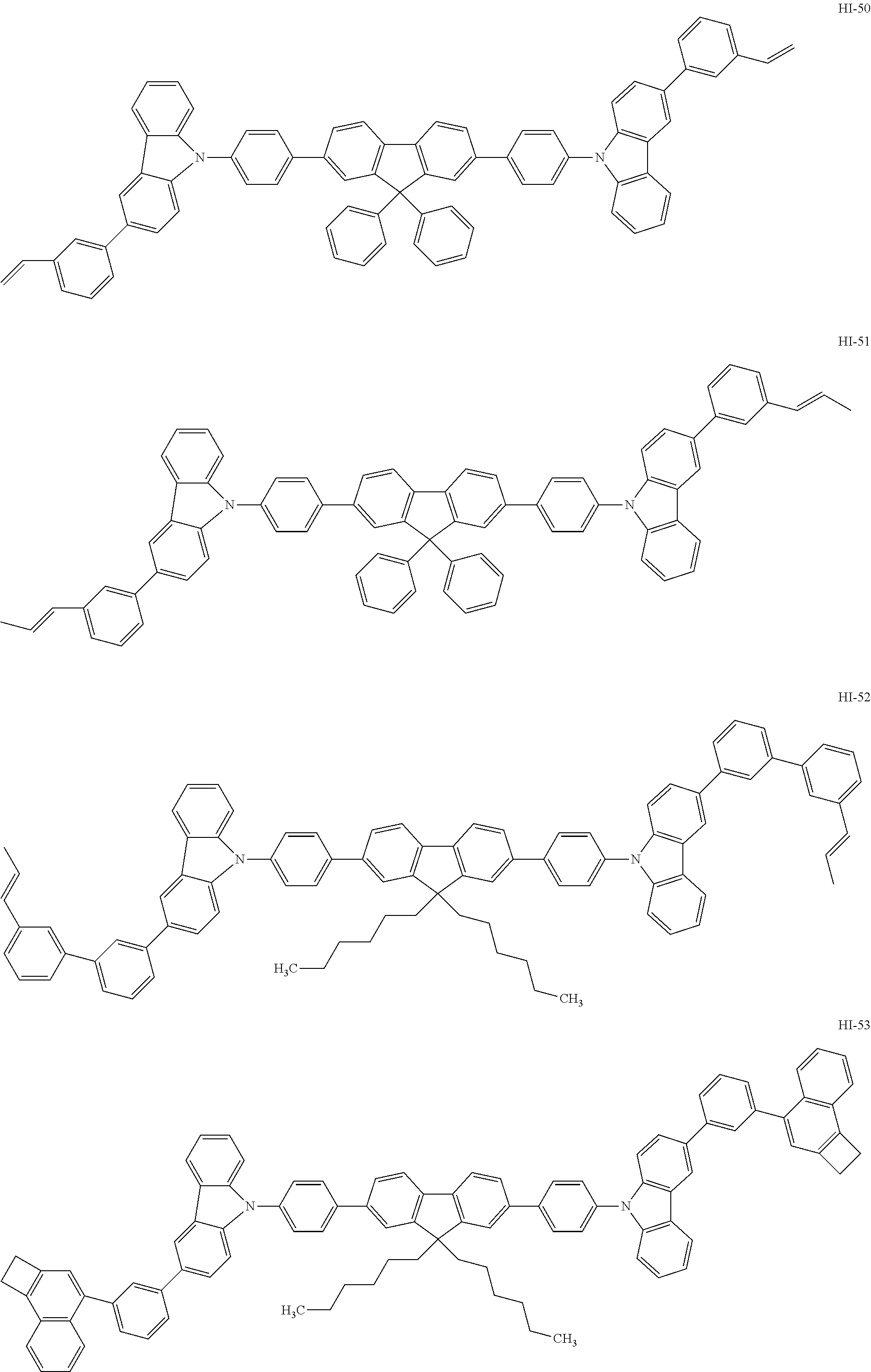

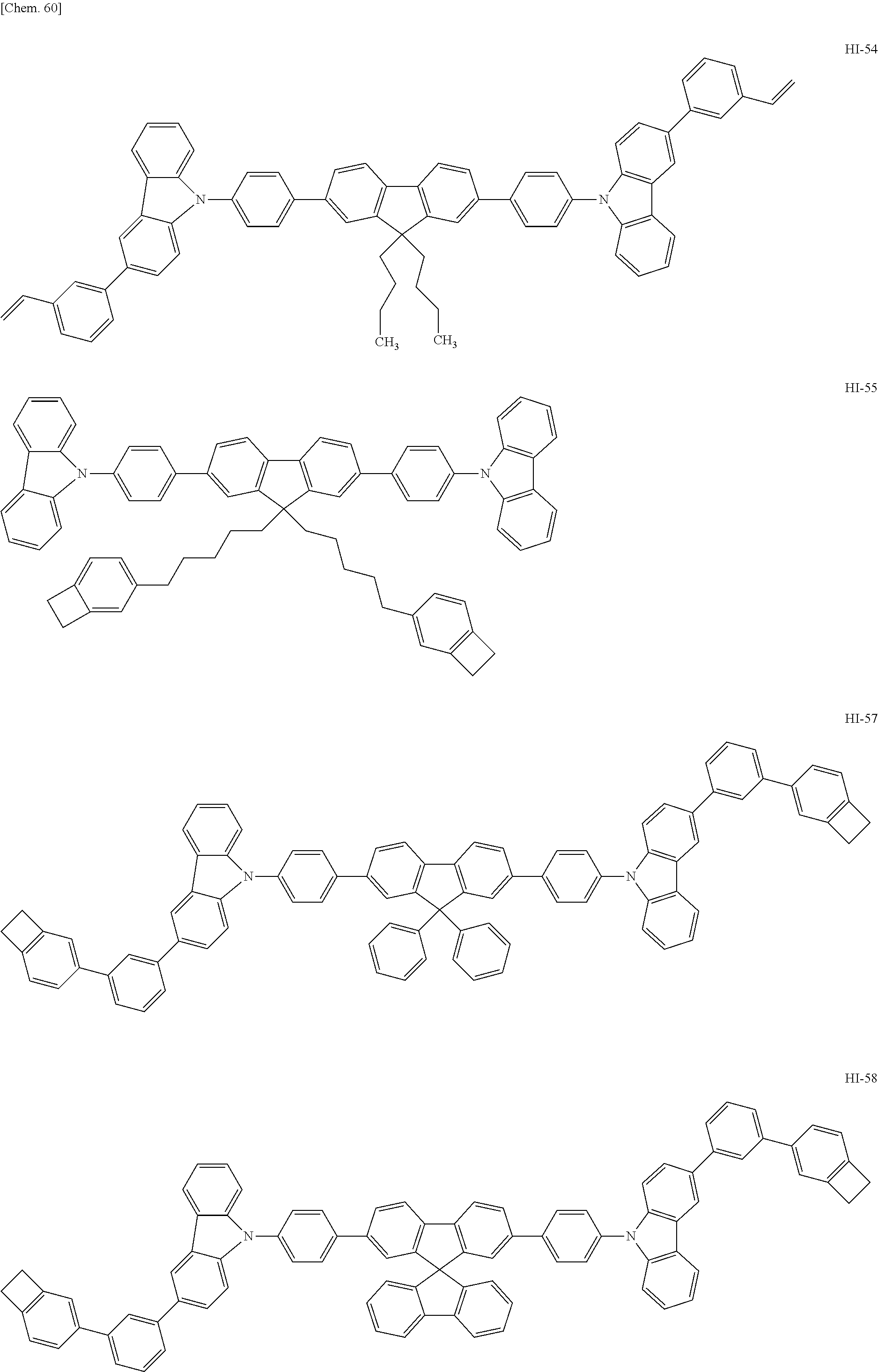

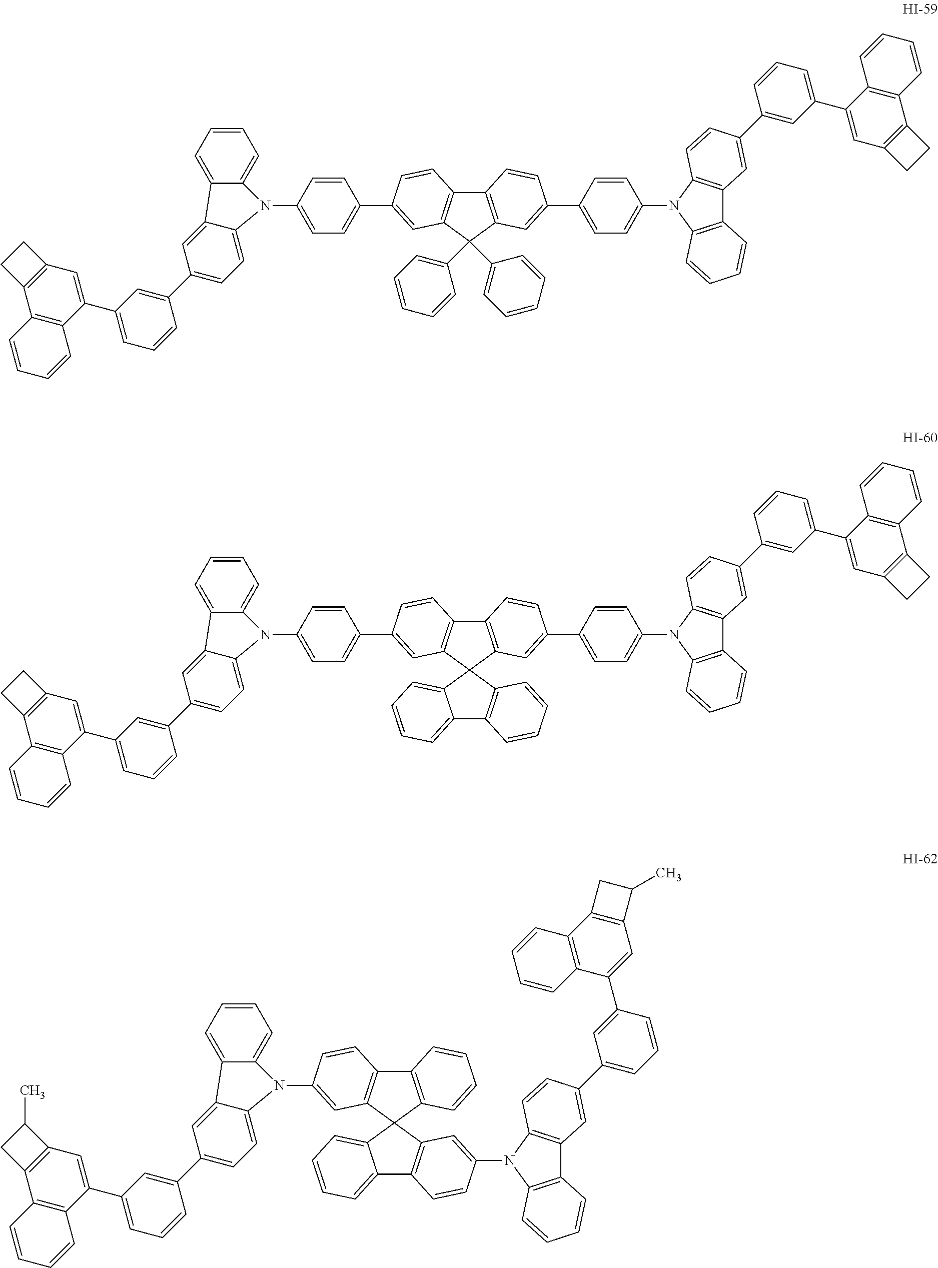

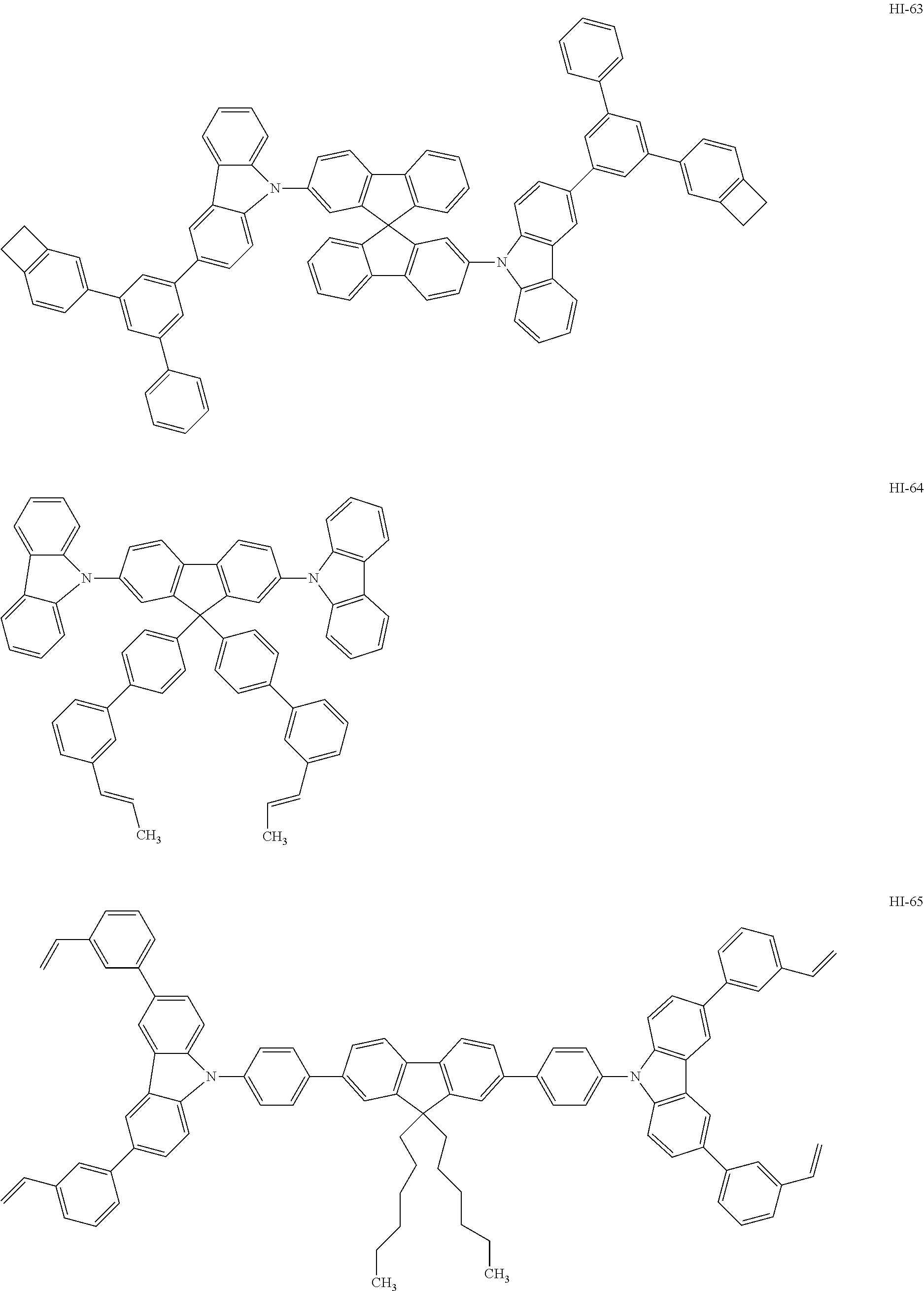 [SMILES: c1ccc(-c2cc(-c3ccc4c(c3)CC4)cc(-c3ccc4c(c3)c3ccccc3n4-c3ccc4c(c3)C3(c5ccccc5-4)c4ccccc4-c4ccc(-n5c6ccccc6c6cc(-c7cc(-c8ccccc8)cc(-c8ccc9c(c8)CC9)c7)ccc65)cc43)c2)cc1 ; C/C=C/c1cccc(-c2ccc(C3(c4ccc(-c5cccc(/C=C/C)c5)cc4)c4cc(-n5c6ccccc6c6ccccc65)ccc4-c4ccc(-n5c6ccccc6c6ccccc65)cc43)cc2)c1 ; C=Cc1cccc(-c2ccc3c(c2)c2cc(-c4cccc(C=C)c4)ccc2n3-c2ccc(-c3ccc4c(c3)C(CCCCCC)(CCCCCC)c3cc(-c5ccc(-n6c7ccc(-c8cccc(C=C)c8)cc7c7cc(-c8cccc(C=C)c8)ccc76)cc5)ccc3-4)cc2)c1]

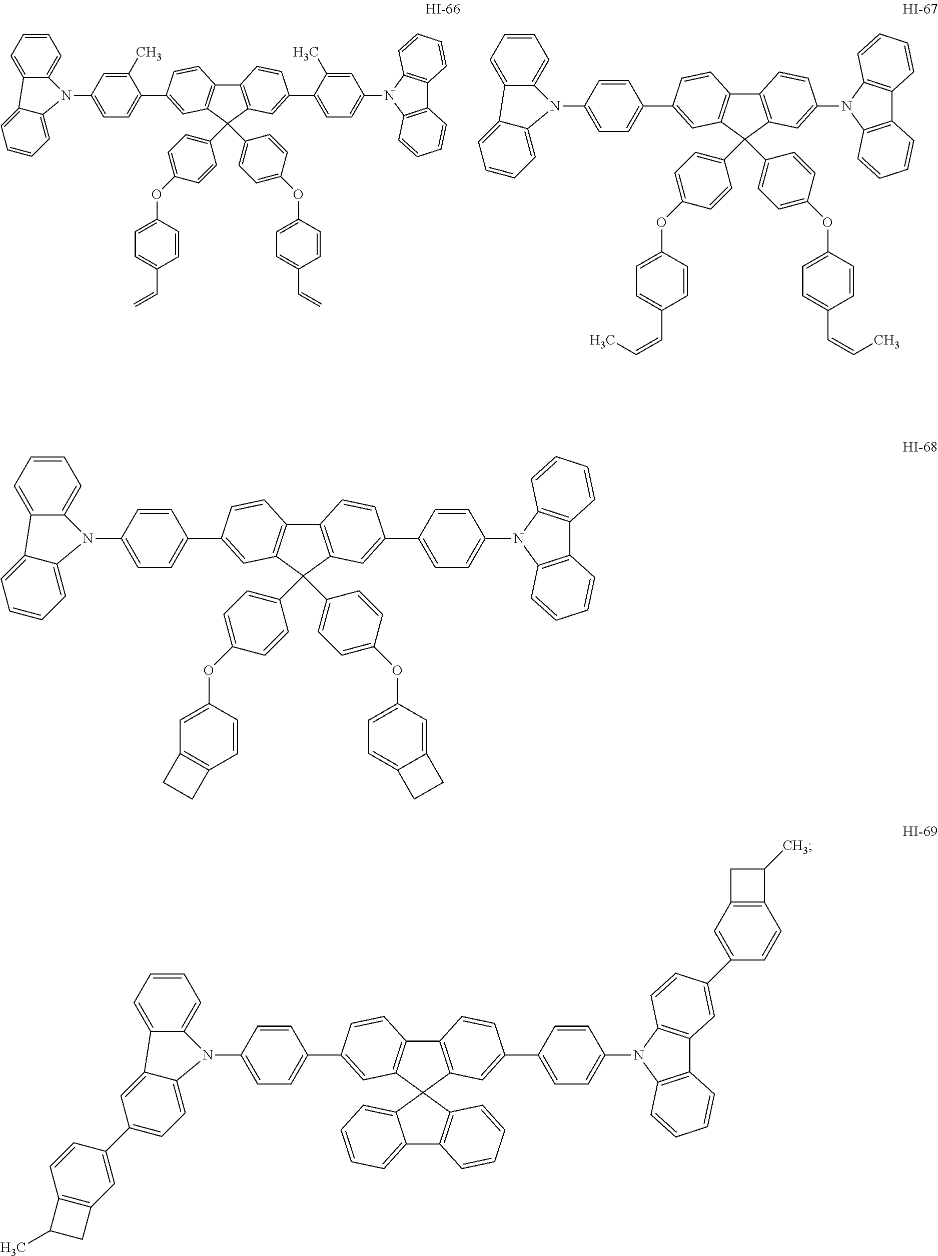

### :Specific Examples of the Carbazole Compound Represented by Formula (72):

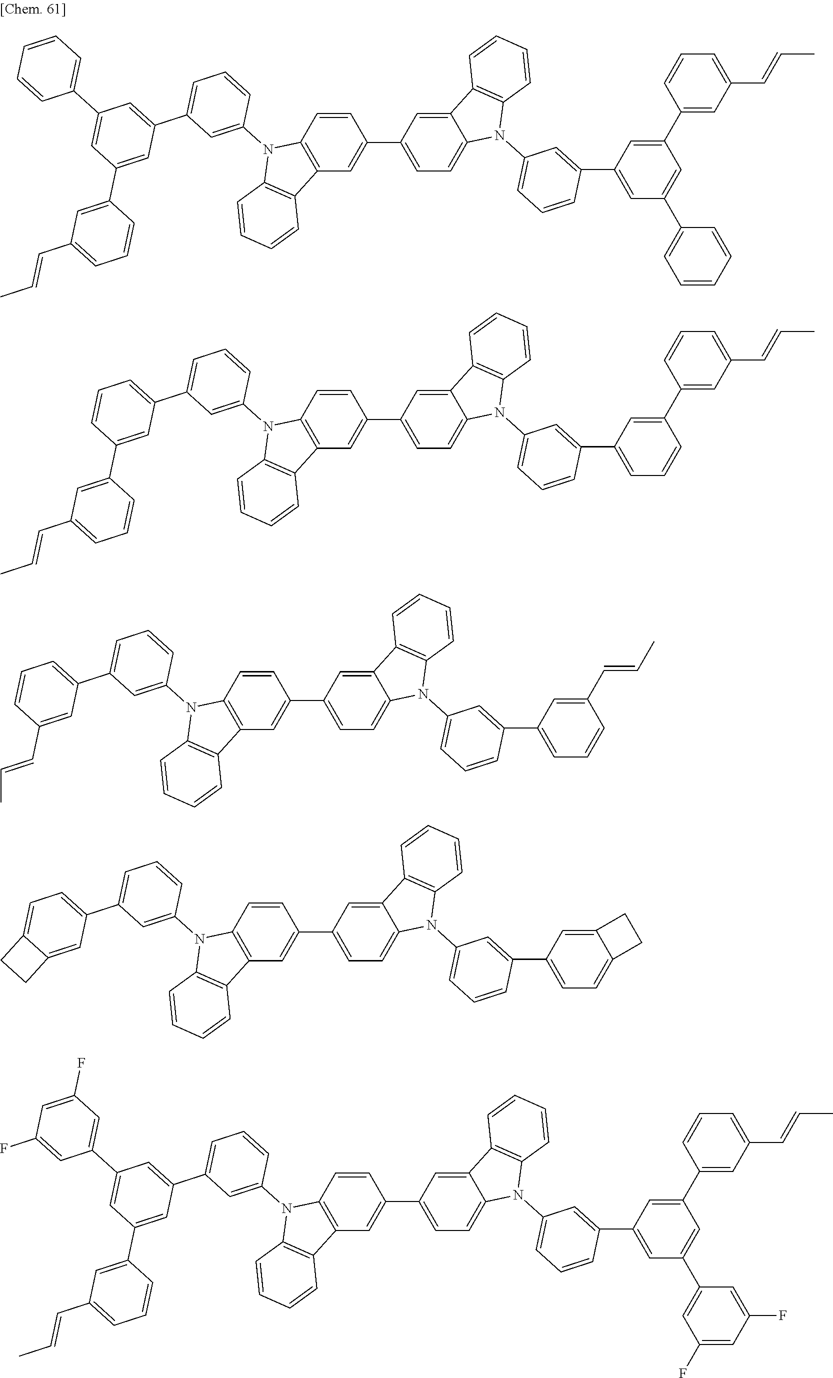

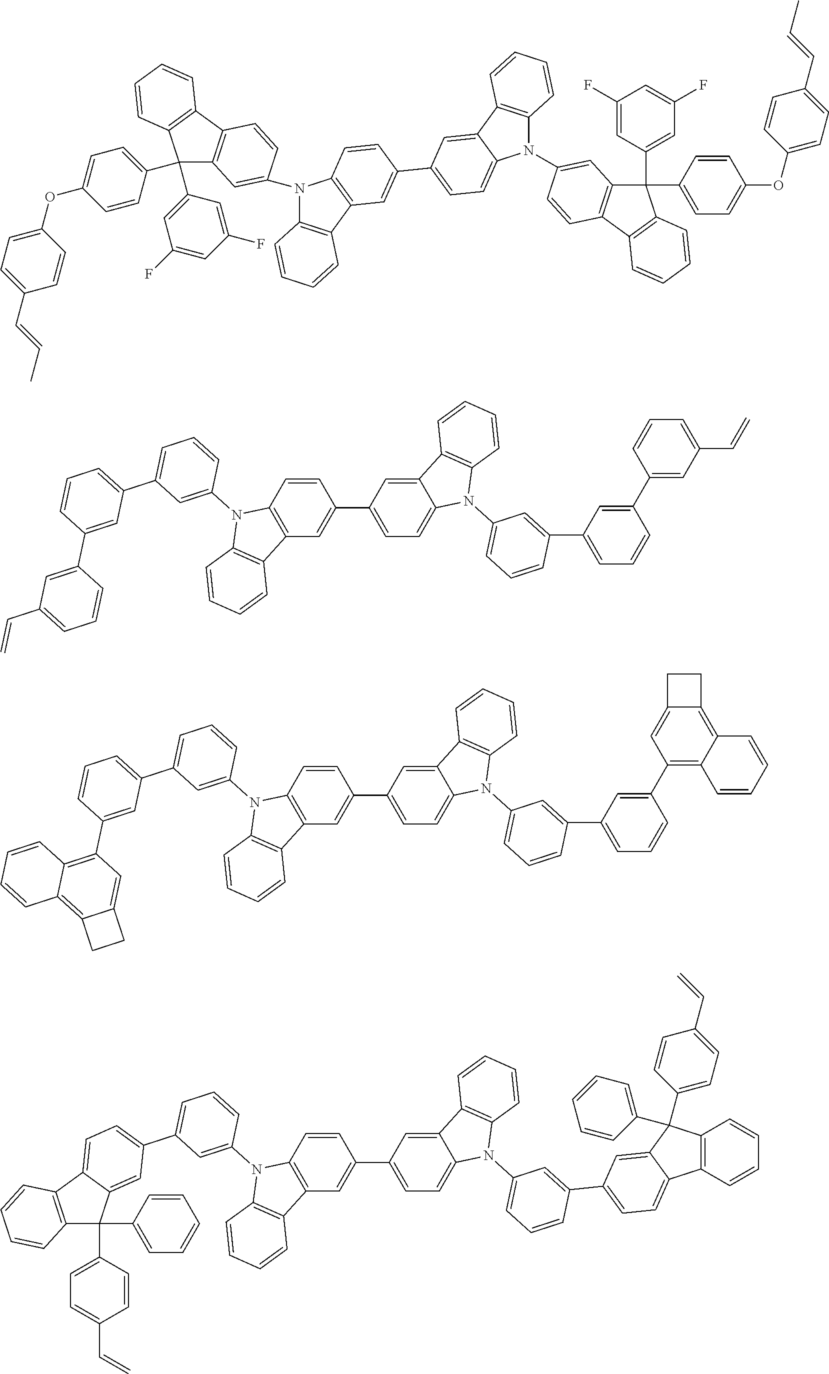

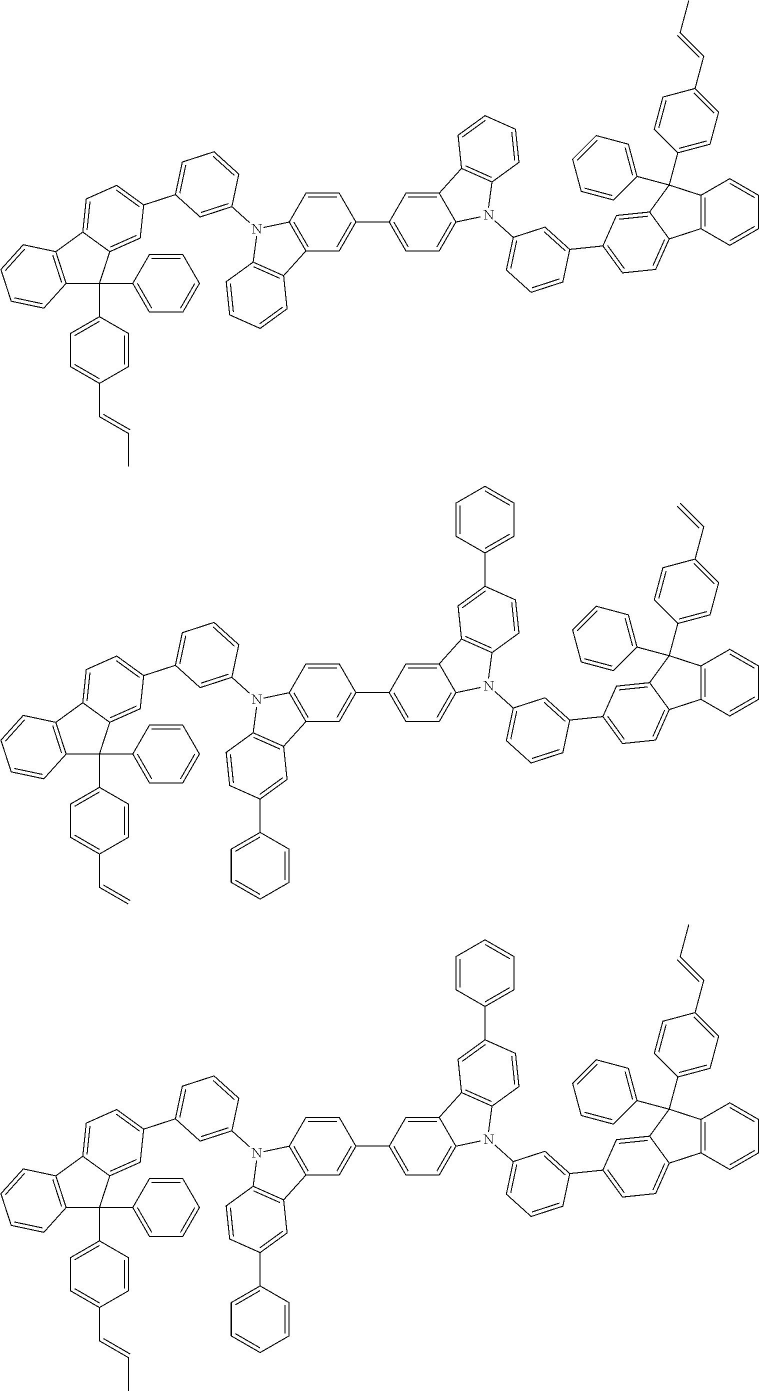

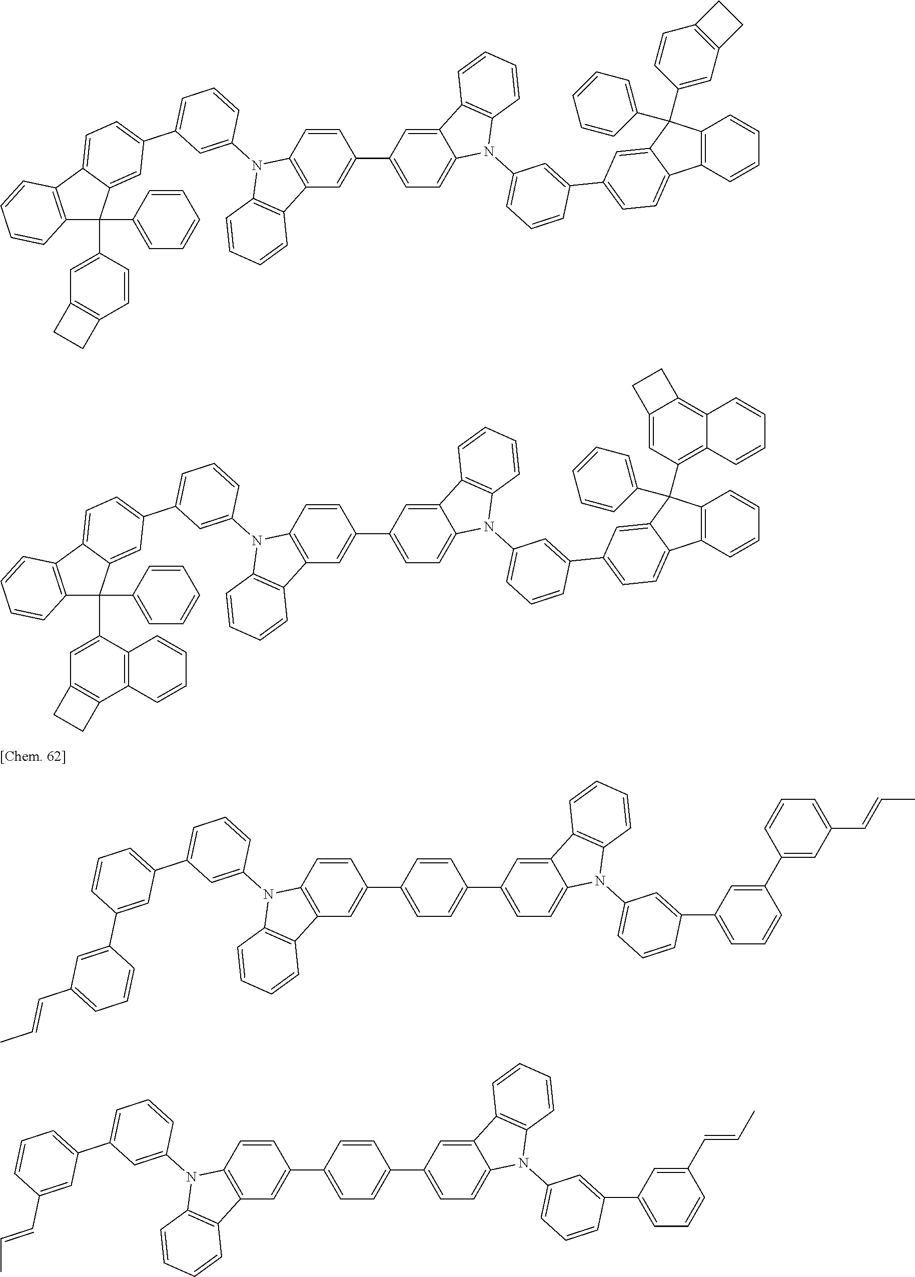

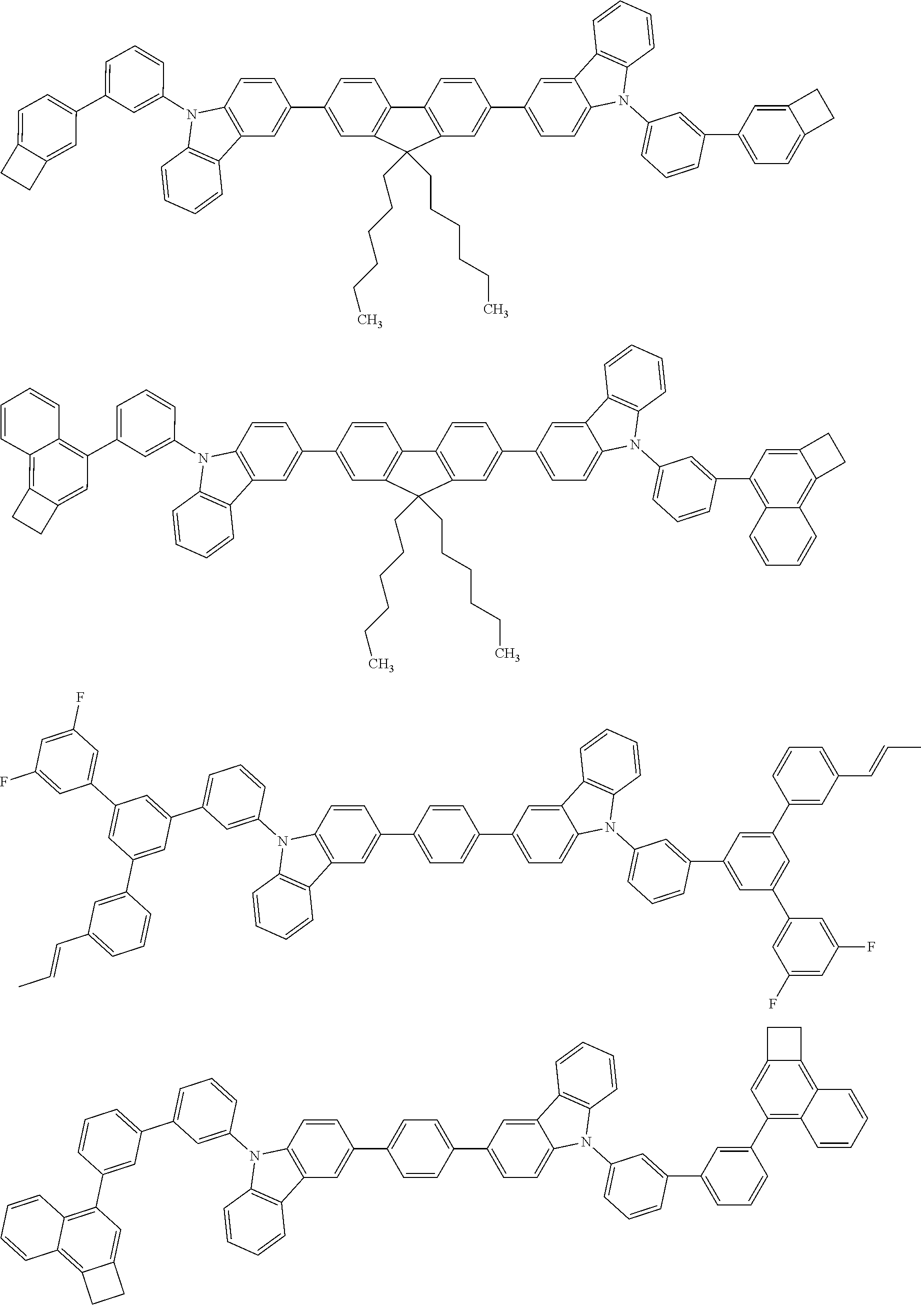

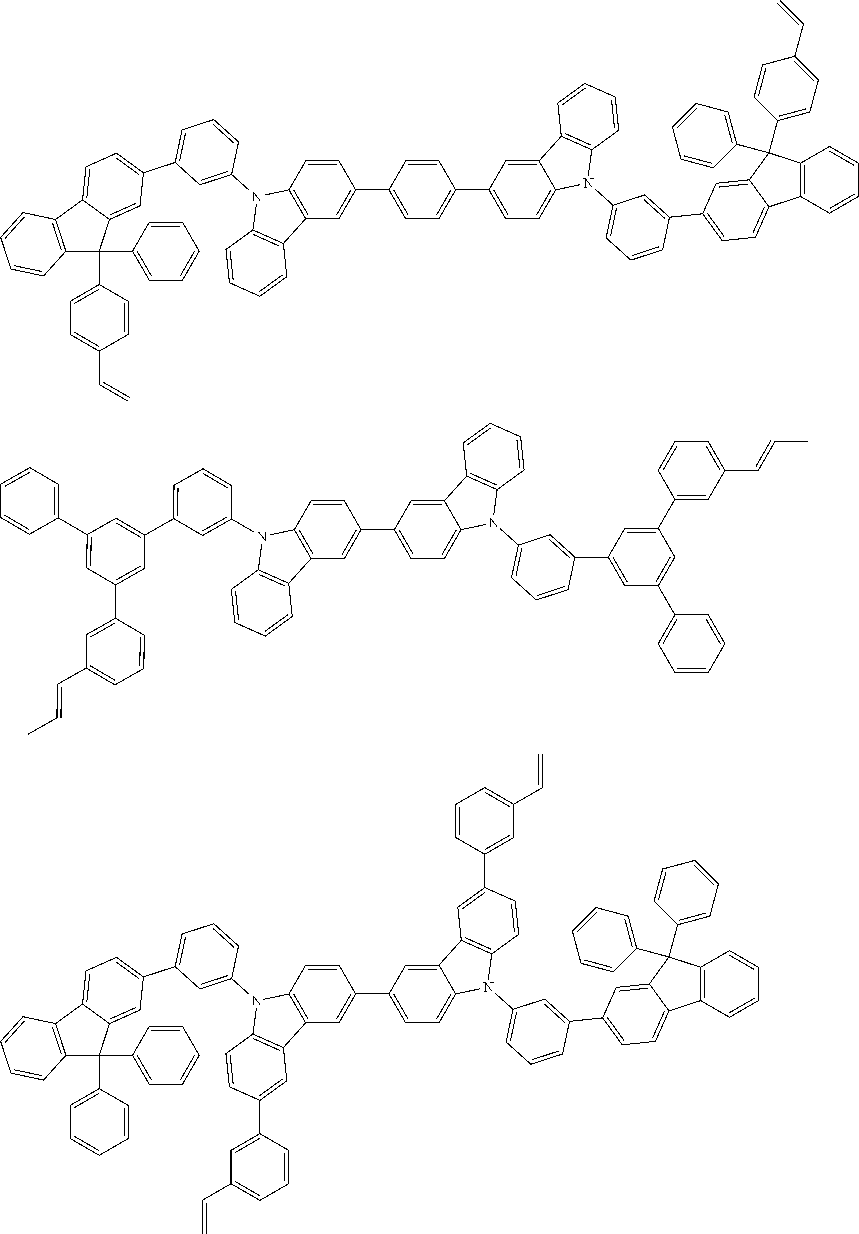

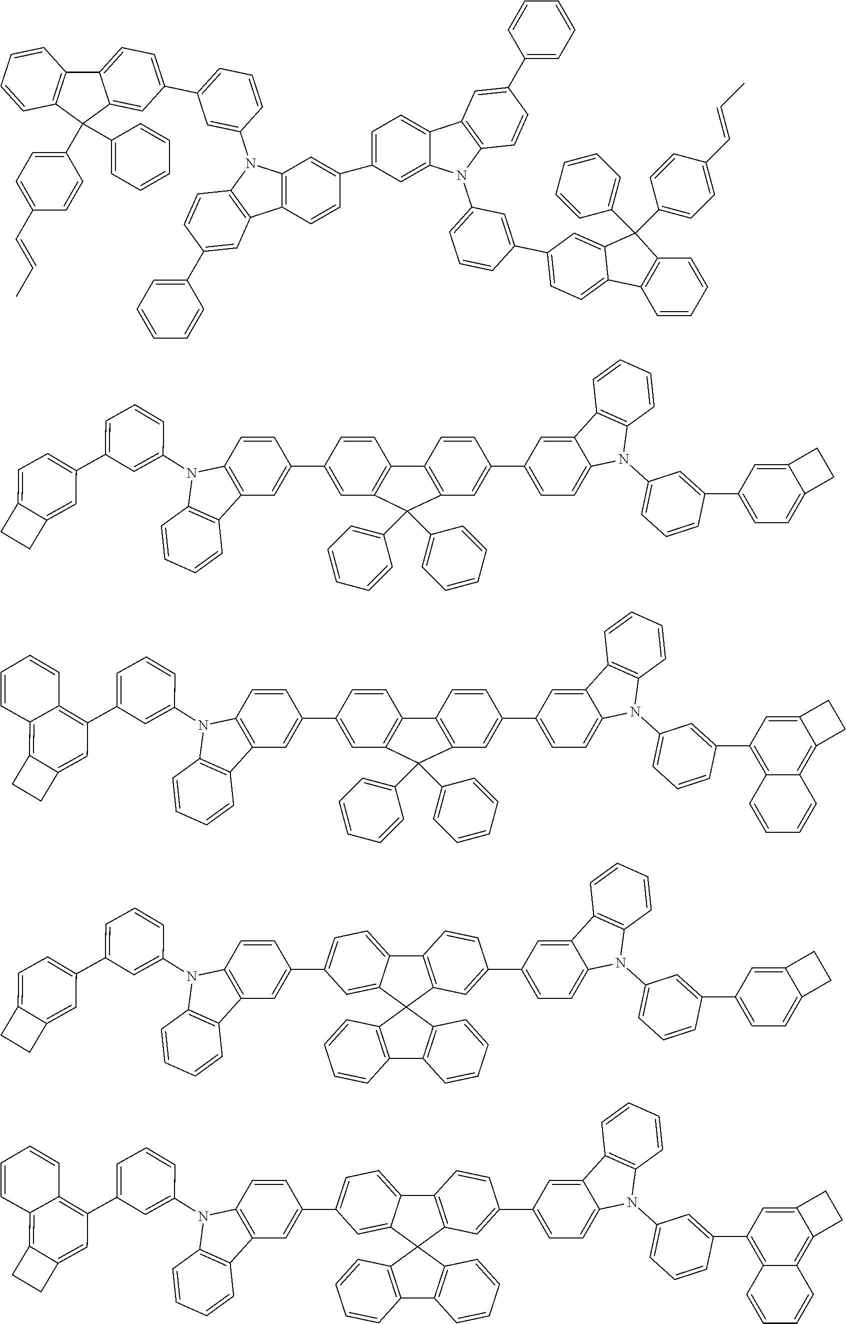

### [Electron Accepting Compound]

To improve hole injectability from the anode to the hole injection layer or the hole transport layer or to improve charge transportability in the hole injection layer or the hole transport layer, it is preferable that the charge transport material contained in the hole injection layer or the hole transport layer includes a cation radical moiety. To form a cation radical from the charge transport material, the electron accepting compound is used when the hole injection layer or the hole transport layer is formed. The mother skeleton of the electron accepting compound is preferably an ionic compound including a tetraarylborate ion, which is an anion with an ionic valence of 1 described later, and a counter cation because of its high stability.

(Formation of Cation Radical from Charge Transport Material)

A cation radical is formed from the charge transport material as follows.

When a compound having a carbazole structure is used as the charge transport material, tetraarylborate with diaryliodonium serving as a counter cation may be used as the electron accepting compound. In this case, the counter cation can be changed from the diaryliodonium to a carbazole cation as represented by the following formula when the hole injection layer or the hole transport layer is formed.

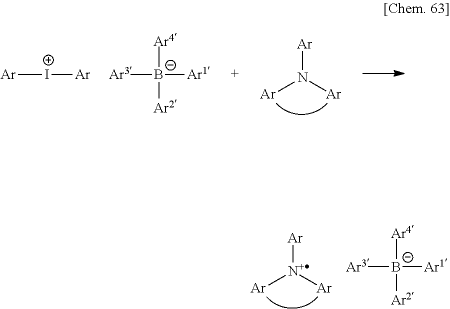

(For example, Ar and Ar¹′ to Ar⁴¹ are each independently an aromatic hydrocarbon group optionally having a substituent, an aromatic heterocyclic group optionally having a substituent, or a monovalent group in which a plurality of structures selected from aromatic hydrocarbon ring groups each optionally having a substituent and aromatic heterocyclic groups each optionally having a substituent are linked together.)

The carbazole cation formed through the above reaction has a singly occupied molecular orbital (SOMO) capable of accepting an electron. Therefore, the tetraarylborate with the carbazole ion serving as a counter cation is an electron accepting compound.

In the present invention, the compound including the charge transport material cation and the tetraarylborate anion is referred to as a charge transport ionic compound. Its details will be described later.

As described later, it is preferable that the hole injection layer and/or the hole transport layer of the organic electroluminescent element of the invention is obtained by subjecting a charge transport film-forming composition to wet deposition, and it is preferable that the charge transport film-forming composition in the invention is a composition obtained through the step of dissolving or dispersing an electron accepting compound having a tetraarylborate ion structure described later and a charge transport material described later in an organic solvent. Then it is preferable that the charge transport layer of the organic electroluminescent element of the invention contains a charge transport ionic compound including the tetraarylborate ion structure in the invention described later as an anion and the charge transport material cation in the invention as a counter cation.

### (Crosslinking Reaction Product)

When the charge transport material in the invention has a crosslinking group, a crosslinking reaction product of the charge transport material and the electron accepting compound having a crosslinking group is intended to encompass the following crosslinking reaction products.
A compound in which molecules of the electron accepting compound are crosslinked together.
A compound in which molecules of the electron accepting compound and molecules of the charge transport material are crosslinked together.
A compound in which molecules of the electron accepting compound and tetraarylborate ions in the invention are crosslinked together.
A compound in which tetraarylborate ions in the invention are crosslinked together.
A compound in which tetraarylborate ions in the invention and molecules of the charge transport material are crosslinked together.

The phrase “the tetraarylborate ion in the invention” is intended to encompass a tetraarylborate ion present in an electron accepting compound that is an ionic compound including the tetraarylborate ion and a counter cation and described later and a tetraarylborate ion present in a charge transport ionic compound including the tetraarylborate ion and a charge transport material cation and described later.

Two crosslinking groups that undergo the crosslinking reaction may be of the same type or different types so long as they can undergo the crosslinking reaction.

### [Electron Accepting Compound]

The electron accepting compound that is an ionic compound including a tetraarylborate ion and a counter cation is an electron accepting ionic compound represented by formula (81) below and including a counter anion that is a non-coordinating anion and a counter cation. Formula (82) below has, as an anion, a tetraarylborate ion represented by formula (83) described later. The electron accepting compound in the invention may be referred to as an electron accepting ionic compound.

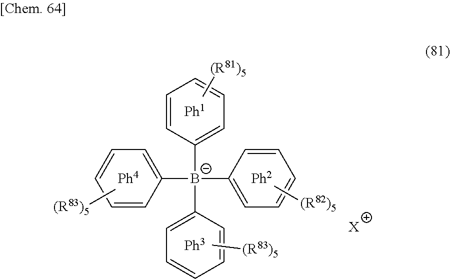

(In formula (81), five R⁸¹'s, five R⁸²'s, five R⁸³'s, and five R³⁴'s are each independent; R⁸¹'s to R³⁴'s each independently represent a hydrogen atom, a deuterium atom, a halogen atom, an aromatic hydrocarbon group having 6 to 50 carbon atoms and optionally having a substituent and/or a crosslinking group, an aromatic heterocyclic group having 3 to 50 carbon atoms and optionally having a substituent and/or a crosslinking group, a fluorine-substituted alkyl group having 1 to 12 carbon atoms, or a crosslinking group;
Ph¹, Ph², Ph³, are Ph⁴ are symbols representing four benzene rings;
the compound represented by formula (81) has a crosslinking group; and
X⁺ represents a counter cation.)

The electron accepting compound represented by formula (81) above has preferably a crosslinking group and has more preferably 2 or more crosslinking groups. Preferably, each crosslinking group is present in an anionic portion of the electron accepting compound represented by formula (81), i.e., in formula (82) described below that is a tetraarylborate ion.

### [Tetraarylborate Ion]

The mother skeleton of the electron accepting compound is preferably an ionic compound including a counter cation and a tetraarylborate ion that is an anion with an ionic valence of 1 in which a boron atom is substituted with four aromatic hydrocarbon rings each optionally having a substituent or four aromatic heterocycles each optionally having a substituent, because the stability of this ionic compound is high.

The tetraarylborate ion is an anion of formula (81) that is represented by the following formula (82).

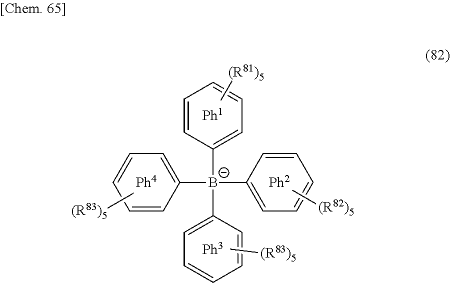

(In formula (82), R⁸¹ to R⁸⁴ are the same as R⁸¹ to R⁸⁴, respectively, in formula (81).

Ph¹ to Ph⁴ are the same as Ph¹ to Ph⁴, respectively, in formula (81) and represent the four benzene rings.)

The number of carbon atoms in each of the aromatic hydrocarbon groups used as R⁸¹ to R⁸⁴ is preferably 6 to 50. The aromatic hydrocarbon ring structure is preferably a monocycle, a condensed ring including 2 to 6 rings, or a structure in which 2 to 8 of them are linked together. Specific examples of the aromatic hydrocarbon group include: monovalent groups each including one of a benzene ring, a naphthalene ring, an anthracene ring, a phenanthrene ring, a perylene ring, a tetracene ring, a pyrene ring, a benzpyrene ring, a chrysene ring, a triphenylene ring, an acenaphthene ring, a fluoranthene ring, a fluorene ring, a biphenyl structure, a terphenyl structure, and a quaterphenyl structure; and monovalent groups in which 2 to 8 groups selected from of the above groups are linked together.

The number of carbon atoms in each of the aromatic heterocyclic groups used as R⁸¹ to R⁸⁴ is preferably 3 to 50. The aromatic heterocyclic structure is preferably a monocycle, a condensed ring including 2 to 6 rings, or a structure in which 2 to 8 of them are linked together. Specific examples of the aromatic heterocyclic group include: monovalent groups each including one of a furan ring, a benzofuran ring, a thiophene ring, a benzothiophene ring, a pyrrole ring, a pyrazole ring, an imidazole ring, an oxadiazole ring, an indole ring, a carbazole ring, a pyrroloimidazole ring, a pyrrolopyrazole ring, a pyrrolopyrrole ring, a thienopyrrole ring, a thienothiophene ring, a furopyrrole ring, a furofuran ring, a thienofuran ring, a benzisoxazole ring, a benzisothiazole ring, a benzimidazole ring, a pyridine ring, a pyrazine ring, a pyridazine ring, a pyrimidine ring, a triazine ring, a quinoline ring, an isoquinoline ring, a cinnoline ring, a quinoxaline ring, a phenanthridine ring, a perimidine ring, a quinazoline ring, a quinazolinone ring, and an azulene ring; and monovalent groups in which 2 to 8 groups selected from of the above groups are linked together. It is only necessary that the aromatic heterocyclic group contain at least one of these single structures, and the linked structure may include an aromatic hydrocarbon ring structure. When the linked structure includes an aromatic hydrocarbon ring structure, the linked structure may include 2 to 8 linked rings including the aromatic heterocycle and the aromatic hydrocarbon ring. The aromatic hydrocarbon ring used can be any of the above-described structures used for R⁸¹ to R⁸⁴ and each including one aromatic hydrocarbon ring.

In particular, a monovalent group including a benzene ring, a naphthalene ring, a fluorene ring, a pyridine ring, or a carbazole ring or a monovalent group in which 2 to 5 of the above groups are linked together such as a biphenyl group is more preferred because of their high stability and heat resistance. A monovalent group including a benzene ring or a group including 2 to 5 benzene rings linked together is particularly preferred, and specific examples thereof include a phenyl group, a biphenyl group, and a terphenyl group.

The number of aromatic hydrocarbon and aromatic heterocyclic groups included in the monovalent group in which a plurality of structures selected from aromatic hydrocarbon groups each optionally having a substituent and aromatic heterocyclic groups each optionally having a substituent are linked together is preferably 2 or more and 8 or less, more preferably 4 or less, and still more preferably 3 or less. When a biphenyl group, a terphenyl group, and a quaterphenyl group are used as aromatic hydrocarbon groups, these aromatic hydrocarbon groups are regarded as structures in which 2, 3, and 4 phenyl groups, respectively, are linked together.

Each of the optional substituents on R⁸¹ to R⁸⁴ is preferably a group selected from the substituent group Z, particularly from the substituent group X.

R⁸¹ to R⁸⁴ are each preferably a fluorine atom or a fluorine-substituted alkyl group because the stability of the anion increases and the effect of stabilizing the cation is improved. It is preferable that two or more fluorine atoms or two or more fluorine-substituted alkyl groups are included. It is more preferable that three or more fluorine atoms or three or more fluorine-substituted alkyl groups are included, and it is most preferable that four fluorine atoms or four fluorine-substituted alkyl groups are included.

Each of the fluorine-substituted alkyl groups used for R⁸¹ to R⁸⁴ is preferably a linear or branched alkyl group having 1 to 12 carbon atoms and substituted with a fluorine atom, more preferably a perfluoroalkyl group, still more preferably a linear or branched perfluoroalkyl group having 1 to 5 carbon atoms, particularly preferably a linear or branched perfluoroalkyl group having 1 to 3 carbon atoms, and most preferably a perfluoromethyl group. The reason for this is that a charge injection layer containing a crosslinked product of the electron accepting compound having a crosslinking group and a coating film formed on this film are stabilized. Preferably, the fluorine-substituted alkyl group is boned at the para position with respect to the boron atom.

Preferably, in the tetraarylborate ion, at least one of -Ph¹-(R⁸¹)₅, -Ph²-(R⁸²)₅, -Ph³-(R⁸³)₅, and -Ph⁴-(R⁸⁴)₅ in formula (82) above is a group represented by formula (84) below and having four fluorine atoms because the stability of the anion further increases and the effect of stabilizing the cation is further improved. It is more preferable that at least two of them are the same group represented by formula (84) because the stability of the anion is improved, and it is most preferable that at least three of them are the same group represented by formula (84) because the stability of the anion is further improved.

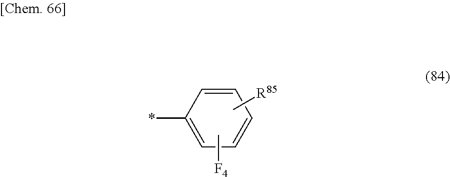

(In formula (84), * represents a bond to boron B in formula (81).

F₄ represents substitution with four fluorine atoms.

R⁸⁵ represents an aromatic hydrocarbon group optionally having a substituent and/or a crosslinking group or a crosslinking group.)

The number of carbon atoms in the aromatic hydrocarbon group usable for R⁸⁵ is preferably 3 to 40. The aromatic hydrocarbon ring structure is preferably a monocycle, a condensed ring including 2 to 6 rings, or a structure including 2 to 5 of these groups linked together. Specific examples include: monovalent groups including one of a benzene ring, a naphthalene ring, an anthracene ring, a phenanthrene ring, a perylene ring, a tetracene ring, a pyrene ring, a benzpyrene ring, a chrysene ring, a triphenylene ring, an acenaphthene ring, a fluoranthene ring, a fluorene ring, a biphenyl structure, a terphenyl structure, and a quaterphenyl structure; and monovalent groups in which 2 to 6 groups selected from of the above groups are linked together. The optional crosslinking group on the aromatic hydrocarbon group is a crosslinking group selected from the group T of crosslinking groups.

The crosslinking group usable for R⁸⁵ is a crosslinking group selected from the group T of crosslinking groups.

Each of the optional substituents on the aromatic hydrocarbon and aromatic hydrocarbon groups is preferably a substituent selected from the substituent group Z, particularly from the substituent group X. In particular, an aromatic hydrocarbon group is preferred in terms of stability, and an alkyl group is preferred in terms of solubility.

### <Electron Accepting Ionic Compound Containing Tetraarylborate Ion>

The tetraarylborate ion is used preferably as an electron accepting ionic compound including the tetraarylborate ion serving as an anion and a counter cation.

### (Counter Cation)

The counter cation is preferably an iodonium cation, a sulfonium cation, a carbocation, an oxonium cation, an ammonium cation, a phosphonium cation, a cycloheptyltrienyl cation, or a ferrocenium cation having a transition metal, more preferably an iodonium cation, a sulfonium cation, a carbocation, or an ammonium cation, and particularly preferably an iodonium cation.

The iodonium cation has preferably a structure represented by formula (83) below, and a more preferred structure is also the same as the structure represented by formula (83).

Specifically, the iodonium cation is preferably a diphenyliodonium cation, a bis(4-tert-butylphenyl)iodonium cation, a 4-tert-butoxyphenylphenyliodonium cation, a 4-methoxyphenylphenyliodonium cation, a 4-isopropylphenyl-4-methylphenyliodonium cation, etc.

Specifically, the sulfonium cation is preferably a triphenylsulfonium cation, a 4-hydroxyphenyldiphenylsulfonium cation, a 4-cyclohexylphenyldiphenylsulfonium cation, a 4-methanesulfonylphenyldiphenylsulfonium cation, a (4-tert-butoxyphenyl)diphenylsulfonium cation, a (4-tert-butoxyphenyl)diphenylsulfonium cation, a bis(4-tert-butoxyphenyl)phenylsulfonium cation, a 4-cyclohexylsulfonylphenyldiphenylsulfonium cation, etc.

Specifically, the carbocation is preferably a trisubstituted carbocation such as a triphenyl carbocation, a tri(methylphenyl) carbocation, or a tri(dimethylphenyl) carbocation.

Specifically, the ammonium cation is preferably: a trialkylammonium cation such as a trimethylammonium cation, a triethylammonium cation, a tripropylammonium cation, a tributylammonium cation, or a tri(n-butyl)ammonium cation; an N,N-dialkylanilinium cation such as an N,N-diethylanilinium cation or an N,N-2,4,6-pentamethylanilinium cation; a dialkylammonium cation such as a di(isopropyl)ammonium cation or a dicyclohexylammonium cation; etc.

Specifically, the phosphonium cation is preferably: a tetraarylphosphonium cation such as a tetraphenylphosphonium cation, a tetrakis(methylphenyl)phosphonium cation, or a tetrakis(dimethylphenyl)phosphonium cation; a tetraalkylphosphonium cation such as a tetrabutylphosphonium cation or a tetrapropylphosphonium cation; etc.

Of these, the iodonium cation, the carbocation, and the sulfonium cation are preferred in terms of the stability of a film of the compound, and the iodonium cation is more preferred.

### (X⁺: (Iodonium Cation))

X⁺, which is the counter cation in formula (81) above, is preferably the iodonium cation having a structure represented by formula (83) below.

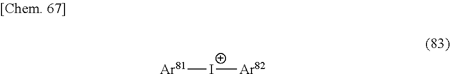

In formula (83), Ar⁸¹ and Ar⁸² are each independently an aromatic hydrocarbon group having 6 to 30 carbon atoms and optionally having a substituent.

The aromatic hydrocarbon group is preferably an aromatic hydrocarbon group having 6 to 18 carbon atoms, more preferably an aromatic hydrocarbon group having 6 to 12 carbon atoms, and most preferably a phenyl group. The optional substituent is a group selected from the substituent group Z and is most preferably an alkyl group.

The aromatic hydrocarbon group is preferably a phenyl group, a biphenyl group, a terphenyl group, a quaterphenyl group, a naphthyl group, a phenanthrenyl group, a triphenylene group, or a naphthylphenyl group and is most preferably a phenyl group in terms of the stability of the compound.

### (Molecular Weight)

The molecular weight of the electron accepting ionic compound having the tetraarylborate ion is generally 900 or more, preferably 1000 or more, and more preferably 1200 or more and is generally 10000 or less, preferably 5000 or less, and more preferably 3000 or less. If the molecular weight is excessively low, delocalization of positive charges and negative charges is insufficient, so that the electron-accepting ability may deteriorate. If the molecular weight is excessively large, the charge transport may be inhibited.

### (Specific Examples)

Specific examples of the ionic compound including an iodonium cation and serving as the electron accepting ionic compound represented by formula (81) are shown below. However, the invention is not limited thereto.

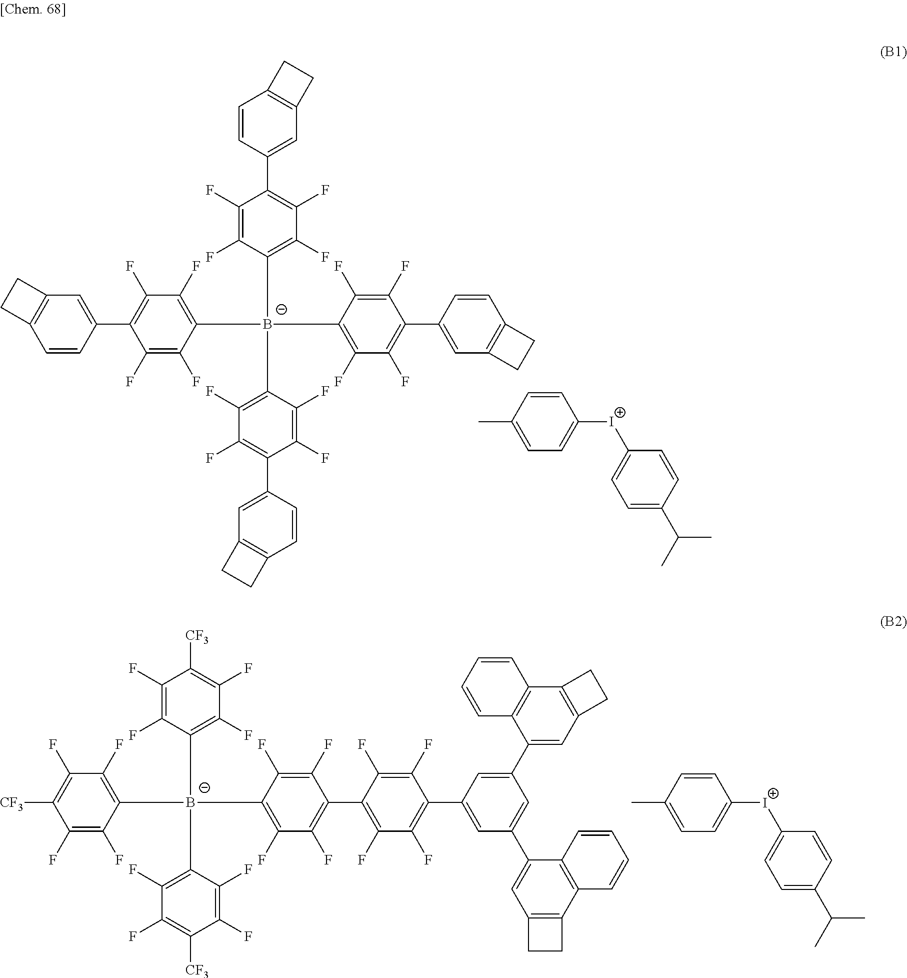 [SMILES: Fc1c(F)c([B-](c2c(F)c(F)c(-c3ccc4c(c3)CC4)c(F)c2F)(c2c(F)c(F)c(-c3ccc4c(c3)CC4)c(F)c2F)c2c(F)c(F)c(-c3ccc4c(c3)CC4)c(F)c2F)c(F)c(F)c1-c1ccc2c(c1)CC2 ; Cc1ccc([I+]c2ccc(C(C)C)cc2)cc1 ; Fc1c(F)c(-c2c(F)c(F)c([B-](c3c(F)c(F)c(C(F)(F)F)c(F)c3F)(c3c(F)c(F)c(C(F)(F)F)c(F)c3F)c3c(F)c(F)c(C(F)(F)F)c(F)c3F)c(F)c2F)c(F)c(F)c1-c1cc(-c2cc3c(c4ccccc24)CC3)cc(-c2cc3c(c4ccccc24)CC3)c1 ; Cc1ccc([I+]c2ccc(C(C)C)cc2)cc1]

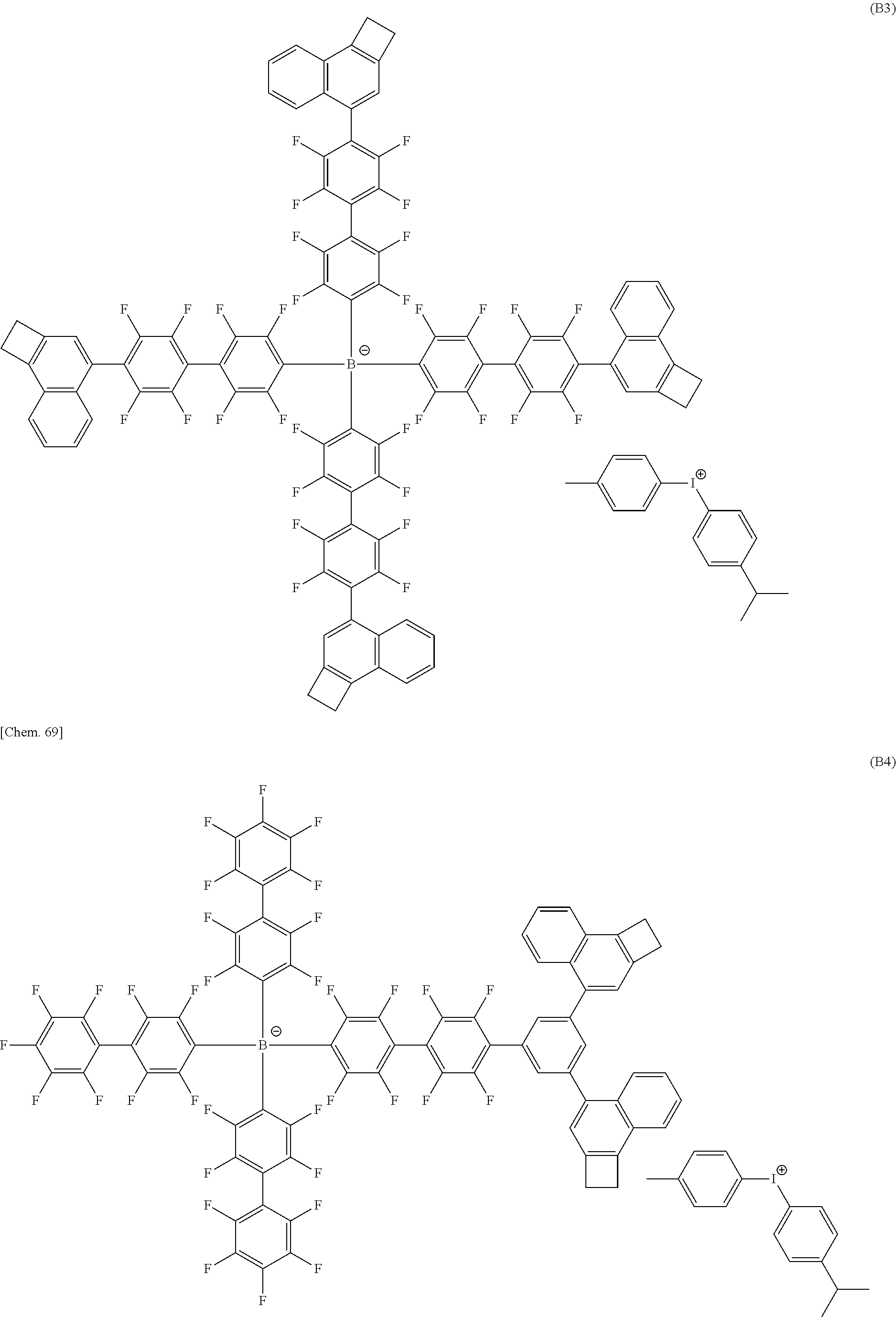

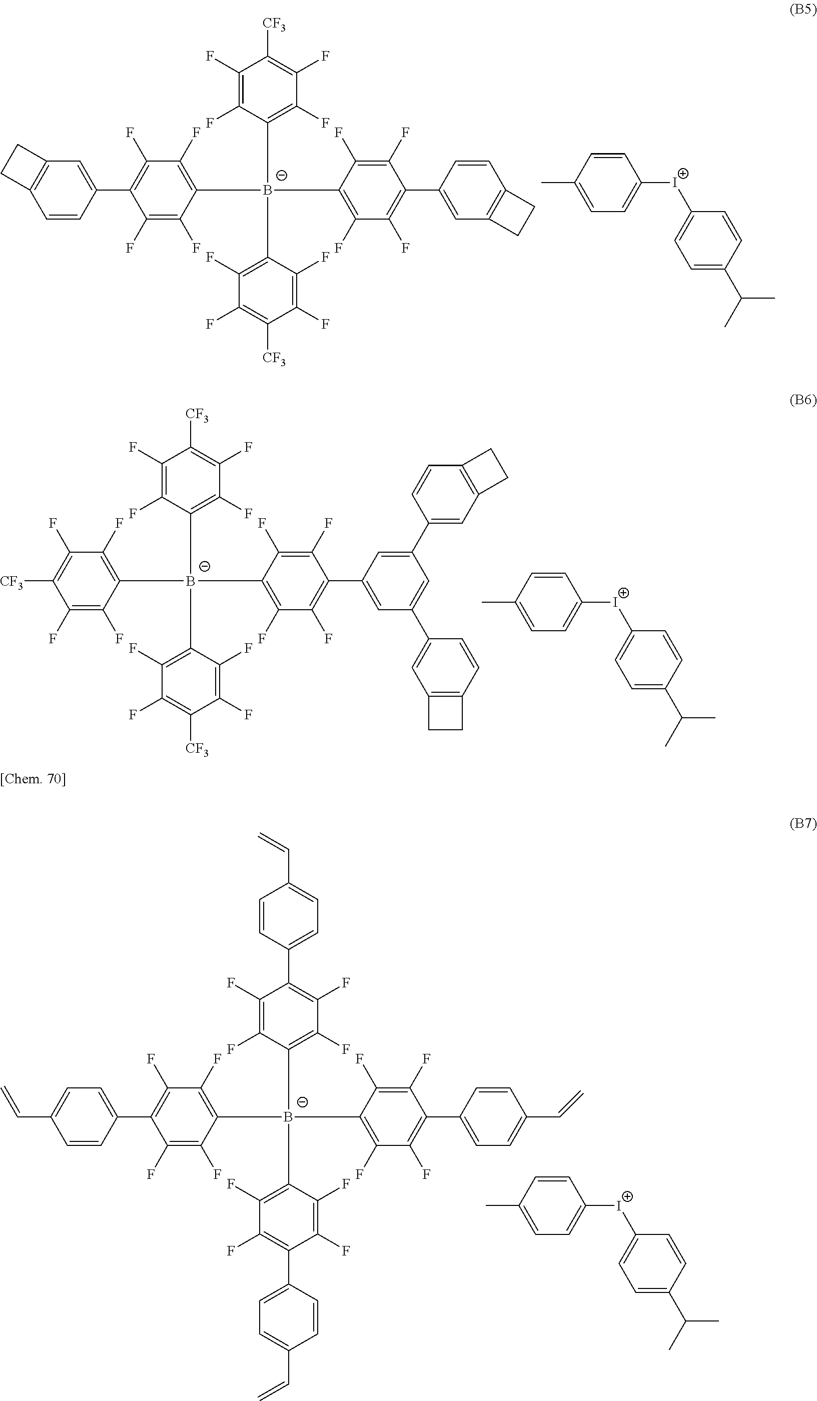

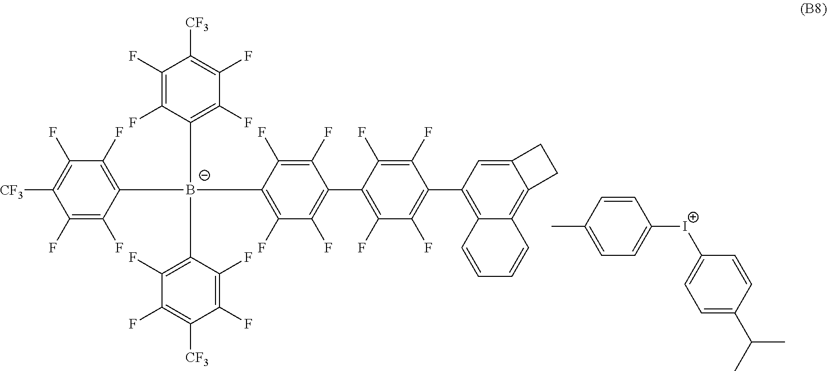 [SMILES: Fc1c(F)c(-c2cc3c(c4ccccc24)CC3)c(F)c(F)c1-c1c(F)c(F)c([B-](c2c(F)c(F)c(C(F)(F)F)c(F)c2F)(c2c(F)c(F)c(C(F)(F)F)c(F)c2F)c2c(F)c(F)c(C(F)(F)F)c(F)c2F)c(F)c1F ; Cc1ccc([I+]c2ccc(C(C)C)cc2)cc1]

### [High-Molecular Weight Charge Transport Compound]

Preferably, the composition of the invention contains a high-molecular weight hole transport compound as the high-molecular weight charge transport compound. The high-molecular weight hole transport compound is generally used to form a hole injection layer or a hole transport layer and is contained in a charge transport film-forming composition described later. In this case, the composition of the invention can be used to form the hole injection layer or the hole transport layer.

### [Polymer Having Arylamine Structure as Repeating Unit]

The high-molecular weight hole transport compound is preferably a polymer having an arylamine structure described below as a repeating unit. More preferably, the polymer has a crosslinking group.

The arylamine structure repeating unit of the polymer having the arylamine structure as a repeating unit is represented by formula (50) below.

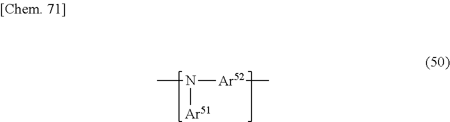

(In formula (50),
Ar⁵¹ represents an aromatic hydrocarbon group, an aromatic heterocyclic group, or a group in which a plurality of groups selected from aromatic hydrocarbon groups and aromatic heterocyclic groups are linked together;
Ar⁵² represents a divalent aromatic hydrocarbon group, a divalent aromatic heterocyclic group, or a divalent group in which a plurality of groups selected from the group consisting of divalent aromatic hydrocarbon groups and divalent aromatic heterocyclic groups are linked together directly or via a linking group;
Ar⁵¹ and Ar⁵² may form a ring via a single bond or a linking group; and
Ar⁵¹ and Ar⁵² may each optionally have a substituent and/or a crosslinking group.)

The optional substituents on Ar⁵¹ and Ar⁵² are preferably substituents selected from the substituent group Z.

The optional crosslinking groups on Ar⁵¹ and Ar⁵² are preferably crosslinking groups selected from the group T of crosslinking groups.

Preferably, the polymer having the arylamine structure represented by formula (50) as a repeating unit has a crosslinking group. The phrase “the polymer having the arylamine structure represented by formula (50) as a repeating unit has a crosslinking group” means that at least one repeating unit that has the arylamine structure represented by formula (50) and is contained in the polymer has a crosslinking group and/or that a repeating unit that is different from the repeating unit represented by formula (50) and is contained in the polymer has a crosslinking group.

Preferably, in the polymer, at least one repeating unit having the arylamine structure represented by formula (50) has a crosslinking group.

When the repeating unit having the arylamine structure represented by formula (50) has a crosslinking group, Ar⁵¹ and/or Ar⁵² has a crosslinking group. Preferably, Ar⁵¹ has a crosslinking group.

### (Terminal Group)

In the present description, the terminal group of a polymer is the structure of a terminal portion of the polymer that is formed by an end-capping agent used at the end of the polymerization of the polymer. In the composition of the invention, the terminal group of the polymer including the repeating unit represented by formula (50) is preferably a hydrocarbon group. From the viewpoint of charge transportability, the hydrocarbon group is preferably a hydrocarbon group having 1 to 60 carbon atoms, more preferably a hydrocarbon group having 1 to 40 carbon atoms, and still more preferably a hydrocarbon group having 1 to 30 carbon atoms.

Examples of the hydrocarbon group include:
linear, branched, and cyclic alkyl groups having generally 1 or more carbon atoms and preferably 4 or more carbon atoms and having generally 24 or less carbon atoms and preferably 12 or less carbon atoms such as a methyl group, an ethyl group, a n-propyl group, an i-propyl group, a n-butyl group, an i-butyl group, a sec-butyl group, a tert-butyl group, a n-hexyl group, a cyclohexyl group, and a dodecyl group;
linear, branched, and cyclic alkenyl groups having generally 2 or more carbon atoms and having generally 24 or less carbon atoms and preferably 12 or less carbon atoms such as a vinyl group;
linear and branched alkynyl groups having generally 2 or more carbon atoms and having generally 24 or less carbon atoms and preferably 12 or less carbon atoms such as an ethynyl group;
aromatic hydrocarbon groups having generally 6 or more carbon atoms and having generally 36 or less carbon atoms and preferably 24 or less carbon atoms such as a phenyl group and a naphthyl group; and
crosslinking groups that are hydrocarbon groups in the group T of crosslinking groups and are preferably crosslinking groups represented by formulas (X1) to (X4).

These hydrocarbon groups may each optionally have an additional substituent, and the optional additional substituent is preferably an alkyl group or an aromatic hydrocarbon group. When a plurality of optional additional substituents are present, they may be bonded together to form a ring. When these hydrocarbon groups are groups different from crosslinking groups, the substituent may further have a crosslinking group selected from the group T of crosslinking groups as an additional substituent.

From the viewpoint of charge transportability and durability, the terminal group is preferably an alkyl group, an aromatic hydrocarbon group, or a crosslinking group selected from the hydrocarbon groups in the group T of crosslinking groups and more preferably an aromatic hydrocarbon group. When the terminal group is not a crosslinking group, it is also preferable that the terminal group has a crosslinking group selected from the group T of crosslinking groups as a substituent.

### (Ar⁵²)

Ar⁵² represents a divalent aromatic hydrocarbon group, a divalent aromatic heterocyclic group, or a divalent group in which a plurality of groups selected from the group consisting of divalent aromatic hydrocarbon groups and divalent aromatic heterocyclic groups are linked together directly or via a linking group. The aromatic hydrocarbon group and the aromatic heterocyclic group may each optionally have a substituent and/or a crosslinking group.

The optional substituent is preferably a substituent selected from the substituent group Z. The optional crosslinking group is preferably a crosslinking group selected from the group T of crosslinking groups.

### (Twisted Structure)

It is preferable that the repeating unit represented by formula (50) above includes a partial structure represented by formula (63) below in the main chain structure represented by Ar⁵² because the main chain has a twisted structure and conjugation is disrupted.

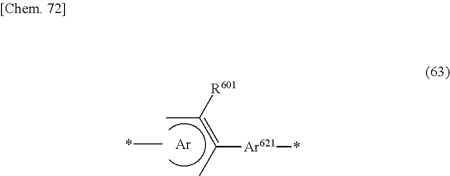

(In formula (63),
R⁶⁰¹ represents an alkyl group optionally having a substituent or a crosslinking group;
Ar⁶²¹ represents a divalent aromatic hydrocarbon group optionally having a substituent and/or a crosslinking group or a divalent aromatic heterocyclic group optionally having a substituent and/or a crosslinking group;
ring Ar represents an aromatic hydrocarbon structure optionally having a substituent and/or a crosslinking group or a divalent aromatic heterocyclic structure optionally having a substituent and/or a crosslinking group; and
* represents a position bonded to an adjacent atom.)

### (Ar⁵¹)

Ar⁵¹ is an aromatic hydrocarbon group, an aromatic heterocyclic group, or a group in which a plurality of groups selected from aromatic hydrocarbon groups and aromatic heterocyclic groups are linked together. The aromatic hydrocarbon group and the aromatic heterocyclic group may each optionally have a substituent and/or a crosslinking group.

The optional substituent is preferably a substituent selected from the substituent group Z, particularly from the substituent group X. The optional crosslinking group is preferably a crosslinking group selected from the group T of crosslinking groups.

From the viewpoint of improving the stability of the film, it is preferable that Ar⁵¹ has a crosslinking group.

### (Preferred Structure of Ar⁵¹ Having Crosslinking Group)

When Ar⁵¹ has a crosslinking group, it is preferable that Ar⁵¹ has a structure in which a monovalent group including 2 to 5 optionally substituted benzene rings linked together has at its terminal end a crosslinking group selected from the group T of crosslinking groups. It is more preferable that Ar⁵¹ has a structure in which a monovalent group including 2 to 5 non-substituted benzene rings linked together has at its terminal end a crosslinking group selected from the group T of crosslinking groups.

### <Preferred Ar⁵¹>

From the viewpoint of obtaining good charge transportability and good durability, Ar⁵¹ is preferably an aromatic hydrocarbon group, more preferably a benzene ring (phenyl group), a group including 2 to 5 benzene rings linked together, or a monovalent group including a fluorene ring (a fluorenyl group), still more preferably a fluorenyl group, and particularly preferably a 2-fluorenyl group. These groups may each optionally have a substituent and/or a crosslinking group. The substituent is preferably a group selected from the substituent group Z, and the crosslinking group is preferably a crosslinking group selected from the group T of crosslinking groups.

No particular limitation is imposed on the optional substituent on each of the aromatic hydrocarbon group and the aromatic heterocyclic group represented by Ar⁵¹ so long as the characteristics of the polymer are not significantly impaired. The substituent is preferably a group selected from the substituent group Z, more preferably an alkyl group, an alkoxy group, an aromatic hydrocarbon group, or an aromatic heterocyclic group, and still more preferably an alkyl group.

From the viewpoint of solubility in a solvent, Ar⁵¹ is preferably a fluorenyl group substituted with an alkyl group having 1 to 24 carbon atoms and particularly preferably a 2-fluorenyl group substituted with an alkyl group having 4 to 12 carbon atoms. Ar⁵¹ is still more preferably a 9-alkyl-2-fluorenyl group obtained by substituting a 2-fluorenyl group with an alkyl group at the 9-position and particularly preferably a 9,9′-dialkyl-2-fluorenyl group substituted with 2 alkyl groups.

When Ar⁵¹ is a fluorenyl group substituted with an alkyl group at at least one of the 9- and 9′-positions, the solubility in a solvent and the durability of the fluorene ring tend to be improved. When Ar⁵¹ is a fluorenyl group substituted with alkyl groups at both the 9- and 9′-positions, the solubility in a solvent and the durability of the fluorene ring tend to be further improved.

From the viewpoint of the solubility in a solvent, Ar⁵¹ is also preferably a spirobifluorenyl group.

In the polymer including the repeating unit represented by formula (50), it is preferable that the repeating unit represented by formula (50) is a repeating unit in which Ar⁵¹ is a group represented by formula (51) below, a group represented by formula (52) below, or a group represented by formula (53) below.

### <Group Represented by Formula (51)>

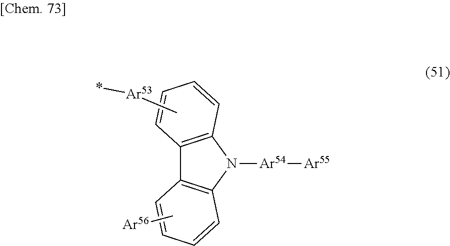

(In formula (51),

* represents a bond to the nitrogen atom in the main chain in formula (50).

Ar⁵³ and Ar⁵⁴ each independently represents a divalent aromatic hydrocarbon group optionally having a substituent and/or a crosslinking group, an aromatic heterocyclic group optionally having a substituent and/or a crosslinking group, or a divalent group in which a plurality of aromatic hydrocarbon groups each optionally having a substituent and/or a crosslinking group or a plurality of aromatic heterocyclic groups each optionally having a substituent and/or a crosslinking group are linked together directly or via a linking group.

Ar⁵⁵ represents an aromatic hydrocarbon group optionally having a substituent and/or a crosslinking group, an aromatic heterocyclic group optionally having a substituent and/or a crosslinking group, or a monovalent group in which aromatic hydrocarbon or aromatic heterocyclic groups each optionally having a substituent and/or a crosslinking group are linked together directly or via a linking group.

Ar⁵⁶ represents a hydrogen atom, a substituent, or a crosslinking group.)

The aromatic hydrocarbon groups and the aromatic heterocyclic groups may each optionally have a substituent and/or a crosslinking group.

The optional substituent is preferably a group selected from the substituent group Z. The optional crosslinking group is preferably a group selected from the group T of crosslinking groups.

### (Ar⁵³)

Ar⁵³ is preferably a group including 1 to 6 divalent aromatic hydrocarbon groups linked together, more preferably a group including 2 to 4 divalent aromatic hydrocarbon groups linked together, still more preferably a group including 1 to 4 phenylene rings linked together, and particularly preferably a biphenylene group including two phenylene rings linked together.

These groups may each optionally have a substituent and/or a crosslinking group. The optional substituent is preferably a group selected from the substituent group Z. The optional crosslinking group is preferably a group selected from the group T of crosslinking groups. Preferably, Ar⁵³ has no substituent and no crosslinking group.

When these divalent aromatic hydrocarbon groups or divalent aromatic heterocyclic groups are linked together, it is preferable that the resulting group is a group in which the plurality of bonded divalent aromatic hydrocarbon groups are bonded so as not to be conjugated to each other. Specifically, the resulting group includes preferably a 1,3-phenylene group or a group having a substituent and having a twisted structure due to a steric effect of the substituent and is more preferably a 1,3-phenylene group having no substituent and no crosslinking group or a group in which a plurality of 1,3-phenylene groups having no substituent and no crosslinking group are linked together.

### (Ar⁵⁴)

From the viewpoint of obtaining good charge transportability and good durability, Ar⁵⁴ is preferably one divalent aromatic hydrocarbon group or a group in which a plurality of divalent aromatic hydrocarbon groups that may be the same or different are linked together. Each divalent aromatic hydrocarbon group may optionally have a substituent. When a plurality of divalent aromatic hydrocarbon groups are linked together, the number of linked groups is preferably 2 to 10, more preferably 6 or less, and particularly preferably 3 or less from the viewpoint of the stability of the film. The aromatic hydrocarbon ring structure is preferably a benzene ring, a naphthalene ring, an anthracene ring, or a fluorene ring and more preferably a benzene ring or a fluorene ring. The group including a plurality of divalent aromatic hydrocarbon groups linked together is preferably a group including 1 to 4 phenylene rings linked together or a group including a phenylene ring and a fluorene ring linked together. It is particularly preferable to use a biphenylene group including two phenylene rings linked together because the LUMO spreads widely.

These groups may each optionally have a substituent and/or a crosslinking group. The optional substituent is preferably a group selected from the substituent group Z. The optional crosslinking group is preferably a group selected from the group T of crosslinking groups. The substituent is more preferably a phenyl group, a naphthyl group, or a fluorenyl group. It is also preferable that no substituent is included.

### (Ar⁵⁵)

Ar⁵⁵ is an aromatic hydrocarbon group optionally having a substituent and/or a crosslinking group, an aromatic heterocyclic group optionally having a substituent and/or a crosslinking group, or a monovalent group in which a plurality of aromatic hydrocarbon or aromatic heterocyclic groups each optionally having a substituent and/or a crosslinking group are linked together directly or via a linking group. Preferably, Ar⁵⁵ is a monovalent aromatic hydrocarbon group or a group including a plurality of monovalent aromatic hydrocarbon groups linked together.

These groups may each optionally have a substituent and/or a crosslinking group. The optional substituent is preferably a group selected from the substituent group Z, particularly from the substituent group X. The optional crosslinking group is preferably a group selected from the group T of crosslinking groups.

When these groups are linked together, the resulting group is a monovalent group including 2 to 10 groups linked together and preferably a monovalent group including 2 to 5 groups linked together. The aromatic hydrocarbon group and the aromatic heterocyclic group used may be the same as the aromatic hydrocarbon group and the aromatic heterocyclic group, respectively, described above for Ar⁵¹.

Preferably, Ar⁵⁵ has a structure represented by any of the following schemes 2A, 2B, and 2C.

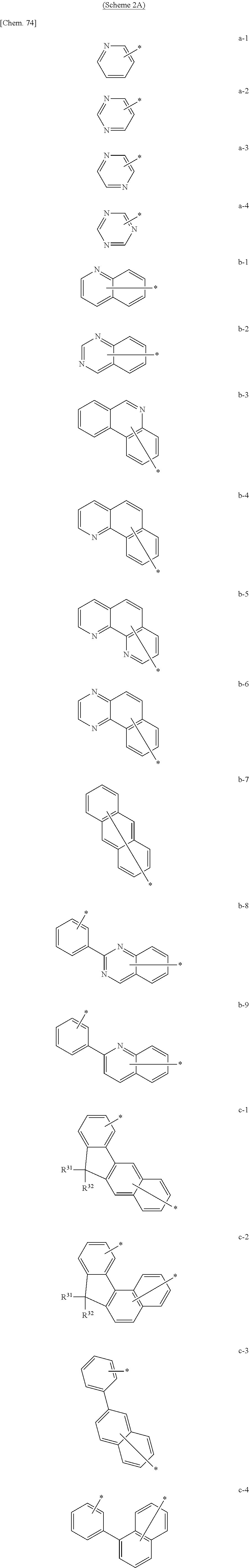

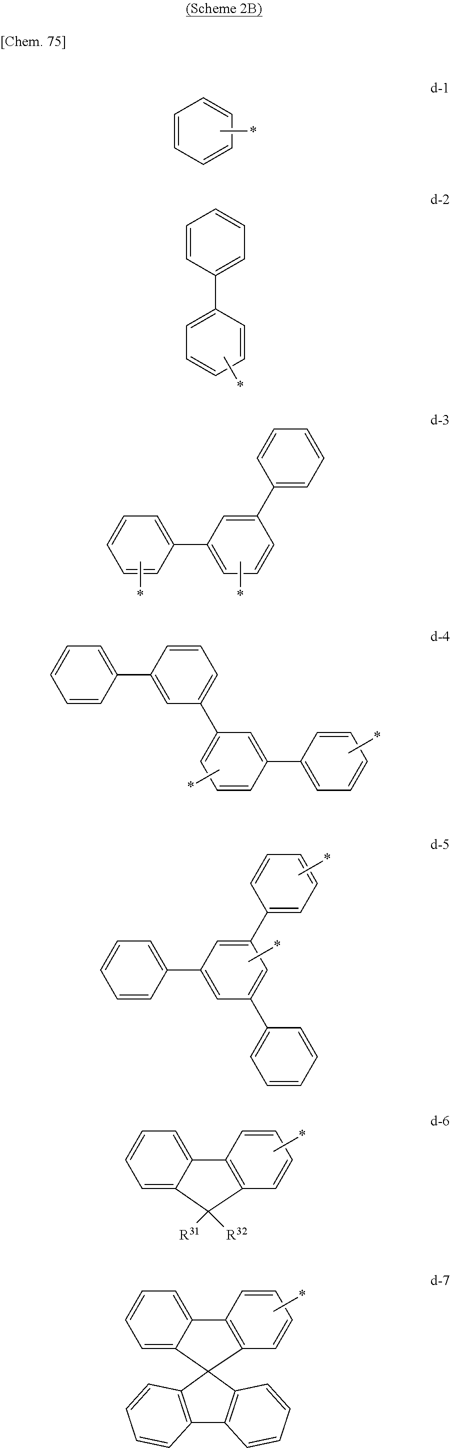

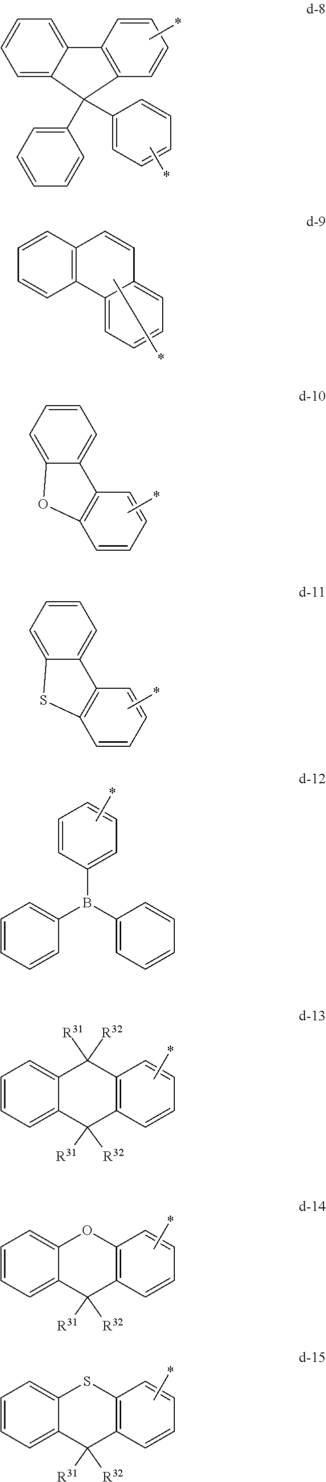

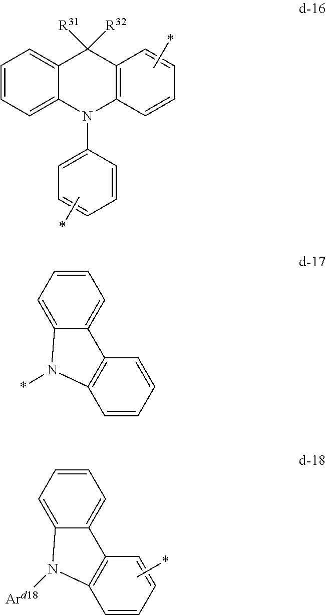

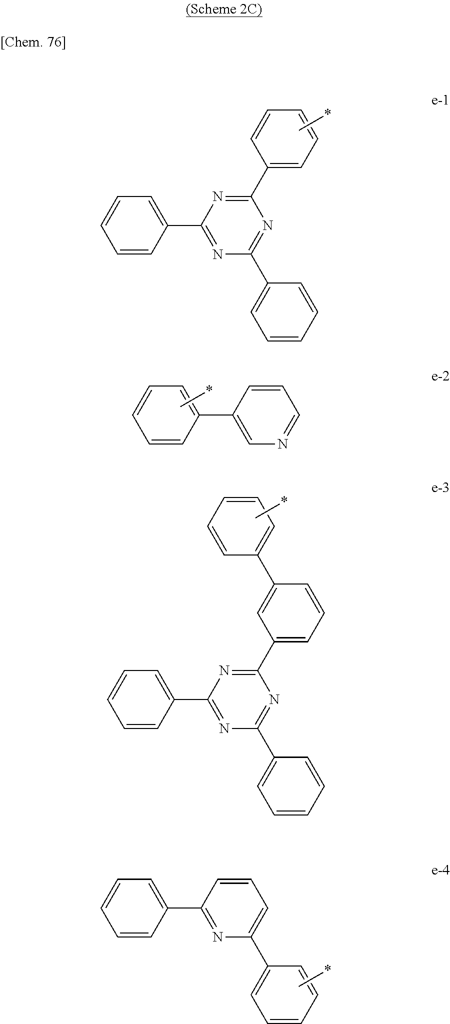

In schemes 2A to 2C above, * represents a position of bonding to Ar⁵⁴. When a plurality of *'s are present, one of them represents a position of bonding to Ar⁵⁴.

These structures may each optionally have a substituent and/or a crosslinking group. The optional substituent on each of these structures is preferably a group selected from the substituent group Z. The optional crosslinking group is preferably a group selected from the group T of crosslinking groups.

(R³¹ and R³²)

R³¹ and R³² in schemes 2A and 2B are each independently preferably a linear, branched, or cyclic alkyl group optionally having a substituent. No particular limitation is imposed on the number of carbon atoms in the alkyl group. To maintain the solubility of the polymer, the number of carbon atoms is preferably 1 or more and 6 or less and more preferably 3 or less, and the alkyl group is still more preferably a methyl group or an ethyl group.

R³¹(s) and R³²(s) may be the same or different. Preferably, R³¹(s) and R³²(s) are all the same because charges can be uniformly distributed around nitrogen atoms and the polymer can be easily synthesized.

### (Ar^{d18})

Ar^{d18} in scheme 2B is each independently an aromatic hydrocarbon group or an aromatic heterocyclic group. From the viewpoint of stability, Ar^{d18} is preferably an aromatic hydrocarbon group and more preferably a phenyl group. These groups may each optionally have a substituent or a crosslinking group. The optional substituent is preferably a group selected from the substituent group Z, particularly from the substituent group X. The optional crosslinking group is preferably a group selected from the group T of crosslinking groups.

From the viewpoint of the distribution of the LUMO of the molecule, Ar⁵⁵ is preferably a structure selected from a-1 to a-4, b-1 to b-9, c-1 to c-4, d-1 to d-18, and e-1 to e-4 shown above. From the viewpoint of facilitating the spreading of the LUMO when an electron-withdrawing group is present, Ar⁵⁵ is preferably a structure selected from a-1 to a-4, b-1 to b-9, d-1 to d-12, d-17, d-18, and e-1 to e-4. From the viewpoint of the effect of confining excitons having a high triplet level and formed in a light-emitting layer, Ar⁵⁵ is preferably a structure selected from a-1 to a-4, d-1 to d-12, d-17, d-18, and e-1 to e-4. From the viewpoint of the ease of synthesis and high stability, Ar⁵⁵ is more preferably a structure selected from d-1, d-10, d-17, d-18, and e-1 and particularly preferably a benzene ring structure in d-1, a fluorene ring structure in d-6, or a carbazole structure in d-17.

When Ar⁵⁵ is a fluorene structure represented by d-6, Ar⁵⁵ is preferably a 2-fluorenyl group. The 2-fluorenyl group may optionally have a substituent and/or a crosslinking group at each of the 9- and 9′-positions. The optional substituent is preferably a group selected from the substituent group Z. The optional crosslinking group is preferably a group selected from the group T of crosslinking groups. The substituent is particularly preferably an alkyl group.

### (Ar⁵⁶)

Ar⁵⁶ represents a hydrogen atom, a substituent, or a crosslinking group. When Ar⁵⁶ is a substituent, no particular limitation is imposed on the substituent. The substituent is preferably an aromatic hydrocarbon group or an aromatic heterocyclic group and may further optionally have a substituent selected from the substituent group Z and/or a crosslinking group selected from the group T of crosslinking groups. When Ar⁵⁶ is a crosslinking group, the crosslinking group is preferably a crosslinking group selected from the group T of crosslinking groups.

When Ar⁵⁶ is a substituent, it is preferable from the viewpoint of improving durability that Ar⁵⁶ is bonded to the 3-position of the carbazole structure in formula (51) to which Ar⁵⁶ is bonded. From the viewpoint of improving durability and of charge transportability, Ar⁵⁶ is preferably an aromatic hydrocarbon group optionally having a substituent or an aromatic heterocyclic group optionally having a substituent and more preferably an aromatic hydrocarbon group optionally having a substituent.

From the viewpoint of the ease of synthesis and charge transportability, Ar⁵⁶ is preferably a hydrogen atom.

### (Specific Examples of Group Represented by Formula (51))

Specific examples of the group represented by formula (51) are shown below. However, the group represented by formula (51) is not limited thereto.

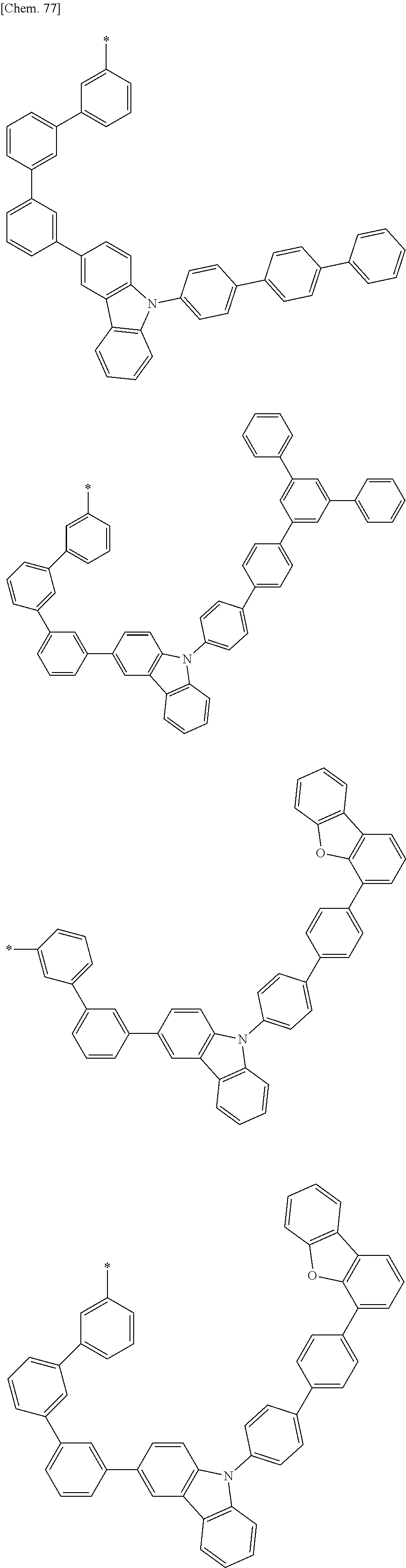 [SMILES: *c1cccc(-c2cccc(-c3cccc(-c4ccc5c(c4)c4ccccc4n5-c4ccc(-c5ccc(-c6ccccc6)cc5)cc4)c3)c2)c1 ; *c1cccc(-c2cccc(-c3cccc(-c4ccc5c(c4)c4ccccc4n5-c4ccc(-c5ccc(-c6cc(-c7ccccc7)cc(-c7ccccc7)c6)cc5)cc4)c3)c2)c1 ; *c1cccc(-c2cccc(-c3ccc4c(c3)c3ccccc3n4-c3ccc(-c4ccc(-c5cccc6c5oc5ccccc56)cc4)cc3)c2)c1 ; *c1cccc(-c2cccc(-c3cccc(-c4ccc5c(c4)c4ccccc4n5-c4ccc(-c5ccc(-c6cccc7c6oc6ccccc67)cc5)cc4)c3)c2)c1]

### <Group Represented by Formula (52)>

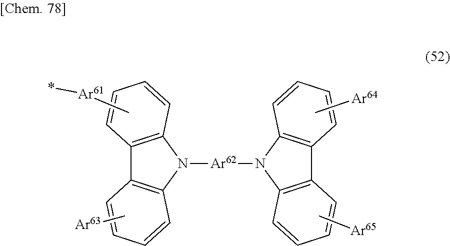

(In formula (52),

Ar⁶¹ and Ar⁶² each independently represent a divalent aromatic hydrocarbon group optionally having a substituent and/or a crosslinking group, a divalent aromatic heterocyclic group optionally having a substituent and/or a crosslinking group, or a divalent group in which a plurality of aromatic hydrocarbon groups each optionally having a substituent and/or a crosslinking group or a plurality of aromatic heterocyclic groups each optionally having a substituent or a crosslinking group are linked together directly or via a linking group.

Ar⁶³ to Ar⁶⁵ are each independently a hydrogen atom, a substituent, or a crosslinking group.

* represents a position of bonding to the nitrogen atom in the main chain in formula (50).)

The optional substituent on each aromatic hydrocarbon group, the optional substituent on each aromatic heterocyclic group, and Ar⁶³ to Ar⁶⁵ when they are each a substituent are each preferably a group selected from the substituent group Z, particularly from the substituent group X.

The optional substituent on each aromatic hydrocarbon group, the optional crosslinking group on each aromatic heterocyclic group, and Ar⁶³ to Ar⁶⁵ when they are each a crosslinking group are each preferably a group selected from the group T of crosslinking groups.

(Ar⁶³ to Ar⁶⁵)

Ar⁶³ to Ar⁶⁵ are each independently the same as Ar⁵⁶ described above.

Ar⁶³ to Ar⁶⁴ are each preferably a hydrogen atom.

### (Ar⁶²)

Ar⁶² is a divalent aromatic hydrocarbon group optionally having a substituent and/or a crosslinking group, a divalent aromatic heterocyclic group optionally having a substituent and/or a crosslinking group, or a divalent group in which a plurality of aromatic hydrocarbon groups each optionally having a substituent and/or a crosslinking group or a plurality of aromatic heterocyclic groups each optionally having a substituent and/or a crosslinking group are linked together directly or via a linking group. Preferably, Ar⁶² is a divalent aromatic hydrocarbon group optionally having a substituent and/or a crosslinking group or a group in which a plurality of divalent aromatic hydrocarbon groups each optionally having a substituent and/or a crosslinking group are linked together. The optional substituent on each aromatic hydrocarbon group and the optional substituent on each aromatic heterocyclic group are each preferably a group selected from the substituent group Z, particularly from the substituent group X. The optional crosslinking group on each aromatic hydrocarbon group and the optional crosslinking group on each aromatic heterocyclic group are each preferably a group selected from the group T of crosslinking groups.

The specific structure of Ar⁶² is the same as that of Ar⁵⁴.

A specific group represented by Ar⁶² is preferably a divalent group including a benzene ring, a naphthalene ring, an anthracene ring, or a fluorene ring or a group in which a plurality of groups selected from these groups are linked together, more preferably a divalent group including a benzene ring or a group in which a plurality of groups each including a benzene ring are linked together, particularly preferably a 1,4-phenylene group including a divalent benzene ring bonded at its 1- and 4-positions, a 2,7-fluorenylene group including a fluorene ring bonded at its 2- and 7-positions, or a group including a plurality of these groups linked together, and most preferably a group including “1,4-phenylene group-2,7-fluorenylene group-1,4-phenylene group-.”

In the preferred structures of Ar⁶², it is preferable that the phenylene group has no substituent and no crosslinking group at positions other than the bonding positions because Ar⁶² is not twisted due to the steric effect of the substituent. It is preferable from the viewpoint of improving the solubility and the durability of the fluorene structure that the fluorenylene group has substituents or crosslinking groups at the 9- and 9′ positions. The substituents are each preferably a substituent selected from the substituent group Z, particularly from the substituent group X and are each more preferably an alkyl group. Each substituent may be further substituted with a crosslinking group. The crosslinking group is preferably a crosslinking group selected from the group T of crosslinking groups. The substituents are preferred.

### (Ar⁶¹)

Ar⁶¹ is the same group as Ar⁵³ described above, and its preferred structures are also the same as those of Ar⁵³.

### (Specific Example of Group Represented by Formula (52))

A specific example of the group represented by formula (52) is shown below. However, the group represented by formula (52) is not limited to the following group.

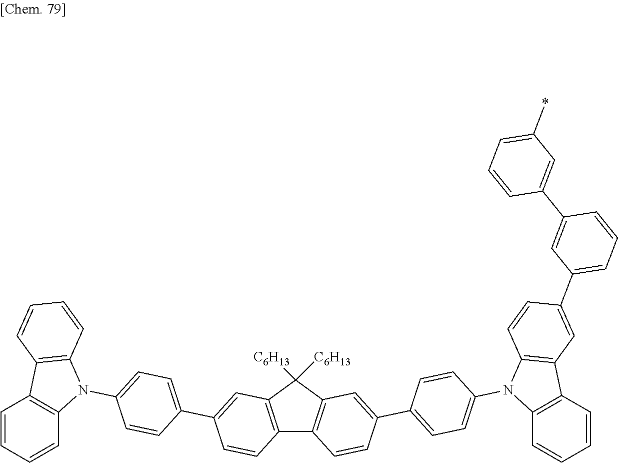

### <Group Represented by Formula (53)>

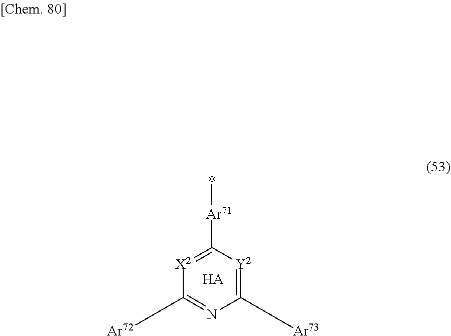

(In formula (53),

* represents a bond to the nitrogen atom in the main chain in formula (50).

Ar⁷¹ represents a divalent aromatic hydrocarbon group.

Ar⁷² and Ar⁷³ each independently represent an aromatic hydrocarbon group, an aromatic heterocyclic group, or a monovalent group in which two or more groups selected from aromatic hydrocarbon groups and aromatic heterocyclic groups are linked together directly or via a linking group. Each of these groups may optionally have a substituent and/or a crosslinking group.

The ring HA is an aromatic heterocycle including a nitrogen atom.

X² and Y² each independently represent a carbon atom or a nitrogen atom. When at least one of X² and Y² is a carbon atom, the carbon atom may optionally have a substituent and/or a crosslinking group.)

The optional substituent is preferably a group selected from the substituent group Z, particularly from the substituent group X. The optional crosslinking group is preferably a group selected from the group T of crosslinking groups.

### <Ar⁷¹>

Ar⁷¹ is the same group as Ar⁵³ described above.

Ar⁷¹ is particularly preferably a group in which 2 to 6 benzene rings each optionally having a substituent are linked together and most preferably a quaterphenylene group in which 4 benzene rings each optionally having a substituent are linked together.

Ar⁷¹ includes preferably at least one benzene ring linked at its 1- and 3-positions, which are non-conjugated sites, and more preferably at least two such benzene rings.

When Ar⁷¹ is a group in which a plurality of divalent aromatic hydrocarbon groups each optionally having a substituent are linked together, it is preferable from the viewpoint of charge transportability or durability that all the divalent aromatic hydrocarbon groups are bonded and linked directly.

Therefore, preferred examples of Ar⁷¹ serving as the structure that connects the nitrogen atom in the main chain of the polymer to the ring HA in formula (53) include those shown in schemes 2-1 and 2-2 below. In schemes 2-1 and 2-2 below, each * represents a position of bonding to the nitrogen atom in the main chain of the polymer or to the ring HA in formula (53). Any one of the two *s may be bonded to the nitrogen atom in the main chain of the polymer or the ring HA.

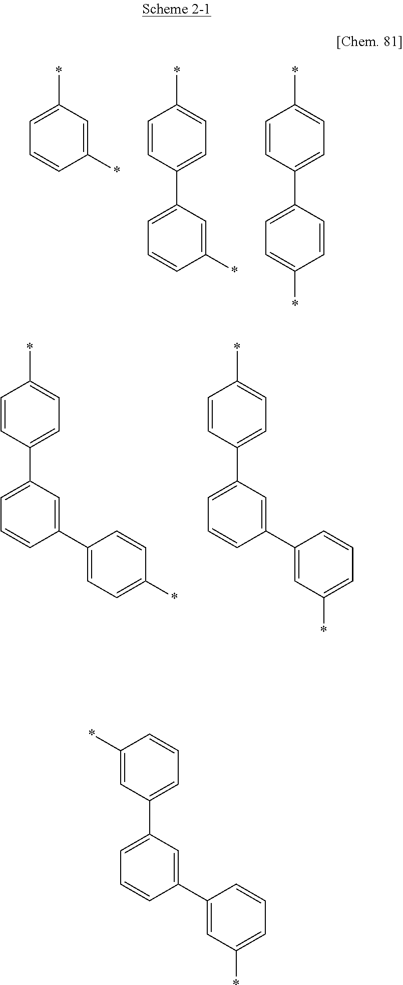

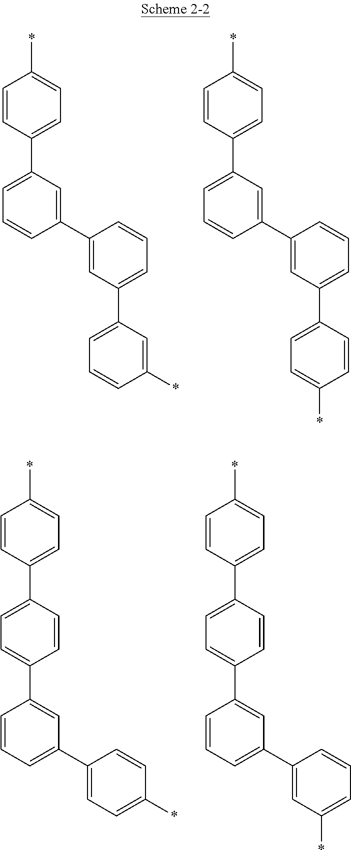 [SMILES: *c1ccc(-c2cccc(-c3cccc(-c4cccc(*)c4)c3)c2)cc1 ; *c1ccc(-c2cccc(-c3cccc(-c4ccc(*)cc4)c3)c2)cc1 ; *c1ccc(-c2ccc(-c3cccc(-c4ccc(*)cc4)c3)cc2)cc1 ; *c1ccc(-c2ccc(-c3cccc(-c4cccc(*)c4)c3)cc2)cc1]

The optional substituent on Ar⁷¹ may be selected from the substituent group Z, particularly from the substituent group X, or may be a combination of substituents selected therefrom. A preferred range of the optional substituent on Ar⁷¹ is the same as that of the optional substituent when Ar⁵³ described above is an aromatic hydrocarbon group.

<X² and Y²>

X² and Y² each independently represent a C (carbon) atom or a N (nitrogen) atom. When at least one of X² and Y² is a carbon atom, the carbon atom may optionally have a substituent.

From the viewpoint of the ease of localizing the LUMO around the ring HA, it is preferable that X² and Y² are each a N atom.

The optional substituent when at least one of X² and Y² is a C atom may be selected from the substituent group Z, particularly from the substituent group X, or may be a combination of substituents selected therefrom. From the viewpoint of charge transportability, it is preferable that X² and Y² have no substituent.

<Ar⁷² and Ar⁷³>

Ar⁷² and Ar⁷³ are each independently an aromatic hydrocarbon group, an aromatic heterocyclic group, or a monovalent group in which two or more groups selected from aromatic hydrocarbon groups and aromatic heterocyclic groups are linked together directly or via a linking group. Each of these groups may optionally have a substituent and/or a crosslinking group. The optional substituent is preferably a group selected from the substituent group Z, particularly from the substituent group X. The optional crosslinking group is preferably a group selected from the group T of crosslinking groups.

From the viewpoint of the distribution of the LUMO, it is preferable that Ar⁷² and Ar⁷³ each independently have a structure selected from a-1 to a-4, b-1 to b-9, c-1 to c-4, d-1 to d-16, and e-1 to e-4 shown in schemes 2A to 2C above.

From the viewpoint of facilitating the spreading of the LUMO when an electron-withdrawing group is present, Ar⁷² and Ar⁷³ are each more preferably a structure selected from a-1 to a-4, b-1 to b-9, c-1 to c-5, d-1 to d-12, and e-1 to e-4.

From the viewpoint of the effect of confining excitons having a high triplet level and formed in a light-emitting layer, Ar⁷² and Ar⁷³ are each more preferably a structure selected from a-1 to a-4, d-1 to d-12, and e-1 to e-4.

To prevent aggregation of molecules, Ar⁷² and Ar⁷³ are each still more preferably a structure selected from d-1 to d-12 and e-1 to e-4. From the viewpoint of the ease of synthesis and high stability, it is preferable that Ar⁷²=Ar⁷³=d-1 or d-10, and the benzene ring structure represented by d-1 is particularly preferred.

Each of these structures may optionally have a substituent.

### (Specific Examples of Group Represented by Formula (53))

Specific examples of the group represented by formula (53) are shown below. However, the group represented by formula (53) is not limited thereto.

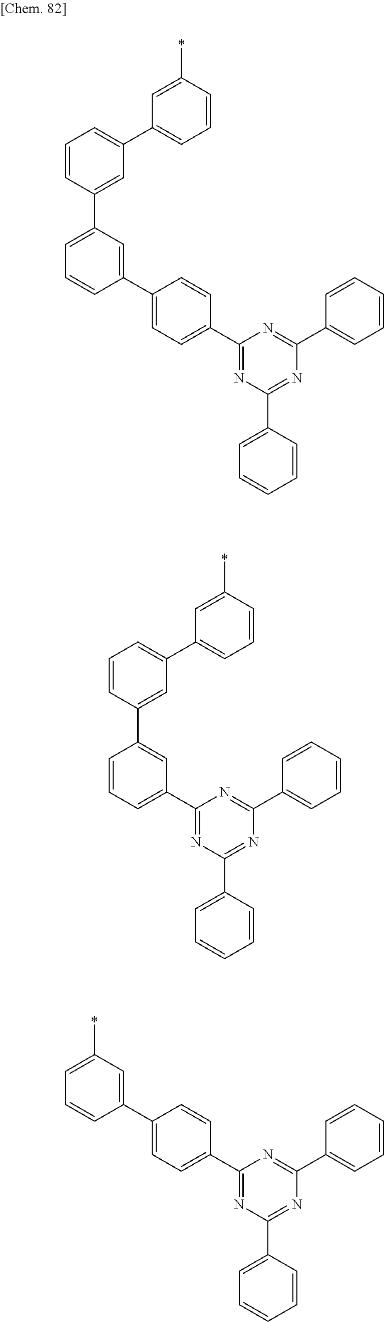 [SMILES: *c1cccc(-c2cccc(-c3cccc(-c4ccc(-c5nc(-c6ccccc6)nc(-c6ccccc6)n5)cc4)c3)c2)c1 ; *c1cccc(-c2cccc(-c3cccc(-c4nc(-c5ccccc5)nc(-c5ccccc5)n4)c3)c2)c1 ; *c1cccc(-c2ccc(-c3nc(-c4ccccc4)nc(-c4ccccc4)n3)cc2)c1]

### (Preferred Repeating Units Represented by Formula (50))

The repeating unit represented by formula (50) is preferably a repeating unit selected from a repeating unit represented by formula (54) below, a repeating unit represented by formula (55) below, a repeating unit represented by formula (56) below, a repeating unit represented by formula (57) below, and a repeating unit represented by formula (60) below.

In particular, the repeating unit represented by formula (54) below is preferred because of high heat resistance due to its structure including aromatic hydrocarbon rings condensed together.

In particular, the repeating unit represented by formula (55) below is preferred because a phenylene ring having R³⁰⁴ and R³⁰⁵ has a structure twisted relative to adjacent phenylene rings and therefore the extension of conjugation of the polymer is suppressed, so that the Ti level of the polymer is improved.

In particular, the repeating unit represented by formula (56) below is preferred because it has a carbazole structure and therefore increases the heat resistance.

In particular, the repeating unit represented by formula (57) below is preferred because the LUMO of the polymer can be easily spread and therefore the electron durability tends to increase.

In particular, the repeating unit represented by formula (60) below is preferred because its hole transportability is high.

The polymer in the invention includes preferably a repeating unit selected from the repeating unit represented by formula (54) below, the repeating unit represented by formula (55) below, the repeating unit represented by formula (56) below, and the repeating unit represented by formula (57) below and includes more preferably the repeating unit represented by formula (54) below or the repeating unit represented by formula (57) below.

Preferably, the polymer in the invention includes at least one selected from the repeating unit represented by formula (54) below, the repeating unit represented by formula (55) below, the repeating unit represented by formula (56) below, and the repeating unit represented by formula (57) below and further includes the repeating unit represented by formula (60) below. More preferably, the polymer in the invention includes the repeating unit represented by formula (54) below or the repeating unit represented by formula (57) below and further includes the repeating unit represented by formula (60) below.

### <Repeating Unit Represented by Formula (54)>

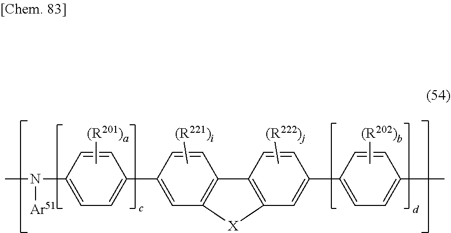

(In formula (54),

Ar⁵¹ is the same as Ar⁵¹ in formula (50) above.

X is —C(R²⁰⁷)(R²⁰⁸)—, —N(R²⁰⁹)—, or —C(R²¹¹)(R²¹²)—C(R²¹³)(R²¹⁴)—.

R²⁰¹, R²⁰², R²²¹, and R²²² are each independently an alkyl group optionally having a substituent and/or a crosslinking group.

R²⁰⁷ to R²⁰¹ and R²¹¹ to R²¹⁴ are each independently a hydrogen atom, an alkyl group optionally having a substituent and/or a crosslinking group, an aralkyl group optionally having a substituent and/or a crosslinking group, or an aromatic hydrocarbon group optionally having a substituent and/or a crosslinking group.

a and b are each independently an integer of 0 to 4.

c is an integer of 0 to 3.

d is an integer of 0 to 4.

i and j are each independently an integer of 0 to 3.)

(R²⁰¹, R²⁰², R²²¹, and R²²²)

R²⁰¹, R²⁰², R²²¹, and R²²² in the repeating unit represented by formula (54) are each independently an alkyl group optionally having a substituent and/or a crosslinking group.

The alkyl group is a linear, branched, or cyclic alkyl group. No particular limitation is imposed on the number of carbon atoms in the alkyl group. To maintain the solubility of the polymer, the number of carbon atoms is preferably 1 or more and 8 or less, more preferably 6 or less, and still more preferably 3 or less. The alkyl group is more preferably a methyl group or an ethyl group.

When a plurality of R²⁰¹'s are present, the plurality of R²⁰¹'s may be the same or different. When a plurality of R²⁰²'s are present, the plurality of R²⁰²'s may be the same or different. Preferably, all of R²⁰¹'s and R²⁰²'s are the same because charges can be distributed uniformly around the nitrogen atom and the polymer can be easily synthesized.

When a plurality of R²²¹'s are present, the plurality of R²²¹'s may be the same or different. When a plurality of R²²²'s are present, the plurality of R²²²'s may be the same or different. Preferably, all of R²²¹'s and R²²²'s are the same because the polymer can be easily synthesized.

(R²⁰⁷ to R²⁰⁹ and R²¹¹ to R²¹⁴)

R²⁰⁷ to R²⁰⁹ and R²¹¹ to R²¹⁴ are each independently a hydrogen atom, an alkyl group optionally having a substituent and/or a crosslinking group, an aralkyl group optionally having a substituent and/or a crosslinking group, or an aromatic hydrocarbon group optionally having a substituent and/or a crosslinking group.

No particular limitation is imposed on the alkyl group. However, the number of carbon atoms in the alkyl group is preferably 1 or more and 24 or less, more preferably 8 or less, and still more preferably 6 or less, because the solubility of the polymer tends to increase. The alkyl group may have a linear, branched, or cyclic structure.

Specific examples of the alkyl group include a methyl group, an ethyl group, a n-propyl group, an i-propyl group, a n-butyl group, an i-butyl group, a sec-butyl group, a tert-butyl group, a n-hexyl group, a n-octyl group, a cyclohexyl group, and a dodecyl group.

No particular limitation is imposed on the aralkyl group. However, the number of carbon atoms in the aralkyl group is preferably 5 or more and 60 or less and more preferably 40 or less, because the solubility of the polymer tends to increase.

Specific examples of the aralkyl group include a 1,1-dimethyl-1-phenylmethyl group, a 1,1-di(n-butyl)-1-phenylmethyl group, a 1,1-di(n-hexyl)-1-phenylmethyl group, a 1,1-di(n-octyl)-1-phenylmethyl group, a phenylmethyl group, a phenylethyl group, a 3-phenyl-1-propyl group, a 4-phenyl-1-n-butyl group, a 1-methyl-1-phenylethyl group, a 5-phenyl-1-n-propyl group, a 6-phenyl-1-n-hexyl group, a 6-naphthyl-1-n-hexyl group, a 7-phenyl-1-n-heptyl group, an 8-phenyl-1-n-octyl group, and a 4-phenylcyclohexyl group.

No particular limitation is imposed on the aromatic hydrocarbon group. However, the number of carbon atoms in the aromatic hydrocarbon group is preferably 6 or more and is preferably 60 or less and 30 or less, because the solubility of the polymer tends to increase.

Specific examples of the aromatic hydrocarbon group include: 6-membered monocyclic rings and monovalent groups including 2 to 5 rings condensed together such as a benzene ring, a naphthalene ring, an anthracene ring, a phenanthrene ring, a perylene ring, a tetracene ring, a pyrene ring, a benzpyrene ring, a chrysene ring, a triphenylene ring, an acenaphthene ring, a fluoranthene ring, and a fluorene ring; and groups in which a plurality of groups selected from the above groups are linked together.

From the viewpoint of improving the charge transportability and durability, R²⁰⁷ and R²⁰⁸ are each preferably a methyl group or an aromatic hydrocarbon group. R²⁰⁷ and R²⁰⁸ are each more preferably a methyl group, and R²⁰⁹ is more preferably a phenyl group.

The alkyl groups represented by R²⁰¹, R²⁰², R²²¹, and R²²² and the alkyl groups, aralkyl groups, and aromatic hydrocarbon groups represented by R²⁰⁷ to R²⁰⁹ and R²¹¹ to R²¹⁴ may each optionally have a substituent and/or a crosslinking group. Examples of the substituent include those described as the preferred groups for the alkyl groups, aralkyl groups, and aromatic hydrocarbon groups represented by R²⁰⁷ to R²⁰⁹ and R²¹¹ to R²¹⁴. The crosslinking group is a crosslinking group selected from the group T of crosslinking groups.

From the viewpoint of lowering voltage, it is most preferable that the alkyl groups represented by R²⁰¹, R²⁰², R²²¹, and R²²² and the alkyl groups, aralkyl groups, and aromatic hydrocarbon groups represented by R²⁰⁷ to R²⁰⁹ and R²¹¹ to R²¹⁴ each have no substituent and no crosslinking group.

When a crosslinking group is bonded to the main chain structure of the repeating unit represented by formula (54), it is preferable that the crosslinking group is bonded to one of R²⁰⁷ to R²⁰⁹, R²¹¹ to R²¹³, and R²¹⁴ that is an alkyl group, an aralkyl group, or an aromatic hydrocarbon group.

(a, b, c, and d)

In the repeating unit represented by formula (54), a and b are each independently an integer of 0 to 4. a+b is preferably 1 or more. Moreover, a and b are each preferably 2 or less, and it is more preferable that both a and b are 1. When b is 1 or more, d is also 1 or more. When c is 2 or more, a plurality of a's may be the same or different. When d is 2 or more, a plurality of b's may be the same or different.

When a+b is 1 or more, the aromatic rings in the main chain are twisted due to steric hindrance. In this case, the solubility of the polymer in a solvent tends to be high, and a coating film formed by a wet deposition method and subjected to heat treatment tends to have high insolubility in a solvent. Therefore, in the case where a+b is 1 or more, when another organic layer (e.g., a light-emitting layer) is formed on this coating film by a wet deposition method, elution of the polymer to the composition for forming the other organic layer containing an organic solvent can be reduced.

In the repeating unit represented by formula (54), c is an integer of 0 to 3, and d is an integer of 0 to 4. c and d are each preferably 2 or less and are more preferably the same. Particularly preferably, both c and d are 1, or both c and d are 2.

When both c and d in the repeating unit represented by formula (54) are 1 or 2 and both a and b are 2 or 1, it is most preferable that R²⁰¹(s) and R²⁰²(s) are bonded to symmetric positions.

The phrase “R²⁰¹(s) and R²⁰²(s) are bonded to symmetric positions” means that the bonding positions of R²⁰¹(s) and R²⁰²(s) are symmetric with respect to the fluorene ring, carbazole ring, or 9,10-dihydrophenanthrene derivative structure in formula (54). In this case, a structure rotated 1800 about the main chain is regarded as the same structure as the original structure.

When R²²¹ and R²²² are present, it is preferable that they are each independently present at the 1-, 3-, 6-, or 8-position with respect to a carbon atom of a benzene ring to which X is bonded. When R²²¹ and/or R²²² is present at any of these positions, the condensed ring to which R²²¹ and/or R²²² is bonded and adjacent benzene rings on the main chain are twisted relative to each other due to steric hindrance. This is preferable because the solubility of the polymer in a solvent becomes high and a coating film formed by a wet deposition method and subjected to heat treatment tends to have good insolubility in a solvent.

(i and j)

In the repeating unit represented by formula (54), i and j are each independently an integer of 0 to 3. i and j are each independently preferably an integer of 0 to 2 and more preferably 0 or 1. Preferably, i and j are the same integer. To twist the main chain of the polymer, it is preferable that i and j are each preferably 1 or 2 and that R²²¹(s) and/or R²²²(s) is(are) bonded to the 1- and/or 3-position(s) of the benzene rings. For ease of synthesis, i and j are each preferably 0. As for the bonding positions of a benzene ring, the position of a carbon atom to which R²²¹ or R²²² can be bonded and which is adjacent to the carbon atom to which X is bonded is defined as the 1-position, and the position of a carbon atom included in the main chain and bonded to an adjacent structure is defined as the 2-position.

### (X)

X in formula (54) above is preferably —C(R²⁰⁷)(R²⁰⁸)— or —N(R²⁰⁹)— and more preferably —C(R²⁰⁷)(R²⁰⁸)— because stability during charge transport is high.

When X has a crosslinking group, it is preferable that at least one of R²⁰⁷ and R²⁰⁸, R²⁰⁹, or at least one of R²¹¹ to R²¹⁴ is an alkyl group having a crosslinking group, an aralkyl group having a crosslinking group, or an aromatic hydrocarbon group having a crosslinking group because aggregation of polymer molecules tends to be prevented.

### (Preferred Repeating Units)

The repeating unit represented by formula (54) is particularly preferably a repeating unit represented by any of the following formulas (54-1) to (54-8).

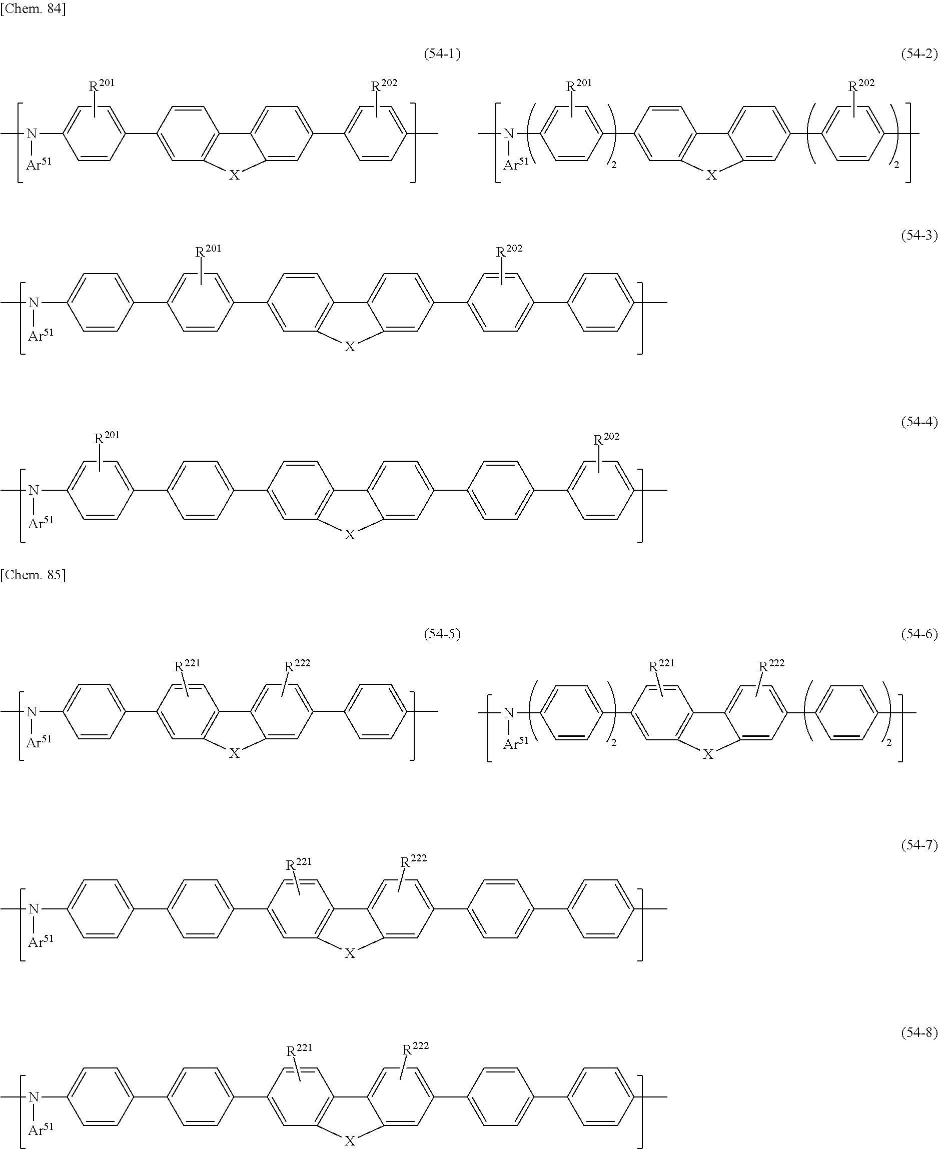

In the above formulas, R²⁰¹ and R²⁰² are the same, and R²⁰¹ and R²⁰² are bonded to positions symmetric to each other.

### <Preferred Examples of Main Chain of Repeating Unit Represented by Formula (54)>

No particular limitation is imposed on the structure of the main chain in formula (54) except for the nitrogen atom. However, for example, the following structures are preferred.

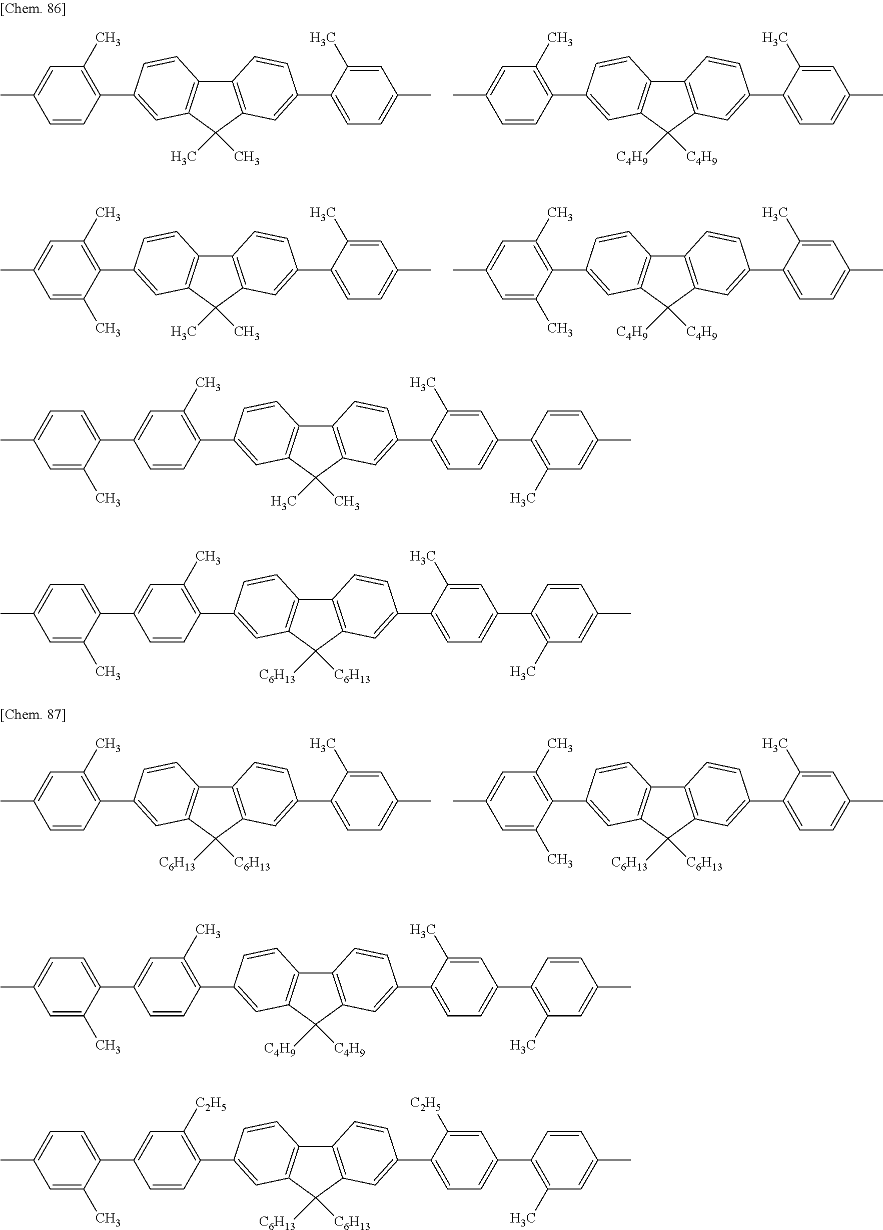

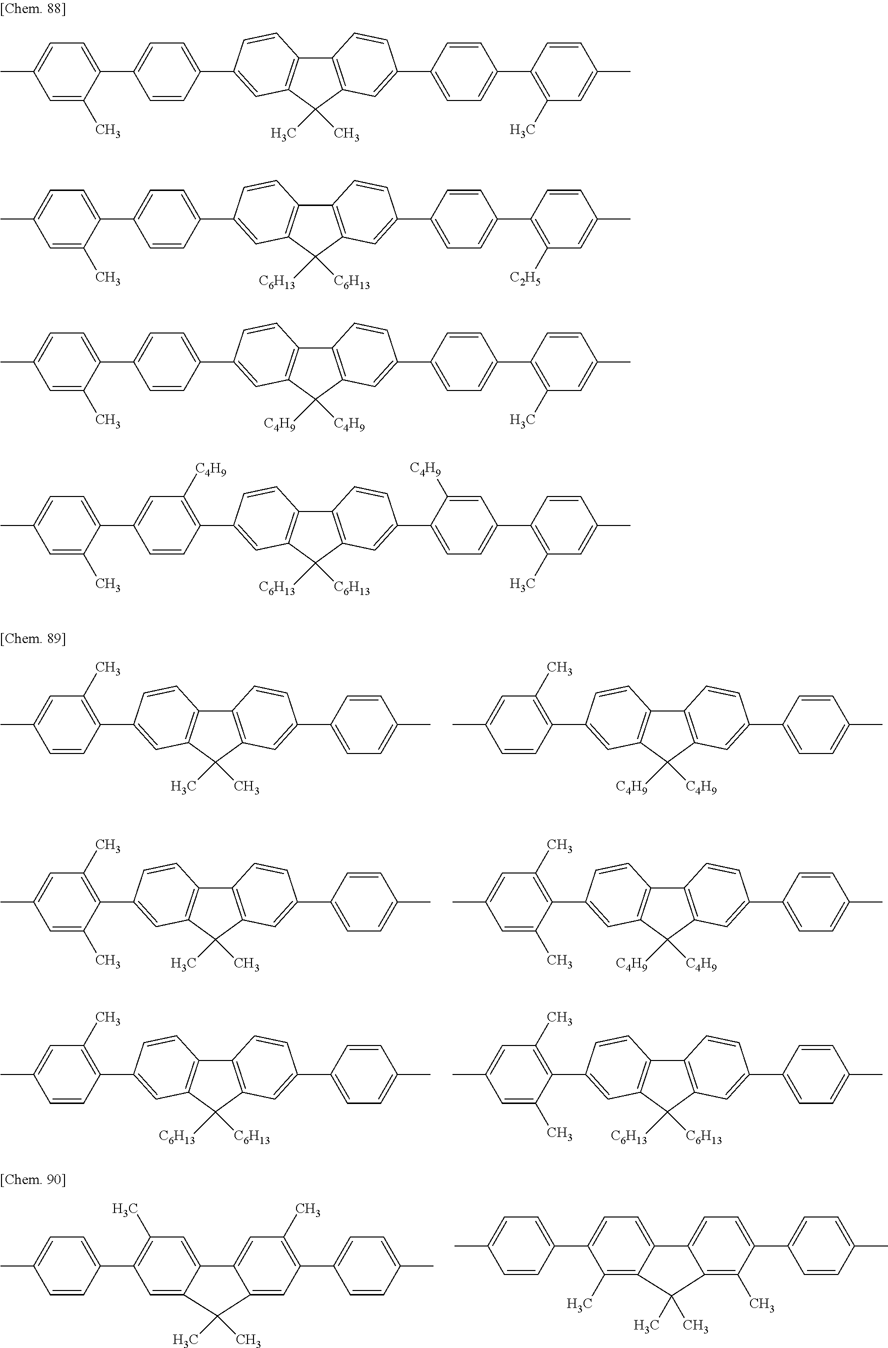

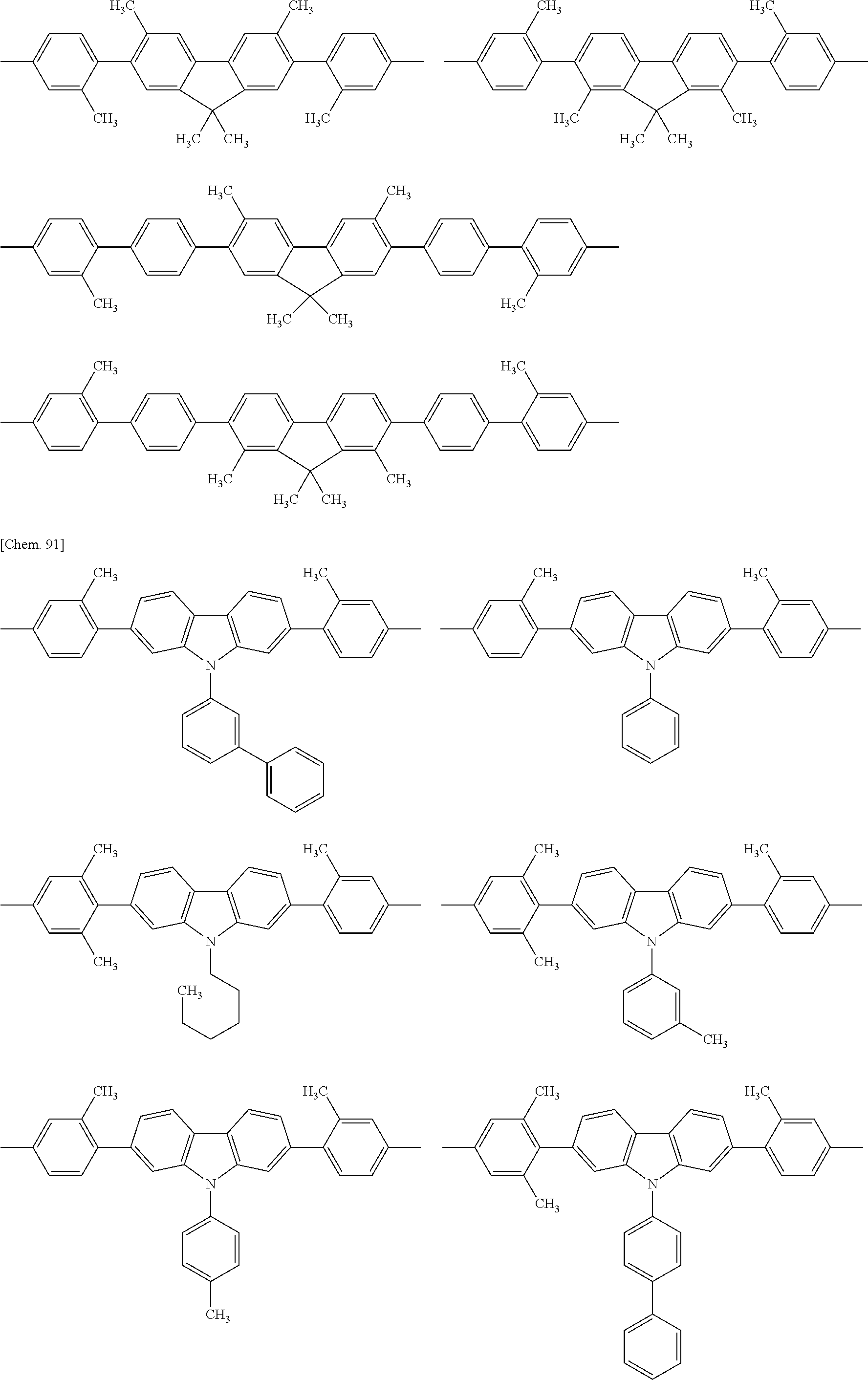

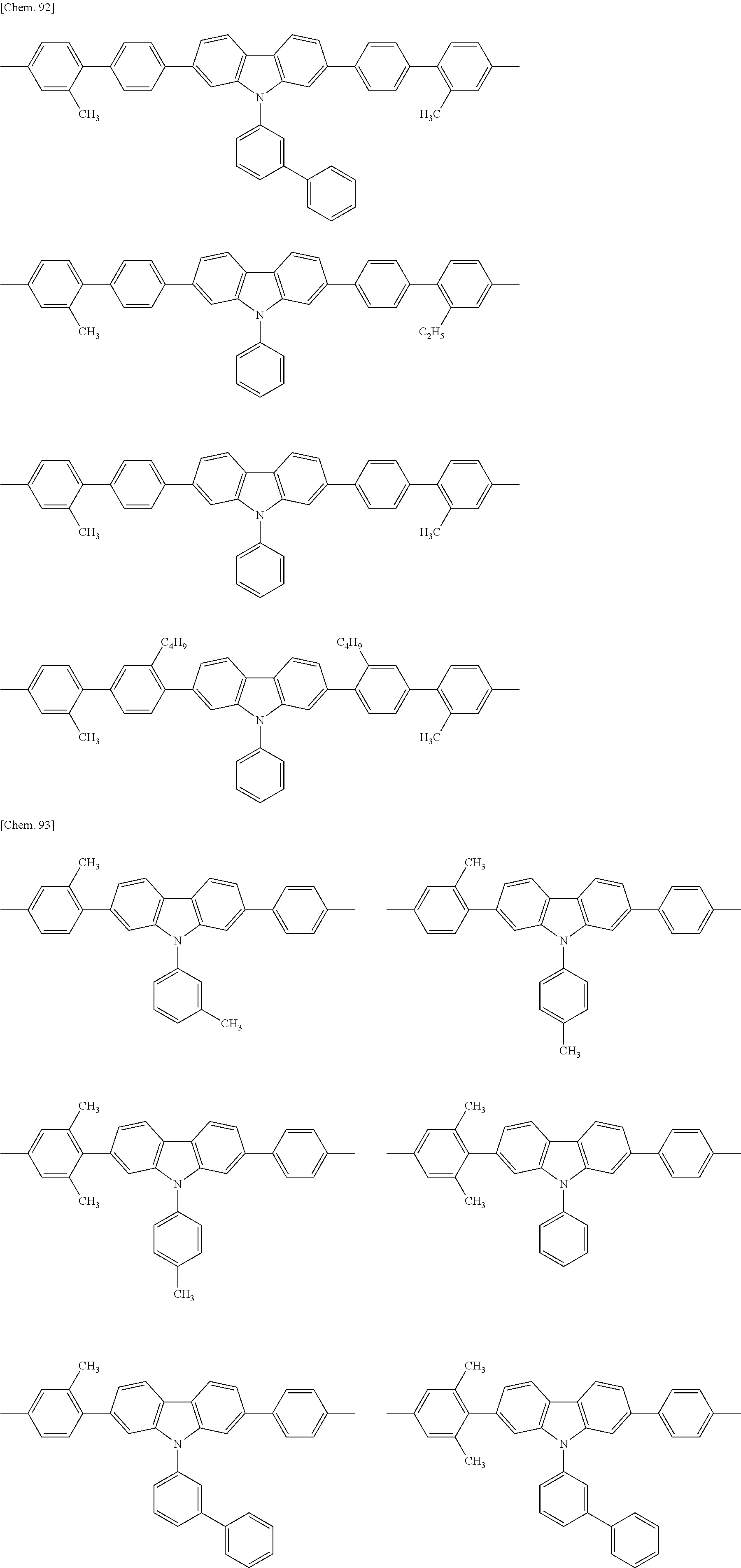

### <Repeating Unit Represented by Formula (55)>

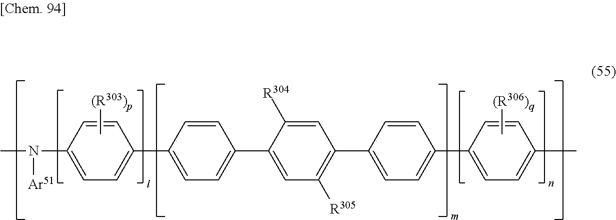

(In formula (55),

Ar⁵¹ is the same as Ar⁵¹ in formula (54) above.

R³⁰³ and R³⁰⁶ each independently represent an alkyl group optionally having a substituent and/or a crosslinking group.

R³⁰⁴ and R³⁰⁵ each independently represent an alkyl group optionally having a substituent and/or a crosslinking group, an alkoxy group optionally having a substituent and/or a crosslinking group, or an aralkyl group optionally having a substituent and/or a crosslinking group.

l is 0 or 1.

m is 1 or 2.

n is 0 or 1.

p is 0 or 1.

q is 0 or 1.)

(R³⁰³ and R³⁰⁶)

In the repeating unit represented by formula (55) above, R³⁰³ and R³⁰⁶ are each independently an alkyl group optionally having a substituent and/or a crosslinking group.

Examples of the alkyl group are the same as those for R²⁰¹ and R²⁰² in formula (54) above, and examples of the optional substituent, the optional crosslinking group, and the preferred structures are also the same as those for R²⁰¹ and R²⁰².

When a plurality of R³⁰³'s are present, the plurality of R³⁰³'s may be the same or different. When a plurality of R³⁰⁶'s are present, the plurality of R³⁰⁶'s may be the same or different.

(R³⁰⁴ and R³⁰⁵)

In the repeating unit represented by formula (55), R³⁰⁴ and R³⁰⁵ are each independently an alkyl group optionally having a substituent and/or a crosslinking group, an alkoxy group optionally having a substituent and/or a crosslinking group, or an aralkyl group optionally having a substituent and/or a crosslinking group. Preferably, R³⁰⁴ and R³⁰⁵ are each independently an alkyl group optionally having a substituent and/or a crosslinking group.

Preferably, R³⁰⁴ and R³⁰⁴ are the same.

The alkyl group is a linear, branched, or cyclic alkyl group. No particular limitation is imposed on the number of carbon atoms in the alkyl group. The number of carbon atoms is preferably 1 or more and 24 or less, more preferably 8 or less, and still more preferably 6 or less because the solubility of the polymer tends to be improved.

Specific examples of the alkyl group include a methyl group, an ethyl group, a n-propyl group, an i-propyl group, a n-butyl group, an i-butyl group, a sec-butyl group, a tert-butyl group, a n-hexyl group, a n-octyl group, a cyclohexyl group, and a dodecyl group.

No particular limitation is imposed on the alkoxy group. The alkyl group represented by R¹⁰ in an alkoxy group (—OR¹⁰) may have a linear, branched, or cyclic structure, and the number of carbon atoms in the alkyl group is preferably 1 or more and 24 or less and more preferably 12 or less because the solubility of the polymer tends to be improved.

Specific examples of the alkoxy group include a methoxy group, an ethoxy group, a n-propoxy group, a n-butoxy group, a hexyloxy group, a 1-methylpentyloxy group, and a cyclohexyloxy group.

No particular limitation is imposed on the aralkyl group. However, the number of carbon atoms in the aralkyl group is preferably 5 or more and is preferably 60 or less and more preferably 40 or less because the solubility of the polymer tends to be improved.

Specific examples of the aralkyl group include a 1,1-dimethyl-1-phenylmethyl group, a 1,1-di(n-butyl)-1-phenylmethyl group, a 1,1-di(n-hexyl)-1-phenylmethyl group, a 1,1-di(n-octyl)-1-phenylmethyl group, a phenylmethyl group, a phenylethyl group, a 3-phenyl-1-propyl group, a 4-phenyl-1-n-butyl group, a 1-methyl-1-phenylethyl group, a 5-phenyl-1-n-propyl group, a 6-phenyl-1-n-hexyl group, a 6-naphthyl-1-n-hexyl group, a 7-phenyl-1-n-heptyl group, an 8-phenyl-1-n-octyl group, and a 4-phenylcyclohexyl group.

Examples of the optional substituents on the alkyl, alkoxy, and aralkyl groups represented by R³⁰⁴ and R³⁰⁵ include those described as the preferred groups for the alkyl, aralkyl, and aromatic hydrocarbon groups represented by R²⁰⁷ to R²⁰¹ and R²¹¹ to R²¹⁴. The optional crosslinking group is a crosslinking group selected from the group T of crosslinking groups.

From the viewpoint of lowering voltage, it is most preferable that the alkyl, alkoxy, and aralkyl groups represented by R³⁰⁴ and R³⁰⁵ each have no substituent and no crosslinking group.

When crosslinking groups are bonded to the main chain structure of the repeating unit represented by formula (55), it is preferable that the crosslinking groups are bonded to R³⁰⁴ and R³⁰⁵

(l, m, and n)

l represents 0 or 1. n represents 0 or 1.

l and n are independently set. l+n is preferably 1 or more, more preferably 1 or 2, and still more preferably 2. When l+n falls within the above range, the solubility of the polymer increases, and precipitation from the composition of the invention containing the polymer tends to be prevented.

m represents 1 or 2 and is preferably 1 because an organic electroluminescent element produced using the composition of the invention can be driven at a low voltage and the hole injectability, hole transportability, and durability tend to be improved.

(p and q)

p represents 0 or 1. q represents 0 or 1. When 1 is 2 or more, a plurality of p's may be the same or different. When n is 2 or more, a plurality of q's may be the same or different. When l=n=1, p and q are not simultaneously 0. When p and q are not simultaneously 0, the solubility of the polymer can be high, and precipitation from the composition of the invention containing the polymer tends to be prevented. For the same reason as for a and b, when p+q is 1 or more, the aromatic rings in the main chain are twisted due to steric hindrance. In this case, the solubility of the polymer in a solvent becomes high, and a coating film formed by a wet deposition method and subjected to heat treatment tends to have good insolubility in a solvent. Therefore, in the case where p+q is 1 or more, when another organic layer (e.g., a light-emitting layer) is formed on this coating film by a wet deposition method, elution of the polymer to the composition for forming the other organic layer containing an organic solvent can be reduced.

### <Specific Examples of Main Chain of Repeating Unit Represented by Formula (55)>

No particular limitation is imposed on the structure of the main chain in formula (55) except for the nitrogen atom. However, for example, the following structures are preferred.

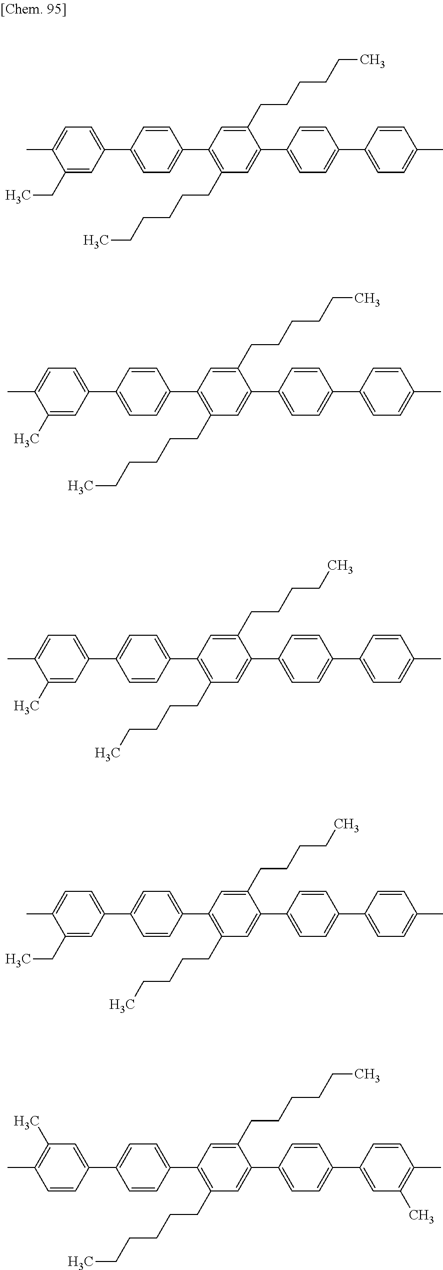

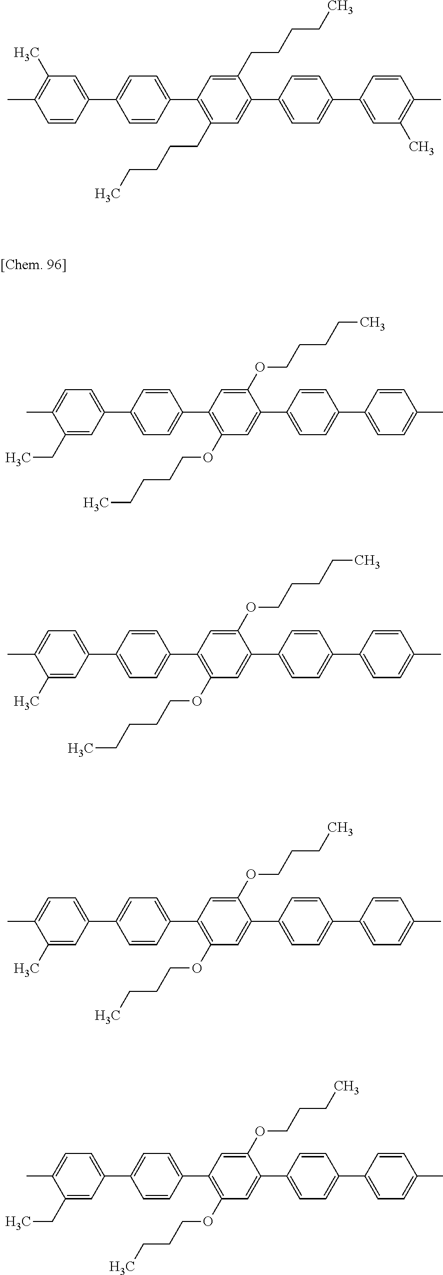

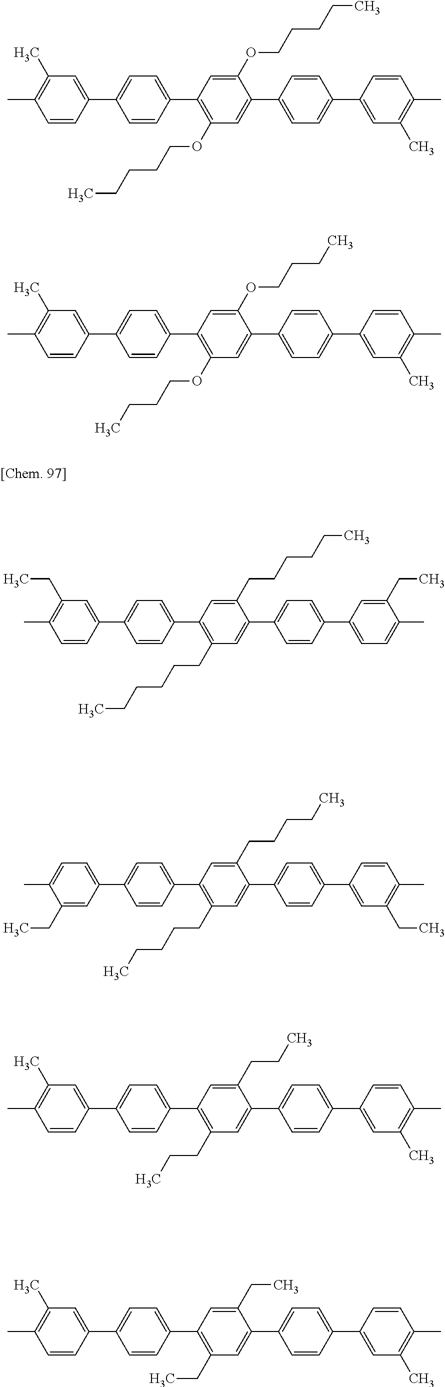

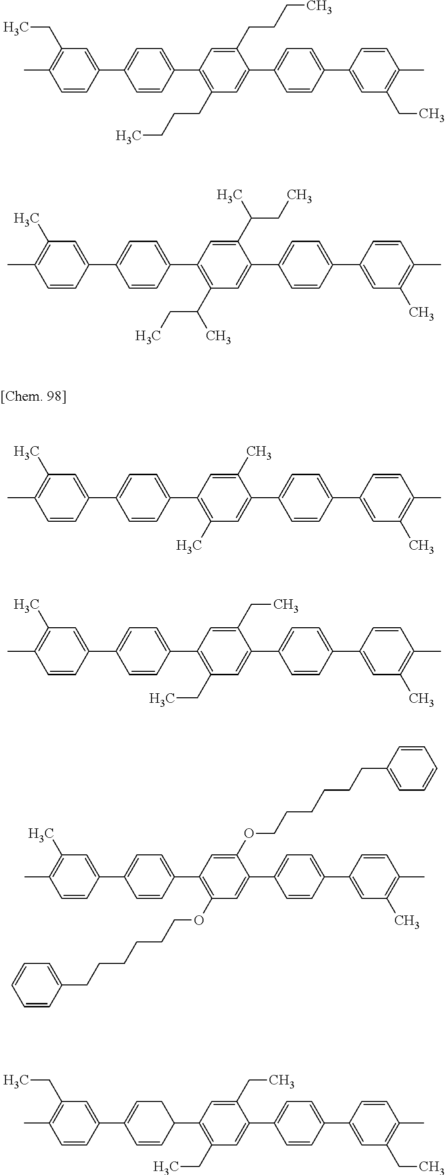

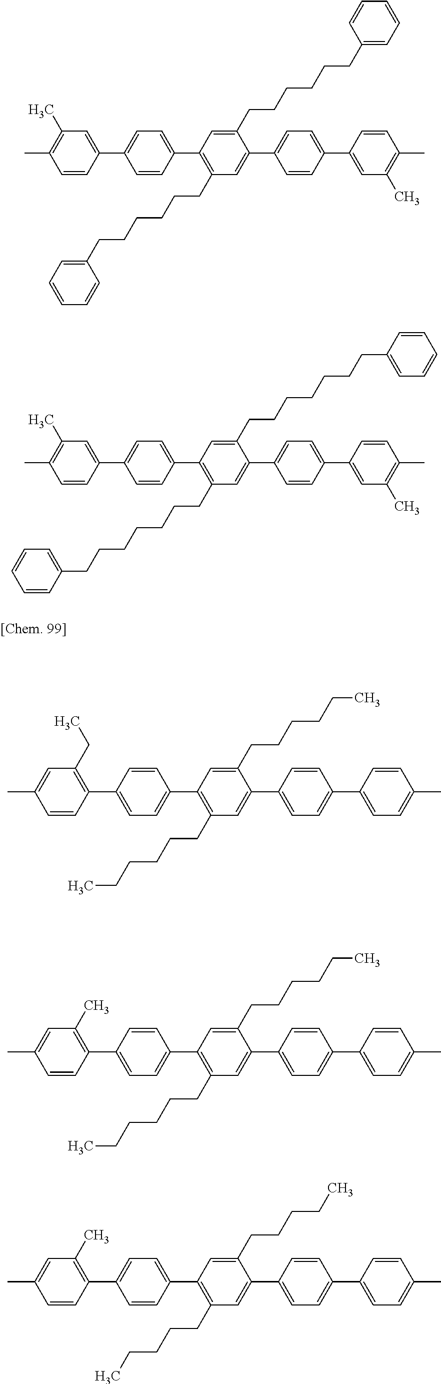

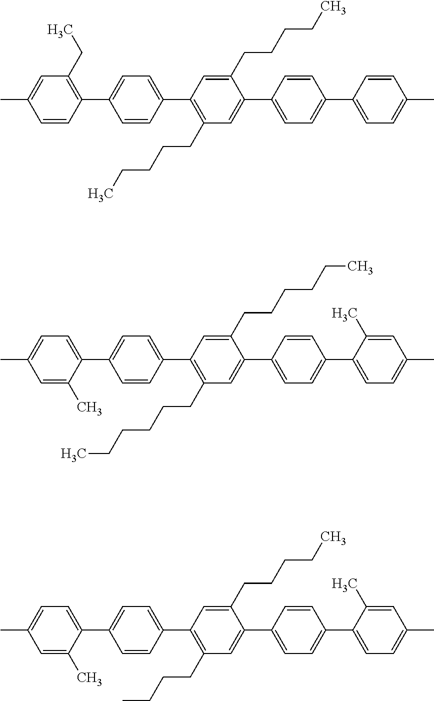

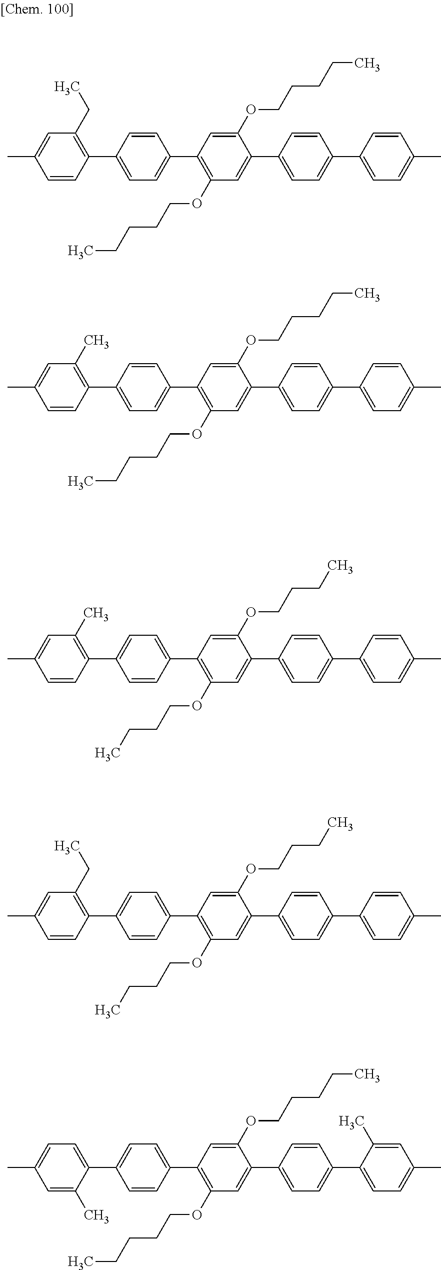

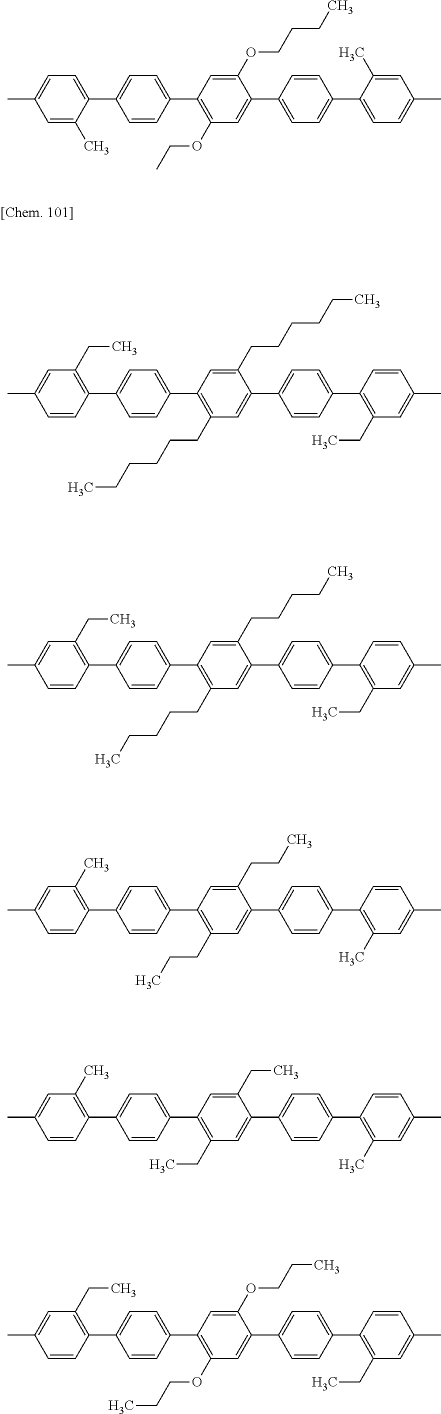

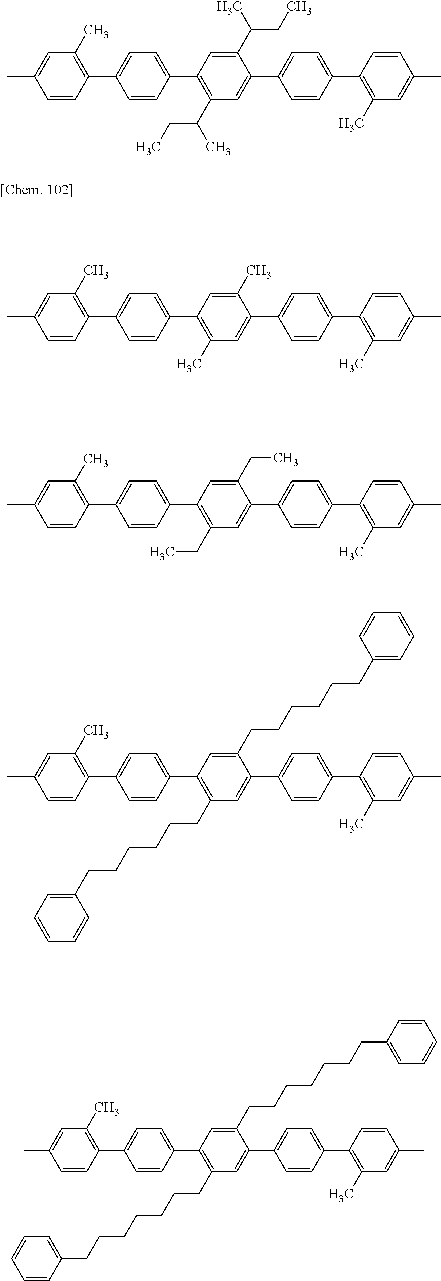

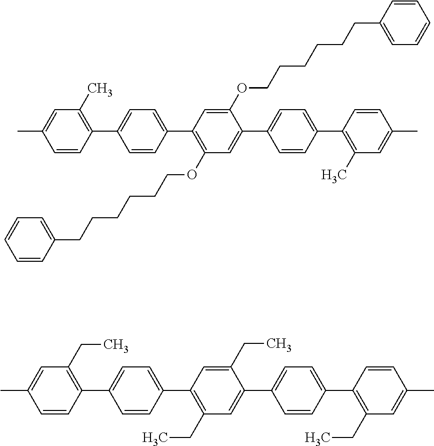

### <Repeating Unit Represented by Formula (56)>

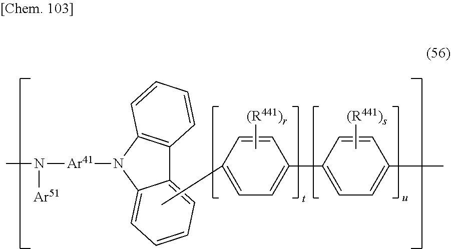

(In formula (56),

Ar⁵¹ is the same as Ar⁵¹ in formula (54) above.

Ar⁴¹ represents a divalent aromatic hydrocarbon group optionally having a substituent, a divalent aromatic heterocyclic group optionally having a substituent, or a divalent group in which a plurality of groups selected from the group consisting of divalent aromatic hydrocarbon groups each optionally having a substituent and divalent aromatic heterocyclic groups each optionally having a substituent are linked together directly or via a linking group.

R⁴⁴¹ and R⁴⁴² each independently represent an alkyl group optionally having a substituent.

t is 1 or 2.

u is 0 or 1.

r and s are each independently an integer of 0 to 4.)

(R⁴⁴¹ and R⁴⁴²)

In the repeating unit represented by formula (56) above, R⁴⁴¹ and R⁴⁴² are each independently an alkyl group optionally having a substituent.

The alkyl group is a linear, branched, or cyclic alkyl group. No particular limitation is imposed on the number of carbon atoms in the alkyl group. To maintain the solubility of the polymer, the number of carbon atoms is preferably 1 or more and 10 or less, more preferably 8 or less, and still more preferably 6 or less. The alkyl group is more preferably a methyl group or a hexyl group.

When a plurality of R⁴⁴¹'s and a plurality of R⁴⁴²'s are present in the repeating unit represented by formula (56), the plurality of R⁴⁴¹'s may be the same or different, and the plurality of R⁴⁴²'s may be the same or different.

(r, s, t, and u)

In the repeating unit represented by formula (56), r and s are each independently an integer of 0 to 4. When t is 2 or more, a plurality of r's may be the same or different. When u is 2 or more, a plurality of s's may be the same or different. r+s is preferably 1 or more, and r and s are each preferably 2 or less. When r+s is 1 or more, the driving lifetime of the organic electroluminescent element may be further extended for the same reason as for a and b in formula (54) above.

In the repeating unit represented by formula (56) above, t is 1 or 2. u is 0 or 1. t is preferably 1. u is preferably 1.

### (Ar⁴¹)

Ar⁴¹ is a divalent aromatic hydrocarbon group optionally having a substituent, a divalent aromatic heterocyclic group optionally having a substituent, or a divalent group in which a plurality of groups selected from the group consisting of divalent aromatic hydrocarbon groups each optionally having a substituent and divalent aromatic heterocyclic groups each optionally having a substituent are linked together directly or via a linking group.

Examples of the aromatic hydrocarbon and aromatic hydrocarbon groups represented by Ar⁴¹ include the same groups as those for Ar⁵² in formula (50) above. The optional substituents on the aromatic hydrocarbon and aromatic hydrocarbon groups are each preferably a group selected from the substituent group Z and the optional additional substituents are each also preferably a group selected from the substituent group Z.

### <Specific Examples of Repeating Unit Represented by Formula (56)>

No particular limitation is imposed on the repeating unit represented by formula (56), and examples thereof include the following structures.

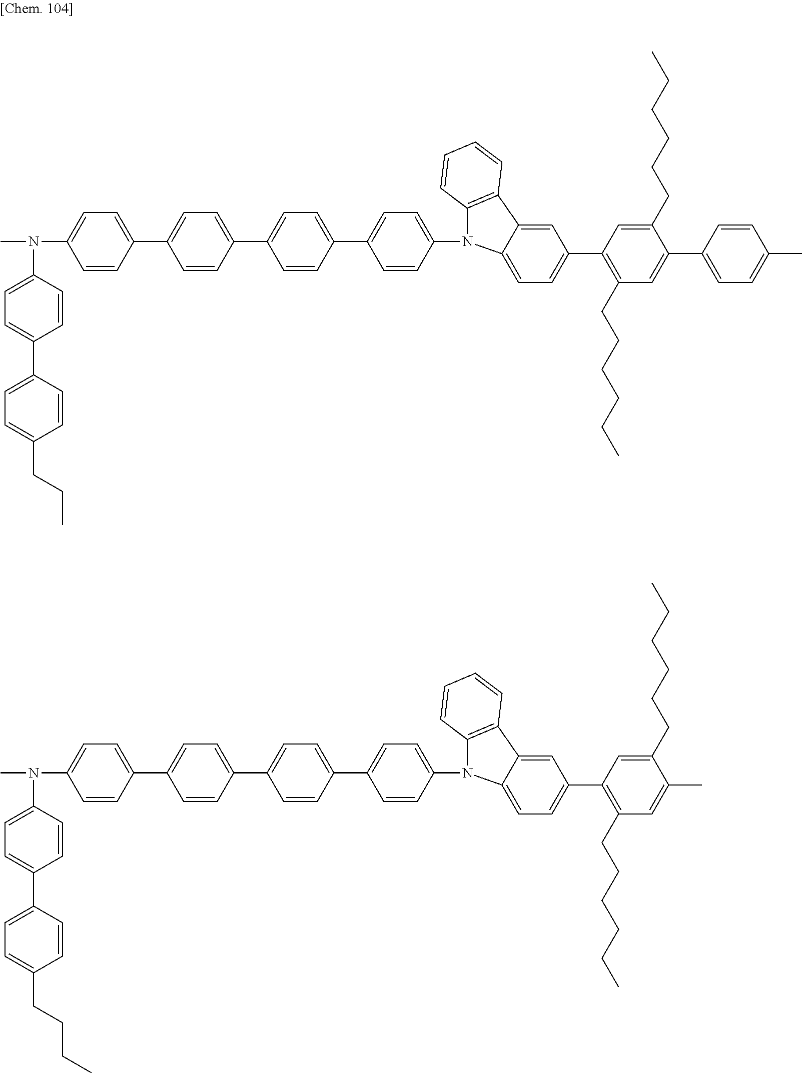

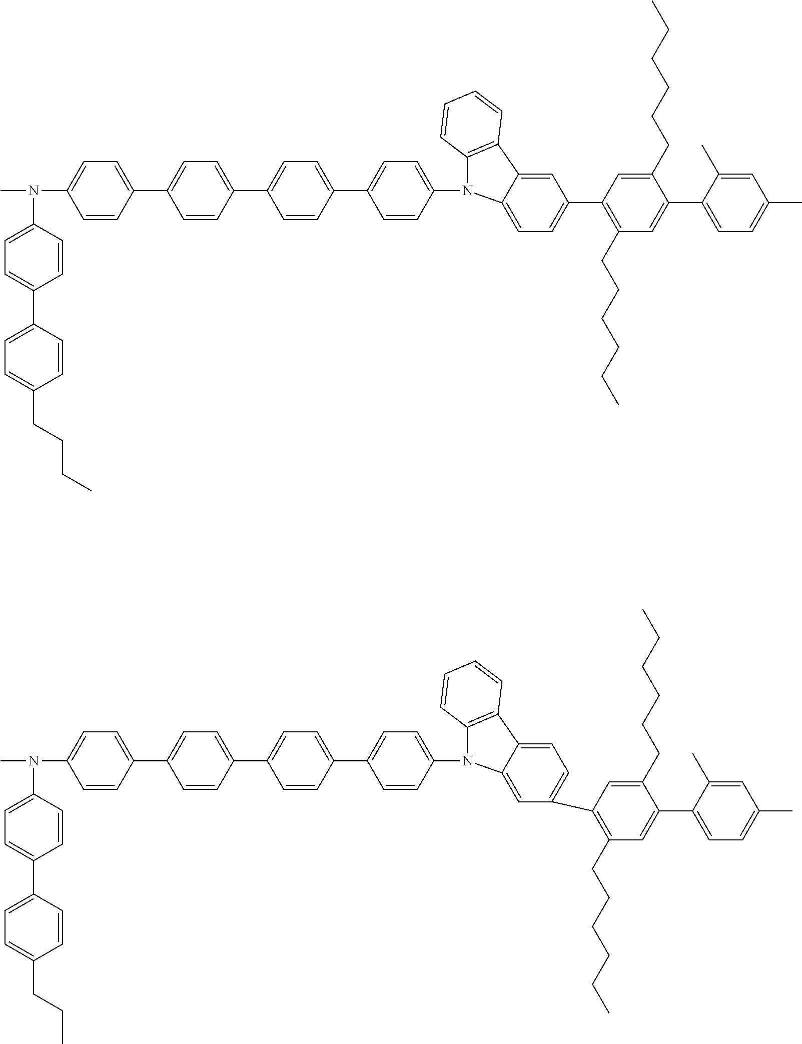

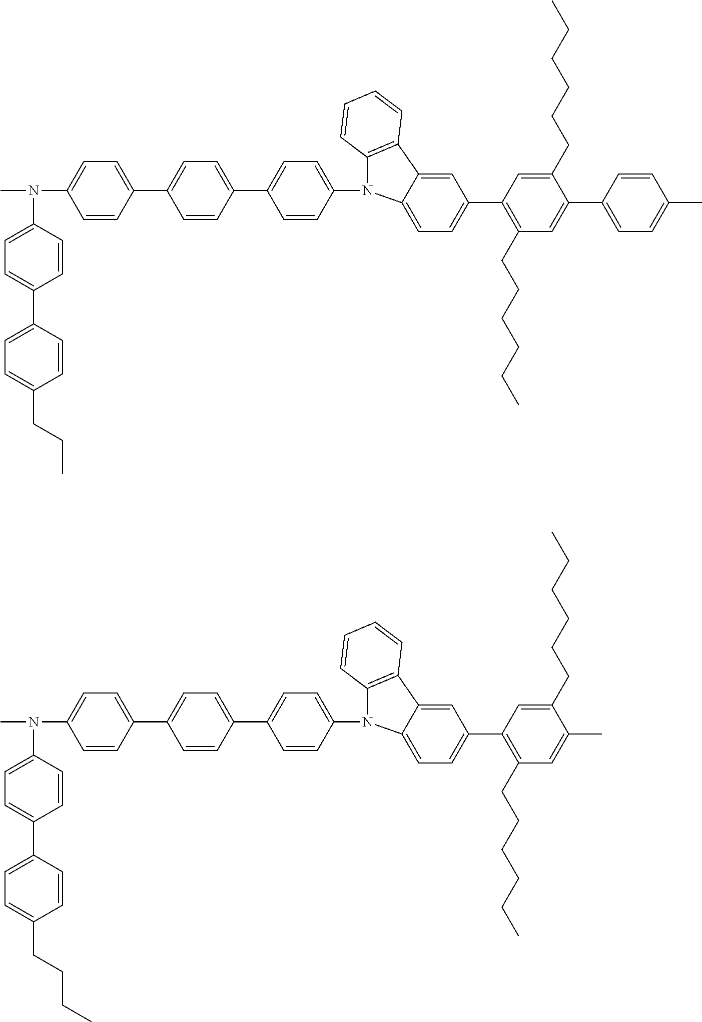

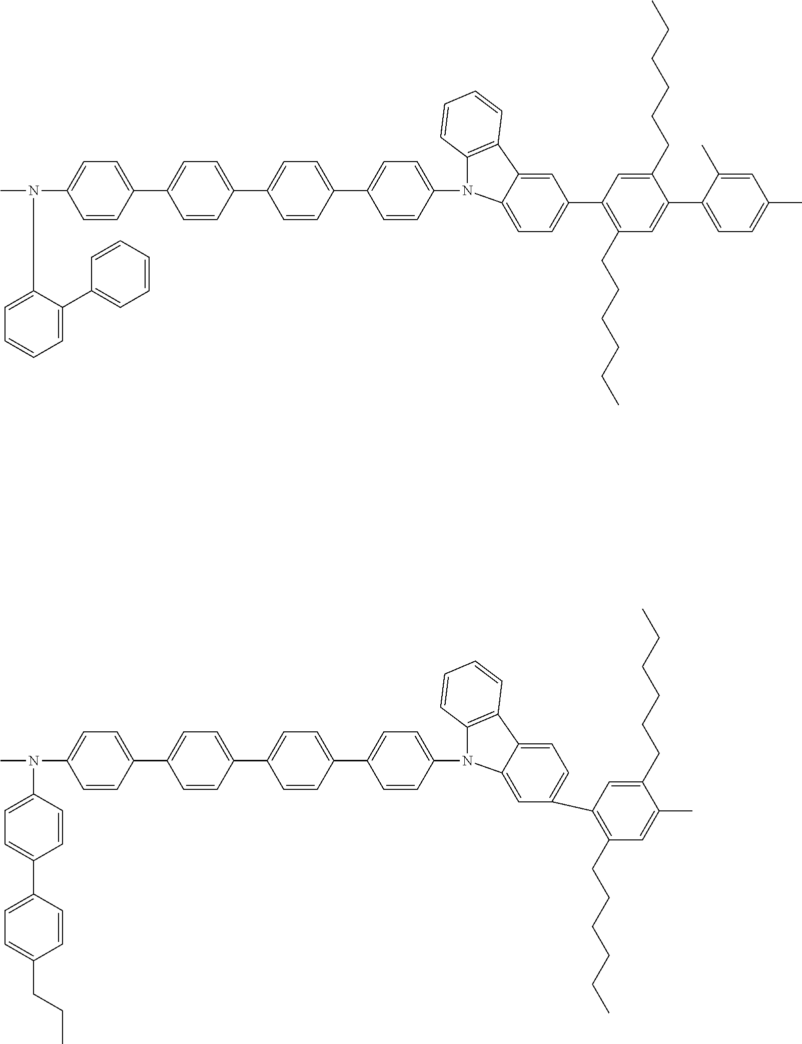

### <Repeating Unit Represented by Formula (57)>

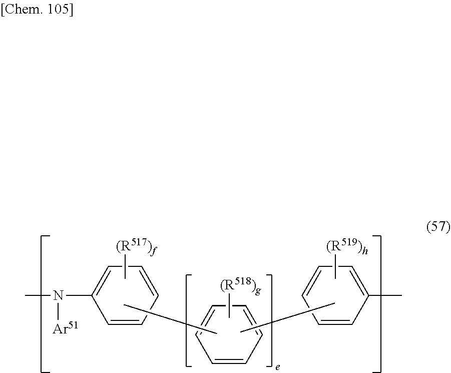

(In formula (57),

Ar⁵¹ is the same as Ar⁵¹ in formula (50) above.

R⁵¹⁷ to R⁵¹⁹ each independently represent an alkyl group optionally having a substituent and/or a crosslinking group, an alkoxy group optionally having a substituent and/or a crosslinking group, an aralkyl group optionally having a substituent and/or a crosslinking group, an aromatic hydrocarbon group optionally having a substituent and/or a crosslinking group, or an aromatic heterocyclic group optionally having a substituent and/or a crosslinking group.

f, g, and h each independently represent an integer of 0 to 4.

e represents an integer of 0 to 3.

When g is 1 or more, e is 1 or more.)

(R⁵¹⁷ to R⁵¹⁹)

The aromatic hydrocarbon and aromatic heterocyclic groups represented by R⁵¹⁷ to R⁵¹⁹ are each independently the same as those described for Ar⁵¹. The optional substituents on these groups are preferably the same groups as those in the substituent group Z, particularly in the substituent group X. The crosslinking groups are each preferably a crosslinking group selected from the group T of crosslinking groups.

Preferably, the alkyl and aralkyl groups represented by R⁵¹⁷ to R⁵¹⁹ are the same as those described above for R²⁰⁷. The optional substituents are each preferably the same group as that for R²⁰⁷ described above, and the crosslinking groups are each preferably a crosslinking group selected from the group T of crosslinking groups.

The alkoxy groups represented by R⁵¹⁷ to R⁵¹⁹ are preferably alkoxy groups selected from the substituent group Z, particularly from the substituent group X, and the optional substituents are each substituents in the substituent group Z and preferably substituents in the substituent group X. The optional crosslinking groups are each preferably a crosslinking group selected from the group T of crosslinking groups.

(f, g, and h)

f, g, and h each independently represent an integer of 0 to 4.

When e is 2 or more, a plurality of g's may be the same or different.

f+g+h is preferably 1 or more.

It is preferable that f+h is 1 or more;
it is more preferable that f+h is 1 or more and that f, g, and h are each 2 or less;
it is still more preferable that f+h is 1 or more and that f and h are each 1 or less; and
it is most preferable that f and h are each 1.

When f and h are each 1, it is preferable that R⁵¹⁷ and R⁵¹⁹ are bonded at positions symmetric to each other.

Preferably, R⁵¹⁷ and R⁵¹⁹ are the same.

More preferably, g is 2.

When g is 2, it is most preferable that the two R⁵¹⁸'s are bonded at para positions.

When g is 2, it is most preferable that the two R⁵¹⁸'s are the same.

The phrase “R⁵¹⁷ and R⁵¹⁹ are bonded at positions symmetric to each other” means bonding positions shown below. However, for notational purposes, a structure rotated 180° about the main chain is regarded as the same structure as the original structure.

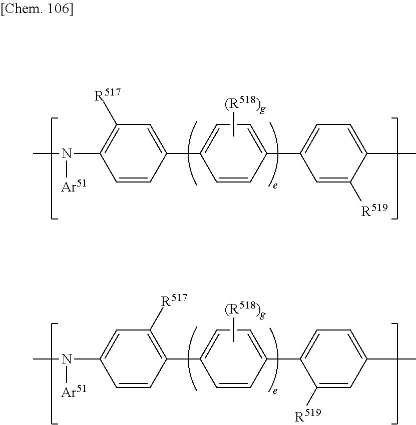

When the polymer in the present embodiment includes the repeating unit represented by formula (54) and the repeating unit represented by formula (57), the ratio of the repeating unit represented by formula (57) to the repeating unit represented by formula (54), i.e., (the number of moles of the repeating unit represented by formula (57)/the number of moles of the repeating unit represented by formula (54)), is preferably 0.1 or more, more preferably 0.3 or more, still more preferably 0.5 or more, yet more preferably 0.9 or more, and particularly preferably 1.0 or more. The ratio is preferably 2.0 or less, more preferably 1.5 or less, and still more preferably 1.2 or less.

The repeating unit represented by formula (57) is preferably a repeating unit represented by the following formula (58).

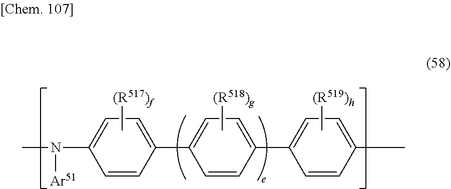

In the repeating unit represented by formula (58), it is preferable that g=0 or 2. When g=2, the bonding positions are the 2- and 5-positions. When g=0, i.e., when there is no steric hindrance by R⁵¹⁸, and when g=2 and the bonding positions are the 2- and 5-positions, i.e., when there is steric hindrance due to the two R⁵¹⁸'s bonded to the diagonal positions of the benzene ring, R⁵¹⁷ and R⁵¹⁹ can be bonded to positions symmetric to each other.

The repeating unit represented by formula (58) is more preferably a repeating unit represented by the following formula (59) with e=3.

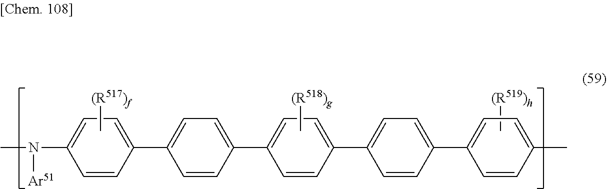

In the repeating unit represented by formula (59) above, it is preferable that g=0 or 2. When g=2, the bonding positions are the 2- and 5-positions. When g=0, i.e., there is no steric hindrance due to R⁵¹⁸, and when g=2 and the bonding positions are the 2- and 5-positions, i.e., when there is steric hindrance due to the two R⁵¹⁸'s bonded to the diagonal positions of the benzene ring, R⁵¹⁷ and R⁵¹⁹ can be bonded to positions symmetric to each other.

### <Specific Examples of Main Chain of Repeating Unit Represented by Formula (57)>

No particular limitation is imposed on the structure of the main chain of the repeating unit represented by formula (57). However, examples thereof include the following structures.

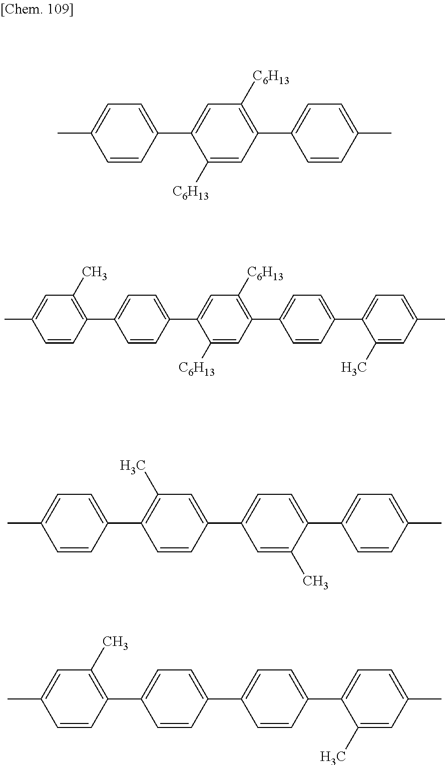

Preferably, the repeating units represented by formulas (50) to (59) above each have no crosslinking group. It is preferable that no crosslinking group is included because the polymer chain is unlikely to be distorted by heat drying or baking (heat firing) after wet deposition. This is because, when crosslinking groups undergo a reaction, a volume change may occur and therefore the polymer chain may be distorted. Even when no volume change occurs, the polymer chain may be distorted.

### <Repeating Unit Represented by Formula (60)>

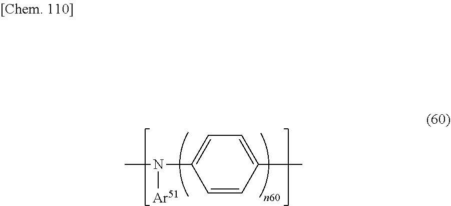

(In formula (60),

Ar⁵¹ is the same as Ar⁵¹ in formula (50) above.

n60 represents an integer of 1 to 5.)

(n60)

n60 represents an integer of 1 to 5 and is preferably an integer of 1 to 4 and more preferably an integer of 1 to 3.

### (Twisted Structure Disrupting Conjugation)

When the high-molecular weight charge transport compound used in the composition of the invention is a polymer having the repeating unit represented by formula (50) above, the repeating unit represented by formula (50) is more preferably the repeating unit represented by formula (54) above, the repeating unit represented by formula (55) above, the repeating unit represented by formula (56) above, or the repeating unit represented by formula (57) above. The partial structure represented by formula (63) above is preferably a partial structure represented by formula (63A) or (63B) below. Therefore, the repeating unit represented by formula (50) above is more preferably the repeating unit represented by formula (54) above and including the partial structure represented by formula (63A) or (63B) below as a main chain structure, the repeating unit represented by formula (55) above and including the partial structure represented by formula (63A) or (63B) below as a main chain structure, the repeating unit represented by formula (56) above and including the partial structure represented by formula (63A) or (63B) below as a main chain structure, or the repeating unit represented by formula (57) above and including the partial structure represented by formula (63A) or (63B) below as a main chain structure.

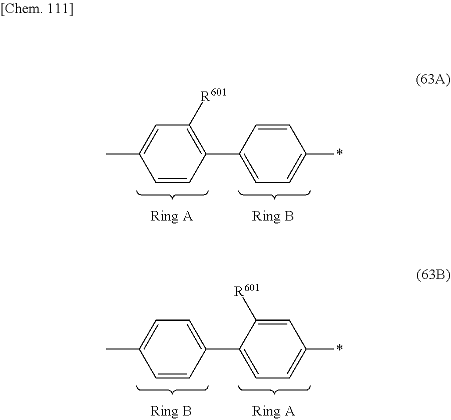

(In formulas (63A) and (63B),
R⁶⁰¹ is the same as R⁶⁰¹ in formula (63);
Ring A is a partial structure of ring Ar in formula (63) or ring Ar; and
Ring B is a partial structure of Ar⁶²¹ in formula (63) or ring Ar⁶²¹.

R⁶⁰¹ represents R²⁰¹, R²⁰², R²²¹, or R²²² in formula (54), R³⁰³, R³⁰⁴, R³⁰⁵, or R⁴⁰⁶ in formula (55), R⁴⁴¹ or R⁴⁴² in formula (56), or R⁵¹⁷, R⁵¹⁸, or R⁵¹⁹ in formula (57).

* represents a bond to an adjacent atom.

When formula (63A) is a partial structure of formula (54) or a partial structure of formula (56), Ring B may be part of a condensed ring.

The partial structure represented by formula (63A) and the partial structure represented by formula (62B) may each have, in addition to R⁶⁰¹,
R²⁰¹, R²⁰², R²²¹, or R²²² in each of Ring A and Ring B when the partial structure is a partial structure of formula (54),
R³⁰³, R³⁰⁴, R³⁰⁵, or R³⁰⁶ in each of Ring A and Ring B when the partial structure is a partial structure of formula (55),
R⁴⁴¹ or R⁴⁴² in each of Ring A and Ring B when the partial structure is a partial structure of formula (56), or
R⁵¹⁷, R⁵¹⁸, or R⁵¹⁹ in each of Ring A and Ring B when the partial structure is a partial structure of formula (57).)

### <Repeating Unit Represented by Formula (62)>

The polymer including the repeating unit represented by formula (50) above is particularly preferably a polymer including a repeating unit represented by formula (62) that is a repeating unit represented by formula (54) above including partial structures represented by formulas (63A) and (63B) above in a main chain structure.

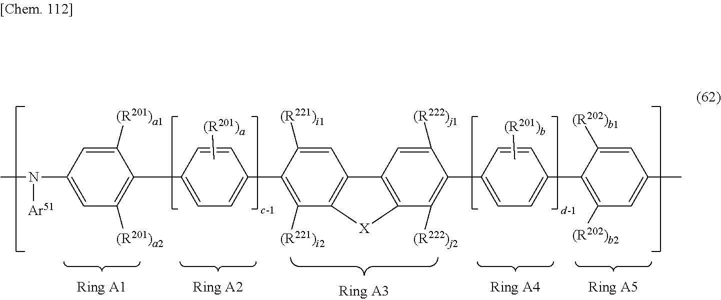

(In formula (62),

Ar⁵¹, X, R²⁰¹, R²⁰², R²²¹, R²²², a, b, c, d, e, i, and j are the same as Ar⁵¹, X, R²⁰¹, R²⁰², R²²¹, R²²², a, b, i, and j in formula (54) above.

c is an integer of 1 to 3.

d is an integer of 1 to 4.

a¹, a², b¹, b², i¹, i², j¹, and j² are each independently 0 or 1. However, any of the following conditions (1) and (2) is satisfied.
(1) At least one of a¹, a², a, b¹, b², and b is 1 or more. When c is 1, a is not defined. When d is 1, b is not defined.
(2) At least one of i¹, i², j¹, and j² is 1.

Ring A1 is a divalent benzene ring optionally having R²⁰¹ at a specific position.

Ring A2 is a divalent group including c−1 benzene rings linked together and each optionally having R²⁰¹. When c=2, Ring A2 is a single divalent benzene ring.

Ring A3 is a divalent condensed ring having a biphenyl structure further bonded through X.

Ring A4 is a divalent group including d−1 benzene rings linked together and each optionally having R²⁰¹. When d=2, Ring A4 is a single divalent benzene ring.

Ring A5 is a divalent benzene ring optionally having R²⁰² at a specific position.)

The phrase “a in formula (54) is 1 or more” is synonymous with the phrase “at least one of a¹, a², and a in formula (62) is 1 or more.” The phrase “b in formula (54) is 1 or more” is synonymous with the phrase “at least one of b¹, b², and b in formula (62) is 1 or more.”

Formula (62) includes formula (63A) or formula (63B) as a partial structure, as described below.

When at least one of a¹, a², and a is 1 or more,
Ring A1 and Ring A2 include a partial structure represented by formula (63A) or (63B) above when at least one of a¹ or a² is 1 and c is 2 or more; Ring A1 and Ring A3 include a partial structure represented by formula (63A) or (63B) above when at least one of a¹ or a² is 1 and c is 1; and
Rings A2 and A1 or Rings A2 and A3 include a partial structure represented by formula (63A) or (63B) above when a is 1.

Similarly, when at least one of b¹, b², and b is 1 or more, formula (63A) or (63B) is included as a partial structure.

When at least one of i¹, i², j¹, and j² is 1,
a ring in Ring A3 to which R²²¹ is bonded and a benzene ring in Ring A2 form formula (63B) as a partial structure when one or both of i¹ and i² are 1; and
a ring in Ring A3 to which R²²² is bonded and a benzene ring in Ring A4 form formula (63A) as a partial structure when one or both of j¹ and j² are 1.

Specifically, Rings A3 and A2 or Rings A3 and A4 form a twisted structure.

Therefore, since formula (62) includes a structure in which aromatic rings in the main chain are twisted, conjugation is disrupted by this structure, which is preferred.

Preferred is a structure in which at least one of a1 and a2 and at least one of b1 and b2 are 1, a structure in which i1 and j1 are 1, a structure in which i2 and j2 are 1, or a structure in which i1, j1, i2, and j2 are 1.

### <Molecular Weights of Polymer>

The molecular weights of the polymer contained in the composition of the invention will be described.

The weight average molecular weight (Mw) of the polymer having the above-described arylamine structure as a repeating unit is generally 1,000,000 or less, preferably 500,000 or less, more preferably 100,000 or less, still more preferably 70,000 or less, and particularly preferably 50,000 or less. The weight average molecular weight is 5,000 or more, more preferably 10,000 or more, still more preferably 12,000 or more, and particularly preferably 15,000 or more.

When the weight average molecular weight of the polymer having the above-described arylamine structure as a repeating unit is equal to or less than the above upper limit, the polymer exhibits solubility in a solvent. Moreover, the film formability tends to be good. When the weight average molecular weight of the polymer is equal to or more than the above lower limit, reductions in the glass transition temperature, melting point, and vaporization temperature of the polymer are prevented, and the heat resistance may be improved. In addition, insolubility of a coating film in an organic solvent after a crosslinking reaction may be sufficient.

The number average molecular weight (Mn) of the polymer having the above-described arylamine structure as a repeating unit is generally 750,000 or less, preferably 250,000 or less, more preferably 100,000 or less, and particularly preferably 50,000 or less. The number average molecular weight is generally 2,000 or more, preferably 4,000 or more, more preferably 6,000 or more, and still more preferably 8,000 or more.

The polydispersity (Mw/Mn) of the polymer having the above-described arylamine structure as a repeating unit is preferably 3.5 or less, more preferably 2.5 or less, and particularly preferably 2.0 or less. The smaller the polydispersity, the better. Therefore, the lower limit of the polydispersity is ideally 1. When the polydispersity of the polymer is equal to or lower than the above upper limit, the polymer can be easily purified, and the solubility in a solvent and the charge transportability are good.

Generally, the weight average molecular weight and number average molecular weight of the polymer are determined by SEC (size exclusion chromatography) measurement. In the SEC measurement, the elution time is shorter for a higher molecular weight component and longer for a lower molecular weight component. A calibration curve computed from the elution times of polystyrenes (standard specimens) with known molecular weights is used to convert the elution time of a sample to its molecular weights. The weight average molecular weight and the number average molecular weight of the sample are thereby computed.

### <Content of Repeating Unit Represented by Formula (50)>

No particular limitation is imposed on the content of the repeating unit represented by formula (50) in the polymer. However, the content of the repeating unit represented by formula (50) with respect to 100% by mole of all the repeating units in the polymer is generally 10% by mole or more, preferably 30% by mole or more, still more preferably 40% by mole or more, and yet more preferably 50% by mole or more.

The polymer may include only the repeating unit represented by formula (50). However, for the purpose of balancing the performance capabilities of the organic electroluminescent element prepared, the polymer may include a repeating unit different from the repeating unit represented by formula (50). In this case, the content of the repeating unit represented by formula (50) in the polymer is generally 99% by mole or less and preferably 95% by mole or less.

### <Repeating Unit Represented by Formula (61)>

The polymer in the invention including the arylamine structure as a repeating unit may further include, in the main chain, a structure represented by the following formula (61).

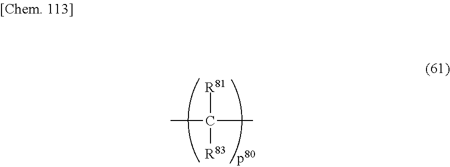

(In formula (61),

R⁸¹ and R⁸² each independently represent a hydrogen atom, an alkyl group, an aromatic hydrocarbon group, or an aromatic heterocyclic group. When a plurality of R⁸¹'s are present, they may be the same or different. When a plurality of R⁸²'s are present, they may be the same or different.

p⁸⁰ represents an integer of 1 to 5.)

When R⁸¹ and R⁸² are each an alkyl group, the alkyl group is a linear, branched, or cyclic alkyl group. No particular limitation is imposed on the number of carbon atoms in the alkyl group. To maintain the solubility of the polymer, the number of carbon atoms is preferably 1 or more and 8 or less, more preferably 6 or less, and still more preferably 3 or less. The alkyl group is more preferably a methyl group or an ethyl group.

When R¹¹ and R¹² are each an aromatic hydrocarbon group or an aromatic heterocyclic group, they are preferably any of the structures described above in the “Definitions” section.

R⁸¹ and R⁸² may each optionally have a substituent and/or a crosslinking group. The substituent is preferably a substituent selected from the substituent group Z, particularly from the substituent group X. The crosslinking group is preferably a crosslinking group selected from the crosslinking group Z.

From the viewpoint of the durability and charge transportability of the polymer, p⁸⁰ is preferably 3 or less, more preferably 2 or less, and most preferably 1.

When the structure represented by formula (61) is included, conjugation in the main chain of the polymer is cut, and the Si energy level and Ti energy level of the polymer increase. Therefore, when a composition containing this polymer is used for a hole transport layer of an organic electroluminescent element, excitons in the light-emitting layer are unlikely to be deactivated. This is preferred because the luminous efficiency may increase.

### <Preferred Repeating Unit Structures in Polymer>

Specific structures of the repeating units represented by the above formulas are referred to as “repeating unit structures.” The specific structure is a structure obtained by assigning specific structures and numerical values to all the symbols in a general formula. Specifically, the polymer having the arylamine structure as a repeating unit may include only one repeating unit structure or two or more repeating unit structures selected from a repeating unit structure included in formula (54) above, a repeating unit structure included in formula (55) above, a repeating unit structure included in formula (56) above, a repeating unit structure included in formula (57) above, and a repeating unit structure included in formula (60) above. When two or more repeating unit structures are included, these two or more repeating unit structures may be repeating unit structures included in the same formula or repeating unit structures included in different formulas. From the viewpoint of charge transportability and durability, it is preferable that the polymer having the arylamine structure as a repeating unit includes 1 or 2 specific repeating unit structures represented by any of the above formulas and includes no other repeating unit structure.

### (Specific Examples)

Specific examples of the polymer are shown below. However, the polymer is not limited to these examples. Numerical values in the chemical formulas represent the molar ratios of repeating units.

These polymers may each be a random copolymer, an alternating copolymer, a block copolymers, or a graft copolymer, and no limitation is imposed on the order of arrangement of monomers.

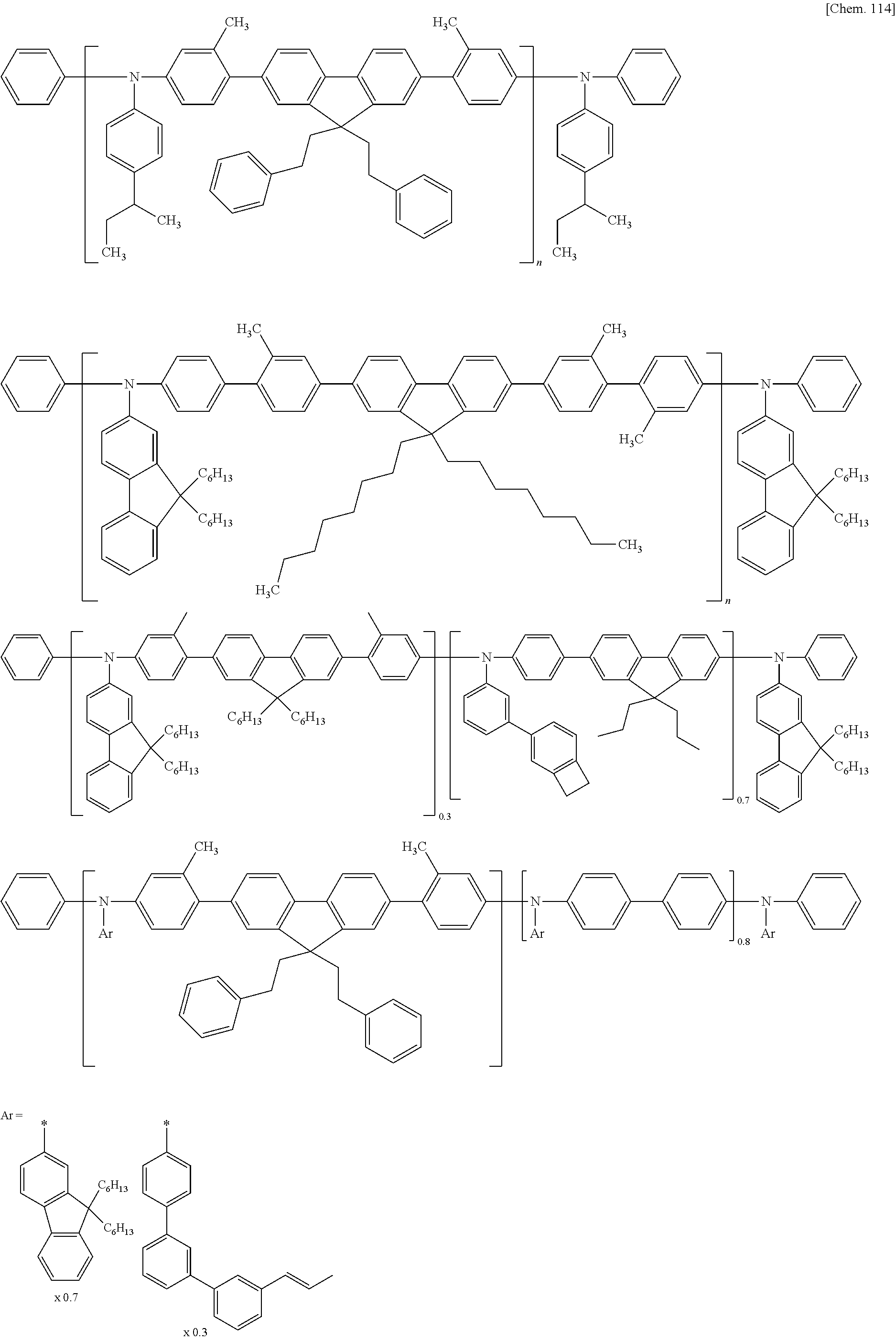

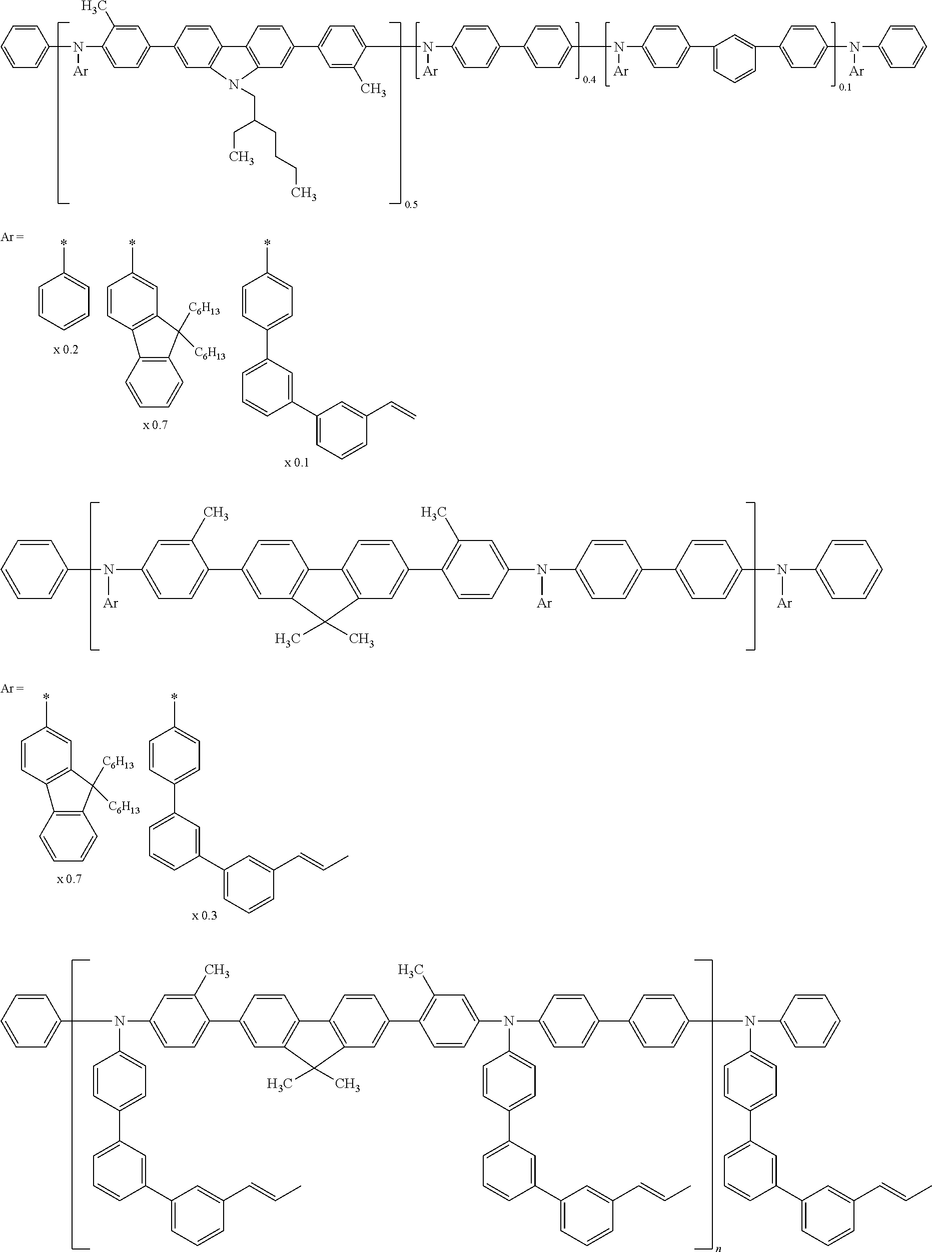

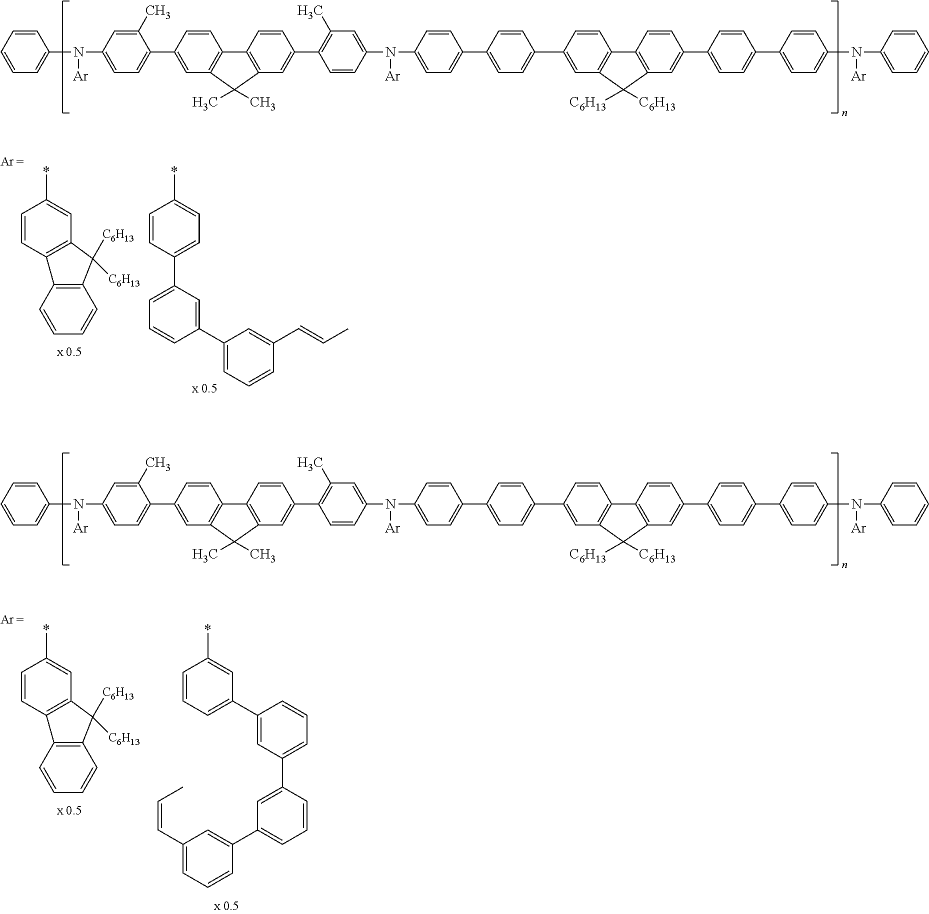

### <Method for Producing Polymer>

No particular limitation is imposed on the method for producing the polymer contained in the composition of the invention, and any method may be used. Examples of the production method include a polymerization method using the Suzuki reaction, a polymerization method using the Grignard reaction, a polymerization method using the Yamamoto reaction, a polymerization method using the Ullmann reaction, and a polymerization method using the Buchwald-Hartwig reaction. The polymer can also be produced using production methods similar to polymer production methods described in WO2019/177175, WO2020/171190, and WO2021/125011.

With the polymerization method using the Ullmann reaction and the polymerization method using the Buchwald-Hartwig reaction, for example, dihalogenated aryl represented by the following formula (2a) (Z represents a halogen atom such as I, Br, Cl, or F) and primary aminoaryl represented by the following formula (2b) are reacted to synthesize a polymer including the repeating unit represented by formula (54) above.

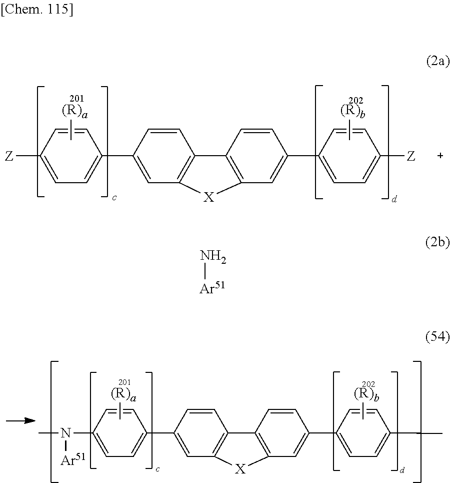

(In the above reaction formula, the definitions of Ar⁵¹, R²⁰¹, R²⁰², X, and a to d are the same as those for formula (54).)

With the polymerization method using the Ullmann reaction and the polymerization method using the Buchwald-Hartwig reaction, for example, dihalogenated aryl represented by formula (3a) (Z represents a halogen atom such as I, Br, Cl, or F) and primary aminoaryl represented by formula (3b) are reacted to synthesize a polymer including the repeating unit represented by formula (55) above.

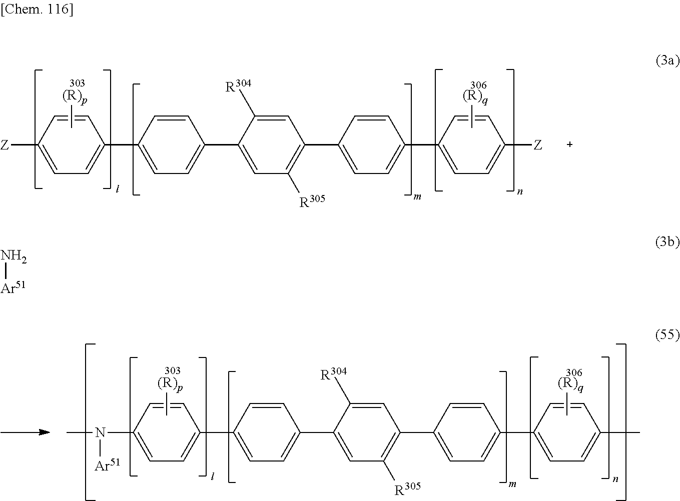

(In the above reaction formula, the definitions of Ar⁵¹, R³⁰³ to R³⁰⁶, n, m, l, p, and q are the same as those for formula (55).)

In the above polymerization methods, the reaction for forming N-aryl bonds is generally performed in the presence of a base such as potassium carbonate, sodium tert-butoxide, or triethylamine. The polymerization methods can also be performed in the presence of a transition metal catalyst such as a copper or palladium complex.

### [Contents of Compounds in Composition of Invention]

### <Content of Carbazole Compound in Invention>

From the viewpoint of reducing the injection barrier into the charge transport layer, the content of the carbazole compound in the invention in the compositional ratio of the solid components of the composition of the invention is preferably 10% by weight or more, more preferably 25% by weight or more, and still more preferably 30% by weight or more. From the viewpoint of maintaining the charge transportability in the charge transport layer, the content of the carbazole compound in the invention in the composition of the invention in the compositional ratio of the solid components of the composition is preferably 99% by weight or less, more preferably 90% by weight or less, and still more preferably 80% by weight or less.

### <Compositional Ratios and Contents of Carbazole Compound and Electron Accepting Compound in Composition>

In the composition of the invention, the content of the carbazole compound in the invention with respect to the total amount of the carbazole compound in the invention and the electron accepting compound in the invention is preferably 99% by weight or less, more preferably 97% by weight or less, and still more preferably 95% by weight or less. The content is preferably 50% by weight or more, more preferably 70% by weight or more, and still more preferably 80% by weight or more. When the content is within the above range, a film formed using the composition of the invention is crosslinked sufficiently and insolubilized, and a film can be formed by wet application directly on the film formed using the composition of the invention. Moreover, when the film formed using the composition of the invention is used as a charge injection layer, the injection barrier into the charge transport layer decreases, and good charge transportability is obtained. Moreover, stability during charge transport is improved, and the durability of an element including the film formed using the composition of the invention may be improved.

### <Content of High-Molecular Weight Charge Transport Compound>

To improve the charge transportability of a film formed of the carbazole compound in the invention having a thermal crosslinking group and the ionic compound including the electron accepting compound in the invention and an iodonium cation, it is preferable that these compounds are used in combination with the high-molecular weight charge transport compound. In this case, from the viewpoint of charge transportability, the content of the high-molecular weight charge transport compound in the composition of the invention in the compositional ratio of the solid components of the composition of the invention is preferably 10% by weight or more and more preferably 20% by weight or more. From the viewpoint of reducing the charge injection barrier between the charge injection layer and the charge transport layer adjacent to the charge injection layer, the content is preferably 95% by weight or less, more preferably 90% by weight or less, and still more preferably 85% by weight or less.

The composition of the invention may further contain a solvent, a polymerization initiator, an additive, etc.

### [Solvent]

Preferably, the composition of the invention further contains a solvent in addition to the carbazole compound in the invention and the electron accepting compound and/or the high-molecular weight charge transport compound. In particular, when the composition of the invention is used to form a charge transport film by a wet deposition method, it is preferable that a solvent is used to dissolve the carbazole compound in the invention and the electron accepting compound in the invention or the high-molecular weight charge transport compound.

No particular limitation is imposed on the type of solvent contained in the composition of the invention, so long as it can dissolve the carbazole compound in the invention and the electron accepting compound in the invention or the high-molecular weight charge transport compound. The solvent capable of dissolving the carbazole compound in the invention and the electron accepting compound in the invention is a solvent capable of dissolving the carbazole compound in the invention at preferably 0.005% by weight or more, more preferably 0.5% by weight or more, and still more preferably 1% by weight or more. The solvent can also dissolve the electron accepting compound at preferably 0.001% by weight or more, more preferably 0.1% by weight or more, and still more preferably 0.2% by weight or more. The solvent can also dissolve the high-molecular weight charge transport compound at preferably 0.005% by weight or more, more preferably 0.5% by weight or more, and still more preferably 1% by weight or more.

Preferred examples of the solvent include aromatic hydrocarbon-based solvents, ether-based solvents, and ester-based solvents.

Specific examples of the aromatic hydrocarbon-based solvent include toluene, xylene, mesitylene, tetralin, and cyclohexylbenzene.

Examples of the ether-based solvent include: aliphatic ethers such as ethylene glycol dimethyl ether, ethylene glycol diethyl ether, and propylene glycol-1-monomethyl ether acetate (PGMEA); and aromatic ethers such as 1,2-dimethoxybenzene, 1,3-dimethoxybenzene, anisole, phenetole, 2-methoxytoluene, 3-methoxytoluene, 4-methoxytoluene, 2,3-dimethylanisole, and 2,4-dimethylanisole.

Examples of the ester-based solvent include: aliphatic esters such as ethyl acetate, n-butyl acetate, ethyl lactate, and n-butyl lactate; and aromatic esters such as phenyl acetate, phenyl propionate, methyl benzoate, ethyl benzoate, propyl benzoate, and n-butyl benzoate.

Any one of these solvents may be used alone, or a combination of two or more may be used at any ratio.

Examples of the usable solvent other than the above ether-based and ester-based solvents include: amide-based solvents such as N,N-dimethylformamide and N,N-dimethylacetamide; and dimethyl sulfoxide. Any one of these solvents may be used alone, or a combination of two or more may be used at any ratio. A combination of one or two or more of these solvents and one or two or more of the above ether-based and ester-based solvents may be used. In particular, since the ability of the aromatic hydrocarbon-based solvents such as benzene, toluene, and xylene to dissolve the electron accepting compound and free carriers (cation radicals) is low, it is preferable that, when such an aromatic hydrocarbon-based solvent is used, it is mixed with an ether-based solvent and an ester-based solvent.

When a solvent is used, the concentration of the solvent with respect to the composition of the invention is preferably 10% by weight or more, more preferably 30% by weight or more, and still more preferably 50% by weight or more. The concentration of the solvent with respect to the composition is preferably 99.999% by weight or less, more preferably 99.99% by weight or less, and still more preferably 99.9% by weight or less. When a mixture of two or more solvents is used, the total amount of these solvents is adjusted so as to satisfy the above range.

The composition of the invention may be used for an organic electroluminescent element. The organic electroluminescent element is formed by stacking a large number of layers formed of organic compounds, and it is necessary that each of the layers be a uniform layer. In this case, when a wet deposition method is used to form a layer, if water is present in a thin film-forming solution (composition), the water is mixed into the coating, and the uniformity of the film is impaired. Therefore, the lower the content of water in the solution, the better. Generally, many materials that deteriorate significantly due to water in the cathode etc. are used in the organic electroluminescent element, and therefore the presence of water is unpreferable also from the viewpoint of deterioration of the element.

Specifically, the content of water in the composition of the invention is preferably 1% by weight or less and is reduced to preferably 0.1% by weight or less and more preferably 0.05% by weight or less.

Examples of the method for reducing the content of water in the composition include sealing with nitrogen gas, using a drying agent, pre-dehydration of the solvent, and using a solvent in which water is poorly soluble. In particular, from the viewpoint of preventing a phenomenon in which a coating film of the solution absorbs water in the air during an application step and is thereby whitened, it is preferable to use a solvent in which water is poorly soluble.

When the composition of the invention is used to form a film by a wet deposition method, the composition contains a solvent in which water is poorly soluble at a concentration of preferably 10% by weight or more, more preferably 30% by weight or more, and particularly preferably 50% by weight or more with respect to the total weight of the composition. Specifically, the solubility of water in the solvent at 25° C. is 1% by weight or less and preferably 0.1% by weight or less.

### [Charge Transport Film Composition]

When the electron accepting compound is the electron accepting ionic compound described above, it is preferable to use a composition containing the electron accepting ionic compound and the carbazole compound in the invention (this composition is hereinafter referred to appropriately as a “charge transport film composition (A)”) or a composition containing a charge transport ionic compound described later and including a cation radical of the carbazole compound in the invention and a counter anion that is part of the electron accepting ionic compound (this composition is hereinafter referred to appropriately as a “charge transport film composition (B)”). The charge transport film composition (A) and the charge transport film composition (B) will be described separately for the sake of convenience. However, a composition containing the electron accepting ionic compound, the carbazole compound in the invention, and the charge transport ionic compound described later and including a cation radical of the carbazole compound in the invention and a counter anion that is part of the electron accepting ionic compound can also be used as a charge transport film composition. The carbazole compound in the invention is the above-described carbazole compound having a crosslinking group.

The charge transport film compositions (A) and (B) are each a composition (charge transport material composition) that can be widely used for charge transport material applications. Generally, this composition is formed into a film and used as a hole injection layer and/or a hole transport layer, i.e., a “charge transport film” that transports holes, i.e., charges. Therefore, in the present description, this composition is referred to particularly as the “charge transport film composition.”

### <Charge Transport Film Composition (A)>

The charge transport film composition (A) contains the carbazole compound in the invention, the electron accepting compound having a crosslinking group, and a solvent. The charge transport film composition (A) may contain one carbazole compound in the invention or may contain two or more carbazole compounds. The charge transport film composition (A) may further contain the high-molecular weight hole transport compound described above.

### <Method for Preparing Charge Transport Film Composition (A)>

The charge transport film composition (A) is prepared by mixing at least the electron accepting compound and the carbazole compound in the invention. In this case, it is preferable that the charge transport film composition (A) contains a solvent and that the electron accepting compound and the carbazole compound in the invention are dissolved in the solvent to mix them.

The content of the electron accepting compound in the charge transport film composition (A) with respect to the amount of the carbazole compound in the invention is generally 0.1% by weight or more and preferably 1% by weight or more and is generally 100% by weight or less and preferably 40% by weight or less. When the content of the electron accepting compound is equal to or more than the above lower limit, free carriers (cation radicals of the carbazole compound in the invention) are generated sufficiently, which is preferred. When the content of the electron accepting compound is equal to or less than the above upper limit, sufficient charge transportability is obtained, which is preferred. When two or more electron accepting compounds are used in combination, the total content of the compounds is adjusted within the above range. The same applies to the charge transport compound.

### <Charge Transport Film Composition (B)>

As described above, the charge transport film composition (B) is the composition containing the charge transport ionic compound including a cation radical of the carbazole compound in the invention and the electron accepting ionic compound serving as a counter anion.

The cation radical of the carbazole compound in the invention that is the cation of the charge transport ionic compound is a chemical species obtained by removing one electron from an electrically neutral compound represented as the carbazole compound in the invention.

The cation radical of the carbazole compound in the invention represented by formula (71) above is an aromatic carbazole compound having a structure represented by formula (110) below.

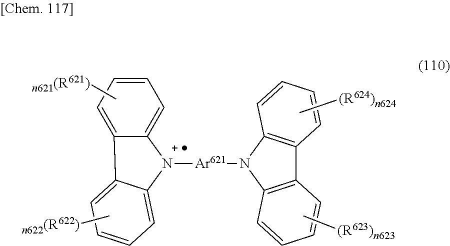

(In formula (110) above, Ar⁶²¹, R⁶²¹, R⁶²², R⁶²³, R⁶²⁴, n621, n622, n623, n624 are the same as Ar⁶²¹, R⁶²¹, R⁶²², R⁶²³, R⁶²⁴, n621, n622, n623, and n624, respectively, in formula (71) above.)

The aromatic carbazole compound having the structure represented by formula (110) is particularly preferably an aromatic carbazole compound having a structure represented by the following formula (110-2) because it has an appropriate oxidation-reduction potential and because a stable charge transport ionic compound is obtained.

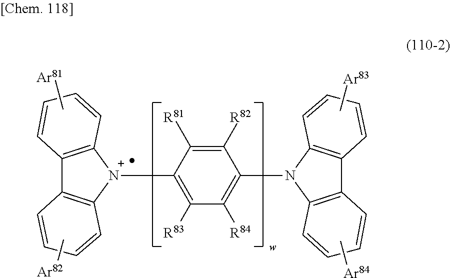

[In formula (110-2) above,

w represents an integer of 1 to 6.

Ar⁸¹ to Ar⁸⁴ each independently represent a hydrogen atom, a deuterium atom, a halogen atom (specifically an I, Br, Cl, or F atom), an aromatic hydrocarbon group having 6 to 30 carbon atoms and optionally having a substituent, or an aromatic heterocyclic group having 3 to 30 carbon atoms and optionally having a substituent.

R⁸¹ to R⁸⁴ each independently represent a substituent and may each be bonded to a substituent on an adjacent phenylene group.]

Ar⁸¹ to Ar⁸⁴ are each preferably an aromatic hydrocarbon group having a substituent, and specific example, preferred examples, examples of the optional substituent, and preferred examples of the substituent are the same as those for R⁶²¹ to R⁶²⁴ in formula (71) above. Ar⁸¹ to Ar⁸⁴ are each particularly preferably an aromatic hydrocarbon group having 6 to 14 carbon atoms and optionally having a substituent.

Preferred substituents and preferred R⁸¹ to R⁸⁴ are each a group selected from the substituent group Z and are each preferably unsubstituted or an alkyl or aromatic hydrocarbon group in the substituent group Z.

From the viewpoint of ease of forming a cation radical from the partial structure represented by formula (110-2) and from the viewpoint of charge transformability, w is preferably 6 or less, more preferably 5 or less, and particularly preferably 4.

The aromatic carbazole compound having the structure represented by formula (110-2) may be a low-molecular weight compound having only one or a plurality of structures represented by formula (110-2) as aromatic carbazole structures.

The cation radial of the carbazole compound in the invention represented by formula (72) above is an aromatic carbazole compound having a structure represented by formula (120) below.

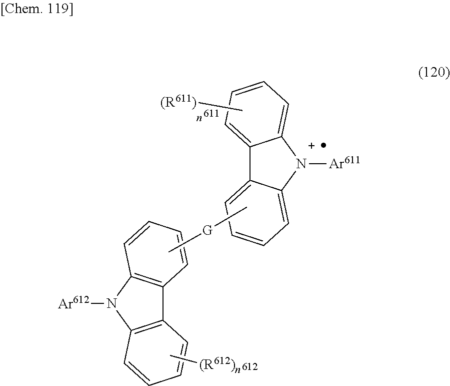

(In formula (120), Ar⁶¹¹, Ar⁶¹², R⁶¹¹, R⁶¹², G, n⁶¹¹, and n⁶¹² are the same as Ar⁶¹¹, Ar⁶¹², R⁶¹¹, R⁶¹², G, n⁶¹¹, and n⁶¹², respectively, in formula (72).)

The aromatic carbazole compound having the structure represented by formula (120) is particularly preferably an aromatic carbazole compound having a structure represented by formula (120-2) below because it has an appropriate oxidation-reduction potential and because a stable charge transport ionic compound is obtained.

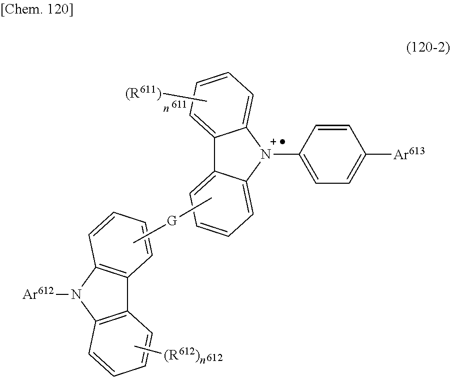

(In formula (120-2) above, Ar⁶¹¹, R⁶¹¹, R⁶¹², G, n⁶¹¹, and n⁶¹² are the same as Ar⁶¹¹, R⁶¹¹, R⁶¹², G, n⁶¹¹, and n⁶¹², respectively, in formula (72) above.

Ar⁶¹³ is a residue obtained by removing a phenylene group from Ar⁶¹² in formula (72) above when Ar⁶¹² is a structure bondable to a carbazole structure via the phenylene group.)

### <Charge Transport Ionic Compound>

The charge transport ionic compound is a compound in which the cation radical of the carbazole compound in the invention and a counter anion that is part of the electron accepting ionic compound are ionically bonded.

The charge transport ionic compound can be obtained by mixing the electron accepting ionic compound and the carbazole compound in the invention and is easily dissolved in various solvents. Specifically, the charge transport ionic compound can be obtained by a method described later in <Method for preparing charge transport film composition (B)>.

The molecular weight of the charge transport ionic compound is generally 1000 or more, preferably 1200 or more, and more preferably 1400 or more and is generally 9000 or less, preferably 5000 or less, and more preferably 4000 or less, but this is not the case when the cation radical is a high-molecular weight compound.

### <Method for Preparing Charge Transport Film Composition (B)>

Preferably, the charge transport ionic compound (B) is obtained by dissolving the electron accepting ionic compound and the carbazole compound in the invention in a solvent and mixing them. In this solution, the electron accepting ionic compound oxidizes the carbazole compound in the invention to form a cation radical, and the charge transport ionic compound is thereby formed, which is an ionic compound including the electron accepting ionic compound serving as a counter anion and the cation radical of the carbazole compound in the invention.

In this case, by mixing the carbazole compound in the invention and the electron accepting ionic compound in the solution, the probability that the electron accepting ionic compound is present in the vicinity of the carbazole nitrogen atoms that are easily-oxidizable sites of the carbazole compound in the invention becomes high. Therefore, the electron accepting ionic compound can easily oxidize the carbazole in the carbazole compound in the invention to form a cation radical, and the ionic compound including the electron accepting ionic compound serving as a counter anion and the cation radical of the carbazole compound in the invention can be easily generated. In this case, from the viewpoint of facilitating the reaction, it is preferable to heat the solution.

It is also preferable that a mixture of the electron accepting ionic compound and the carbazole compound in the invention is heated to prepare the charge transport film composition (B). This mixture is preferably a film formed by dissolving a mixture of the electron accepting ionic compound and the carbazole compound in the invention in a solvent, applying the solution, and drying the applied solution. By heating the mixture, mutual diffusion of the electron accepting ionic compound and the carbazole compound in the invention occurs in the mixture, and the probability that the electron accepting ionic compound is present in the vicinity of the carbazole nitrogen atoms that are easily-oxidizable sites of the carbazole compound in the invention becomes high, so that the ionic compound including the electron accepting ionic compound serving as a counter anion and the cation radical of the carbazole compound in the invention can be easily generated. The heating temperature in this case is preferably a temperature at which the crosslinking groups in the composition do not undergo the crosslinking reaction. However, even when the heating temperature is a temperature at which the crosslinking groups undergo the crosslinking reaction, the crosslinking reaction occurs while the diffusion occurs, and therefore the electron accepting ionic compound is formed without any problem.

The charge transport film composition (B) may contain one charge transport ionic compound alone or may contain two or more charge transport ionic compounds. It is preferable to contain one or two charge transport ionic compounds, and it is more preferable to contain one charge transport ionic compound alone. This is because variations in ionization potential of the charge transport ionic compound are small, and the hole transportability is high.

The composition containing only one or two charge transport ionic compounds is a composition prepared using only a total of two or three compounds including at least one electron accepting ionic compound and at least one carbazole compound in the invention and is a compound prepared using at least one electron accepting ionic compound and at least one carbazole compound in the invention.

It is also preferable that the charge transport film composition (B) further contains a charge transport compound in addition to the charge transport ionic compound. The charge transport compound is particularly preferably a polymer including, as a repeating unit, the above-described arylamine structure, i.e., the repeating unit represented by formula (50) above, and the polymer is the high-molecular weight hole transport compound described above.

When the charge transport film composition (B) is prepared, the content, i.e., the amount used, of the carbazole compound in the invention with respect to the amount of the charge transport ionic compound is preferably 10% by weight or more, more preferably 20% by weight or more, and still more preferably 30% by weight or more. The content is preferably 10000% by weight or less and more preferably 1000% by weight or less.

In a charge transport film formed using the charge transport film composition (B), positive charges move from the charge transport ionic compound to the nearby neutral charge transport compound, and high hole injection-transport ability is obtained. Therefore, the mass ratio of the charge transport ionic compound to the neutral carbazole compound in the invention is preferably about 1:100 to about 100:1 and more preferably about 1:20 to about 20:1.

### <Relation Between Charge Transport Film Compositions (A) and (B)>

The charge transport film formed using the charge transport film composition (A) has high heat resistance and high hole injection-transport ability. The reason for these good characteristics will be described below.

The charge transport film composition (A) contains the above-described electron accepting compound and the above-described charge transport compound. The cation in the electron accepting ionic compound has a hypervalent central atom, and its positive charge is widely delocalized, so that its electron acceptability is high. This allows electron transfer from the charge transport compound to the cation of the electron accepting ionic compound, and the charge transport ionic compound including the cation radical of the charge transport compound and a counter anion is thereby formed. The cation radical of the charge transport compound serves as a charge carrier, and the electric conductivity of the charge transport film can thereby be increased. Specifically, when the charge transport film composition (A) is prepared, the charge transport ionic compound including the cation radical of the charge transport compound and the electron accepting ionic compound serving as the counter anion may be formed at least partially.

For example, when an electron transfers from a charge transport compound represented by formula (7) below to an electron accepting compound represented by formula (6), a charge transport ionic compound represented by formula (9) and including the cation radical of the charge transport compound and a counter anion J- is formed.

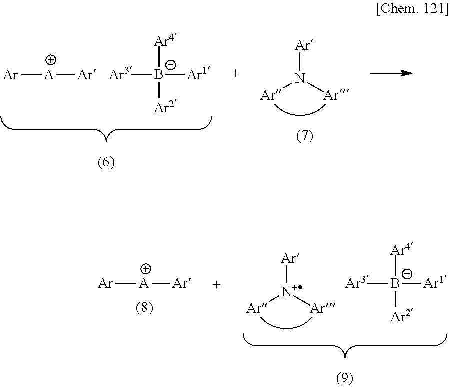

### [Preparation of Composition]

The composition of the invention can be prepared by mixing a solvent and functional materials including the carbazole compound in the invention and the electron accepting compound and/or the polymer and heating the mixture for a prescribed time to dissolve or disperse the functional materials. To dissolve or disperse the functional materials uniformly in the solvent, the heating temperature is generally room temperature or equal to or higher than room temperature and is preferably 80° C. or higher, more preferably 90° C. or higher, and still more preferably 100° C. or higher, e.g., 100 to 115° C. The heating time is preferably 30 minutes or longer, more preferably 45 minutes or longer, and still more preferably 60 minutes or longer, e.g., 60 to 180 minutes.

The composition after heating is filtered using a membrane filter, a depth filter, etc. to remove coarse particles before use. Since the composition is ejected from a nozzle of an inkjet head for the application of the composition, the pore diameter of the filter is preferably 0.5 μm or less, more preferably 0.2 μm or less, and still more preferably 0.1 μm or less.

### [Film Formation Method Using Composition]

When a film is formed using the composition of the invention, the composition of the invention is preferably a solution containing a solvent, and it is preferable to subject the composition of the invention to wet deposition.

The wet deposition method is a method including coating a substrate with a composition containing a solvent and drying the composition to remove the solvent to thereby form a film. No particular limitation is imposed on the coating method, and examples of the method include a spin coating method, a dip coating method, a die coating method, a bar coating method, a blade coating method, a roll coating method, a spray coating method, a capillary coating method, an inkjet method, a screen printing method, a gravure printing method, and a flexographic printing method.

To remove the solvent by drying, heat drying is generally performed. Examples of the heating means used in the heating process include a clean oven, a hot plate, and infrared heating. A halogen heater, a ceramic-coated halogen heater, a ceramic heater, etc. can be used for infrared heating.

With the infrared heating, thermal energy is directly applied to the substrate or the film, and therefore the time required for drying can be shorter than that with heating using an oven or a hot plate. Therefore, the influence of gases (moisture and oxygen) in the heating atmosphere and the influence of small dust can be minimized, and the productivity is improved, which is preferred.

The heating temperature is generally 80° C. or higher, preferably 100° C. or higher, and more preferably 150° C. or higher and is generally 300° C. or lower, preferably 280° C. or lower, and more preferably 260° C. or lower.

The heating time is generally 10 seconds or longer, preferably 60 seconds or longer, and more preferably 90 seconds or longer and is generally 120 minutes or shorter, preferably 60 minutes or shorter, and more preferably 30 minutes or shorter.

It is also preferable to perform vacuum drying before the heat drying.

The thickness of the organic layer formed using the composition of the invention by the wet deposition method is generally 5 nm or more, preferably 10 nm or more, and more preferably 20 nm or more. The thickness is generally 1000 nm or less, preferably 500 nm or less, and more preferably 300 nm or less.

### [Organic Electroluminescent Element]

A film using the composition of the invention and a film formed using the composition of the invention can each be preferably used as a charge transport layer. This charge transport layer is particularly preferably used as a charge transport film of an organic electroluminescent element.

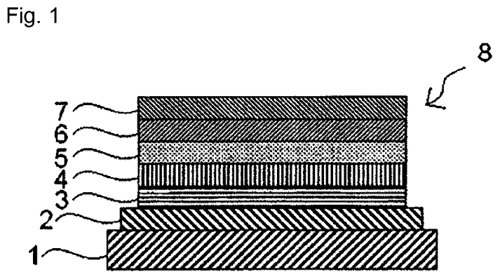

As an example of the organic electroluminescent element of the present invention, FIG. 1 shows a schematic illustration (cross section) of an example of the structure of an organic electroluminescent element 8. In FIG. 1, 1 represents a substrate; 2 represents an anode; 3 represents a hole injection layer; 4 represents a hole transport layer; 5 represents a light-emitting layer; 6 represents an electron transport layer; and 7 represents a cathode.

### [Substrate]

The substrate 1 is a support member for supporting the organic electroluminescent element. Usually, the substrate 1 is a quartz or glass plate, a metal plate, a metal foil, a plastic film, or a plastic sheet, etc. Among these, a glass plate or a plate of a transparent synthetic resin such as polyester, polymethacrylate, polycarbonate, or polysulfone is preferred. It is preferable that the substrate is made of a material having high gas barrier properties because deterioration of the organic electroluminescent element due to outside air passing through the substrate is suppressed. Therefore, when a material having low gas barrier properties such as a synthetic resin substrate is used, it is preferable to provide a dense silicon oxide film or the like on at least one side of the substrate to improve the gas barrier properties.

### [Anode]

The anode 2 has a function of injecting holes into the layers on the light-emitting layer 5 side.

The anode 2 is usually made of metals such as aluminum, gold, silver, nickel, palladium, platinum, and the like; metal oxides such as indium and/or tin oxide, and the like; metal halides such as copper iodide, and the like; carbon black; and conductive polymers such as poly(3-methylthiophene), polypyrrole, polyaniline, and the like.

The anode 2 is usually formed by a dry method such as sputtering method, vacuum vapor deposition method, and the like. When forming the anode using fine metal particles such as silver, and the like; fine particles such as copper iodide, and the like; carbon black; fine electrically conductive metal oxide particles; fine conductive polymer particles; and the like, it can also be formed by dispersing them in a suitable binder resin solution and applying it to the substrate. In the case of conductive polymers, a thin film can be formed directly on the substrate by electrolytic polymerization, or the anode can be formed by applying the conductive polymer to the substrate (Appl. Phys. Lett., Vol. 60, p. 2711, 1992).

The anode 2 has generally a monolayer structure but may have a layered structure. When the anode 2 has a layered structure, a different conductive material may be laminated on the first anode layer.

The thickness of the anode 2 may be determined according to the required transparency and material. When particularly high transparency is required, a thickness that provides a visible light transmittance of 60% or more is preferable, and a thickness that provides a visible light transmittance of 80% or more is even more preferable. The thickness of the anode 2 is usually 5 nm or more, and preferably 10 nm or more, and usually 1000 nm or less, and preferably 500 nm or less. When transparency is not required, the thickness of the anode 2 may be arbitrarily set according to the required strength, and the like. In this case, the anode 2 may have the same thickness as the substrate.

When forming another layer on the surface of the anode 2, it is preferable to remove impurities on the anode 2 and improve hole injection by adjusting its ionization potential by performing a treatment such as ultraviolet light/ozone, oxygen plasma, or argon plasma before the film formation.

### [Hole Injection Layer]

The layer that transports holes from the anode 2 side to the light-emitting layer 5 side is usually called a hole injection layer or hole transport layer. When there are two or more layers that transport holes from the anode 2 side to the light-emitting layer 5 side, the layer closer to the anode side may be called the hole injection layer 3. The hole injection layer 3 is preferably formed in order to strengthen the function of transporting holes from the anode 2 to the light-emitting layer 5 side. When the hole injection layer 3 is formed, it is usually formed on the anode 2.

The hole injection layer 3 formed using the composition of the invention contains a crosslinking reaction product of the carbazole compound in the invention and the electron accepting compound described above.

No particular limitation is imposed on the method for forming the hole injection layer 3. Examples of the method include a vacuum vapor deposition method and a wet deposition method. When the wet deposition method is used to form the layer, the composition of the invention is prepared, applied to the anode 2 by the wet deposition method such as a spin coating method or a dip coating method, and dried to thereby form the hole injection layer 3.

Particularly preferably, a composition containing the carbazole compound in the invention and the above-described electron accepting compound is used, and a film formed using the composition containing the carbazole compound in the invention and the above-described electron accepting compound is used.

The thickness of the hole injection layer 3 formed as described above is generally 5 nm or more and preferably 10 nm or more and is generally 1000 nm or less and preferably 500 nm or less.

The method for forming the hole injection layer may be a vacuum vapor deposition method or may be a wet deposition method. It is preferable to form the hole injection layer using the wet deposition method because of its good film formability.

Examples of the solvent include ether-based solvents, ester-based solvents, aromatic hydrocarbon-based solvents, and amide-based solvents.

Examples of the ether-based solvents include: aliphatic ethers such as ethylene glycol dimethyl ether, ethylene glycol diethyl ether, and propylene glycol-1-monomethyl ether acetate (PGMEA); and aromatic ethers such as 1,2-dimethoxybenzene, 1,3-dimethoxybenzene, anisole, phenetole, 2-methoxytoluene, 3-methoxytoluene, 4-methoxytoluene, 2,3-dimethylanisole, and 2,4-dimethylanisole.

Examples of the ester-based solvents include aromatic esters such as phenyl acetate, phenyl propionate, methyl benzoate, ethyl benzoate, propyl benzoate, and n-butyl benzoate.

Examples of the aromatic hydrocarbon-based solvents include toluene, xylene, cyclohexylbenzene, 3-isopropylbiphenyl, 1,2,3,4-tetramethylbenzene, 1,4-diisopropylbenzene, cyclohexylbenzene, and methylnaphthalene.

Examples of the amide-based solvents include N,N-dimethylformamide and N,N-dimethylacetamide.

In addition, dimethyl sulfoxide etc. may be used.

Generally, when the hole injection layer 3 is formed by the wet deposition method, a hole injection layer-forming composition is prepared and then applied to a layer (generally the anode 2) corresponding to a layer disposed below the hole injection layer 3 to thereby form a film, and then the film is dried.

Generally, after the formation of the coating film, the film is dried by heating, vacuum drying, etc. to thereby obtain the hole injection layer 3.

### [Hole Transport Layer]

The hole transport layer 4 is a layer having the function of transporting holes from the anode 2 side to the light-emitting layer 5 side. The hole transport layer 4 is not an essential layer of the organic electroluminescent element of the invention. However, it is preferable to form this layer in order to enhance the function of transporting holes from the anode 2 to the light-emitting layer 5. When the hole transport layer 4 is formed, the hole transport layer 4 is generally formed between the anode 2 and the light-emitting layer 5. When the above-described hole injection layer 3 is present, the hole transport layer 4 is formed between the hole injection layer 3 and the light-emitting layer 5.

The thickness of the hole transport layer 4 is generally 5 nm or more and preferably 10 nm or more and is generally 300 nm or less and preferably 100 nm or less.

A material for forming the hole transport layer 4 is preferably a material having high hole transportability and capable of transporting injected holes efficiently. Therefore, it is preferable that the material forming the hole transport layer 4 has a low ionization potential, is highly transparent to visible light, has large hole mobility, and is highly stable and that impurities serving as traps are unlikely to be mixed into the material during production and use. In many cases, the hole transport layer 4 is in contact with the light-emitting layer 5. It is therefore preferable that the hole transport layer 4 does not attenuate the light emitted from the light-emitting layer 5 and does not allow exciplexes to be formed between the hole transport layer 4 and the light-emitting layer 5 so that the efficiency is not reduced.

The material of the hole transport layer 4 may be any material conventionally used as the material forming the hole transport layer. Examples thereof include those shown as the examples of the hole transport compound used for the hole injection layer 3. Other examples include arylamine derivatives, fluorene derivatives, spiro derivatives, carbazole derivatives, pyridine derivatives, pyrazine derivatives, pyrimidine derivatives, triazine derivatives, quinoline derivatives, phenanthroline derivatives, phthalocyanine derivatives, porphyrin derivatives, silole derivatives, oligothiophene derivatives, polycyclic aromatic ring derivatives, and metal complexes.

Still other examples include polyvinylcarbazole derivatives, polyarylamine derivatives, polyvinyltriphenylamine derivatives, polyfluorene derivatives, polyarylene derivatives, polyarylene ether sulfone derivatives containing tetraphenylbenzidine, polyarylenevinylene derivatives, polysiloxane derivatives, polythiophene derivatives, and poly(p-phenylenevinylene) derivatives. These may each be an alternating copolymer, a random polymer, a block polymer, of a graft copolymer. Moreover, polymers having a branched main chain with three or more terminal ends and so-called dendrimers may also be used.

In particular, a polyarylamine derivative or a polyarylene derivative is preferred.

The polyarylamine derivative is preferably a polymer including a repeating unit represented by formula (I) below. A polymer formed from the repeating unit represented by formula (I) below is particularly preferred. In this case, Ar^{a}'s or Ar^{b}'s in different repeating units may differ from each other.

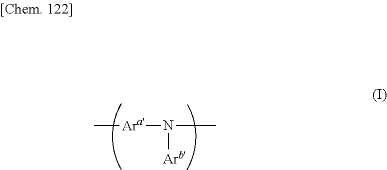

(In formula (I), Ar^{a}′ and Ar^{b}′ each independently represent an aromatic hydrocarbon group optionally having a substituent or an aromatic heterocyclic group optionally having a substituent.)

Examples of the polyarylene derivative include polymers having a repeating unit including an arylene group such as an aromatic hydrocarbon group optionally having a substituent or an aromatic heterocyclic group optionally having a substituent.

The polyarylene derivative is preferably a polymer having a repeating unit represented by formula (II-1) below and/or a repeating unit represented by formula (II-2) below.

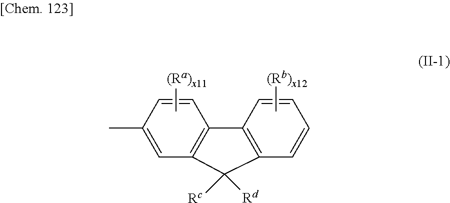

(In formula (II-1), R^{a}, R^{b}, R^{c}, and R^{d} each independently represent an alkyl group, an alkoxy group, a phenylalkyl group, a phenylalkoxy group, a phenyl group, a phenoxy group, an alkylphenyl group, an alkoxyphenyl group, an alkylcarbonyl group, an alkoxycarbonyl group, or a carboxy group. x11 and x12 each independently represent an integer of 0 to 3. When x11 or x12 is 2 or more, a plurality of Ra's or R^{b}'s in one molecule may be the same or different. Adjacent R^{a}'s or R^{b}'s may together form a ring.)

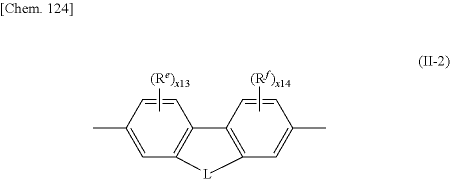

(In formula (II-2), R^{e} and R^{f} are each independently the same as R^{a}, R^{b}, R^{c}, or R^{d} in formula (11-2) above. x13 and x14 each independently represent an integer of 0 to 3. When x13 or x14 is 2 or more, a plurality of R^{e}'s or R^{f}'s included in one molecule may be the same or different. Adjacent R^{e}'s or R^{f}'s may together form a ring. L represents an atom or an atomic group included in a 5- or 6-membered ring.)

Specific examples of L include an oxygen atom, a boron atom optionally having a substituent, a nitrogen atom optionally having a substituent, a silicon atom optionally having a substituent, a phosphorus atom optionally having a substituent, a sulfur atom optionally having a substituent, a carbon atom optionally having a substituent, and groups formed by bonding any of these atoms.

Preferably, the polyarylene derivative further includes a repeating unit represented by formula (III-3) below in addition to the repeating unit represented by formula (II-1) above and/or the repeating unit represented by formula (II-2) above.

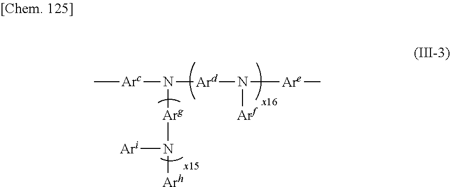

(In formula (III-3), Ar^{c} to Ar¹ each independently represent an aromatic hydrocarbon group optionally having a substituent or an aromatic heterocyclic group optionally having a substituent. x15 and x16 each independently represent 0 or 1.)

Specific examples of formulas (III-1) to (III-3) above and specific examples of the polyarylene derivative include those described in JP2008-98619A.

When the hole transport layer 4 is formed by a wet deposition method, a hole transport layer-forming composition is prepared. Then wet deposition is performed, and the resulting layer is heated and dried, as in the formation of the hole injection layer 3.

The hole transport layer-forming composition contains a solvent in addition to the hole transport compound described above. The solvent used is the same as the solvent used for the hole injection layer-forming composition described above. The film deposition conditions, the heat drying conditions, etc. are also the same as those for the formation of the hole injection layer 3.

When a vacuum vapor deposition method is used to form the hole transport layer, the film deposition conditions etc. are also the same as those for the formation of the hole injection layer 3.

The hole transport layer 4 may further contain, in addition to the hole transport compound described above, various light-emitting materials, an electron transport compound, a binder resin, an applicability improver, etc.

The hole transport layer 4 may be a layer formed by crosslinking a crosslinkable compound. The crosslinkable compound is a compound having a crosslinkable group and forms a high-molecular weight network compound when crosslinked.

Examples of the crosslinkable group include: groups derived from cyclic ethers such as oxetane and epoxies; groups derived from an unsaturated double bond such as a vinyl group, a trifluorovinyl group, a styryl group, an acryl group, methacryloyl, and cinnamoyl; and groups derived from benzocyclobutene.

The crosslinkable compound may be any of a monomer, an oligomer, and a polymer.

Only one crosslinkable compound may be included, or a combination of two or more at any ratio may be included.

The crosslinkable compound used is preferably a hole transport compound having a crosslinkable group. Examples of the hole transport compound include those shown above. The crosslinkable compound is, for example, a hole transport compound including a crosslinkable group bonded to its main or side chain. In particular, it is preferable that the crosslinkable group is bonded to the main chain via a linking group such as an alkylene group. In particular, the hole transport compound is preferably a polymer including a repeating unit having a crosslinkable group and more preferably a polymer including a repeating unit in which a crosslinkable group is bonded to one of formulas (I) and (II-1) to (III-3) directly or via a linking group.

To form the hole transport layer 4 by crosslinking the crosslinkable compound, the crosslinkable compound is generally dissolved or dispersed in a solvent to prepare a hole transport layer-forming composition. Then a film is formed by wet deposition, and the hole transport layer-forming composition is crosslinked.

The thickness of the hole transport layer 4 formed as described above is generally 5 nm or more and preferably 10 nm or more and is generally 300 nm or less and preferably 150 nm or less.

### [Light-Emitting Layer]

The light-emitting layer 5 is a layer having the function of emitting light. Specifically, when an electric field is applied between the pair of electrodes, the light-emitting layer 5 is excited by recombination of holes injected from the anode 2 and electrons injected from the cathode 7 to thereby emit light. The light-emitting layer 5 is formed between the anode 2 and the cathode 7. When the hole injection layer is present on the anode, the light-emitting layer is formed between the hole injection layer and the cathode. When the hole transport layer is present on the anode, the light-emitting layer is formed between the hole transport layer and the cathode.

Preferably, the organic electroluminescent element in the invention contains a light-emitting layer-forming material suitable for the light-emitting layer.

The light-emitting layer 5 can have any thickness so long as the effects of the invention are not significantly impaired. The larger the thickness, the better from the viewpoint that defects are unlikely to be formed in the film. However, the smaller the thickness, the better from the viewpoint that the driving voltage can be easily reduced. Therefore, the thickness of the light-emitting layer 5 is preferably 3 nm or more and more preferably 5 nm or more and is generally preferably 200 nm or less and more preferably 100 nm or less.

The light-emitting layer 5 contains at least a material capable of emitting light (a light-emitting material) and contains preferably one or a plurality of host materials.

### [Preferred Light-Emitting Layer-Forming Material]

The light-emitting layer in the invention contains the light-emitting material and a charge transport material. The light-emitting material may be a phosphorescent light-emitting material or may be a fluorescent light-emitting material. Preferably, a red light-emitting material and a green light-emitting material are each a phosphorescent light-emitting material, and a blue light-emitting material is a fluorescent light-emitting material.

### <Phosphorescent Light-Emitting Material>

The phosphorescent light-emitting material is a material that emits light from an excited triplet state. Typical examples thereof include metal complex compounds containing Ir, Pt, Eu, etc., and it is preferable that the structure of the material contains a metal complex.

Among the metal complexes are phosphorescent light-emitting organic metal complexes that emit light through a triplet state, and examples thereof include Werner-type complex and organometallic complex compounds that contain, as the central metal, a metal selected from groups 7 to 11 of the long-form periodic table (hereinafter, the term “periodic table” refers to the long-form periodic table unless otherwise specified). Examples of such a phosphorescent light-emitting material include phosphorescent light-emitting materials described in WO2014/024889, WO2015-087961, WO2016/194784, and JP2014-074000A. The phosphorescent light-emitting material is preferably a compound represented by formula (201) below or a compound represented by formula (205) below and more preferably the compound represented by formula (201) below.

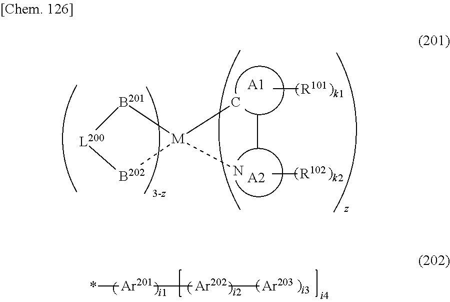

In formula (201), ring A1 represents an aromatic hydrocarbon ring structure optionally having a substituent or an aromatic heterocyclic structure optionally having a substituent.

Ring A2 represents an aromatic heterocyclic structure optionally having a substituent.

R¹⁰¹ and R¹⁰² are each independently a structure represented by formula (202).

* represents a position of bonding to ring A1 or A2.

R¹⁰¹ and R¹⁰² may be the same or different. When a plurality of R¹⁰¹'s are present, they may be the same or different. When a plurality of R¹⁰²'s are present, they may be the same or different.

Ar²⁰¹ and Ar²⁰³ each independently represent an aromatic hydrocarbon ring structure optionally having a substituent or an aromatic heterocyclic structure optionally having a substituent.

Ar²⁰² represents an aromatic hydrocarbon ring structure optionally having a substituent, an aromatic heterocyclic structure optionally having a substituent, or an aliphatic hydrocarbon structure optionally having a substituent.

Substituents bonded to ring A1, substituents bonded to ring A2, or a substituent bonded to ring A1 and a substituent bonded to ring A2 may be bonded together to form a ring.

B²⁰¹-L²⁰⁰-B²⁰² represents an anionic bidentate ligand. B²⁰¹ and B²⁰² each independently represent a carbon atom, an oxygen atom, or a nitrogen atom, and these atoms may each be an atom included in a ring. L²⁰⁰ represents a single bond or an atomic group that, together with B²⁰¹ and B²⁰², forms the bidentate ligand. When a plurality of B²⁰¹-L²⁰⁰-B²⁰² ligands are present, they may be the same or different.

In formulas (201) and (202),

i1 and i2 each independently represent an integer of 0 or more and 12 or less.

i3 represents an integer of 0 or more, and its upper limit is set to the number of substituents that Ar²⁰² can have.

i4 represents an integer of 0 or more, and its upper limit is set to the number of substituents that Ar²⁰¹ can have.

k1 and k2 are each independently an integer of 0 or more, and their upper limits are set to the numbers of substituents that rings A1 and A2 can have.

z represents an integer of 1 to 3.

### (Substituent)

Preferably, the substituent is a group selected from the following substituent group S, unless otherwise specified.

### <Substituent Group S>

Alkyl groups, preferably alkyl groups having 1 to 20 carbon atoms, more preferably alkyl groups having 1 to 12 carbon atoms, still more preferably alkyl group having 1 to 8 carbon atoms, and particularly preferably alkyl groups having 1 to 6 carbon atoms.
Alkoxy groups, preferably alkoxy groups having 1 to 20 carbon atoms, more preferably alkoxy groups having 1 to 12 carbon atoms, and still more preferably alkoxy groups having 1 to 6 carbon atoms.
Aryloxy groups, preferably aryloxy groups having 6 to 20 carbon atoms, more preferably aryloxy groups having 6 to 14 carbon atoms, still more preferably aryloxy groups having 6 to 12 carbon atoms, and particularly preferably aryloxy groups having 6 carbon atoms.
Heteroaryloxy groups, preferably heteroaryloxy groups having 3 to 20 carbon atoms, and more preferably heteroaryloxy groups having 3 to 12 carbon atoms.
Alkylamino groups, preferably alkylamino groups having 1 to 20 carbon atoms, and more preferably alkylamino groups having 1 to 12 carbon atoms.
Arylamino groups, preferably arylamino groups having 6 to 36 carbon atoms, and more preferably arylamino groups having 6 to 24 carbon atoms.
Aralkyl groups, preferably aralkyl groups having 7 to 40 carbon atoms, more preferably aralkyl groups having 7 to 18 carbon atoms, and still more preferably aralkyl groups having 7 to 12 carbon atoms.
Heteroaralkyl groups, preferably heteroaralkyl groups having 7 to 40 carbon atoms, and more preferably heteroaralkyl groups having 7 to 18 carbon atoms.
Alkenyl groups, preferably alkenyl groups having 2 to 20 carbon atoms, more preferably alkenyl groups having 2 to 12 carbon atoms, still more preferably alkenyl groups having 2 to 8 carbon atoms, and particularly preferably alkenyl groups having 2 to 6 carbon atoms.
Alkynyl groups, preferably alkynyl groups having 2 to 20 carbon atoms, and more preferably alkynyl groups having 2 to 12 carbon atoms.
Aryl groups, preferably aryl groups having 6 to 30 carbon atoms, more preferably aryl groups having 6 to 24 carbon atoms, still more preferably aryl groups having 6 to 18 carbon atoms, and particularly preferably aryl groups having 6 to 14 carbon atoms.
Heteroaryl groups, preferably heteroaryl groups having 3 to 30 carbon atoms, more preferably heteroaryl groups having 3 to 24 carbon atoms, still more preferably heteroaryl groups having 3 to 18 carbon atoms, and particularly preferably heteroaryl groups having 3 to 14 carbon atoms.
Alkylsilyl groups, preferably alkylsilyl groups each having an alkyl group having 1 to 20 carbon atoms, and more preferably alkylsilyl groups each having an alkyl group having 1 to 12 carbon atoms.
Arylsilyl groups, preferably arylsilyl groups each having an aryl group having 6 to 20 carbon atoms, and more preferably arylsilyl groups each having an aryl group having 6 to 14 carbon atoms.
Alkylcarbonyl groups and preferably alkylcarbonyl groups having 2 to 20 carbon atoms.
Arylcarbonyl groups and preferably arylcarbonyl groups having 7 to 20 carbon atoms.

In the above groups, at least one hydrogen atom may be substituted with a fluorine atom, or at least one hydrogen atom may be substituted with a deuterium atom.

The aryl is an aromatic hydrocarbon ring, and the heteroaryl is an aromatic heterocycle, unless otherwise specified.
A hydrogen atom, a deuterium atom, a fluorine atom, a cyano group, and —SF₅.

The substituent selected from the substituent group S is preferably an alkyl group, an alkoxy group, an aryloxy group, an arylamino group, an aralkyl group, an alkenyl group, an aryl group, a heteroaryl group, an alkylsilyl group, an arylsilyl group, one of these groups in which at least one hydrogen atom is substituted with a fluorine atom, a fluorine atom, a cyano group, or —SF₅,
more preferably an alkyl group, an arylamino group, an aralkyl group, an alkenyl group, an aryl group, a heteroaryl group, one of these groups in which at least one hydrogen atom is substituted with a fluorine atom, a fluorine atom, a cyano group, or —SF₅,
still more preferably an alkyl group, an alkoxy group, an aryloxy group, an arylamino group, an aralkyl group, an alkenyl group, an aryl group, a heteroaryl group, an alkylsilyl group, or an arylsilyl group,
particularly preferably an alkyl group, an arylamino group, an aralkyl group, an alkenyl group, an aryl group, or a heteroaryl group, and
most preferably an alkyl group, an arylamino group, an aralkyl group, an aryl group, or a heteroaryl group.

The groups in the substituent group S may each optionally have a substituent selected from the substituent group S as a substituent. Preferred groups, more preferred groups, still more preferred groups, particularly preferred groups, and most preferred groups used as the optional substituents are the same as the preferred groups in the substituent group S.

### (Ring A1)

Ring A1 represents an aromatic hydrocarbon ring structure optionally having a substituent or an aromatic heterocyclic structure optionally having a substituent.

The aromatic hydrocarbon ring is preferably an aromatic hydrocarbon ring having 6 to 30 carbon atoms. Specifically, the aromatic hydrocarbon ring is preferably a benzene ring, a naphthalene ring, an anthracene ring, a triphenylyl ring, an acenaphthene ring, a fluoranthene ring, or a fluorene ring.

The aromatic heterocycle is preferably an aromatic heterocycle having 3 to 30 carbon atoms and containing, as a heteroatom, a nitrogen atom, an oxygen atom, or a sulfur atom. The aromatic heterocycle is more preferably a furan ring, a benzofuran ring, a thiophene ring, or a benzothiophene ring.

Ring A1 is more preferably a benzene ring, a naphthalene ring, or a fluorene ring, particularly preferably a benzene ring or a fluorene ring, and most preferably a benzene ring.

### (Ring A2)

Ring A2 represents an aromatic heterocyclic structure optionally having a substituent.

Preferably, the aromatic heterocycle is an aromatic heterocycle having 3 to 30 carbon atoms and containing, as a heteroatom, a nitrogen atom, an oxygen atom, or a sulfur atom. Specific examples of the aromatic heterocycle include a pyridine ring, a pyrimidine ring, a pyrazine ring, a triazine ring, an imidazole ring, an oxazole ring, a thiazole ring, a benzothiazole ring, a benzoxazole ring, a benzimidazole ring, a quinoline ring, an isoquinoline ring, a quinoxaline ring, a quinazoline ring, a naphthyridine ring, and a phenanthridine ring. The aromatic heterocycle is preferably a pyridine ring, a pyrazine ring, a pyrimidine ring, an imidazole ring, a benzothiazole ring, a benzoxazole ring, a quinoline ring, an isoquinoline ring, a quinoxaline ring, or a quinazoline ring, more preferably a pyridine ring, an imidazole ring, a benzothiazole ring, a quinoline ring, an isoquinoline ring, a quinoxaline ring, or a quinazoline ring, and most preferably a pyridine ring, an imidazole ring, a benzothiazole ring, a quinoline ring, a quinoxaline ring, or a quinazoline ring.

### (Combination of Ring A1 and Ring A2)

Preferred examples of the combination of ring A1 and ring A2 (denoted as ring A1-ring A2) include (benzene ring-pyridine ring), (benzene ring-quinoline ring), (benzene ring-quinoxaline ring), (benzene ring-quinazoline ring), (benzene ring-benzothiazole ring), (benzene ring-imidazole ring), (benzene ring-pyrrole ring), (benzene ring-diazole ring), and (benzene ring-thiophene ring).

### (Substituents on Ring A1 and Ring A2)

Optional substituents on rings A1 and A2 can be freely selected but are each preferably one or a plurality of substituents selected from the substituent group S.

(Ar²⁰¹, Ar²⁰², and Ar²⁰³)

Ar²⁰¹ and Ar²⁰³ each independently represent an aromatic hydrocarbon ring structure optionally having a substituent or an aromatic heterocyclic structure optionally having a substituent.

Ar²⁰² represents an aromatic hydrocarbon ring structure optionally having a substituent, an aromatic heterocyclic structure optionally having a substituent, or an aliphatic hydrocarbon structure optionally having a substituent.

When any of Ar²⁰¹, Ar²⁰², and Ar²⁰³ is an aromatic hydrocarbon ring structure optionally having a substituent, the aromatic hydrocarbon ring structure is preferably an aromatic hydrocarbon ring having 6 to 30 carbon atoms. Specifically, the aromatic hydrocarbon ring structure is preferably a benzene ring, a naphthalene ring, an anthracene ring, a triphenylyl ring, an acenaphthene ring, a fluoranthene ring, or a fluorene ring, more preferably a benzene ring, a naphthalene ring, or a fluorene ring, and most preferably a benzene ring.

When any of Ar²⁰¹ and Ar²⁰² is a benzene ring optionally having a substituent, it is preferable that at least one benzene ring is bonded to adjacent structures at its ortho or meta positions, and it is more preferable that at least one benzene ring is bonded to adjacent structures at its meta positions.

When any of Ar²⁰¹, Ar²⁰², and Ar²⁰³ is a fluorene ring optionally having a substituent, it is preferable that the fluorene ring has substituents at the 9- and 9′-positions or bonded to adjacent structures at the 9- and 9′-positions.

When any of Ar²⁰¹, Ar²⁰², and Ar²⁰³ is an aromatic heterocyclic structure optionally having a substituent, the aromatic heterocyclic structure is preferably an aromatic heterocycle having 3 to 30 carbon atoms and containing, as a heteroatom, a nitrogen atom, an oxygen atom, or a sulfur atom. Specific examples of the aromatic heterocyclic structure include a pyridine ring, a pyrimidine ring, a pyrazine ring, a triazine ring, an imidazole ring, an oxazole ring, a thiazole ring, a benzothiazole ring, a benzoxazole ring, a benzimidazole ring, a quinoline ring, an isoquinoline ring, a quinoxaline ring, a quinazoline ring, a naphthyridine ring, a phenanthridine ring, a carbazole ring, a dibenzofuran ring, and a dibenzothiophene ring. The aromatic heterocyclic structure is preferably a pyridine ring, a pyrimidine ring, a triazine ring, a carbazole ring, a dibenzofuran ring, or a dibenzothiophene ring.

When any of Ar²⁰¹, Ar²⁰², and Ar²⁰³ is a carbazole ring optionally having a substituent, the carbazole ring has a substituent at the N-position or bonded to an adjacent structure at the N-position.

When Ar²⁰² is an aliphatic hydrocarbon structure optionally having a substituent, the aliphatic hydrocarbon structure is a linear, branched, or cyclic structure-containing aliphatic hydrocarbon structure, and the number of carbon atoms in the aliphatic hydrocarbon structure is preferably 1 or more and 24 or less, more preferably 1 or more and 12 or less, and still more preferably 1 or more and 8 or less.

(i1, i2, i3, i4, k1, and k2)

i1 and i2 each independently represent an integer of 0 to 12 and is preferably 1 to 12, more preferably 1 to 8, and still more preferably 1 to 6. When they are within the above range, the solubility and charge transportability are expected to be improved.

i3 represents preferably an integer of 0 to 5 and is more preferably 0 to 2 and still more preferably 0 or 1.

i4 represents preferably an integer of 0 to 2 and is more preferably is 0 or 1.

k1 and k2 each independently represent preferably an integer of 0 to 3 and is more preferably 1 to 3, still more preferably 1 or 2, and particularly preferably 1.

(Preferred Substituents on Ar²⁰¹, Ar²⁰², and Ar²⁰³)

The optional substituents on Ar²⁰¹, Ar²⁰², and Ar²⁰³ can be freely selected but are each preferably one or a plurality of substituents selected from the substituent group S. Preferred groups are also selected from the substituent group S. More preferably, Ar²⁰¹, Ar²⁰², and Ar²⁰³ are unsubstituted (the substituents are each a hydrogen atom) or each substituted with an alkyl group or an aryl group. Particularly preferably, they are unsubstituted (the substituents are each a hydrogen atom) or each substituted with an alkyl group. Most preferably, they are unsubstituted (the substituents are each a hydrogen atom) or each substituted with a tertiary butyl group. It is preferable that, when Ar²⁰³ is present, Ar²⁰³ is substituted with a tertiary butyl group, that, when Ar²⁰³ is not present, Ar²⁰² is substituted with a tertiary butyl group, and that, when Ar²⁰² and Ar²⁰³ are not present, Ar²⁰¹ is substituted with a tertiary butyl group.

### (Preferred Modes of Compound Represented by Formula (201))

Preferably, the compound represented by formula (201) above is a compound satisfying at least one of the following (I) to (IV).

### (I) Phenylene Linked Type

The structure represented by formula (202) is preferably a structure having a group including benzene rings linked together, i.e., a benzene ring structure in which i¹ is 1 to 6 and at least one of the benzene rings is bonded to adjacent structures at its ortho or metal positions.

With this structure, the solubility is expected to be improved, and the charge transportability is also expected to be improved.

### (II) (Phenylene)-Aralkyl(Alkyl)

The compound represented by formula (201) has a structure in which an aromatic hydrocarbon or aromatic heterocyclic group to which an alkyl group or an aralkyl group is bonded is bonded to ring A1 or ring A2. Specifically, this is a structure in which Ar²⁰¹ is an aromatic hydrocarbon structure or an aromatic heterocyclic structure, in which i1 is 1 to 6, in which Ar²⁰² is an aliphatic hydrocarbon structure, in which i2 is 1 to 12 and preferably 3 to 8, in which Ar²⁰³ is a benzene ring structure, and in which i3 is 0 or 1. Preferably, in this structure, Ar²⁰¹ is an aromatic hydrocarbon structure. More preferably, Ar²⁰¹ includes 1 to 5 benzene rings linked together. Still more preferably, the number of benzene rings is 1.

With this structure, the solubility is expected to be improved, and the charge transportability is also expected to be improved.

### (III) Dendron

The compound represented by formula (201) has a structure in which a dendron is bonded to ring A1 or ring A2. For example, Ar²⁰¹ and Ar²⁰² each have a benzene ring structure, and Ar²⁰³ has a biphenyl or terphenyl structure. i1 and i2 are each 1 to 6. i3 is 2, and j is 2.

With this structure, the solubility is expected to be improved, and the charge transportability is also expected to be improved.

(IV) B²⁰¹-L²⁰⁰-B²⁰²

The structure represented by B²⁰¹-L²⁰⁰-B²⁰² is preferably a structure represented by the following formula (203) or (204).

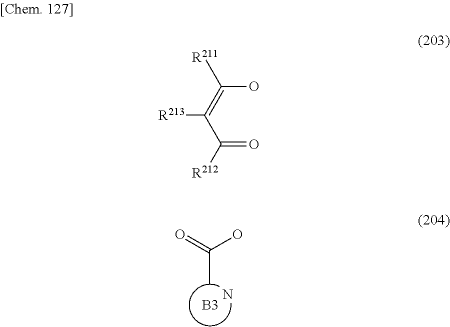

In formula (203), R²¹¹, R²¹², and R²¹³ each independently represent a substituent.

In formula (204), ring B3 represents an aromatic heterocyclic structure containing a nitrogen atom and optionally having a substituent. Ring B3 is preferably a pyridine ring.

### (Preferred Phosphorescent Light-Emitting Material)

No particular limitation is imposed on the phosphorescent light-emitting material represented by formula (201) above. Preferred examples thereof include the following materials.

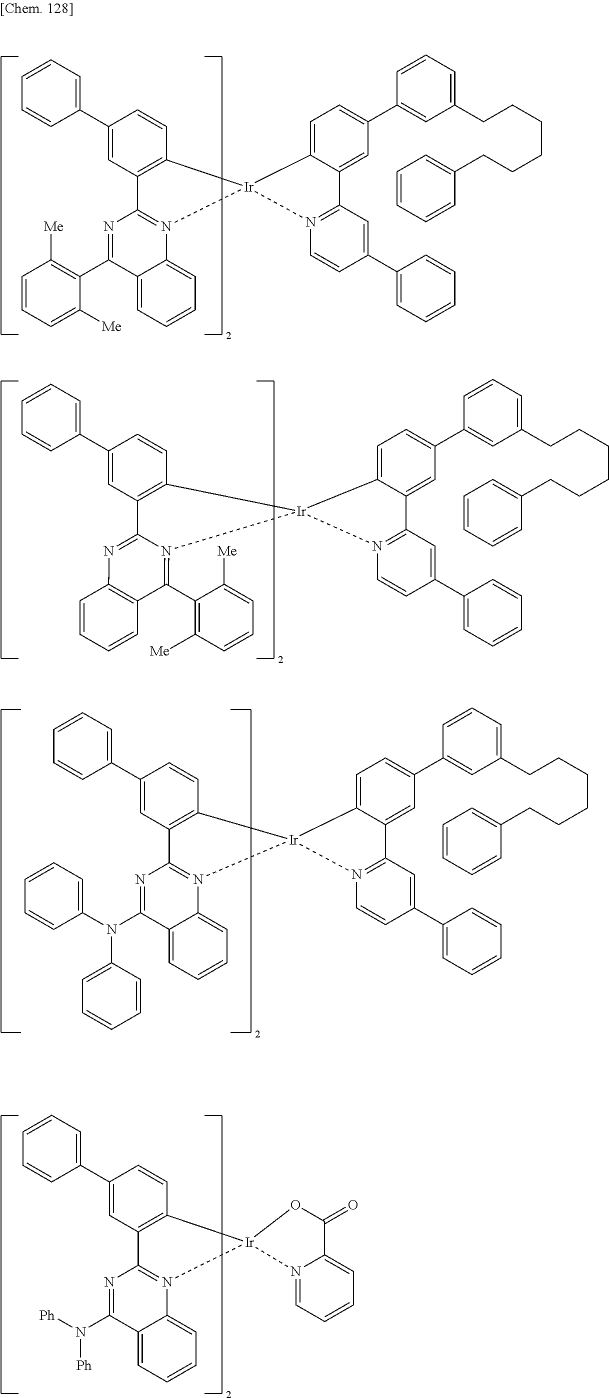

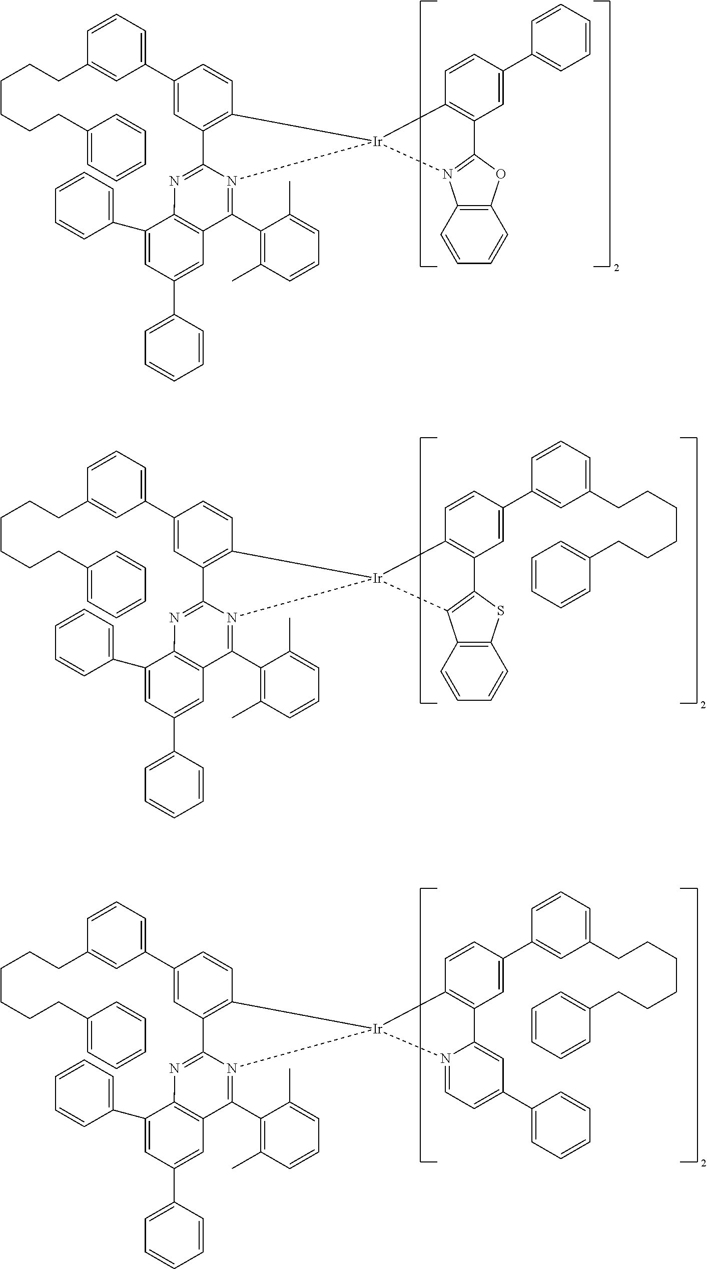

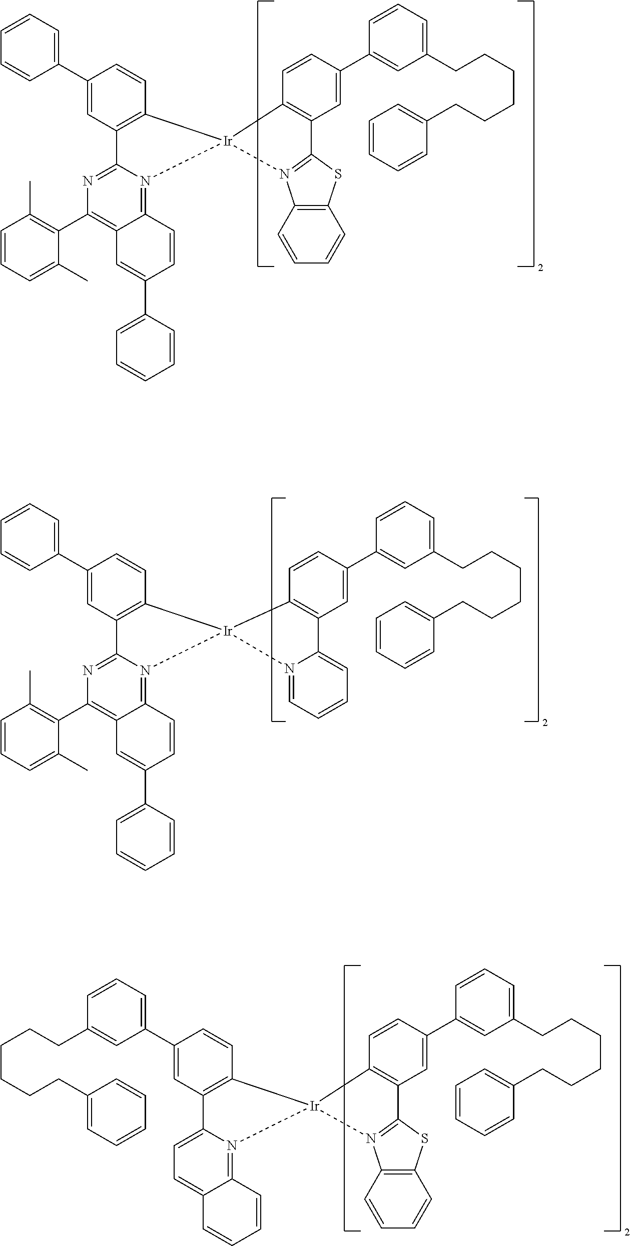

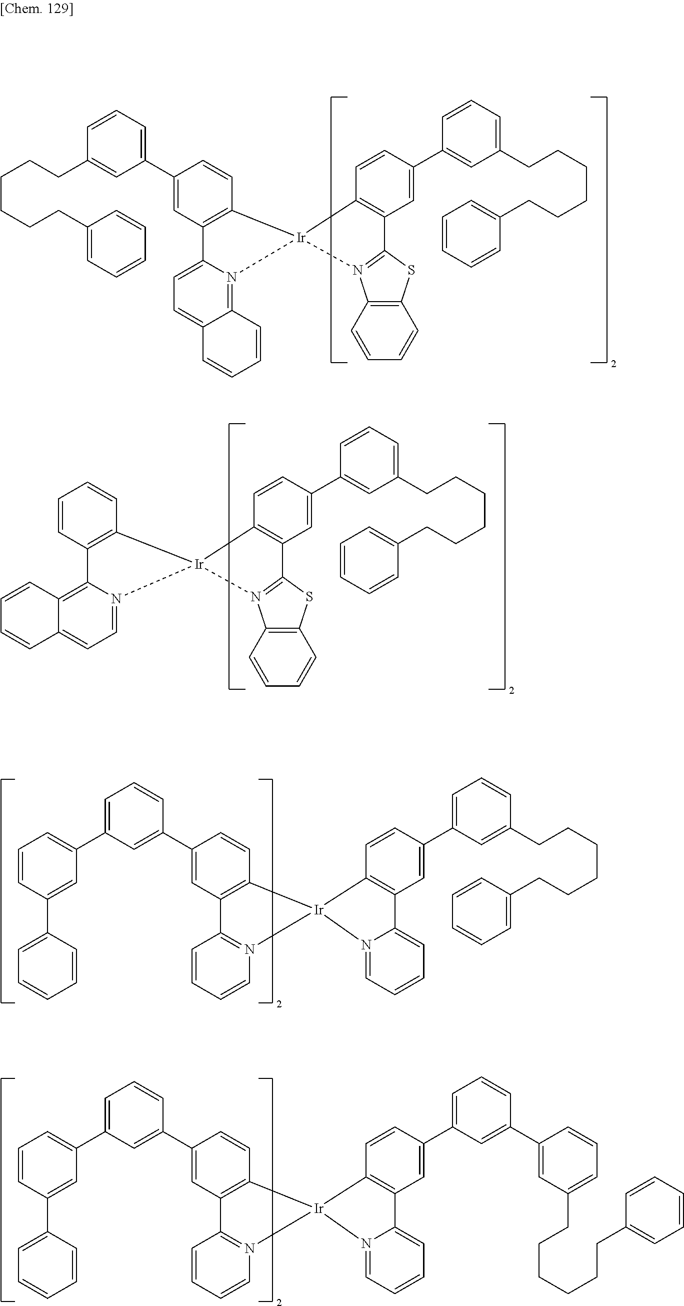

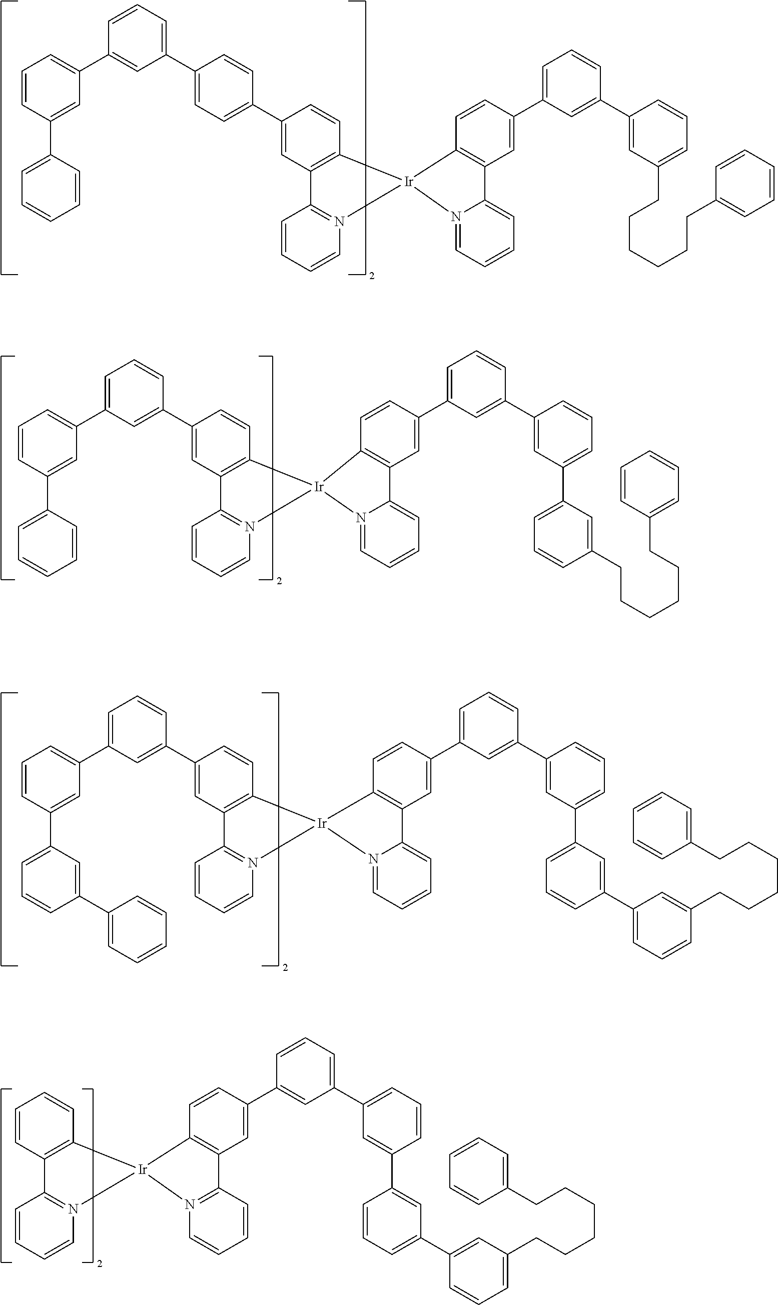

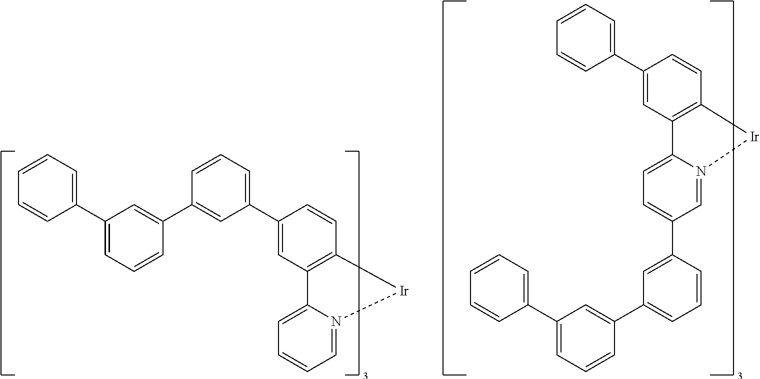

A phosphorescent light-emitting material represented by the following formula (205) is also preferred.

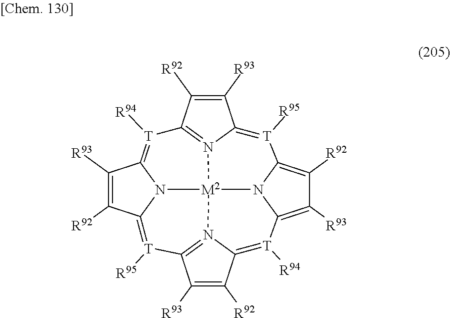

[In formula (205), M² represents a metal, and T represents a carbon atom or a nitrogen atom. R⁹² to R⁹⁵ each independently represent a substituent. When T is a nitrogen atom, R⁹⁴ and R⁹⁵ are not present.]

Specific examples of M² in formula (205) include metals selected from 7 to 11 groups in the periodic table. In particular, ruthenium, rhodium, palladium, silver, rhenium, osmium, iridium, platinum, and gold are preferred, and divalent metals such as platinum and palladium are particularly preferred.

In formula (205), R⁹² and R⁹³ each independently represent a hydrogen atom, a halogen atom, an alkyl group, an aralkyl group, an alkenyl group, a cyano group, an amino group, an acyl group, an alkoxycarbonyl group, a carboxyl group, an alkoxy group, an alkylamino group, an aralkylamino group, a haloalkyl group, a hydroxy group, an aryloxy group, an aromatic hydrocarbon group, or an aromatic heterocyclic group.

When T is a carbon atom, R⁹⁴ and R⁹⁵ each independently represent a substituent selected from the examples of R⁹² and R⁹³. When T is a nitrogen atom, no R⁹⁴ or R⁹⁵ is bonded directly to the T. R⁹² to R⁹⁵ may each optionally have a substituent. The substituent may be any of the above described substituents. Two or more selected from R⁹² to R⁹⁵ may be linked together to form a ring.

### (Molecular Weight)

The molecular weight of the phosphorescent light-emitting material is preferably 5000 or less, more preferably 4000 or less, and particularly preferably 3000 or less. The molecular weight of the phosphorescent light-emitting material is preferably 800 or more, more preferably 1000 or more, and still more preferably 1200 or more. When the molecular weight is within the above range, the phosphorescent light-emitting material are not aggregated, and the phosphorescent light-emitting material is mixed uniformly with the charge transport material, so that a light-emitting layer with high luminous efficiency may be obtained.

It is preferable that the molecular weight of the phosphorescent light-emitting material is high because the Tg, melting point, decomposition temperature, etc. are high and the phosphorescent light-emitting material and a light-emitting layer to be formed have high heat resistance and because deterioration in the quality of the film due to the generation of gas, recrystallization, and migration of molecules and an increase in the concentration of impurities caused by thermal decomposition of the material are unlikely to occur. However, it is preferable that the molecular weight of the phosphorescent light-emitting material is small because the organic compound can be easily purified.

### <Charge Transport Material>

The charge transport material used for the light-emitting layer is a material having a skeleton with good charge transportability and is selected preferably from electron transport materials, hole transport materials, and bipolar materials that can transport both electrons and holes.

Specific examples of the skeleton with good charge transportability include an aromatic structure, an aromatic amine structure, a triarylamine structure, a dibenzofuran structure, a naphthalene structure, a phenanthrene structure, a phthalocyanine structure, a porphyrin structure, a thiophene structure, a benzylphenyl structure, a fluorene structure, a quinacridone structure, a triphenylene structure, a carbazole structure, a pyrene structure, an anthracene structure, a phenanthroline structure, a quinoline structure, a pyridine structure, a pyrimidine structure, a triazine structure, an oxadiazole structure, and an imidazole structure.

The electron transport material is more preferably a compound having a pyridine structure, a pyrimidine structure, or a triazine structure and still more preferably a compound having a pyrimidine structure or a triazine structure because such a material has good electron transportability and has a relatively stable structure.

The hole transport material is a compound having a structure with good hole transportability. Among the above-described central skeletons with good charge transportability, a carbazole structure, a dibenzofuran structure, a triarylamine structure, a naphthalene structure, a phenanthrene structure or a pyrene structure is preferred as the structure with good hole transportability, and a carbazole structure, a dibenzofuran structure, or a triarylamine structure is more preferred.

The charge transport material used for the light-emitting layer has preferably a condensed ring structure including 3 or more rings and is more preferably a compound having two or more condensed ring structures each including 3 or more rings or a compound having at least one condensed ring including 5 or more rings. With these compounds, the rigidity of the molecule increases, and the effect of reducing the extent of molecular motion in response to heat can be easily obtained. Moreover, from the viewpoint of charge transportability and the durability of the material, it is preferable that the condensed ring including 3 or more rings and the condensed ring including 5 or more rings each include an aromatic hydrocarbon ring or an aromatic heterocycle.

Specific examples of the condensed ring structure including 3 or more rings include an anthracene structure, a phenanthrene structure, a pyrene structure, a chrysene structure, a naphthacene structure, a triphenylene structure, a fluorene structure, a benzofluorene structure, an indenofluorene structure, an indolofluorene structure, a carbazole structure, an indenocarbazole structure, an indolocarbazole structure, a dibenzofuran structure, and a dibenzothiophene structure. From the viewpoint of the charge transportability and solubility, it is preferable that the condensed ring structure is at least one selected from the group consisting of a phenanthrene structure, a fluorene structure, an indenofluorene structure, a carbazole structure, an indenocarbazole structure, an indolocarbazole structure, a dibenzofuran structure, and a dibenzothiophene structure. From the viewpoint of durability against electric charges, a carbazole structure or an indolocarbazole structure is more preferred.

In the present invention, from the viewpoint of the durability of the organic electroluminescent element against electric charges, it is preferable that at least one charge transport material in the light-emitting layer is a material having a pyrimidine skeleton or a triazine skeleton.

From the viewpoint of obtaining good flexibility, the charge transport material in the light-emitting layer is preferably a high-molecular weight material. A light-emitting layer formed using a material having high flexibility is preferred as a light-emitting layer of an organic electroluminescent element formed on a flexible substrate. When the charge transport material contained in the light-emitting layer is a high-molecular weight material, its molecular weight is preferably 5,000 or more and 1,000,000 or less, more preferably 10,000 or more and 500,000 or less, and still more preferably 10,000 or more and 100,000 or less.

From the viewpoint of the ease of synthesis and purification of the charge transport material in the light-emitting layer, the ease of designing its electron transport performance and hole transport performance, and the ease of adjusting the viscosity when it is dissolved in a solvent, the charge transport material is preferably a low-molecular weight material. When the charge transport material contained in the light-emitting layer is a low-molecular weight material, its molecular weight is preferably 5,000 or less, still more preferably 4,000 or less, particularly preferably 3,000 or less, and most preferably 2,000 or less and is preferably 300 or more, more preferably 350 or more, and still more preferably 400 or more.

### <Fluorescent Light-Emitting Material>

No particular limitation is imposed on the fluorescent light-emitting material, but the fluorescent light-emitting material is preferably a compound represented by the following formula (211).

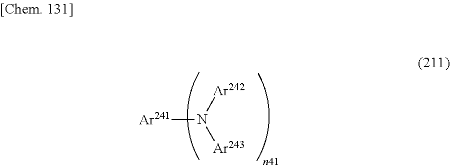

In formula (211) above, Ar²⁴¹ represents a condensed aromatic hydrocarbon ring structure optionally having a substituent. Ar²⁴² and Ar²⁴³ each independently represent an alkyl group optionally having a substituent, an aromatic hydrocarbon group optionally having a substituent, an aromatic heterocyclic group optionally having a substituent, or a group including any of these groups bonded together. n41 is an integer of 1 to 4.

Ar²⁴¹ represents preferably a condensed aromatic hydrocarbon ring structure having 10 to 30 carbon atoms. Specific examples of the ring structure include naphthalene, acenaphthene, fluorene, anthracene, phenanthrene, fluoranthene, pyrene, tetracene, chrysene, and perylene.

Ar²⁴¹ is more preferably a condensed aromatic hydrocarbon ring structure having 12 to 20 carbon atoms, and specific examples of the ring structure include acenaphthene, fluorene, anthracene, phenanthrene, fluoranthene, pyrene, tetracene, chrysene, and perylene.

Ar²⁴¹ is still more preferably a condensed aromatic hydrocarbon ring structure having 16 to 18 carbon atoms, and specific examples of the ring structure include fluoranthene, pyrene, and chrysene.

n41 is 1 to 4, preferably 1 to 3, still more preferably 1 to 2, and most preferably 2.

The alkyl groups represented by Ar²⁴² and Ar²⁴³ are each preferably an alkyl group having 1 to 12 carbon atoms and more preferably an alkyl group having 1 to 6 carbon atoms.

The aromatic hydrocarbon groups represented by Ar²⁴² and Ar²⁴³ are each preferably an aromatic hydrocarbon group having 6 to 30 carbon atoms, more preferably an aromatic hydrocarbon group having 6 to 24 carbon atoms, and most preferably a phenyl group or a naphthyl group.

The aromatic heterocyclic groups represented by Ar²⁴² and Ar²⁴³ are each preferably an aromatic heterocyclic group having 3 to 30 carbon atoms and more preferably an aromatic hydrocarbon group having 5 to 24 carbon atoms group. Specifically, each aromatic heterocyclic group is preferably a carbazolyl group, a dibenzofuranyl group, or a dibenzothiophenyl group and more preferably a dibenzofuranyl group.

The optional substituents on Ar²⁴¹, Ar²⁴², and Ar²⁴³ are each preferably a group selected from the substituent group S, more preferably a hydrocarbon group included in the substituent group S, and still more preferably a hydrocarbon group selected from the preferred groups in the substituent group S.

No particular limitation is imposed on the charge transport material used together with the fluorescent light-emitting material, but the charge transport material is preferably a material represented by the following formula (212).

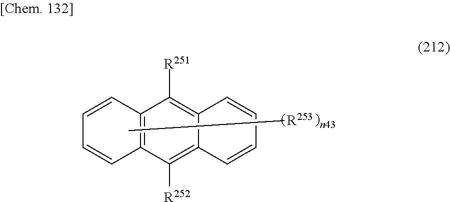

In formula (212) above, R²⁵¹ and R²⁵² are each independently a structure represented by formula (213). R²⁵³ represents a substituent. When a plurality of R²⁵³'s are present, they may be the same or different. n43 is an integer of 0 to 8.

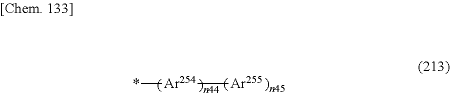

In formula (213) above, * represents a direct bond to the anthracene ring in formula (212). Ar²⁵⁴ and Ar²⁵⁵ each independently represent an aromatic hydrocarbon structure optionally having a substituent or an aromatic heterocyclic structure optionally having a substituent. When a plurality of Ar²⁵⁴'s are present, they may be the same or different. When a plurality of Ar²⁵⁵'s are present, they may be the same or different. n44 is an integer of 1 to 5, and n45 is an integer of 0 to 5.

Ar²⁵⁴ is preferably an aromatic hydrocarbon structure that is a monocycle or condensed ring having 6 to 30 carbon atoms and optionally having a substituent and more preferably an aromatic hydrocarbon structure that is a monocycle or condensed ring having 6 to 12 carbon atoms and optionally having a substituent.

Ar²⁵⁵ is preferably an aromatic hydrocarbon structure that is a monocycle or condensed ring having 6 to 30 carbon atoms and optionally having a substituent or an aromatic heterocyclic structure that is a condensed ring having 6 to 30 carbon atoms and optionally having a substituent. Ar²⁵⁵ is more preferably an aromatic hydrocarbon structure that is a monocycle or condensed ring having 6 to 12 carbon atoms and optionally having a substituent or an aromatic heterocyclic structure that is a condensed ring having 12 carbon atoms and optionally having a substituent.

n44 is preferably an integer of 1 to 3 and more preferably 1 or 2.

n45 is preferably an integer of 0 to 3 and more preferably 0 to 2.

The optional substituents on R²⁵³, Ar²⁵⁴, and Ar²⁵⁵ serving as substituents are each preferably a group selected from the substituent group S. These are each more preferably a hydrocarbon group included in the substituent group S and still more preferably a hydrocarbon group selected from the preferred groups in the substituent group S.

The molecular weights of the fluorescent light-emitting material and the charge transport material are each preferably 5,000 or less, still more preferably 4,000 or less, particularly preferably 3,000 or less, and most preferably 2,000 or less. The molecular weights are each preferably 300 or more, more preferably 350 or more, and still more preferably 400 or more.

### [Hole Blocking Layer]

The hole blocking layer may be disposed between the light-emitting layer 5 and the electron injection layer described later. The hole blocking layer is a layer disposed on the light-emitting layer 5 so as to be in contact with the cathode 7-side interface of the light-emitting layer 5.

The hole blocking layer has the function of preventing holes moving from the anode 2 from reaching the cathode 7 and the function of efficiently transporting electrons injected from the cathode 7 toward the light-emitting layer 5. Examples of the physical properties required for the material forming the hole blocking layer include high electron mobility, low hole mobility, a large energy gap (the difference between the HOMO and LUMO), and a high excited triplet energy level (Ti).

Examples of the material of the hole blocking layer 6 that satisfies these requirements include: meatal complexes such as mixed ligand complexes such as bis(2-methyl-8-quinolinolato) (phenolato)aluminum and bis(2-methyl-8-quinolinolato) (triphenylsilanolato)aluminum and binuclear metal complexes such as bis(2-methyl-8-quinolato)aluminum-p-oxo-bis-(2-methyl-8-quinolinolato)aluminum; styryl compounds such as distyrylbiphenyl derivatives (JP11-242996A); triazole derivatives such as 3-(4-biphenylyl)-4-phenyl-5(4-tert-butylphenyl)-1,2,4-triazole (JP7-41759A); and phenanthroline derivatives such as bathocuproine (JP10-79297A). Moreover, a compound having at least one pyridine ring substituted at the 2-, 4-, and 6-positions and described in WO2005/022962 is also preferred as the material of the hole blocking layer.

No particular limitation is imposed on the method for forming the hole blocking layer. The hole blocking layer 6 can be formed using a wet deposition method, a vapor deposition method, or another method.

The hole blocking layer can have any thickness so long as the effects of the invention are not significantly impaired. The thickness of the hole blocking layer is generally 0.3 nm or more and preferably 0.5 nm or more and is generally 100 nm or less and preferably 50 nm or less.

### [Electron Transport Layer]

The electron transport layer 6 is disposed between the light-emitting layer 5 and the cathode 7 for the purpose of further improving the current efficiency of the element.

The electron transport layer 6 is formed of a compound capable of efficiently transporting electrons injected from the cathode 7 toward the light-emitting layer 5 disposed between the electrodes between which an electric field is applied. It is necessary that the electron transport compound used for the electron transport layer 6 be a compound having high electron injection efficiency from the cathode 7 and high electron mobility and capable of efficiently transporting the injected electrons.

Examples of the electron transport compound used for the electron transport layer include metal complexes such as an aluminum complex of 8-hydroxyquinoline (JP59-194393A), metal complexes of 10-hydroxybenzo[h]quinoline, oxadiazole derivatives, distyrylbiphenyl derivatives, silole derivatives, 3-hydroxyflavone metal complexes, 5-hydroxyflavone metal complexes, benzoxazole metal complexes, benzothiazole metal complexes, trisbenzimidazolylbenzene (U.S. Pat. No. 5,645,948), quinoxaline compounds (JP6-207169A), phenanthroline derivatives (JP5-331459A), 2-tert-butyl-9,10-N,N′-dicyanoanthraquinonediimine, n-type hydrogenated amorphous silicon carbide, n-type zinc sulfide, and n-type zinc selenide.

The thickness of the electron transport layer 6 is generally 1 nm or more and preferably 5 nm or more and is generally 300 nm or less and preferably 100 nm or less.

The electron transport layer 6 is stacked on the hole blocking layer and formed by a wet deposition method or a vacuum vapor deposition method in the same manner as described above. Generally, the vacuum vapor deposition method is used.

In the present invention, as described above, the electron transport layer may be formed on the light-emitting layer containing a preferred light-emitting layer-forming material by a wet deposition method.

### [Electron Injection Layer]

The electron injection layer may be disposed in order to efficiently inject the electron injected from the cathode 7 into the electron transport layer 6 or the light-emitting layer 5.

To inject electrons efficiently, it is preferable that the material forming the electron injection layer is a metal having a low work function. Examples of such a material include: alkali metals such as sodium and cesium; and alkaline earth metals such as barium and calcium. The thickness of the electron injection layer is generally preferably 0.1 nm or more and 5 nm or less.

Moreover, a material prepared by doping an organic electron transport material typified by a nitrogen-containing heterocyclic compound such as bathophenanthroline or a metal complex such as an aluminum complex of 8-hydroxyquinoline with an alkali metal such as sodium, potassium, cesium, lithium, or rubidium (described in JP10-270171A, JP2002-100478A, JP2002-100482A, etc.) is also preferred because electron injectability and transportability can be improved and good film quality can also be achieved.

The thickness of the electron injection layer is generally 5 nm or more and preferably 10 nm or more and is generally 200 nm or less and preferably 100 nm or less.

The electron injection layer is stacked on the light-emitting layer 5 or on the hole blocking layer or the electron transport layer 6 on the light-emitting layer 5 and formed by a wet deposition method or a vacuum vapor deposition method.

The details of the wet deposition method are the same as those for the light-emitting layer.

The hole blocking layer, the electron transport layer, and the electron injection layer may be formed as a single layer by co-doping with an electron transport material and a lithium complex.

### [Cathode]

The cathode 7 is an electrode that functions to inject electrons into a layer on the light-emitting layer 5 side (such as the electron injection layer or the light-emitting layer).

Any material that can be used for the anode 2 can be used as the material of the cathode 7. To efficiently inject electrons, the material of the cathode 7 is preferably a metal having a low work function, and examples thereof include metals such as tin, magnesium, indium, calcium, aluminum, and silver and alloys thereof. Specific examples include electrodes of low-work function alloys such as magnesium-silver alloys, magnesium-indium alloys, and aluminum-lithium alloys.

In terms of the stability of the organic electroluminescent element, it is preferable that a metal layer having a high work function and stable to air is stacked on the cathode to protect the cathode formed of a low-work function metal. Examples of the stacked metal include metals such as aluminum, silver, copper, nickel, chromium, gold, and platinum.

The thickness of the cathode is generally the same as the thickness of the anode.

### [Additional Layer]

The organic electroluminescent element of the invention may include an additional layer so long as the effects of the invention are not significantly impaired. Specifically, an additional layer other than the layers described above may be disposed between the anode and the cathode.

### [Different Element Structure]

The organic electroluminescent element of the invention may have a structure obtained by reversing the structure described above. Specifically, for example, the cathode, the electron injection layer, the electron transport layer, the hole blocking layer, the light-emitting layer, the hole transport layer, the hole injection layer, the anode may be stacked in this order on the substrate.

When the organic electroluminescent element of the invention is applied to an organic electroluminescent device, one organic electroluminescent element may be used. A plurality of organic electroluminescent elements may be arranged in an array or may be used for a structure in which anodes and cathodes are arranged in an X-Y matrix.

### [Display Device]

The display device (organic electroluminescent element display device) of the invention includes the organic electroluminescent element of the invention. No particular limitation is imposed on the type and structure of the display device of the invention. The organic electroluminescent display device can be assembled by an ordinary method using the organic electroluminescent element of the invention.

The organic EL display device can be formed using, for example, a method described in “Yuki EL Disupurei (Organic EL Display)” (Ohmsha, Ltd., published on August 20, Heisei 16, written by TOKITO Shizuo, ADACHI Chihaya, and MURATA Hideyuki).

### [Lighting Device]

The lighting device (organic electroluminescent element lighting device) of the invention includes the organic electroluminescent element of the invention. No particular limitation is imposed on the type and structure of the lighting device of the invention. The lighting device of the invention can be assembled by an ordinary method using the organic electroluminescent element of the invention.

### EXAMPLES

The present invention will next be specifically described by way of Examples. However, the invention is not limited to the following Examples and can be embodied in various forms within the spirit of the invention.

### <Synthesis of Intermediates>

### [Synthesis of Compound 1]

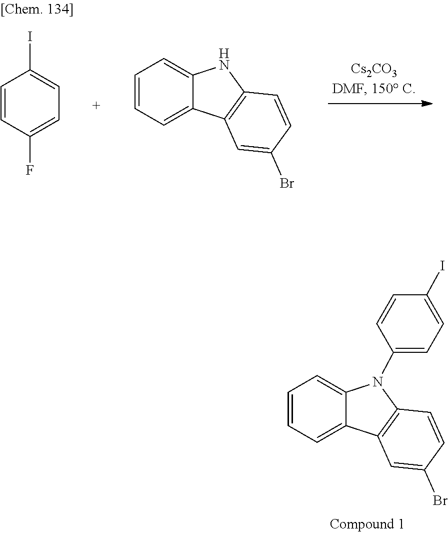 [SMILES: Fc1ccc(I)cc1 ; Brc1ccc2[nH]c3ccccc3c2c1 ; Brc1ccc2c(c1)c1ccccc1n2-c1ccc(I)cc1]

A flask was charged with 1-fluoro-4-iodobenzene (25.0 g, 112.6 mmol), 3-bromocarbazole (6.93 g, 28.15 mmol), cesium carbonate (36.7 g, 112.6 mmol), and dehydrated N,N-dimethylformamide (100 mL) in a nitrogen flow. The inside of the system was thoroughly replaced with nitrogen, and the mixture was heated to 150° C. Then the mixture was stirred at this temperature for 12 hours. Water was added to the reaction mixture at room temperature, and the resulting mixture was stirred for 15 minutes and extracted with 100 mL of methylene chloride. The organic layer was dried over anhydrous magnesium sulfate. The crude product was purified by column chromatography (eluent: hexane:methylene chloride=700:300) to thereby obtain compound 1 (6.4 g, yield: 50.7%).

### [Synthesis of Compound 2]

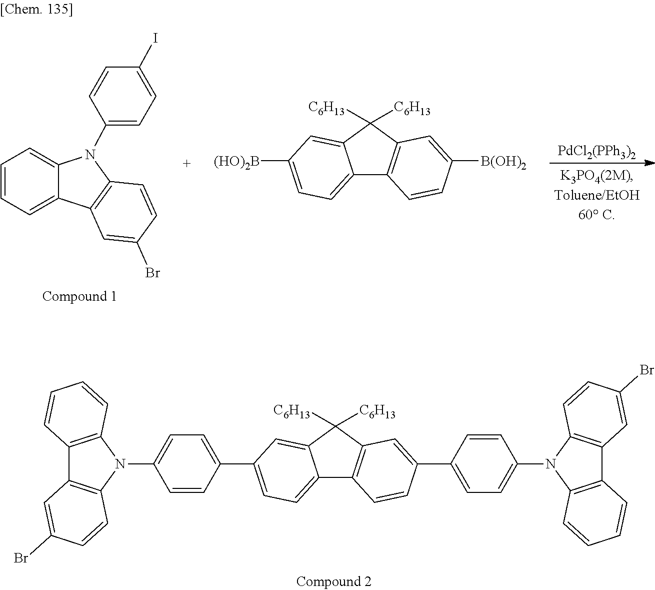

Next, a flask was charged with compound 1 (6.4 g, 14.28 mmol), 9,9-dihexylfluorene-2,7-diboronic acid (3.0 g, 7.14 mmol), potassium phosphate (2M aqueous solution, 20 mL), toluene (40 mL), and ethanol (20 mL). The inside of the system was thoroughly replaced with nitrogen, and the mixture was heated to 60° C. Bis(triphenylphosphine)palladium(II) dichloride (0.050 g, 0.071 mmol) was added, and the resulting mixture was stirred at 60° C. for 3 hours. Water was added to the reaction mixture, and the resulting mixture was extracted with toluene. The organic layer was dried over anhydrous magnesium sulfate and partially purified with activated clay. The crude product was purified by column chromatography (eluent: hexane methylene chloride=750:250) to thereby obtain compound 2 (5.5 g, yield: 39.5%).

### [Synthesis of Compound 3]

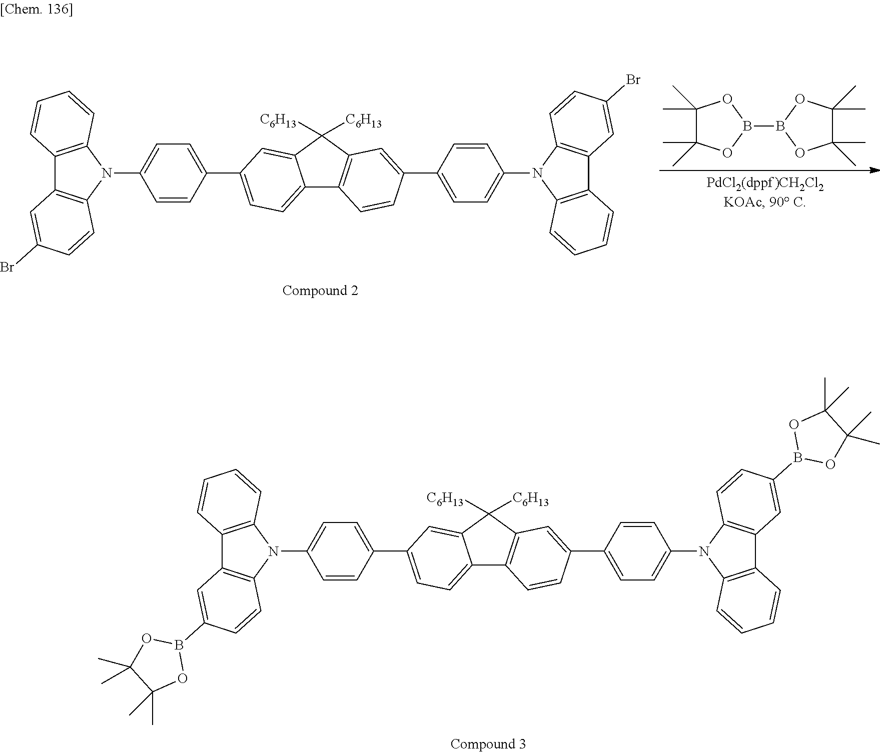

A 200 mL flask was charged with 60 mL of dimethyl sulfoxide, compound 2 (5.5 g, 5.64 mmol), bis(pinacolato)diboron (4.3 g, 16.92 mmol), and potassium acetate (3.3 g, 33.84 mmol) in a nitrogen flow, and the mixture was stirred at 60° C. for 30 minutes. Then 1,1′-bis(diphenylphosphino)ferrocene-palladium(II) dichloride-dichloromethane [PdCl₂(dppf)CH₂Cl₂](0.92 g, 1.13 mmol) was added, and the mixture was allowed to react at 95° C. for 4 hours.

Pure water was added dropwise at room temperature, and the precipitate was vacuum-filtered. The residue was dissolved in toluene, dried over anhydrous magnesium sulfate, and partially purified with activated clay. The resulting mixture was vacuum-filtered, and the filtrate was concentrated, dissolved in methylene chloride, and then reprecipitated in methanol. The precipitate was vacuum-filtered to obtain compound 3 (4.4 g, yield: 73.0%).

### [Synthesis of Compound 4]

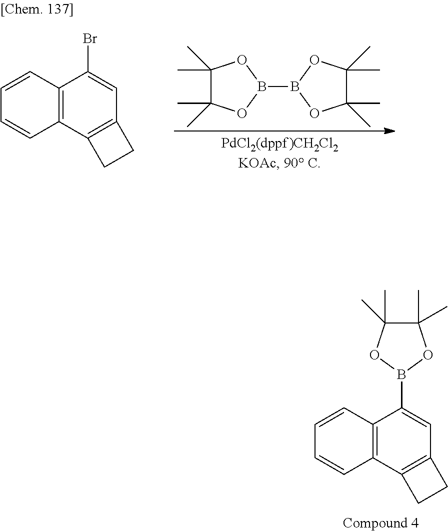

A 200 mL flask was charged with 100 mL of dimethyl sulfoxide, 4-bromo-1,2-dihydrocyclobuta[α]naphthalene (15.0 g, 64.35 mmol), bis(pinacolato)diboron (19.6 g, 77.22 mmol), and potassium acetate (18.9 g, 193.05 mmol) in a nitrogen flow, and the mixture was stirred at 60° C. for 30 minutes. Then 1,1′-bis(diphenylphosphino)ferrocene-palladium(II) dichloride-dichloromethane [PdCl₂(dppf)CH₂Cl₂](2.6 g, 3.22 mmol) was added, and the mixture was allowed to react at 95° C. for 5 hours.

Pure water was added at room temperature, and the precipitate was vacuum-filtered. The residue was dissolved in toluene, dried over anhydrous magnesium sulfate, and partially purified with activated clay. The resulting mixture was vacuum-filtered, and the filtrate was concentrated and recrystallized with acetonenitrile. The resulting mixture was vacuum-filtered, and the residue was dried to thereby obtain compound 4 (10.3 g, yield: 57.0%).

### [Synthesis of Compound 5]

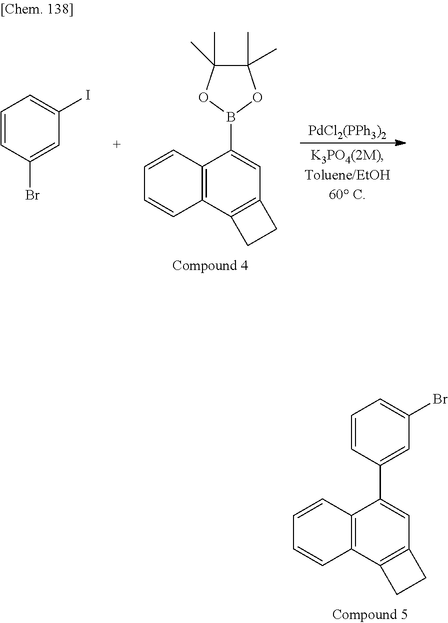 [SMILES: Brc1cccc(I)c1 ; CC1(C)OB(c2cc3c(c4ccccc24)CC3)OC1(C)C ; Brc1cccc(-c2cc3c(c4ccccc24)CC3)c1 ; "PdCl2(PPh3)2"]

Next, a flask was charged with 1-bromo-3-iodobenzene (7.5 g, 26.51 mmol), compound 4 (6.19 g, 22.09 mmol), potassium phosphate (2M aqueous solution, 30 mL), toluene (60 mL), and ethanol (30 mL). The inside of the system was thoroughly replaced with nitrogen, and the mixture was heated to 60° C. Bis(triphenylphosphine)palladium(II) dichloride (0.078 g, 0.11 mmol) was added, and the mixture was stirred at 60° C. for 3 hours. Water was added to the reaction mixture, and the resulting mixture was extracted with toluene. The organic layer was dried over anhydrous magnesium sulfate and partially purified with activated clay. The crude product was purified by column chromatography (eluent: hexane:methylene chloride=980:20) to thereby obtain compound 5 (4.4 g, yield: 64.4%).

### [Synthesis of Compound 7]

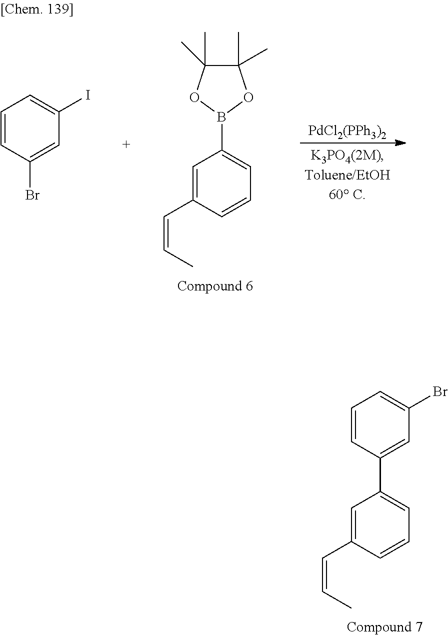 [SMILES: Brc1cccc(I)c1 ; C/C=C\c1cccc(B2OC(C)(C)C(C)(C)O2)c1 ; C/C=C\c1cccc(-c2cccc(Br)c2)c1 ; "PdCl2(PPh3)2"]

A flask was charged with 1-bromo-3-iodobenzene (17.4 g, 61.5 mmol), commercial compound 6 (10.0 g, 41.0 mmol), potassium phosphate (2M aqueous solution, 62 mL), toluene (120 mL), and ethanol (60 mL). The inside of the system was thoroughly replaced with nitrogen, and the mixture was heated to 60° C. Bis(triphenylphosphine)palladium(II) dichloride (0.147 g, 0.21 mmol) was added, and the mixture was stirred at 60° C. for 4.5 hours. Water was added to the reaction mixture, and the resulting mixture was extracted with toluene. The organic layer was dried over anhydrous magnesium sulfate and partially purified with activated clay. The crude product was purified by column chromatography (eluent: hexane) to thereby obtain compound 7 (9.0 g, yield: 80.4%).

### [Synthesis of Compound 8]

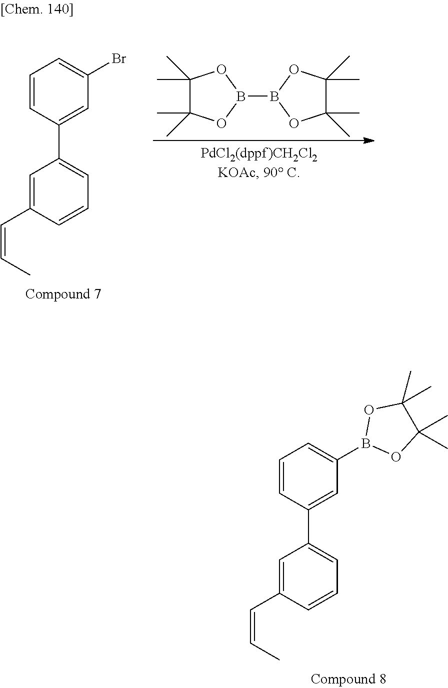

A 200 mL flask was charged with 100 mL of dimethyl sulfoxide, compound 7 (9.0 g, 32.9 mmol), bis(pinacolato)diboron (12.5 g, 49.4 mmol), and potassium acetate (9.7 g, 98.7 mmol) in a nitrogen flow, and the mixture was stirred at 60° C. for 30 minutes. Then 1,1′-bis(diphenylphosphino)ferrocene-palladium(II) dichloride-dichloromethane [PdCl₂(dppf)CH₂Cl₂](1.3 g, 1.6 mmol) was added, and the mixture was allowed to react at 95° C. for 5 hours.

Pure water was added at room temperature, and the precipitate was vacuum-filtered. The residue was dissolved in toluene, dried over anhydrous magnesium sulfate, and partially purified with activated clay. The product was dried to thereby obtain compound 8 (8.9 g, yield: 90.5%).

### [Synthesis of HI-52]

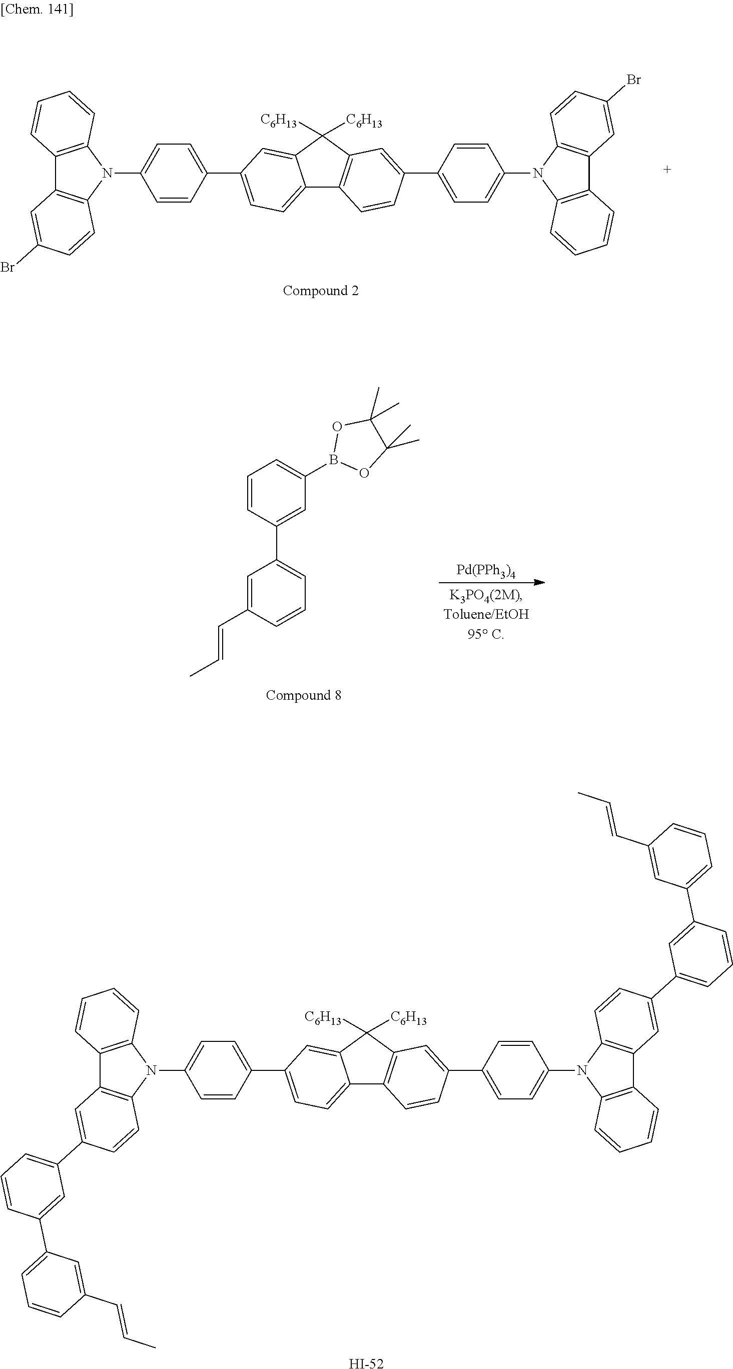

A flask was charged with compound 2 (2.8 g, 2.87 mmol), compound 8 (2.8 g, 8.61 mmol), potassium phosphate (2M aqueous solution, 9 mL), toluene (18 mL), and ethanol (90 mL). The inside of the system was thoroughly replaced with nitrogen, and the mixture was heated to 60° C. Tetrakis(triphenylphosphine)palladium(0) (0.196 g, 0.17 mmol) was added, and the mixture was stirred at 95° C. for 4.5 hours. Water was added to the reaction mixture, and the resulting mixture was extracted with toluene. The organic layer was dried over anhydrous magnesium sulfate and partially purified with activated clay. The crude product was purified by column chromatography (eluent: hexane:methylene chloride=750:250) to thereby obtain the target product HI-52 (2.5 g, yield: 72.0%).

### [Synthesis of HI-53]

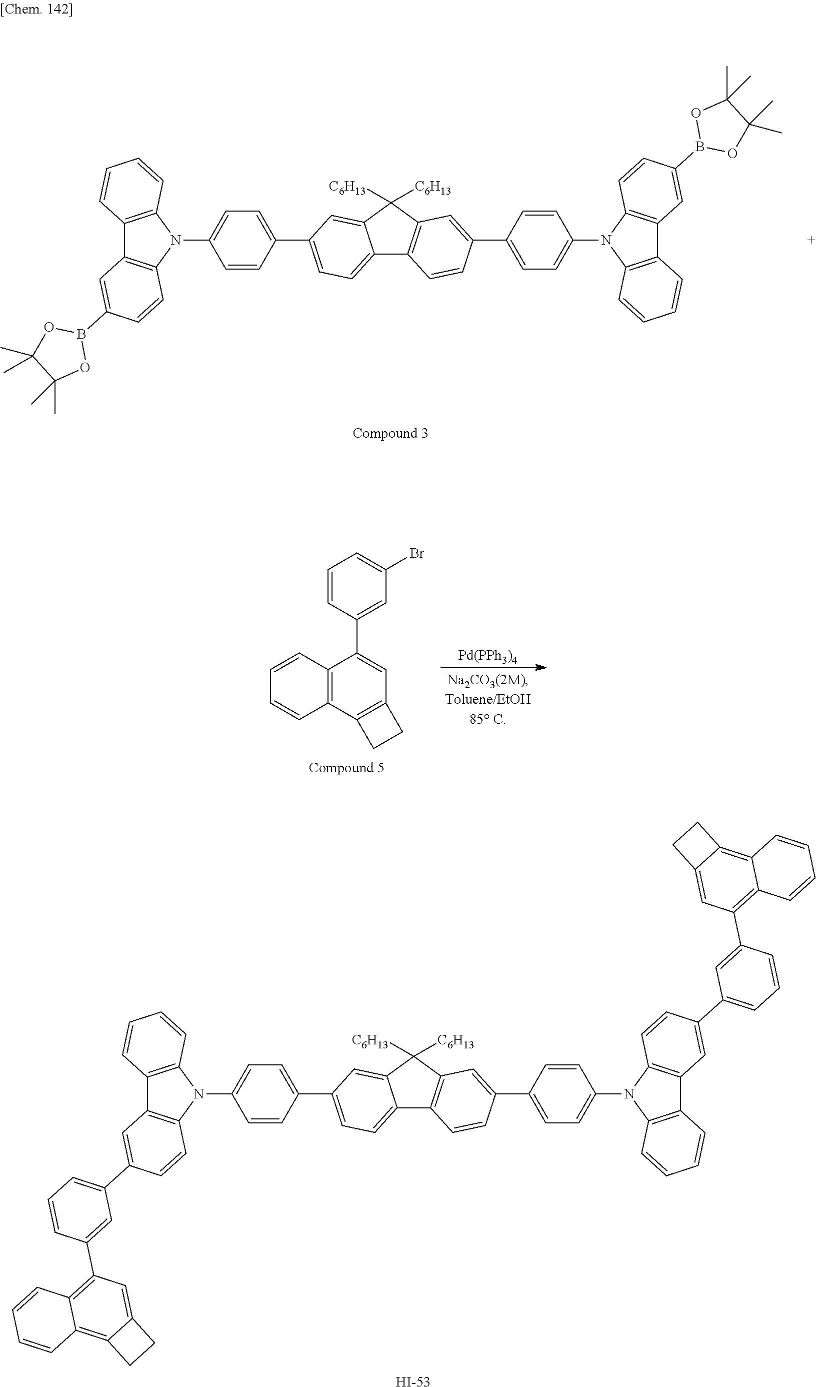

A flask was charged with compound 3 (1.47 g, 1.38 mmol), compound 5 (1.3 g, 4.14 mmol), sodium carbonate (2M aqueous solution, 4.2 mL), toluene (20 mL), and ethanol (10 mL). The inside of the system was thoroughly replaced with nitrogen, and the mixture was heated to 60° C. Tetrakis(triphenylphosphine)palladium(0) (0.064 g, 0.055 mmol) was added, and the mixture was stirred at 85° C. for 7 hours. Water was added to the reaction mixture, and the resulting mixture was extracted with toluene. The organic layer was dried over anhydrous magnesium sulfate and partially purified with activated clay. The crude product was purified by column chromatography (eluent: hexane:methylene chloride=750:250) to thereby obtain the target compound HI-53 (0.82 g, yield: 46.8%).

### <Synthesis of Compound I>

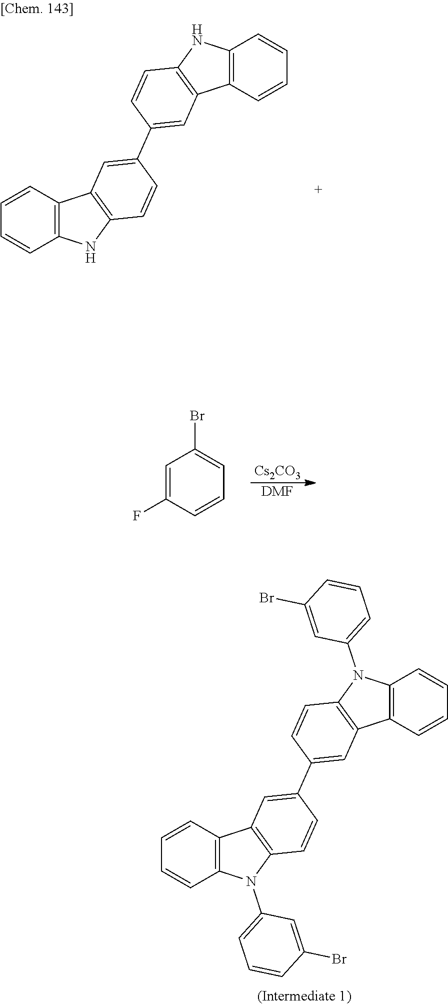

A 500 mL round bottom flask was charged with bicarbazole (7.39 g), 3-bromofluorobenzene (24.9 g), cesium carbonate (101 g), and N,N-dimethylformamide (100 mL), and the mixture was stirred in a nitrogen atmosphere at 150° C. for 16 hours. The reaction mixture was cooled to room temperature and poured in water (800 mL), and the resulting mixture was suction-filtered. The residue was washed with dichloromethane (500 mL), ethyl acetate (100 mL), and ethanol (50 mL) and dried to thereby obtain 10.1 g of intermediate 1 as a white solid.

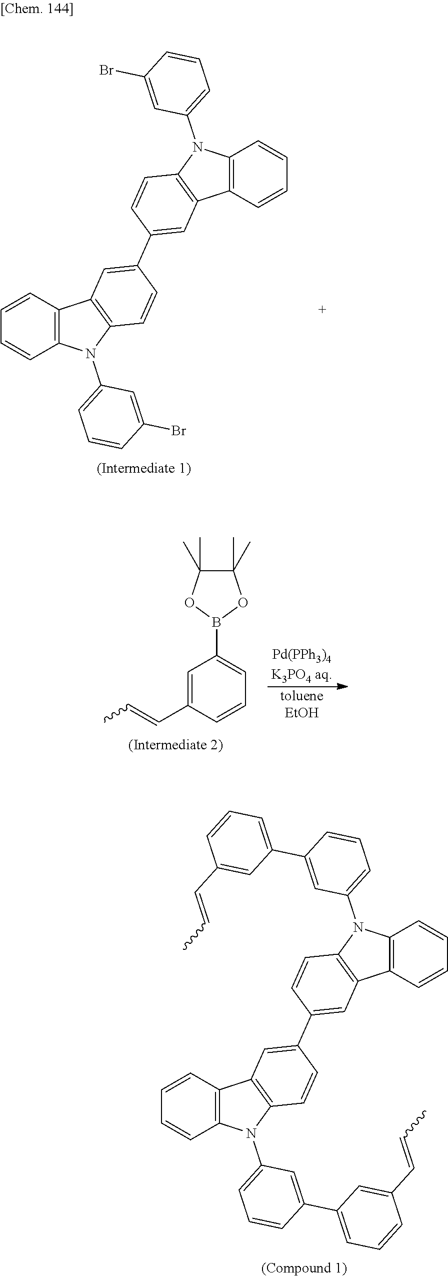

A 200 mL flask was charged with intermediate 1 (2.01 g), intermediate 2 (1.52 g), toluene (55 mL), ethanol (30 mL), and a 2 mol/L aqueous tripotassium phosphate solution (8 mL) in a nitrogen atmosphere, and the mixture was stirred at 100° C. for 3.5 hours. The mixture was cooled to room temperature. Then water (100 mL) and ethyl acetate (100 mL) were added, and the mixture was subjected to separation and washing. The oil phase was collected, and the solvent was removed under reduced pressure. The resulting residue was purified by silica gel column chromatography (neutral silica gel, hexane/dichloromethane=4/1) to thereby obtain 1.46 g of compound I as a white solid.

### [Exemplary Embodiment]

The substrate used can be prepared by depositing an indium-tin oxide (ITO) transparent conductive film to a thickness of, for example, 50 nm on a glass substrate.

The hole injection layer-forming composition can be prepared by dissolving, in anisole, 1.3% by weight of a high-molecular weight hole transport compound having a repeating structure represented by formula (P-1), 1.3% by weight of a hole transport compound having a structure represented by formula (M-1), and 0.4% by weight of an electron accepting compound (HI-1).

The substrate is spin-coating with the prepared solution and dried on a hot plate in air at 230° C. for 30 minutes, and a hole injection layer having a thickness of, for example, 50 nm can thereby be formed.

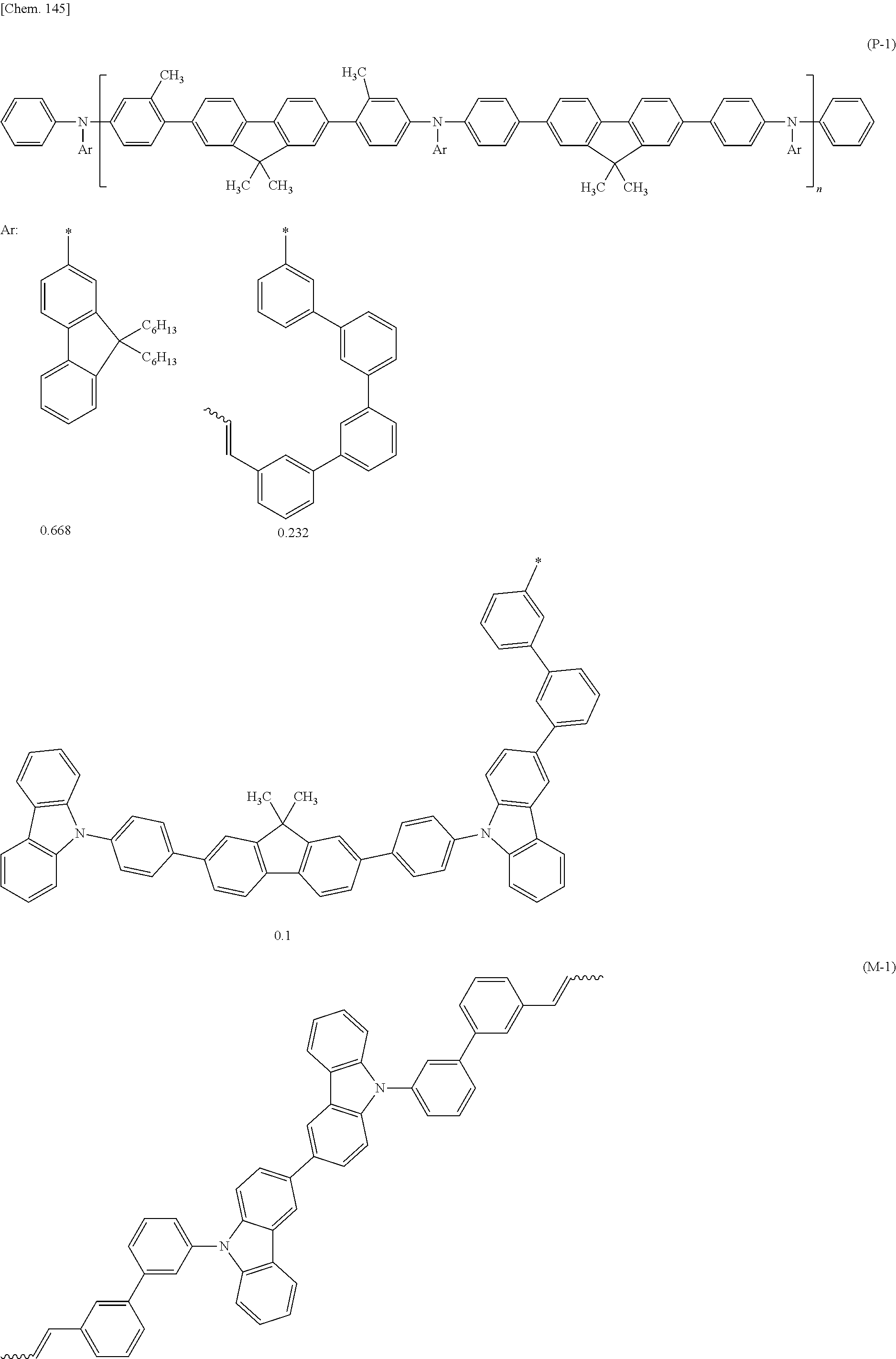

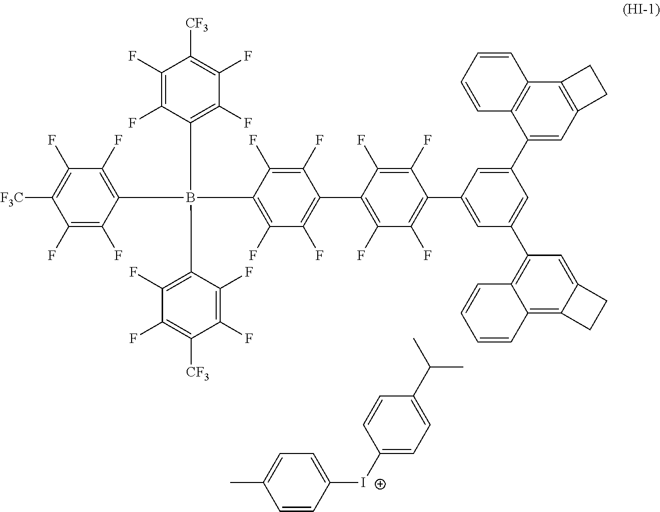

Next, a hole transport layer, a light-emitting layer, an electron transport layer, and a cathode are formed on the substrate with the hole injection layer formed thereon by application, and finally these layers are sealed. An organic electroluminescent element can thereby be produced.

The element produced as described above is expected to exhibit good element characteristics.

### [Examples of Organic Electroluminescent Element]

### Example 1

A glass substrate with an indium-tin oxide (ITO) transparent conductive film deposited to a thickness of 50 nm (a sputtered film product manufactured by GEOMATEC Co., Ltd.) was subjected to patterning using an ordinary photolithography technique and etching with hydrochloric acid to thereby form a 2 mm-wide stripe anode. The substrate with the patterned ITO formed thereon was subjected to ultrasonic cleaning with an aqueous surfactant solution, washed with ultrapure water, subjected to ultrasonic cleaning with ultrapure water, and washed with ultrapure water in this order, then dried using compressed air, and finally subjected to UV ozone cleaning.

A composition used as the hole injection layer-forming composition was prepared by dissolving, in anisole, 1.3% by weight of a high-molecular weight hole transport compound having a repeating structure represented by formula (P-2) below, 1.3% by weight of a hole transport carbazole compound having a structure represented by formula (M-2) below (HI-53 described above), and 0.4% by weight of an electron accepting compound (HI-1) shown below.

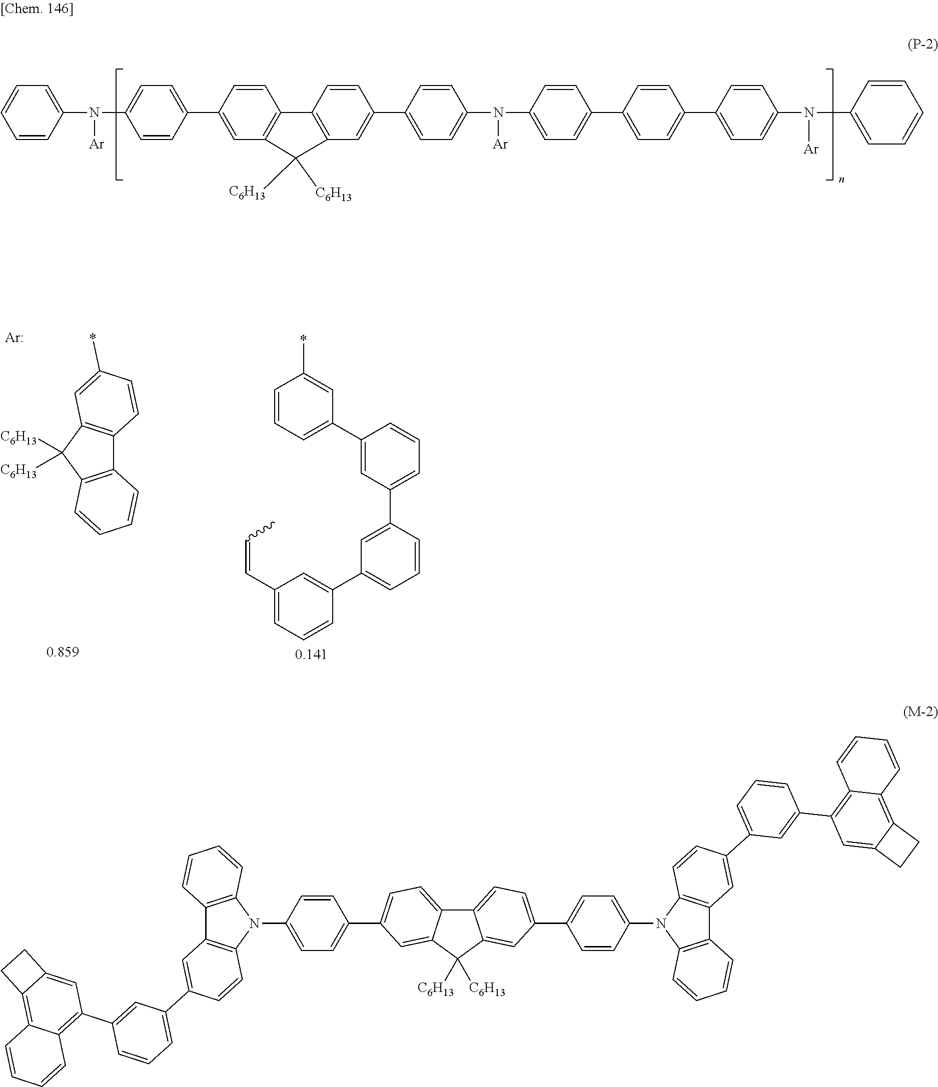

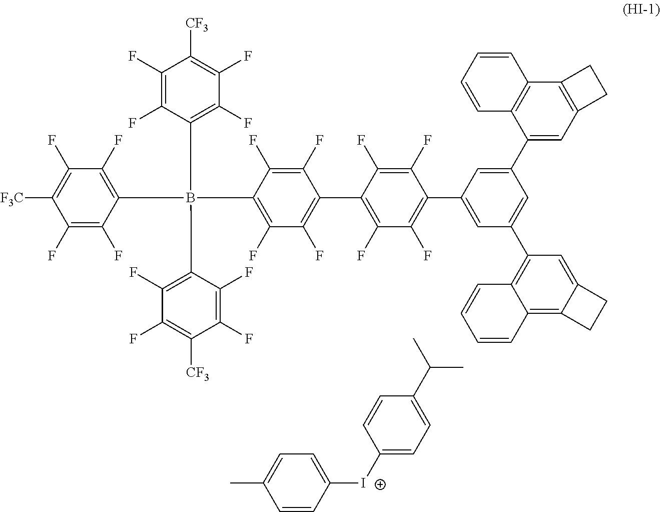

The substrate was spin-coated with the prepared solution in air, and the resulting substrate was dried on a hot plate at 230° C. in air for 30 minutes to form a uniform thin film having a thickness of 50 nm and used as a hole injection layer.

Next, 100 parts by weight of a high-molecular weight charge transport compound having structural formula (HT-1) below was dissolved in 1,3,5-trimethylbenzene to prepare a 2.0% by weight solution.

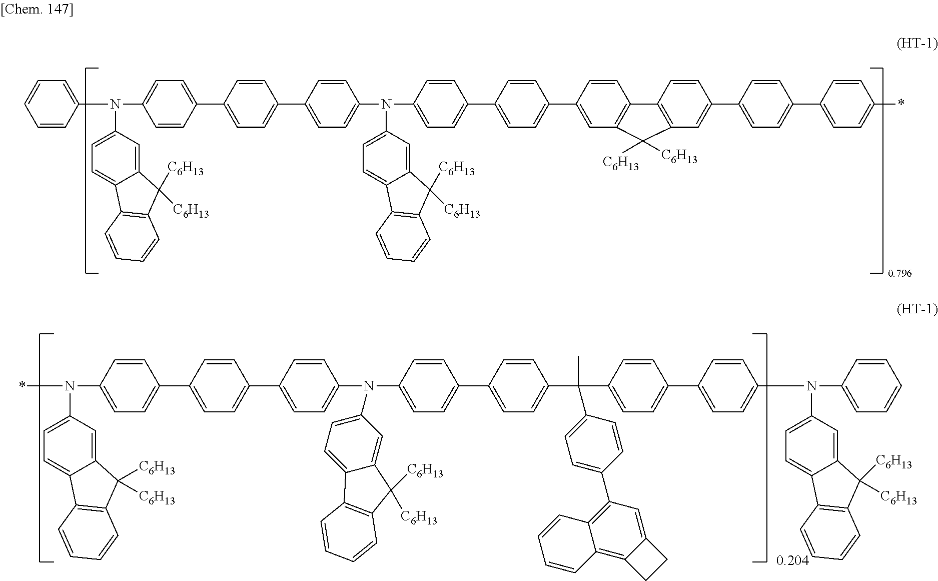

The substrate with the hole injection layer formed thereon by application was spin-coated with the prepared solution in a nitrogen glove box, and the resulting substrate was dried on a hot plated at 230° C. for 30 minutes in the nitrogen glove box to thereby form a uniform film having a thickness of 40 nm and used as a hole transport layer.

Next, a host compound represented by structural formula (BH-1) below and a dopant compound having structural formula (BD-1) below were dissolved at a parts by weight ratio of 100:10 in cyclohexylbenzene to prepare a 4.2% by weight solution.

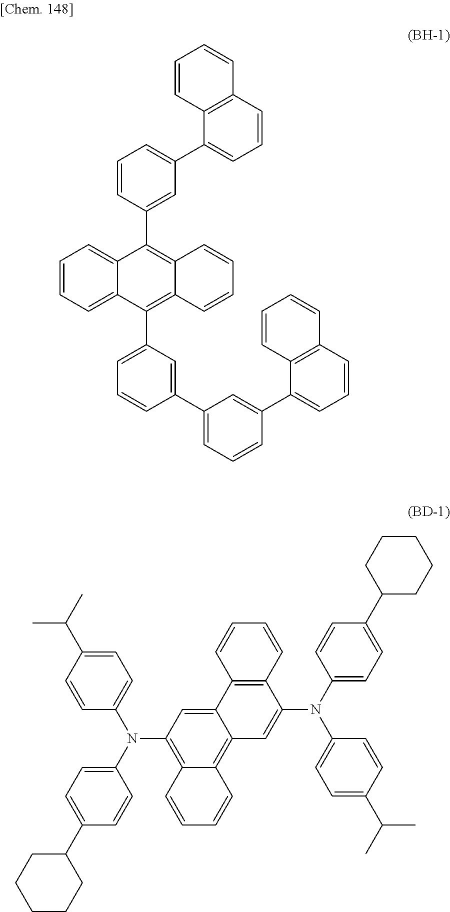

The substrate with the hole transport laver formed thereon by application was spin-coated in a nitrogen glove box to form a uniform 40 nm thin film used as a light-emitting layer. The resulting substrate was dried on a hot plate at 120° C. for 20 minutes in the nitrogen glove box to thereby obtain the light-emitting layer.

The substrate with the light-emitting layer formed thereon was placed in a vacuum vapor deposition apparatus, and the apparatus was evacuated to 2×10⁻⁴ Pa or lower.

Next, structural formula (ET-1) below and 8-hydroxyquinolinolato-lithium were co-deposited at a thickness ratio of 2:3 on the light-emitting layer using a vacuum vapor deposition method to thereby form an electron transport layer having a thickness of 30 nm.

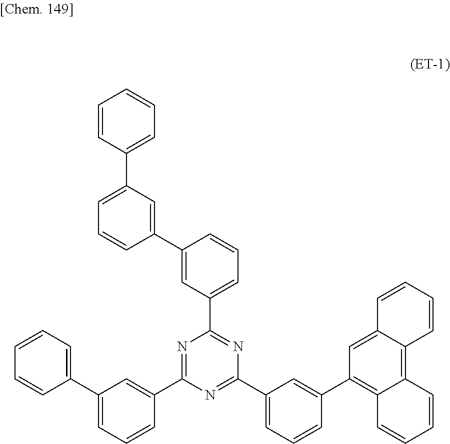 [SMILES: c1ccc(-c2cccc(-c3cccc(-c4nc(-c5cccc(-c6ccccc6)c5)nc(-c5cccc(-c6cc7ccccc7c7ccccc67)c5)n4)c3)c2)cc1]

Next, a 2 mm-wide stripe-shaped shadow mask used as a cathode deposition mask was brought into close contact with the substrate so as to be orthogonal to the ITO stripe anode. Then aluminum in a molybdenum boat was heated to form an aluminum layer having a thickness of 80 nm by a vacuum vapor deposition method, and a cathode was thereby formed. An organic electroluminescent element having a light-emitting portion having an area of 2 mm×2 mm was obtained in the manner described above.

In a space filled with nitrogen, a moisture-oxygen adsorbent was applied to the inner side of a glass substrate having a hollow structure, and the glass substrate with the organic electroluminescent element formed thereon was disposed such that its surface with the organic electroluminescent element formed thereon faced the surface of the hollow glass with the moisture-oxygen adsorbent applied thereto. An ultraviolet curable resin was applied so as to surround the outer circumference of the organic electroluminescent element to bond these surfaces together. The ultraviolet curable resin was irradiated with ultraviolet rays to form a structure for isolating the organic electroluminescent element from the outside space. In this manner, the surface of the organic electroluminescent element can be isolated from moisture and oxygen while no structure is in direct contact with the surface of the organic electroluminescent element, and the performance of the organic electroluminescent element can be evaluated with the influences of moisture and oxygen eliminated.

### Example 2

An element was produced using the same procedure as in Example 1 except that a composition used as the hole injection layer-forming composition was prepared by dissolving, in anisole, only 2.6% by weight of the hole transport carbazole compound having the structure of formula (M-2) above and 0.4% by weight of the electron accepting compound (HI-1).

### Example 3

An element was produced using the same procedure as in Example 1 except that a composition used as the hole injection layer-forming composition was prepared by dissolving, in anisole, 1.3% by weight of a high-molecular weight hole transport compound having a repeating structure of formula (P-3) below, 1.3% by weight of the hole transport carbazole compound having the structure of formula (M-2) above, and 0.4% by weight of the electron accepting compound (HI-1).

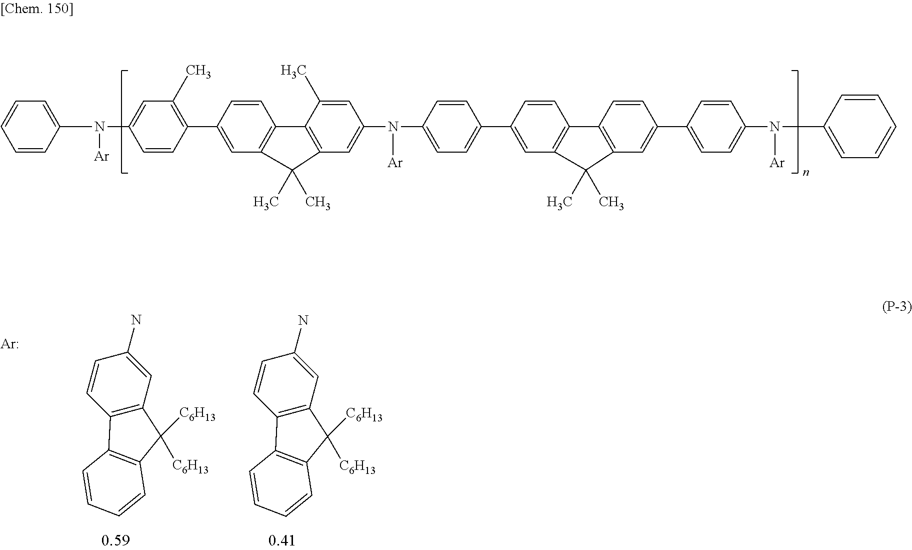

### Example 4

An element was produced using the same procedure as in Example 1 except that a composition used as the hole injection layer-forming composition was prepared by dissolving, in butyl benzoate, 1.3% by weight of a high-molecular weight hole transport compound having a repeating structure of formula (P-4) below, 1.3% by weight of the hole transport carbazole compound having the structure of formula (M-2) above, and 0.4% by weight of the electron accepting compound (HI-1) and that vacuum drying was performed after spin coating.

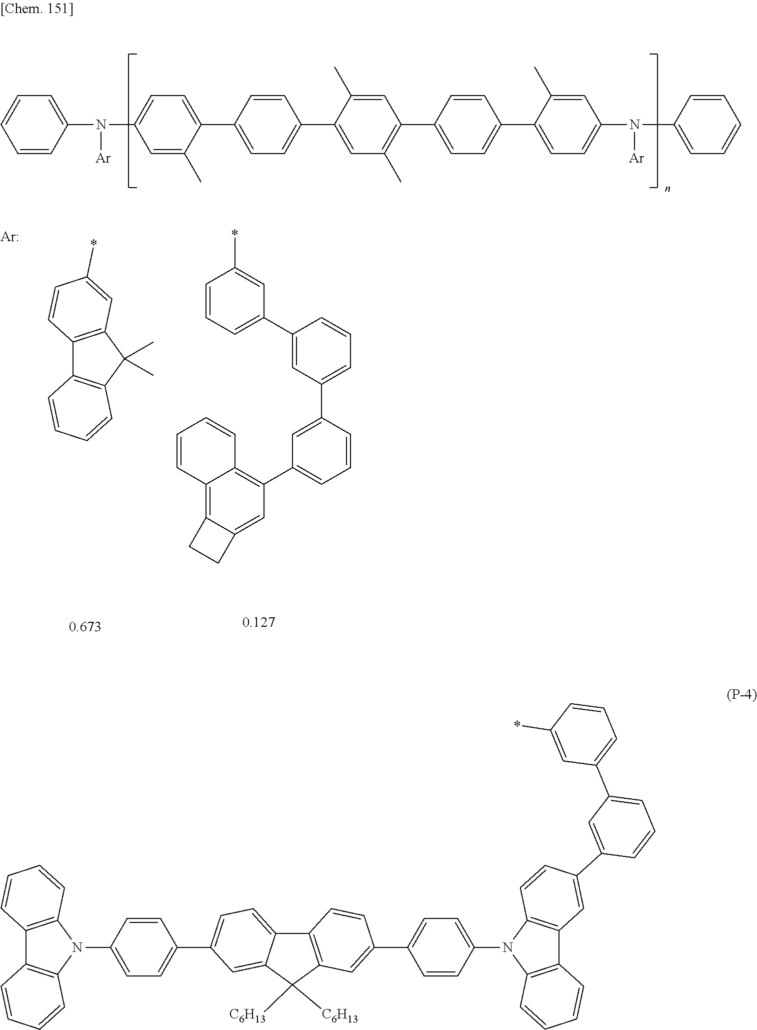

### Comparative Example 1

An element was produced using the same procedure as in Example 1 except that a composition used as the hole injection layer-forming composition was prepared by dissolving, in anisole, 1.3% by weight of the high-molecular weight hole transport compound having the repeating structure of formula (P-2) above, 1.3% by weight of a hole transport compound having a structure of formula (M-3) below, and 0.4% by weight of the electron accepting compound (HI-1).

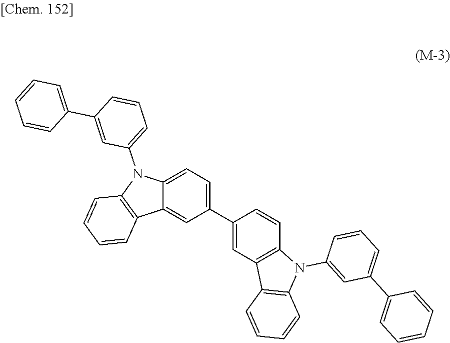 [SMILES: c1ccc(-c2cccc(-n3c4ccccc4c4cc(-c5ccc6c(c5)c5ccccc5n6-c5cccc(-c6ccccc6)c5)ccc43)c2)cc1]

### Comparative Example 2

An element was produced using the same procedure as in Example 1 except that a composition used as the hole injection layer-forming composition was prepared by dissolving, in anisole, 1.3% by weight of the high-molecular weight hole transport compound having the repeating structure of formula (P-2) above, 1.3% by weight of the hole transport carbazole compound having the structure of formula (M-2) above, and 0.4% by weight of an electron accepting compound (HI-2) shown below.

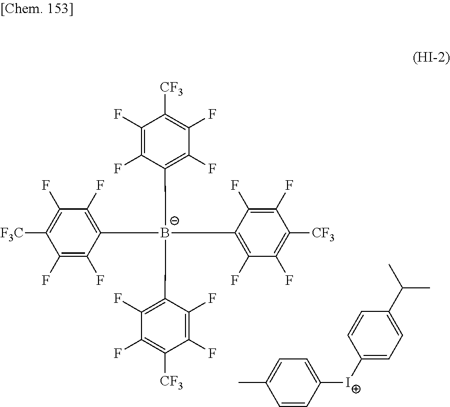 [SMILES: Fc1c(F)c(C(F)(F)F)c(F)c(F)c1[B-](c1c(F)c(F)c(C(F)(F)F)c(F)c1F)(c1c(F)c(F)c(C(F)(F)F)c(F)c1F)c1c(F)c(F)c(C(F)(F)F)c(F)c1F ; Cc1ccc([I+]c2ccc(C(C)C)cc2)cc1]

### Comparative Example 3

An element was produced using the same procedure as in Example 1 except that a composition used as the hole injection layer-forming composition was prepared by dissolving, in anisole, 2.6% by weight of the hole transport carbazole compound having the structure of formula (M-2) above and 0.4% by weight of the electron accepting compound (HI-2).

### Comparative Example 4

An element was produced using the same procedure as in Example 1 except that a composition used as the hole injection layer-forming composition was prepared by dissolving, in anisole, 2.6% by weight of a hole transport carbazole compound having a structure of formula (M-4) below and 0.4% by weight of the electron accepting compound (HI-1).

However, the hole injection layer dissolved in the step of forming the hole transport layer, and the element could not be produced.

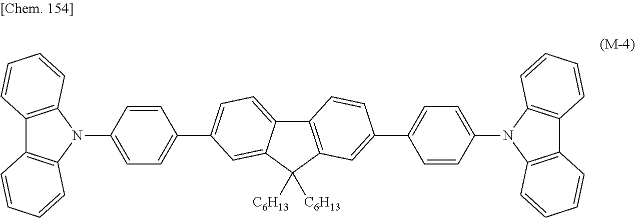

### [Evaluation of Elements]

The organic electroluminescent elements obtained in Examples 1 to 4 and Comparative Examples 1 to 3 were caused to emit light, and the light obtained was blue light with a peak wavelength of 468 nm.

Each of the elements was caused to emit light at 1,000 cd/m², and the current efficiency (cd/A) in this case was measured. The element was continuously energized at a current density of 20 mA/cm², and a luminance reduction lifetime (when the reduction in luminance reached 90%) was measured. This value is defined as LT90.

The ratio of the current luminous efficiency (cd/A) of the organic electroluminescent element in one of the Examples and Comparative Examples other than Comparative Example 1 relative to that of the organic electroluminescent element in Comparative Example 1, i.e., “the current luminous efficiency of one of the organic electroluminescent elements other than that in Comparative Example 1/the current luminous efficiency of the organic electroluminescent element in Comparative Example 1,” (which is hereinafter referred to as the “relative current luminous efficiency”) is shown in Table 1.

The ratio of the LT90 of the organic electroluminescent element in one of the Examples and Comparative Examples other than Comparative Example 1 relative to that of the organic electroluminescent element in Comparative Example 1, i.e., “the LT90 of one of the organic electroluminescent elements other than that in Comparative Example 1/the LT90 of the organic electroluminescent element in Comparative Example 1,” (which is hereinafter referred to as the “relative lifetime”) was determined and is shown in Table 1.

TABLE 1

| | | | | Relative | |
|---|---|---|---|---|---|
| | | | Electron | current | |
| | | Carbazole | accepting | luminous | Relative |
| | Polymer | compound | compound | efficiency | lifetime |
| Example 1 | P-2 | M-2 | HI-1 | 1.13 | 3.8 |
| | ∘ | ∘ | ∘ | | |
| Example 2 | — | M-2 | HI-1 | 1.16 | 2.3 |
| | — | ∘ | ∘ | | |
| Example 3 | P-3 | M-2 | HI-1 | 1.19 | 2.4 |
| | x | ∘ | ∘ | | |
| Example 4 | P-4 | M-2 | HI-1 | 1.14 | 5.1 |
| | ∘ | ∘ | ∘ | | |
| Comparative | P-2 | M-3 | HI-1 | 1.00 | 1.0 |
| Example 1 | ∘ | x | ∘ | | |
| Comparative | P-2 | M-2 | HI-2 | 0.67 | 0.7 |
| Example 2 | ∘ | ∘ | x | | |
| Comparative | — | M-2 | HI-2 | 1.07 | 1.5 |
| Example 3 | — | ∘ | x | | |
| Comparative | — | M-4 | HI-1 | — | — |
| Example 4 | — | x | ∘ | | |

In Table 1, the symbols o and x shown below the materials have the following meanings. The symbol o is assigned to a material having a crosslinking group, and the symbol x is assigned to a material having no crosslinking group.

As can be seen from the results in Table 1, in the organic electroluminescent elements produced using the compositions of the invention each containing the carbazole compound having a crosslinking group and the electron accepting compound having a crosslinking group, the current luminous efficiency and the lifetime were good.

### Example 5

An element was produced using the same procedure as in Example 1 except that a composition used as the hole injection layer-forming composition was prepared by dissolving, in anisole, 2.6% by weight of the hole transport carbazole compound having the structure of formula (M-1) above and 0.4% by weight of the electron accepting compound (HI-1).

### Example 6

An element was produced using the same procedure as in Example 1 except that a composition used as the hole injection layer-forming composition was prepared by dissolving, in butyl benzoate, 1.3% by weight of the high-molecular weight hole transport compound having the repeating structure of formula (P-1) above, 1.3% by weight of the hole transport carbazole compound having the structure of formula (M-1) above, and 0.4% by weight of the electron accepting compound (HI-1) and that vacuum drying was performed after spin coating.

### Example 7

An element was produced using the same procedure as in Example 1 except that a composition used as the hole injection layer-forming composition was prepared by dissolving, in anisole, 1.3% by weight of the high-molecular weight hole transport compound having the structure of formula (P-3) above, 1.3% by weight of the hole transport carbazole compound having the structure of formula (M-1) above, and 0.4% by weight of the electron accepting compound (HI-1).

### Example 8

An element was produced using the same procedure as in Example 1 except that a composition used as the hole injection layer-forming composition was prepared by dissolving, in butyl benzoate, 1.3% by weight of a high-molecular weight hole transport compound having a repeating structure of formula (P-5) below, 1.3% by weight of the hole transport carbazole compound having the structure of formula (M-1) above, and 0.4% by weight of the electron accepting compound (HI-1) and that vacuum drying was performed after spin coating.

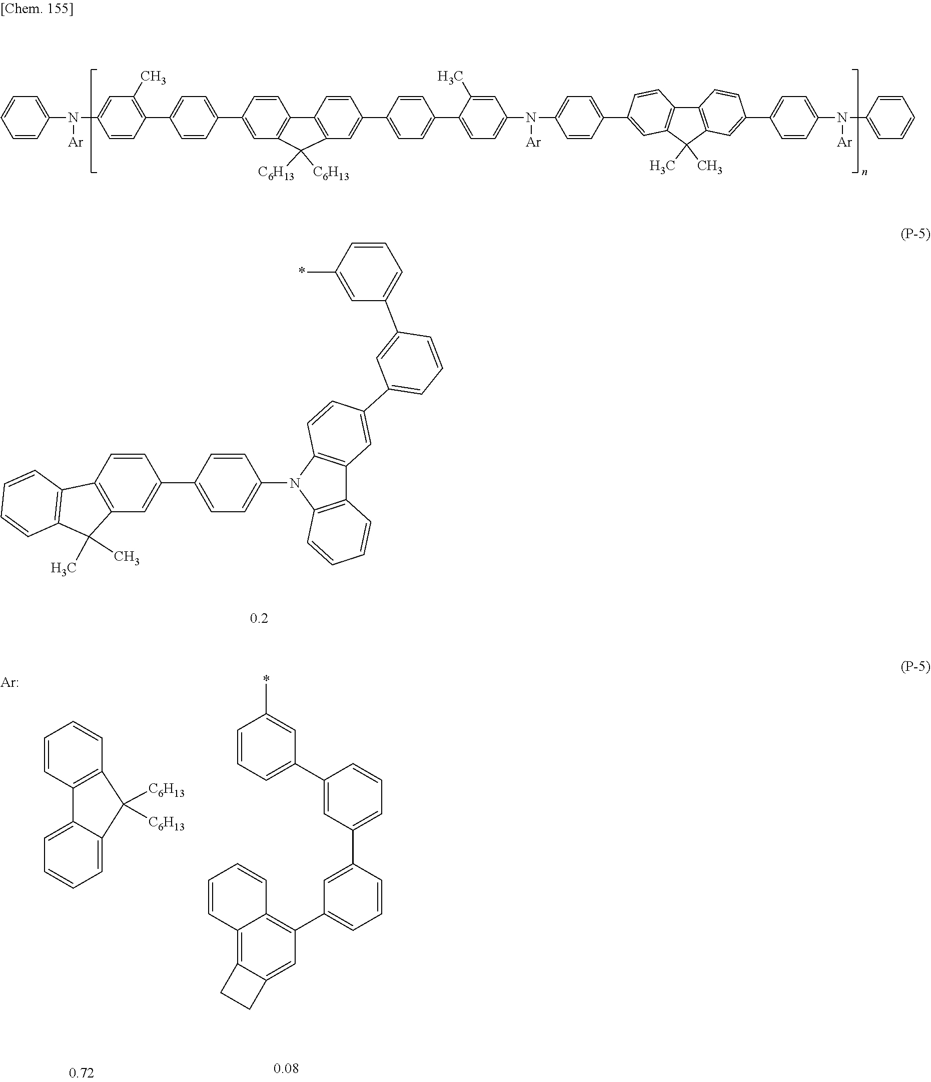

### Comparative Example 5

An element was produced using the same procedure as in Example 1 except that a composition used as the hole injection layer-forming composition was prepared by dissolving, in anisole, 2.6% by weight of the hole transport carbazole compound having the structure of formula (M-1) above and 0.4% by weight of the electron accepting compound (HI-2).

### [Evaluation of Elements]

The organic electroluminescent elements obtained in Examples 5 to 8 and Comparative Examples 1 and 5 were caused to emit light, and the light obtained was blue light with a peak wavelength of 468 nm.

Each of the elements was caused to emit light at 1,000 cd/m², and the voltage (V) and the current efficiency (cd/A) in this case were measured. The element was continuously energized at a current density of 20 mA/cm², and a luminance reduction lifetime (when the reduction in luminance reached 90%) was measured. This value is defined as LT90.

A value obtained be subtracting the voltage (V) of the organic electroluminescent element in Comparative Example 1 from the voltage (V) of an organic electroluminescent element other than that in Comparative Example 1, i.e., “the voltage (V) of an organic electroluminescent element other than that in Comparative Example 1−the voltage (V) of the organic electroluminescent element in Comparative Example 1,” was determined as the “voltage difference” and is shown in Table 2.

The ratio of the current luminous efficiency (cd/A) of the organic electroluminescent element in one of the Examples and Comparative Examples other than Comparative Example 1 relative to that of the organic electroluminescent element in Comparative Example 1, i.e., “the current luminous efficiency of one of the organic electroluminescent elements other than that in Comparative Example 1/the current luminous efficiency of the organic electroluminescent element in Comparative Example 1,” was determined as the “relative current luminous efficiency” and is shown in Table 1.

The ratio of the LT90 of the organic electroluminescent element in one of the Examples and Comparative Examples other than Comparative Example 1 relative to that of the organic electroluminescent element in Comparative Example 1, i.e., “the LT90 of one of the organic electroluminescent elements other than that in Comparative Example 1/the LT90 of the organic electroluminescent element in Comparative Example 1,” was determined as the “relative lifetime” and is shown in Table 2.

TABLE 2

| | | | | | Relative | |
|---|---|---|---|---|---|---|
| | | | Electron | | current | |
| | | Carbazole | accepting | Voltage | luminous | Relative |
| | Polymer | compound | compound | difference | efficiency | lifetime |
| Example 5 | None— | M-1 | HI-1 | −0.1 | 1.11 | 2.8 |
| | — | ∘ | ∘ | | | |
| Example 6 | P-1 | M-1 | HI-1 | −0.2 | 1.13 | 3.3 |
| | ∘ | ∘ | ∘ | | | |
| Example 7 | P-3 | M-1 | HI-1 | −0.1 | 1.07 | 3.3 |
| | ∘ | ∘ | ∘ | | | |
| Example 8 | P-5 | M-1 | HI-1 | −0.3 | 1.19 | 5.5 |
| | ∘ | ∘ | ∘ | | | |
| Comparative Example 1 | P-2 | M-3 | HI-1 | 0 | 1.00 | 1.0 |
| | ∘ | x | ∘ | | | |
| Comparative Example 5 | None | M-1 | HI-2 | 0.4 | 0.94 | 0.4 |
| | — | ∘ | x | | | |

In Table 2, the symbols o and x shown below the materials have the following meanings. The symbol o is assigned to a material having a crosslinking group, and the symbol x is assigned to a material having no crosslinking group.

As can be seen from the results in Table 2, in the organic electroluminescent elements produced using the compositions of the invention each containing the carbazole compound having a crosslinking group and the electron accepting compound having a crosslinking group, the voltage, the current luminous efficiency, and the lifetime were good.

### Example 9

A hole injection layer was formed using the same procedure as in Example 1 except that a composition used as the hole injection layer-forming composition was prepared by dissolving, in anisole, 2.6% by weight of the high-molecular weight hole transport compound having the repeating structure of formula (P-1) above and 0.4% by weight of the electron accepting compound (HI-2).

Next, a composition was prepared by dissolving, in butyl benzoate, 1.5% by weight of the high-molecular weight hole transport compound having the repeating structure of formula (P-5) above and 1.5% by weight of the hole transport carbazole compound having the structure of formula (M-1) above. The substrate with the hole injection layer formed thereon by application was spin-coated with the prepared solution in a nitrogen glove box, and the resulting substrate was vacuum-dried and dried on a hot plate at 230° C. for 30 minutes in the nitrogen glove box to thereby form a uniform thin film having a thickness of 40 nm and used as a hole transport layer.

Next, a host compound having structural formula (GH-1) below, the low-molecular weight charge transfer compound (M-3), and a dopant compound having structural formula (GD-1) below were dissolved at a weight ratio of 50:50:42 in cyclohexylbenzene to thereby prepare a 7.1% by weight solution.

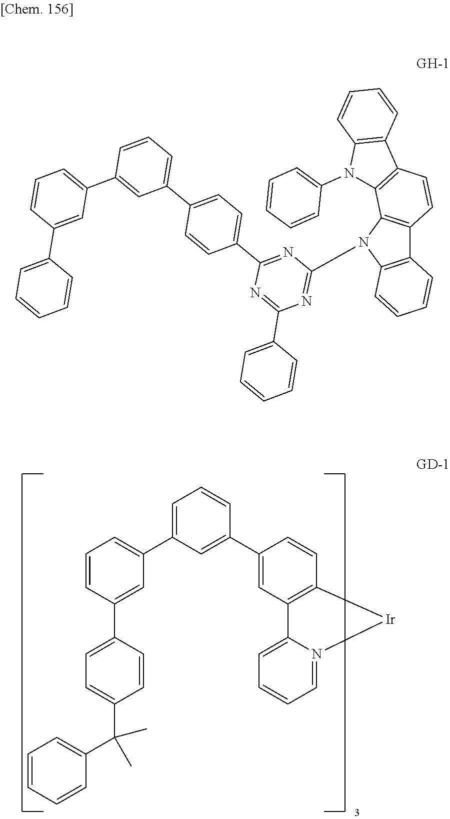

The substrate with the hole transport layer formed thereon by application was spin-coated with the prepared solution in a nitrogen glove box to form a uniform 60 nm thin film. The thin film was dried on a hot plate at 120° C. in the nitrogen glove box for 20 minutes to obtain a light-emitting layer.

Then the same procedure as in Example 1 was repeated to produce an element.

### Comparative Example 6

An element was produced using the same procedure as in Example 9 except that a composition was prepared by dissolving, in butyl benzoate, 1.5% by weight of the high-molecular weight hole transport compound having the repeating structure of formula (P-5) above and 1.5% by weight of the hole transport compound having the structure of formula (M-3) above and that this composition was used to form a hole transport layer.

### [Evaluation of Elements]

The organic electroluminescent elements obtained in Example 9 and Comparative Example 6 were caused to emit light, and the light obtained was green light with a peak wavelength of 523 nm.

Each of the elements was caused to emit light at 1,000 cd/m², and the voltage (V) and the current efficiency (cd/A) in this case were measured. The element was continuously energized at a current density of 20 mA/cm², and a luminance reduction lifetime (when the reduction in luminance reached 90%) was measured. This value is defined as LT90.

The difference between the voltage (V) of the organic electroluminescent element in Comparative Example 6 and the voltage (V) of the organic electroluminescent element in Example 9, i.e., “the voltage (V) of the organic electroluminescent element in Example 9−the voltage (V) of the organic electroluminescent element in Comparative Example 6,” was determined as the “voltage difference” and is shown in Table 3.

The ratio of the current luminous efficiency (cd/A) of the organic electroluminescent element in Example 9 relative to that of the organic electroluminescent element in Comparative Example 6, i.e., “the current luminous efficiency of the organic electroluminescent element in Example 9/the current luminous efficiency of the organic electroluminescent element in Comparative Example 6,” was determined as the “relative current luminous efficiency” and is shown in Table 3.

The ratio of the LT90 of the organic electroluminescent element in Example 9 relative to that of the organic electroluminescent element in Comparative Example 6, i.e., “the LT90 of the organic electroluminescent element in Example 9/the LT90 of the organic electroluminescent element in Comparative Example 6,” was determined as the “relative lifetime” and is shown in Table 2.

TABLE 3

| | | | | | Relative | |
|---|---|---|---|---|---|---|
| | | | Electron | | current | |
| | | Carbazole | accepting | Voltage | luminous | Relative |
| | Polymer | compound | compound | difference | efficiency | lifetime |
| Example 9 | P-5 | M-1 | None | −0.1 | 1.39 | 1.6 |
| | ∘ | ∘ | — | | | |
| Comparative Example 6 | P-5 | M-3 | None | 0.0 | 1.00 | 1.0 |
| | ∘ | x | — | | | |

In Table 3, the symbols o and x shown below the materials have the following meanings. The symbol o is assigned to a material having a crosslinking group, and the symbol x is assigned to a material having no crosslinking group.

As can be seen from the results in Table 3, in the organic electroluminescent element produced using the composition of the invention containing the carbazole compound having a crosslinking group and the electron accepting compound having a crosslinking group, the voltage, the current luminous efficiency, and the lifetime were good.

Although the present invention has been described in detail by way of the specific modes, it is apparent for those skilled in the art that various changes can be made without departing from the spirit and scope of the present invention.

The present application is based on Japanese Patent Application No. 2021-184743 filed on Nov. 12, 2021 and Japanese Patent Application No. 2021-184745 filed on Nov. 12, 2021, the entire contents of which are incorporated herein by reference.

### REFERENCE SIGNS LIST

1 substrate
2 anode
3 hole injection layer
4 hole transport layer
5 light-emitting layer
6 electron transport layer
7 cathode
8 organic electroluminescent element

1. A composition comprising: a carbazole compound having a crosslinking group and represented by formula (71) or (72); and an electron accepting compound having a crosslinking group and represented by formula (81): 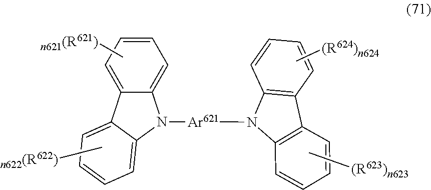
wherein, in formula (71),
Ar⁶²¹ represents a divalent aromatic hydrocarbon group having 6 to 50 carbon atoms and optionally having a substituent;
R⁶²¹, R⁶²², R⁶²³, and R⁶²⁴ are each independently a deuterium atom, a halogen atom, a monovalent aromatic hydrocarbon group having 6 to 50 carbon atoms and optionally having a substituent and/or a crosslinking group, or a crosslinking group;
n621, n622, n623, and n624 are each independently an integer of 0 to 4,
provided that the sum of n621, n622, n623 n433, and n624 is 1 or more; and
the compound represented by formula (71) has at least two crosslinking groups, 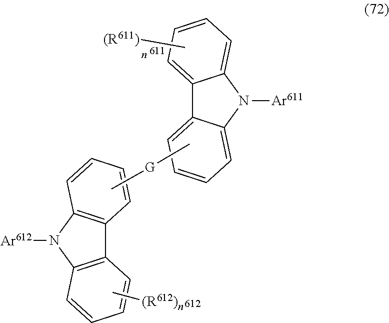
wherein, in formula (72),
Ar⁶¹¹ and Ar⁶¹² each independently represent a divalent aromatic hydrocarbon group having 6 to 50 carbon atoms and optionally having a substituent and/or a crosslinking group;
R⁶¹¹ and R⁶¹² are each independently a deuterium atom, a halogen atom, a monovalent aromatic hydrocarbon group having 6 to 50 carbon atoms and optionally having a substituent and/or a crosslinking group, or a crosslinking group;
G represents a single bond or a divalent aromatic hydrocarbon group having 6 to 50 carbon atoms and optionally having a substituent and/or a crosslinking group;
n⁶¹¹ and n⁶¹² are each independently an integer of 0 to 4; and
the compound represented by formula (72) has at least two crosslinking groups, 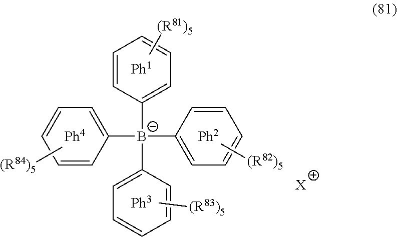
wherein, in formula (81), five R⁸¹'s, five R⁸²'s, five R⁸³'s, and five R⁸⁴'s are each independent; R⁸¹'s to R⁸⁴'s each independently represent a hydrogen atom, a deuterium atom, a halogen atom, an aromatic hydrocarbon group having 6 to 50 carbon atoms and optionally having a substituent and/or a crosslinking group, an aromatic heterocyclic group having 3 to 50 carbon atoms and optionally having a substituent and/or a crosslinking group, a fluorine-substituted alkyl group having 1 to 12 carbon atoms, or a crosslinking group;
Ph¹, Ph², Ph¹, are Ph⁴ are symbols representing four benzene rings;
the compound represented by formula (81) has a crosslinking group; and
X⁺ represents a counter cation.

2. The composition according to claim 1, wherein Ar⁶²¹ in formula (71) is a divalent group formed by bonding a plurality of structures selected from 1 to 4 benzene rings each optionally having a substituent and 1 or 2 fluorene rings each optionally having a substituent in any order in a linear or branched manner.

3. The composition according to claim 1, wherein Ar⁶²¹ in formula (71) has at least one partial structure selected from the group consisting of formulas (71-1) to (71-11) and (71-21) to (71-24): 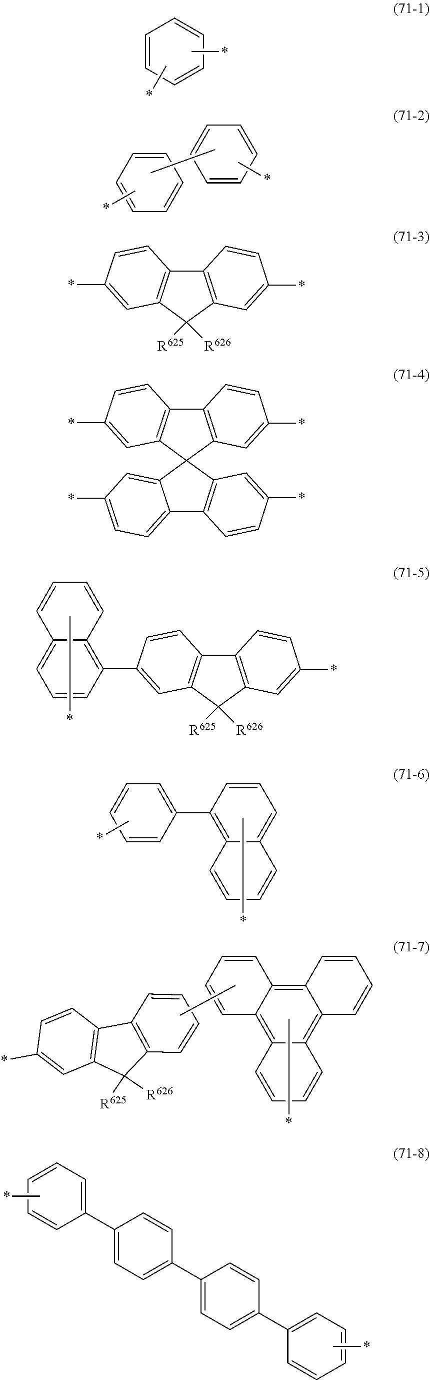 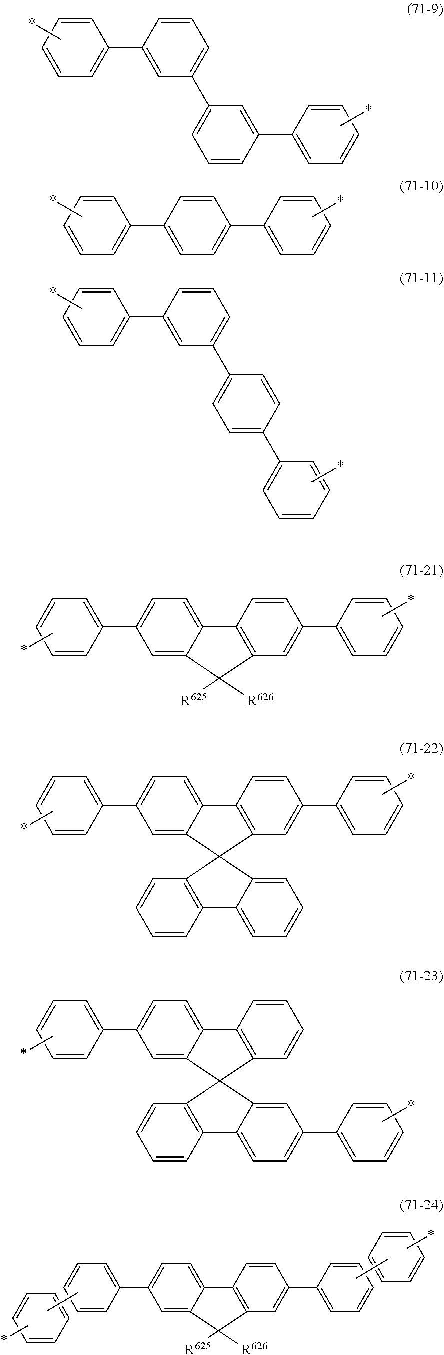
wherein, in each of formulas (71-1) to (71-11) and (71-21) to (71-24),
each * represents a bond to an adjacent structure or a hydrogen atom; when two *'s are present, at least one of the two *'s represents a position of bonding to an adjacent structure; when four *'s are present, at least one of any two of the four *s represents a position of bonding to an adjacent structure;
R⁶²⁵ and R⁶²⁶ each independently represent an alkyl group having 6 to 12 carbon atoms, an alkenyl group, an alkynyl group, an alkoxy group, an aryloxy group, an alkoxycarbonyl group, an acyl group, a halogen atom, a haloalkyl group, an alkylthio group, an arylthio group, a silyl group, a siloxy group, a cyano group, an aralkyl group, or a monovalent aromatic hydrocarbon group having 6 to 30 carbon atoms; and R⁶²⁵ and R⁶²⁶ may be bonded together to form a ring.

4. The composition according to claim 1, wherein R⁶²¹, R⁶²², R⁶²³, and R⁶²⁴ in formula (71) are each independently an aromatic hydrocarbon group having 6 to 50 carbon atoms and optionally having a crosslinking group or a crosslinking group.

5. The composition according to claim 1, wherein, in formula (71), n621 and n623 are each 1; n622 and n624 are each 0; and R⁶²¹ and R⁶²³ are each independently an aromatic hydrocarbon group having 6 to 50 carbon atoms and substituted with a crosslinking group or a crosslinking group.

6. The composition according to claim 1, wherein Ar⁶¹¹ and Ar⁶¹² in formula (72) are each independently a phenyl group having a crosslinking group or a monovalent group that includes a plurality of benzene rings bonded together in a linear or branched manner and that has a crosslinking group.

7. The composition according to claim 1, wherein at least one of Ar⁶¹¹ and Ar⁶¹² in formula (72) has at least one partial structure selected from the group consisting of formulas (72-1) to (72-6): 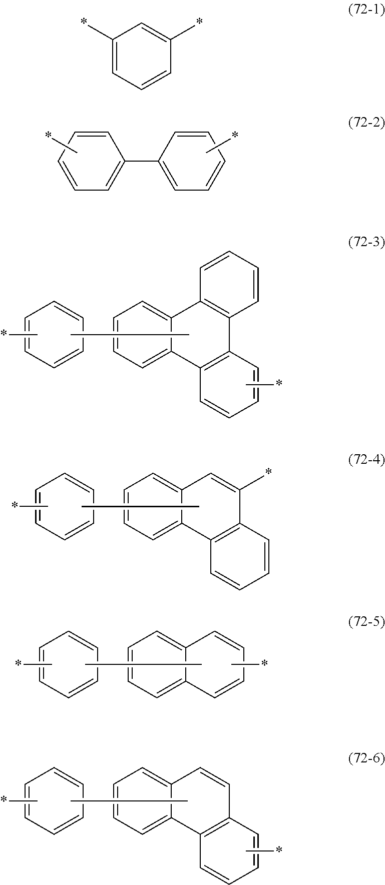
wherein, in each of formulas (72-1) to (72-6), each * represents a bond to an adjacent structure or a hydrogen atom; and at least one of two *'s represents a position of bonding to an adjacent structure.

8. The composition according to claim 1, wherein, in formula (72), n⁶¹¹ and n⁶¹² are each 0.

9. The composition according to claim 1, wherein, in formula (72), G is a single bond.

10. The composition according to claim 1, wherein, in formula (81), at least one of -Ph¹-(R⁸¹)₅, -Ph²-(R⁸²)₅, -Ph³-(R⁸³)₅, and -Ph⁴-(R⁸⁴)₅ is a group represented by formula (84) and having four fluorine atoms: 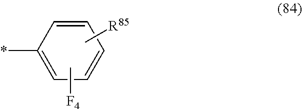
wherein, in formula (84), * represents a bond to boron B in formula (81);
F₄ represents substitution with four fluorine atoms; and
R⁸⁵ represents an aromatic hydrocarbon group optionally having a substituent and/or a crosslinking group or a crosslinking group.

11. The composition according to claim 1, wherein the electron accepting compound represented by formula (81) has at least two crosslinking groups.

12. The composition according to claim 1, further comprising a polymer having an arylamine structure as a repeating unit, the repeating unit being represented by formula (50): 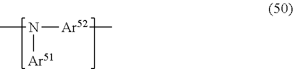
wherein, in formula (50),
Ar⁵¹ represents an aromatic hydrocarbon group, an aromatic heterocyclic group, or a group in which a plurality of groups selected from aromatic hydrocarbon groups and aromatic heterocyclic groups are linked together;
Ar² represents a di valent aromatic hydrocarbon group, a divalent aromatic heterocyclic group, or a divalent group in which a plurality of groups selected from the group consisting of divalent aromatic hydrocarbon groups and divalent aromatic heterocyclic groups are linked together directly or via a linking group;
Ar⁵¹ and Ar⁵² may form a ring via a single bond or a linking group; and
Ar⁵¹ and Ar⁵¹ may each optionally have a substituent and/or a crosslinking group.

13. The composition according to claim 12, wherein the polymer has a crosslinking group.

14. The composition according to claim 12, wherein each crosslinking group included in the compound represented by formula (71) or (72), each crosslinking group included in the compound represented by formula (81), and each crosslinking group included in the polymer having the repeating unit represented by formula (50) are each independently selected from formulas (X1) to (X18) in group T of crosslinking groups:
<group T of crosslinking groups> 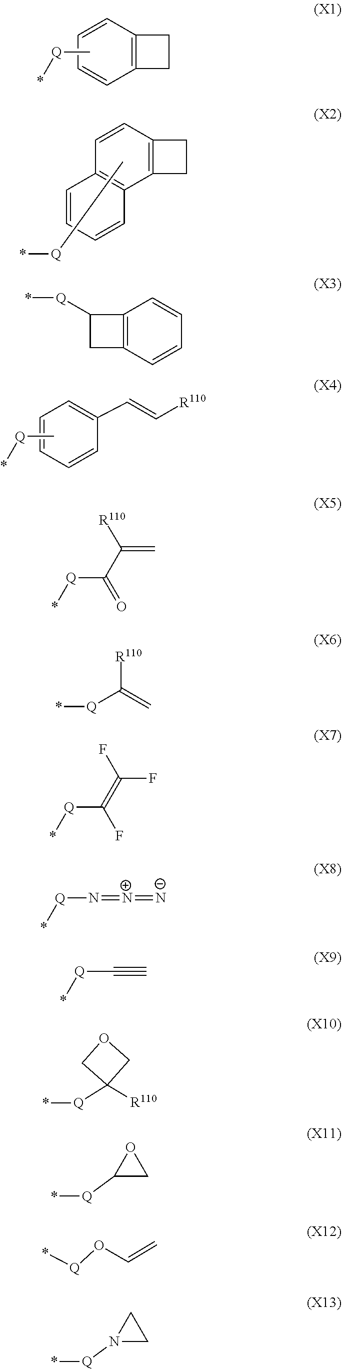 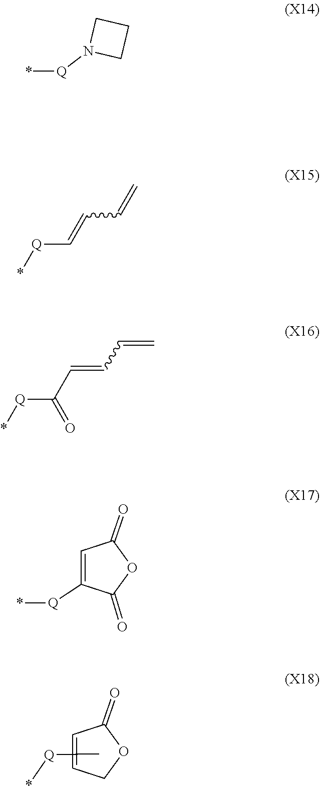
wherein, in formulas (X1) to (X18), Q represents a direct bond or a linking group;
* represents a bonding position;
R¹¹⁰ in each of formulas (X4), (X5), (X6), and (X10) represents a hydrogen atom or an alkyl group optionally having a substituent;
each of the benzene rings and the naphthalene ring in formula (X1) to (X4) may optionally have a substituent, and any of the substituents may be banded together to form a ring; and
each cyclobutene ring in formula (X1) to (X3) may optionally have a substituent.

15. The composition according to claim 14, wherein each crosslinking group included in the compound represented by formula (71) or (72), each crosslinking group included in the compound represented by formula (81), and each crosslinking group included in the polymer having an arylamine structure as a repeating unit are each independently selected from formulas (X1) to (X4) in the group T of crosslinking groups.

16. The composition according to claim 12, wherein the repeating unit represented by formula (50) is a repeating unit represented by formula (54), (55), (56), or (57): 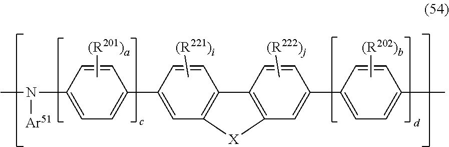
wherein, in formula (54),
Ar⁵¹ is the same as Ar⁵¹ in formula (50) above;
X is —C(R²⁰⁷)(R²⁰⁸)—, —N(R²⁰⁹)—, or —C(R²¹¹)(R²¹²)—C(R²¹³)(R²¹⁴)—;
R²⁰¹, R²⁰², R²²¹, and R²²² are each independently an alkyl group optionally having a substituent and/or a crosslinking group;
R²⁰⁷ to R²⁰⁹ and R²¹¹ to R²¹⁴ are each independently a hydrogen atom, an alkyl group optionally having a substituent and/or a crosslinking group, an aralkyl group optionally having a substituent and/or a crosslinking group, or an aromatic hydrocarbon group optionally having a substituent and/or a crosslinking group;
a and b are each independently an integer of 0 to 4;
c is an integer of 0 to 3;
d is an integer of 0 to 4; and
i and j are each independently an integer of 0 to 3, 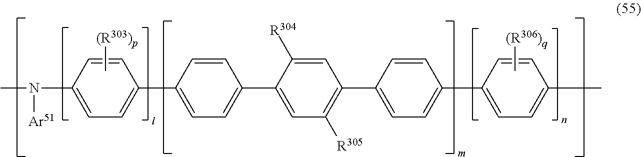
wherein, in formula (55),
Ar⁵¹ is the same as Ar⁵¹ in formula (54);
R³⁰³ and R³⁰⁶ each independently represent an alkyl group optionally having a substituent and/or a crosslinking group;
R³⁰⁴ and R³⁰⁵ each independently represent an alkyl group optionally having a substituent and/or a crosslinking group, an alkoxy group optionally having a substituent and/or a crosslinking group, or an aralkyl group optionally having a substituent and/or a crosslinking group;
l is 0 or 1;
m is 1 or 2;
n is 0 or 1;
p is 0 or 1; and
q is 0 or 1, 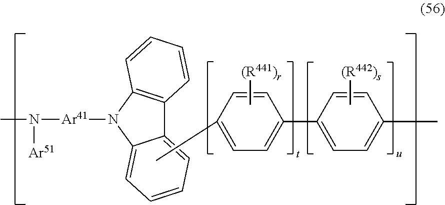
wherein, in formula (56),
Ar⁵¹ is the same as Ar⁵¹ in formula (54) above;
Ar⁴¹ represents a divalent aromatic hydrocarbon group optionally having a substituent, a divalent aromatic heterocyclic group optionally having a substituent, or a divalent group in which a plurality of groups selected from the group consisting of divalent aromatic hydrocarbon groups and divalent aromatic heterocyclic groups are linked together directly or via a linking group;
R⁴⁴¹ and R⁴⁴² each independently represent an alkyl group optionally having a substituent;
t is 1 or 2;
u is 0 or 1;
r and s are each independently an integer of 0 to 4; and
when a twisted structure is essential, r×t+s×u is 1, 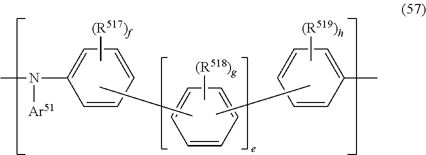
wherein, in formula (57),
Ar⁵¹ is the same as Ar⁵¹ in formula (50) above;
R⁵¹⁷ to R⁵¹⁹ each independently represent an alkyl group optionally having a substituent and/or a crosslinking group, an alkoxy group optionally having a substituent and/or a crosslinking group, an aralkyl group optionally having a substituent and/or a crosslinking group, an aromatic hydrocarbon group optionally having a substituent and/or a crosslinking group, or an aromatic heterocyclic group optionally having a substituent and/or a crosslinking group;
f, g, and h each independently represent an integer of 0 to 4;
e represents an integer of 0 to 3,
provided that, when g is 1 or more, e is 1 or more; and
f+e×g+h is 1 on more.

17. The composition according to claim 16, wherein the polymer having an arylamine structure as a repeating unit further includes a repeating unit represented by formula (60) as the repeating unit represented by formula (50) in addition to the repeating unit represented by formula (54), (55), (56), or (57): 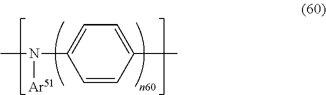
wherein, in formula (60),
Ar⁵¹ is the same as Ar⁵¹ in formula (50) above; and
n60 represents an integer of 1 to 5.

18. The composition according to claim 14, wherein each substituent included in the compound represented by formula (71) or (72), each substituent included in the compound represented by formula (81), each substituent including in the polymer having the repeating unit represented by formula (50), and each substituent included in the group T of crosslinking group are each independently selected from the group consisting of substituent group Z:
<substituent group Z>
alkyl groups having 1 to 24 carbon atoms,
alkenyl group having 2 to 24 carbon atoms,
alkynyl groups having 2 to 24 carbon atoms,
alkoxy groups having 1 to 24 carbon atoms,
aryloxy groups and heteroaryloxy groups having 4 to 36 carbon atoms,
alkoxycarbonyl groups having 2 to 24 carbon atoms,
dialkylamino groups having 2 to 24 carbon atoms,
diarylamino groups having 10 to 36 carbon atoms,
arylalkylamino groups having 7 to 36 carbon atoms,
acyl groups having 2 to 24 carbon atoms,
halogen atoms,
haloalkyl groups having 1 to 12 carbon atoms,
alkylthio groups having 1 to 24 carbon atoms,
arylthio groups having 4 to 36 carbon atoms,
silyl groups having 2 to 36 carbon atoms,
siloxy groups having 2 to 36 carbon atoms,
a cyano group,
aromatic hydrocarbon groups having 6 to 36 carbon atoms, and
aromatic heterocyclic groups having 4 to 36 carbon atoms,
wherein each of the above substituents may have a linear, branched, or cyclic structure, and wherein, when any of the substituents are adjacent to each other, the substituents adjacent to each other may be bonded together to form a ring.

19. The composition according to claim 1, further comprising a solvent.

20. A composition comprising: a carbazole compound having a crosslinking group and represented by formula (71) or (72); and a polymer having an arylamine structure as a repeating unit, wherein the polymer having an arylamine structure as a repeating unit has a structure represented by formula (50) as a repeating unit and also has a crosslinking group, and wherein the structure represented by formula (50) has a partial structure represented by formula (63): 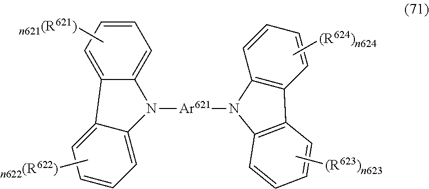
wherein, in formula (71),
Ar⁶²³ represents a divalent aromatic hydrocarbon group having 6 to 50 carbon atoms and optionally having a substituent;
R⁶²¹, R⁶²², R⁶²³, and R⁶²⁴ each independently represent a deuterium atom, halogen atom, a monovalent aromatic hydrocarbon group having 6 to 50 carbon atoms and optionally having a substituent and/or a crosslinking group, or a crosslinking group;
n621, n622, n623, and n624 are each independently an integer of 0 to 4,
provided that the sum of n621, n622, n623, and n624 is 1 or more; and
the compound represented by formula (71) has at least two crosslinking groups, 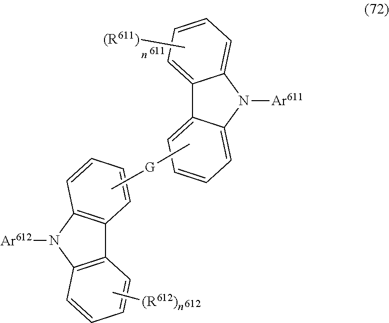
wherein, in formula (72),
Ar⁶¹¹ and Ar⁶¹² each independently represent a divalent aromatic hydrocarbon group having 6 to 50 carbon atoms and optionally having a substituent and/or a crosslinking group;
R⁶¹¹ and R⁶¹² are each independently a deuterium atom, a halogen atom, a monovalent aromatic hydrocarbon group having 6 to 50 carbon atoms and optionally having a substituent and/or a crosslinking group, or a crosslinking group;
G represents a single bond or a divalent aromatic hydrocarbon group having 6 to 50 carbon atoms and optionally having a substituent and/or a crosslinking group;
n⁶¹¹ and n⁶¹² are each independently an integer of 0 to 4; and
the compound represented by formula (72) has at least two crosslinking groups, 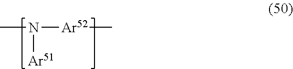
wherein, in formula (50),
Ar⁵¹ represents an aromatic hydrocarbon group, an aromatic heterocyclic group, or a group in which a plurality of groups selected from aromatic hydrocarbon groups and aromatic heterocyclic groups are linked together;
Ar⁵² represents a divalent aromatic hydrocarbon group, a divalent aromatic heterocyclic group, or a divalent group in which a plurality of groups selected from the group consisting of divalent aromatic hydrocarbon groups and divalent aromatic heterocyclic groups are linked together directly or via a linking group;
Ar⁵¹ and Ar⁵² may form a ring via a single bond or a linking group;
Ar⁵¹ and Ar⁵² may each optionally have a substituent and/or a crosslinking group; and
Ar⁵² has the partial structure represented by formula (63), 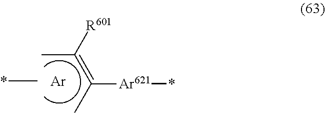
wherein, in formula (63),
R⁶⁰¹ represents an alkyl group optionally having a substituent or a crosslinking group;
Ar⁶²¹ represents a divalent aromatic hydrocarbon group optionally having a substituent and/or a crosslinking group or a divalent aromatic heterocyclic group optionally having a substituent and/or a crosslinking group;
ring Ar represents an aromatic hydrocarbon structure optionally having a substituent and/or a crosslinking group or a divalent aromatic heterocyclic structure optionally having a substituent and/or a crosslinking group; and
* represents a position bonded to an adjacent atom.

21. The composition according to claim 20, wherein the repeating unit represented by formula (50) is a repeating unit represented by formula (54), (55), (56), or (57): 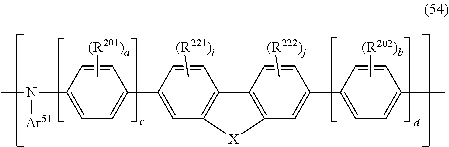
wherein, in formula (54),
Ar⁵¹ is the same as Ar⁵¹ in formula (50) above;
X is —C(R²⁶⁷)(R²⁶⁸)—, —N(R²⁰⁹)—, or —C(R²¹¹)(R²¹²)—C(R²¹³)(R²¹⁴)—;
R²⁰¹, R²⁰², R²²¹, and R²²² are each independently an alkyl group optionally having a substituent and/or a crosslinking group;
R²⁰⁷ to R²⁰⁹ and R²¹¹ to R²¹⁴ are each independently a hydrogen atom, an alkyl group optionally having a substituent and/or a crosslinking group, an aralkyl group optionally having a substituent and/or a crosslinking group, or an aromatic hydrocarbon group optionally having a substituent and/or a crosslinking group;
a and are each independently an integer of 0 to 4;
c is an integer of 0 to 3;
d is an integer of 0 to 4;
i and j are each independently an integer of 0 to 3; and
a×c+b×d+i+j is 1 or more, 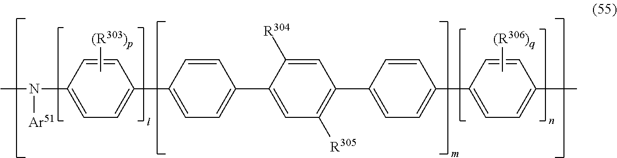
wherein, in formula (55),
Ar⁵¹ is the same as Ar⁵¹ in formula (54);
R³⁰³ and R³⁰⁶ each independently represent an alkyl group optionally having a substituent and/or a crosslinking group;
R³⁰⁴ and R³⁰⁵ each independently represent an alkyl group optionally having a substituent and/or a crosslinking group, an alkoxy group optionally having a substituent and/or a crosslinking group, or an aralkyl group optionally having a substituent and/or a crosslinking group;
l is 0 or 1;
m is 1 or 2;
n is 0 or 1;
p is 0 or 1; and
q is 0 or 1, 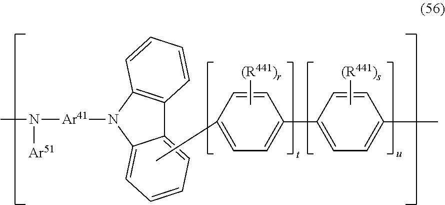
wherein, in formula (56),
Ar⁵¹ is the same as Ar in formula (54);
Ar⁴¹ represents a divalent aromatic hydrocarbon group optionally having a substituent, a divalent aromatic heterocyclic group optionally having a substituent, or a divalent group in which a plurality of groups selected from the group consisting of diva ent aromatic hydrocarbon groups and divalent aromatic heterocyclic groups are linked together directly or via a linking group;
R⁴⁴¹ and R⁴⁴² each independently represent an alkyl group optionally having a substituent;
t is 1 or 2;
u is 0 or 1;
r and s are each independently an integer of 0 to 4; and
when a twisted structure is essential, r×t+s×u is 1, 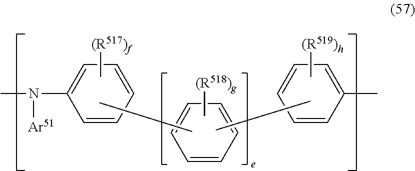
wherein, in formula (57),
Ar⁵¹ is the same as Ar⁵¹ in formula (50);
R⁵¹⁷ to R⁵¹⁹ each independently represent an alkyl group optionally having a substituent and/or a crosslinking group, an alkoxy group optionally having a substituent and/or a crosslinking croup, an aralkyl group optionally having a substituent and/or a crosslinking group, an aromatic hydrocarbon group optionally having a substituent and/or a crosslinking group, or an aromatic heterocyclic group optionally having a substituent and/or a crosslinking group;
f, g, and h each independently represent an integer of 0 to 4;
e represents an integer of 0 t 3,
provided that, when g is 1 or more, e is 1 or more; and
f+e×g+h is 1 or more.

22. The composition according to claim 21, wherein the repeating unit represented by formula (54) is a repeating unit represented by formula (62): 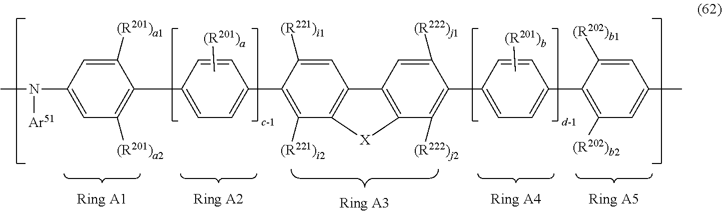
wherein, in formula (62),
Ar⁵¹, X, R²⁰¹, R²⁰², R²²¹, R²²², a, and b are the same as Ar⁵¹, X, R²⁰¹, R²⁰², R²²¹, R²²², a, and b in formula (54);
c is an integer of 1 to 3;
d is an integer of 1 to 4;
a¹, a¹, b¹, b², i¹, i², j¹, and j² are each independently 0 or 1; and
any of the following conditions (1) and (2) is satisfied:
(1) at least one of a¹, a², a, b¹, b², and b is 1 or more, provided that, when c is 1, a is not defined and that, when d is 1, b is not defined;
(2) at least one of i¹, i², j¹, and j² is 1, and
wherein Ring A1 is a divalent benzene ring optionally having R²⁰¹ at a specific position;
Ring A2 is a divalent group including c−1 benzene rings linked together and each optionally having R²⁰¹, provided that, when c=2, Ring A2 is a single divalent benzene ring;
Ring A3 is a divalent condensed ring having a biphenyl structure further bonded through X;
Ring A4 is a divalent group including d−1 benzene rings linked together and each optionally having R²⁰¹, provided that, when d=2, Ring A4 is a single divalent benzene ring; and
Ring A5 is divalent benzene ring optionally having R²⁰² at a specific position.

23. The composition according to claim 21, wherein the polymer having an arylamine structure as a repeating unit further includes a repeating unit represented by formula (60) as the repeating unit represented by formula (50) in addition to the repeating unit represented by formula (54), (55), (56), or (57): 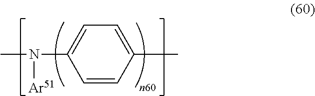
wherein, in formula (60),
Ar⁵¹ is the same as Ar⁵¹ in formula (50); and
n60 represents an integer of 1 to 5.

24. The composition according to claim 20, wherein Ar⁶²¹ formula (71) is a divalent group formed by bonding a plurality of structures selected from 1 to 4 benzene rings each optionally having a substituent and 1 or 2) fluorene ring each optionally having a substituent in any order in a linear or branched manner.

25. The composition according to claim 20, wherein Ar⁶²¹ in formula (71) has at least one partial structure selected from the group consisting of formulas (71-1) to (71-11) and (71-21) to (71-24): 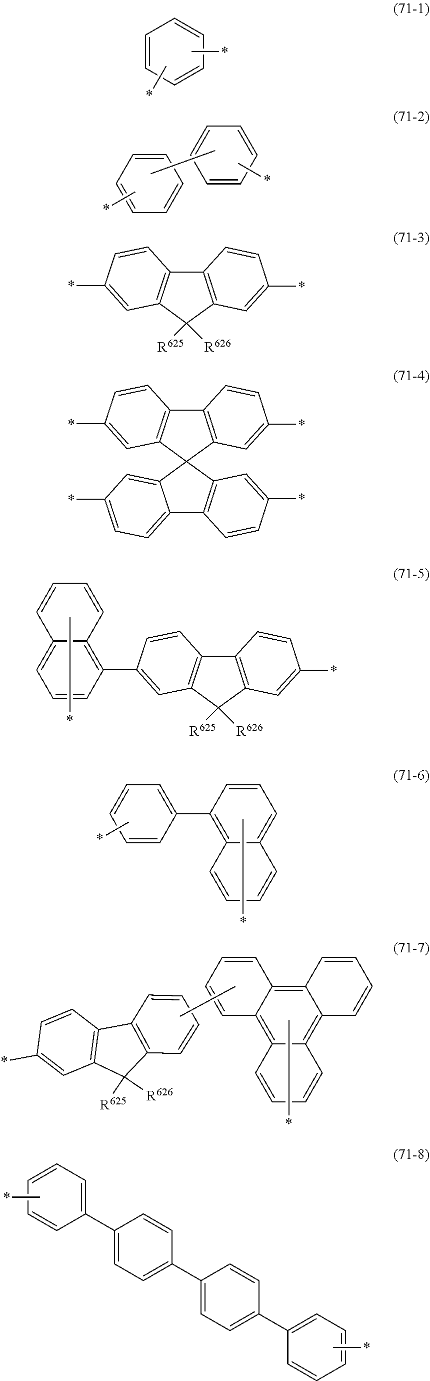 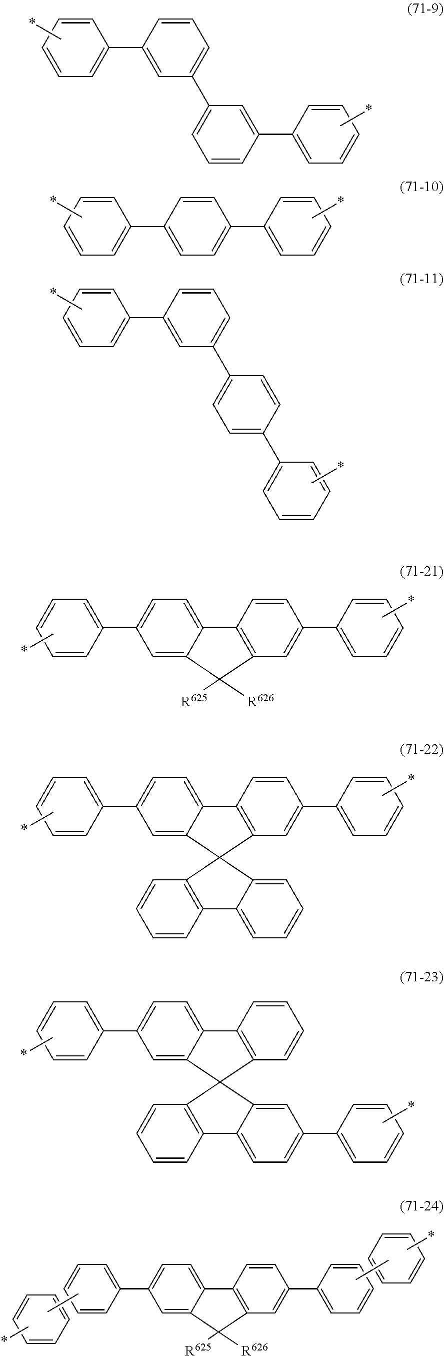
wherein, in each of formulas (71-1) to (71-11) and (71-21) to (71-24),
each * represents a bond to an adjacent structure or a hydrogen atom; when two *'s are present, at least one of the two *'s represents a position of bonding to an adjacent structure; when four *'s are present, at least one of any two of the four *'s represents a position of bonding to an adjacent structure;
R⁶²⁵ and R⁶²⁶ each independently represent an alkyl group having 6 to 12 carbon atoms, an alkenyl group, an alkynyl group, an alkoxy group, an aryoxy group, an alkoxycarbonyl group, an acyl group, a halogen atom, haloalkyl group, an alkylthio group, an arylthio group, a silyl group, a siloxy group, a cyano group, an aralkyl group, or a monovalent aromatic hydrocarbon group having 6 to 30 carbon atoms; and R⁶²⁵ and R⁶²⁶ may be bonded together to form a ring.

26. The composition according to claim 20, wherein R⁶²¹, R⁶²², R⁶²³, and R⁶²⁴ in formula (71) are each independently an aromatic hydrocarbon group having 6 to 50 carbon atoms and optionally having a crosslinking group or a crosslinking group.

27. The composition according to claim 20, wherein, in formula (71), n621 and n623 are each 1; n622 and n624 are each 0; and R⁶²¹ and R⁶²³ are each independently an aromatic hydrocarbon group having 6 to 50 carbon atoms and substituted with a crosslinking group or a crosslinking group.

28. The composition according to claim 20, wherein Ar⁶¹¹ and Ar⁶¹² in formula (72) are each independently a phenyl group having a crosslinking group or a monovalent group that includes a plurality of benzene rings bonded together in a linear or branched manner and that has a crosslinking group.

29. The composition according to claim 20, wherein at least one of Ar⁶¹¹ and Ar⁶¹² in formula (72) has at least one partial structure selected from formulas (72-1) to (72-6): 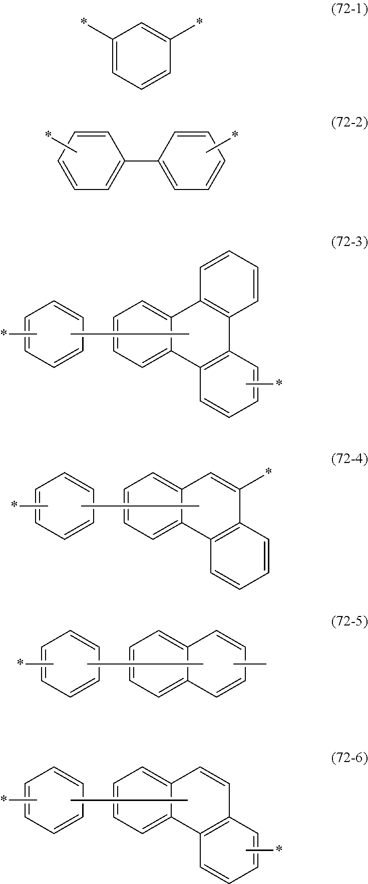
wherein, in each of formulas (72-1) to (72-6), each * represents a bond to an adjacent structure or a hydrogen atom; and at least one of two *'s represents a position of bonding to an adjacent structure.

30. The composition according to claim 20, wherein, in formula (72), n⁶¹¹ and n⁶¹² are each 0.

31. The composition according to claim 20, wherein, in formula (72), G is a single bond.

32. The composition according to claim 20, further comprising an electron accepting compound having a crosslinking group and represented by formula (81): 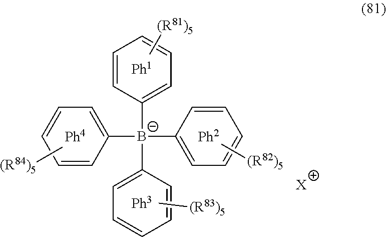
wherein, in formula (81), five R⁸¹'s, five R⁸²'s, five R⁸³'s, and five R⁸⁴'s are each independent; R⁸¹'s to R⁸⁴'s each independently represent a hydrogen atom, a deuterium atom, a halogen atom, an aromatic hydrocarbon group having 6 to 50 carbon atom and optionally having a substituent and/or a crosslinking group, an aromatic heterocyclic group having 3 to 50 carbon atoms and optionally having a substituent and/or a crosslinking group, a fluorine-substituted alkyl group having 1 to 12 carbon atoms, or a crosslinking group;
Ph¹, Ph², Ph³, are Ph⁴ are symbols representing four benzene rings;
the compound represented by formula (81) has a cross linking group; and
X⁺ represents a counter cation.

33. The composition according to claim 32, wherein, in formula (81), at least one of -Ph¹-(R⁸¹)₅, -Ph²-(R⁸²)₅, -Ph³-(R⁸³)₅, and -Ph⁴-(R⁸⁴)₅ is a group represented by formula (84) and having four fluorine atoms: 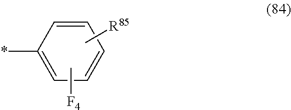
wherein, in formula (84), * represents a bond to boron B in formula (81);
F₄ represents substitution with four fluorine atoms; and
R⁸⁵ represents an aromatic hydrocarbon, group optionally having a substituent and/or a crosslinking group or a crosslinking group.

34. The composition according to claim 32, wherein the electron accepting compound represented by formula (81) has at least two crosslinking groups.

35. The composition according to claim 32, wherein each crosslinking group included in the compound represented by formula (71) or (71), each crosslinking group included in the compound represented by formula (81), and each crosslinking group included in the polymer having an arylamine structure as a repeating unit are each independently selected from the group consisting of formulas (X1) to (X18) in group T of crosslinking groups:
<group T of crosslinking groups> 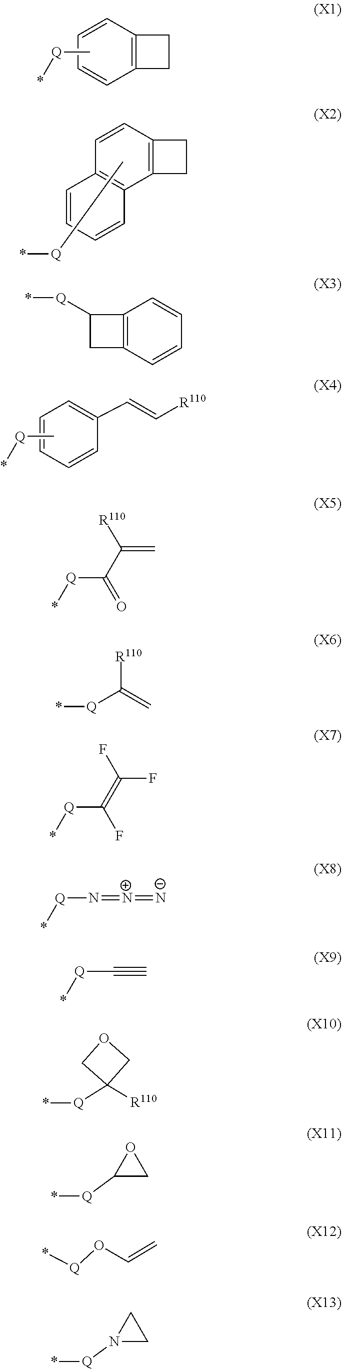 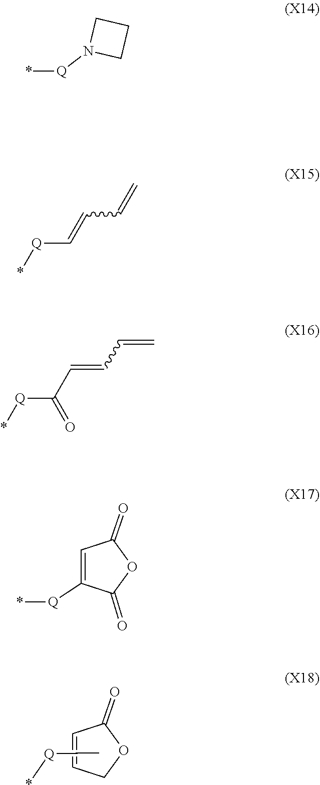
wherein, in formulas (X1) to (X18), Q represents a direct bond or a linking group;
* represents a bonding position;
R¹¹⁰ in each of formulas (X4), (X5), (X6), and (X10) represents a hydrogen atom or an alkyl group optionally having a substituent;
each of the benzene rings and the naphthalene ring in formula (X1) to (X4) may optionally have a substituent, and any of the substituents may be bonded together to form a ring; and
each cyclobutene ring in formula (X1) to (X3) may optionally have a substituent.

36. The composition according to claim 35, wherein each crosslinking group included in the compound represented by formula (71) or (72), each crosslinking group included in the compound represented by formula (81), and each crosslinking group included in the polymer having an arylamine structure as a repeating unit are each independently selected from the group consisting of formulas (X1) to (X4) in the group T of crosslinking groups.

37. The composition according to claim 35, wherein each substituent included in the compound represented by formula (71) or (72), each substituent included in the compound represented by formula (31), each substituent included in the polymer having an arylamine structure as a repeating unit, and each substituent included in the group T of crosslinking groups are each independently selected from the group consisting of substitutent group Z:
<substituent group Z>
alkyl groups having 1 to 24 carbon atoms,
alkenyl group having 2 to 24 carbon atoms,
alkynyl groups having 2 to 24 carbon atoms,
alkoxy groups having 1 to 24 carbon atoms,
aryloxy groups and heteroaryloxy groups having 4 to 36 carbon atoms,
alkoxycarbonyl groups having 2 to 24 carbon atoms,
dialkylamino groups having 2 to 24 carbon atoms,
diarylamino groups having 10 to 36 carbon atoms,
arylalkylamino groups having 7 to 36 carbon atoms,
acyl groups having 2 to 24 carbon atoms,
halogen atoms,
haloalkyl groups having 1 to 12 carbon atoms,
alkylthio groups having 1 to 24 carbon atoms,
arylthio groups having 4 to 36 carbon atoms,
silyl groups having 2 to 36 carbon atoms,
siloxy groups having 2 to 36 carbon atoms,
a cyano group,
aromatic hydrocarbon groups having 6 to 36 carbon atoms, and
aromatic heterocyclic groups having 4 to 36 carbon atoms,
wherein each of the above substituents may have a linear, branched, or cyclic structure, and wherein, when any of the substituents are adjacent to each other, the substituents adjacent to each other may be bonded together to form a ring.

38. The composition according to claim 20, further comprising a solvent.

39. A method for producing an organic electroluminescent element including an anode and a cathode that are disposed on a substrate and further including an organic layer between the anode and the cathode, the method comprising forming the organic layer by a wet deposition method with the composition according to claim 19.

40. The method for producing an organic electroluminescent element according to claim 39, wherein the organic layer is disposed between the anode and a light-emitting layer.

41. An organic electroluminescent element produced by the organic electroluminescent element production method according to claim 39.

42. An organic electroluminescent element comprising an anode and a cathode that are disposed on a substrate and further comprising an organic layer between the anode and the cathode, wherein the organic layer contains a crosslinking reaction product of a carbazole compound having a crosslinking group represented by formula (71) or (72) and an electron accepting compound having a crosslinking group represented by formula (81): 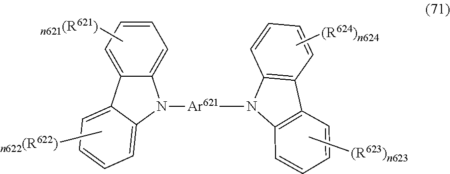
wherein, in formula (71),
Ar⁶²¹ represents a divalent aromatic hydrocarbon group having 6 to 50 carbon atoms and optionally having a substituent;
R⁶²¹, R⁶²², R⁶²³, and R⁶²⁴ each independently represent a deuterium atom, a halogen atom, a monovalent aromatic hydrocarbon group having 6 to 50 carbon atoms and optionally having a substituent and/or a crosslinking group, or a crosslinking group;
n621, n622, n623, and n624 are each independently an integer of 0 to 4,
provided that the sum of n611, n622, n623, and n624 is 1 or more; and
the compound represented by formula (71) has at least two crosslinking groups, 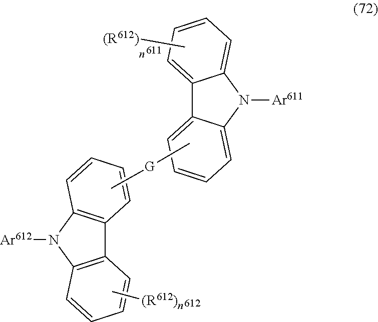
wherein, in formula (72),
Ar⁶¹¹ and Ar⁶¹² each independently represent a divalent aromatic hydrocarbon group having 6 to 50 carbon atoms and optionally having a substituent and/or a crosslinking group;
R⁶¹¹ and R⁶¹² are each independently a deuterium atom, a halogen atom, a monovalent aromatic hydrocarbon group having 6 to 50 carbon atoms and optionally having a substituent and/or a crosslinking group, or a crosslinking group;
G represents a single bond or a divalent aromatic hydrocarbon group having 6 to 50 carbon atom and optionally having a substituent and/or a crosslinking group;
n⁶¹¹ and n⁶¹² are each independently an integer of 0 to 4; and
the compound represented by formula (72) has at least two crosslinking groups, 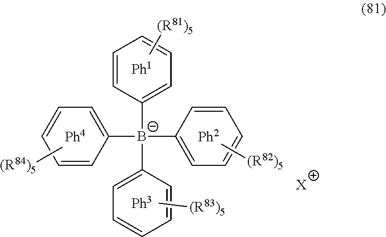
wherein in formula (81), five R⁸¹'s, five R⁸²'s, five R⁸³'s, and five R⁸⁴'s are each independent; R⁸¹'s to R⁸⁴'s each independently represent a hydrogen atom, a deuterium atom, a halogen atom, an aromatic hydrocarbon group having 6 to 50 carbon atom and optionally having a substituent and/or a crosslinking group, an aromatic heterocyclic group having 3 to 50 carbon atoms and optionally having a substituent and/or a crosslinking group, a fluorine-substituted alkyl group having 1 to 12 carbon atoms, or a crosslinking group;
Ph¹, Ph², Ph³, are Ph⁴ are symbol representing four benzene rings;
the compound represented by formula (81) has a crosslinking group; and
X⁺ represents a counter cation.

43. An organic electroluminescent element comprising an anode and a cathode that are disposed on a substrate and further comprising an organic layer between the anode and the cathode, wherein the organic layer contains a crosslinking reaction product of a carbazole compound having a crosslinking group and represented by formula (71) or (72) and a polymer having an arylamine structure as a repeating unit and having a crosslinking group, the repeating unit being a repeating structure represented by formula (50), wherein the structure represented by formula (50) has a partial structure represented by formula (63): 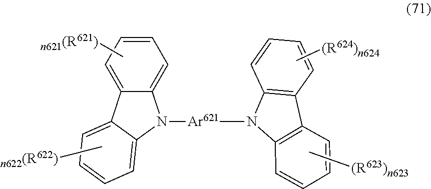
wherein, in formula (71),
Ar⁶²¹ represents a divalent aromatic hydrocarbon group having 6 to 50 carbon atoms and optionally having a substituent;
R⁶²¹, R⁶²², R⁶²³, and R⁶²⁴ each independently represent a deuterium atom, a halogen atom, a monovalent aromatic hydrocarbon group having 6 to 50 carbon atoms and optionally having a substituent and/or a crosslinking group, or a crosslinking group;
n621, n622, n623, and n624 are each independently an integer of 0 to 4,
provided that the sum of n621, n622, n623, and n624 is 1 or more; and
the compound represented by formula (71) has at least two crosslinking groups, 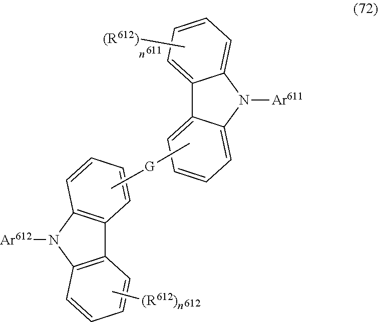
wherein, in formula (72),
Ar⁶¹¹ and Ar⁶¹² each independently represent a divalent aromatic hydrocarbon group having 6 to 50 carbon atoms and optionally having a substituent and/or a crosslinking group;
R⁶¹¹ and R⁶¹² are each independently a deuterium atom, a halogen atom, a monovalent aromatic hydrocarbon group having 6 to 50 carbon atoms and optionally having a substituent and/or a crosslinking group, or a crosslinking group;
G represents a single bond or a divalent aromatic hydrocarbon group having 6 to 50 carbon atoms and optionally having a substituent and/or a crosslinking group;
n⁶¹¹ and n⁶¹² are each independently an integer of 0 to 4; and
the compound represented by formula (72) has at least two crosslinking groups, 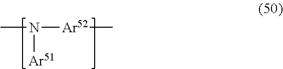
wherein, in formula (50),
Ar⁵¹ represents an aromatic hydrocarbon group, an aromatic heterocyclic group, or a group in which a plurality of groups selected from aromatic hydrocarbon groups and aromatic heterocyclic groups are linked together;
Ar⁵² represents a divalent aromatic hydrocarbon group, a divalent aromatic heterocyclic group, or a divalent group in which a plurality of groups selected from the group consisting of divalent aromatic hydrocarbon groups and divalent aromatic heterocyclic groups are linked together directly or via a linking group;
Ar⁵¹ and Ar⁵² may form a ring via a single bond or a linking group;
Ar⁵¹ and Ar⁵² may each optionally have a substituent and/or a crosslinking group; and
Ar⁵² has the partial structure represented by formula (63), 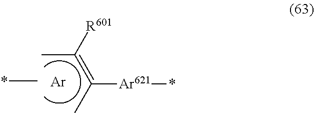
wherein, in formula (63),
R⁶⁰¹ represents an alkyl group optionally having a substituent or a crosslinking group;
Ar⁶²¹ represents a divalent aromatic hydrocarbon group optionally having a substituent and/or a crosslinking group or a divalent aromatic heterocyclic group optionally having a substituent and/or a crosslinking group;
ring Ar represents an aromatic hydrocarbon structure optionally having a substituent and/or a crosslinking group or a divalent aromatic heterocyclic structure optionally having a substituent and/or a crosslinking group; and
* represents a position bonded to an adjacent atom.

44. A display device comprising the organic electroluminescent element according to claim 42.

45. A lighting device comprising the organic electroluminescent element according to claim 42.

46. A method for producing an organic electroluminescent element including an anode and a cathode that are disposed on a substrate and further including an organic layer between the anode and the cathode, the method comprising forming the organic layer by a wet deposition method with the composition according to claim 33.

47. A display device comprising the organic electroluminescent element according to claim 43.

48. A lighting device comprising the organic electroluminescent element according to claim 43.
